(19) United States
(12) Patent Application Publication
(10) Pub. No.: US 2026/0317758 A1
(43) Pub. Date: Oct. 15, 2026

(54) CHEMICALLY MODIFIED GUIDE RNAS FOR GENOME EDITING WITH CAS12B

(71) Applicant: Verve Therapeutics, Inc., Boston, MA (US)

(72) Inventors: Kallanthottathil G. Rajeev, Wayland, MA (US); Alexandra Chadwick, Somerville, MA (US); Caroline Reiss, Somerville, MA (US)

(21) Appl. No.: 19/657,840

(22) Filed: Apr. 24, 2026

Related U.S. Application Data
(63) Continuation of application No. 17/918,090, filed on Apr. 9, 2021, now Pat. No. 12,649,921.
(60) Provisional application No. 63/007,814, filed on Apr. 9, 2020.

(51) Int. Cl. C12N 15/11; C12N 9/22; C12N 15/90

(52) U.S. Cl. CPC C12N 15/11; C12N 9/22; C12N 15/907; C12N 2310/20; C12N 2310/315; C12N 2310/321; C12N 2310/331; C12N 2800/80

(57) ABSTRACT

Provided herein are compositions related to Cas12b guide RNAs. Also provided herein are methods for modifying target polynucleotide sequences and methods of treating or preventing a condition in a subject in need thereof, such as conditions resultant from the expression of ANGPTL3 or mutations thereto.

### CROSS REFERENCE TO RELATED APPLICATIONS

This application is a continuation of U.S. patent application Ser. No. 17/918,090 filed on Oct. 10, 2022, which claims benefit under 35 U.S.C. § 119 from Provisional Application Ser. No. 63/007,814, filed Apr. 9, 2020, the disclosures of which are incorporated herein by reference in their entirety.

### SEQUENCE LISTING

The instant application contains a Sequence Listing which has been submitted electronically in XML file format and is hereby incorporated by reference in its entirety. Said XML copy, created on Apr. 30, 2026, is named 53989-709_301_SL.xml and is 6,525,086 bytes in size.

### FIELD OF DISCLOSURE

Described herein are compositions related to chemically modified guide polynucleotides (e.g., guide RNAs, single guide RNAs, crRNAs, tracrRNAs, etc.) including compositions that comprise such guide polynucleotides. Also described herein are methods of using guide polynucleotides in gene modification of a target gene and methods of treating a disease or a condition.

### BACKGROUND

All publications herein are incorporated by reference to the same extent as if each individual publication or patent application was specifically and individually indicated to be incorporated by reference. The following description includes information that may be useful in understanding the present disclosure. It is not an admission that any of the information provided herein is prior art or relevant to the presently claimed invention, or that any publication specifically or implicitly referenced is prior art. In the event of inconsistent usages between this document and those documents incorporated by reference, the usage in the incorporated reference(s) should be considered supplementary to that of this document; for any irreconcilable inconsistencies, the usage in this document controls.

### SUMMARY OF THE DISCLOSURE

In one aspect, provided herein is a single guide RNA that comprises (i) a spacer sequence and (ii) a scaffold or tracr sequence, wherein the spacer sequence is designed to be complementary to a target polynucleotide sequence in a gene of interest when in close proximity to the target complementary polynucleotide, wherein the scaffold sequence is a substrate for a Cas12b protein to facilitate the formation of Cas12b protein-guide RNA complex, the ribonucleoprotein (RNP) complex, and wherein the single guide RNA comprises one or more chemical modification(s). In some embodiments, the scaffold sequence is of about 97 nucleotides long and constitutes or encompasses the 5′-end of the single guide RNA, and the spacer sequence, is about 20 to 40 nucleotides long, which constitutes or encompasses the 3′-end of the single guide RNA. In one aspect of the invention, the guide RNA Ca12b protein complex exerts modifications to the gene of interest to modulate the encoded protein production or to modify functional property of the protein. In one aspect the gene of interest is PCSK9 and in another aspect the target gene of interest is ANGPTL3.

In some embodiments, the chemical modification is in the spacer sequence. In some embodiments, the chemical modification is in the scaffold or tracr sequence. In some embodiments, the chemical modification is in a stem loop structure of the scaffold sequence. In yet other aspects, the target gene of interest may be selected from a group of genes that are capable of being modified by a Cas12b nuclease combined with a chemically modified gRNA scaffold as described herein and a spacer sequence that is complementary to DNA sequence of a target gene of interest, including, but not limited to, for example the gene sequences disclosed herein for PCSK9, APOC3, ANGPTL3 and/or Lp(a).

In some embodiments, the single guide RNA comprises an unmodified nucleotide. In some embodiments, the unmodified nucleotide comprises a 2′-hydroxyl that, when the guide RNA in close proximity to the Cas12b protein facilitates the interaction between the guide RNA and Cas12b protein. In some embodiments, the unmodified nucleotide comprises a 2′-hydroxyl that is in contact with the Cas12b protein when contacted with the Cas12b protein. In some embodiments, the unmodified nucleotide comprises a 2′-hydroxyl that is in close proximity with a second unmodified nucleotide of the single guide RNA. In some embodiments, the second unmodified nucleotide is at the 5′-end of the first unmodified nucleotide and in some embodiments the second unmodified nucleotide is at the 3′-end of the first unmodified nucleotide. In some embodiments, the unmodified nucleotide comprises a 2′-hydroxyl that is in close proximity with one or more modified nucleotide(s). In the embodiment the modified nucleotide is at the 5′-end and/or at the 3′-end of the first unmodified nucleotide.

In some embodiments, the unmodified nucleotide is at a nucleotide position selected from positions 8-10, 12-15, 22-24, 32-38, 40, 41, 43, 44, 53-56, 63, 66-69, 88-97, 99-103, 106-108, 111-116 as numbered in SEQ ID NO: 1 or a corresponding position thereof. In some embodiments, the chemical modification is at a nucleotide position selected from positions 1-7, 11, 16-21, 25-31, 39, 42, 45-52, 57-62, 64, 65, 70-87, 98, 104, 105, 109, and 110 as numbered in SEQ ID NO: 1 or a corresponding position thereof.

In some embodiments, the chemical modification(s) comprise(s) one or more 2′-OMe sugar modification(s). In some embodiments, the chemical modification comprises one or more nebularin, deoxynebularin or 2′-O-methylnebularine a combination thereof. In some embodiments, the nebularine, the 2′-O-methylnebularine, or the 2′-deoxynebularine replaces an adenosine in the single guide RNA as compared to an otherwise unmodified guide RNA. In some embodiments, the nebularine, the 2′-O-methylnebularine, or the 2′-deoxynebularine replaces a guanosine in the single guide RNA as compared to an otherwise unmodified guide RNA. In some embodiments, the nebularine, the 2′-O-methylnebularine, or the 2′-deoxynebularine replaces a chemically modified or unmodified adenosine in the chemically modified single guide RNA. In some embodiments, the nebularine, the 2′-O-methylnebularine, or the 2′-deoxynebularine replaces a chemically modified or unmodified adenosine guanosine in the chemically modified single guide RNA.

In some embodiments, the chemical modification(s) comprise(s) one or more phosphorothioate linkage(s). In some embodiments, the chemically modified single guide RNA comprises a phosphorothioate linkage at a 5′ end or at a 3′ end. In some embodiments, the chemically modified single guide RNA comprises two and no more than two phosphorothioate linkages at the 5′ end. In some embodiments, the single guide RNA comprises two and no more than two contiguous phosphorothioate linkages at the 3′ end. In some embodiments, the single guide RNA comprises three phosphorothioate linkages at the 3′ end. In some embodiments, the single guide RNA comprises three phosphorothioate linkages at the 5′ end.

In some embodiments, the chemically modified single guide RNA comprises the sequence 5′-NsNsN-3′, 5′-NsNsNsS-3′, 5′-nsnsnsn-3′, or 5′-nsnsn-3′ at the 3′ end, wherein, each uppercase N independently indicates unmodified nucleotide adenosine, cytidine, guanosine and/or uridine; and lowercase letters indicates modified nucleotides including but not limited to 2′-H, 2′-OMe and base modification; and each s independently indicates phosphorothioate backbone modification. In some embodiments, each one of the last four nucleotides at the 3′end of the single guide RNA comprises a 2′-OMe modification.

In some embodiments, the chemically modified single guide RNA comprises the sequence 5′-UsUsU-3′, 5′-UsUsUsU-3′, 5′-usususu-3′, or 5′-ususu-3′ at the 3′ end. In some embodiments, each one of the last four nucleotides at the 3′end of the single guide RNA comprises a 2′-OMe modification.

In some embodiments, the target polynucleotide sequence is in a PCSK9 gene. In some embodiments, the target polynucleotide sequence is in a ANTPLT3 gene.

In some embodiments, the chemical modification on the single guide RNA enhances binding of the modified guide RNA to the Cas12b protein as compared to an unmodified single guide RNA.

In another aspect, provided herein is the chemically modified single guide RNA comprising a sequence selected from any one of SEQ ID NOs: 2-89, wherein the guide RNA comprises one or more chemical modifications.

In another aspect, provided herein is the chemically modified single guide RNA comprising a sequence of SEQ ID NO: 2, wherein the guide RNA comprises chemical modifications of the guide RNA of GB0002, GB0003, GB0007 or GB0008 of Table 1.

In another aspect, provided wherein the chemical modification on a single guide RNA comprising a sequence of Table 1, wherein a, u, g, and c indicate 2′-OMe modified adenine, uridine, guanine, and cytidine at structure directed select positions in combination with s, X, x and dX; wherein each ‘s’, each ‘X’, each ‘x’ and ‘dX’ respectively indicate a phosphorothioate linkage, a nebularine, a 2′-O-methylnebularine, and a 2′-deoxynebularine. In some embodiments, the nebularine, the 2′-O-methylnebularine, or the 2′-deoxynebularine replaces a adenosine in the single guide RNA as compared to an otherwise unmodified guide RNA. In some embodiments, the nebularine, the 2′-O-methylnebularine, or the 2′-deoxynebularine replaces a guanosine in the single guide RNA as compared to an otherwise unmodified guide RNA.

In another aspect, provided herein is a ribonucleoprotein complex comprising the chemically modified single guide RNA and a Cas12b protein, wherein the complex comprises increased stability as compared to a complex with an unmodified single guide RNA and a Cas12b protein, wherein the stability is measured by half-life of the complex ex vivo or in vitro.

In another aspect, provided herein is a composition for gene modification comprising the chemically modified single guide RNA as provided herein and a Cas12b protein or a nucleic acid sequence encoding the Cas12b protein, wherein the nucleic acid sequence is an mRNA. In some embodiments, the composition further comprises a vector that comprises the nucleic acid sequence encoding the Cas12b protein. In some embodiments, the composition further comprises a pharmaceutically acceptable carrier.

In another aspect, provided herein is a lipid nanoparticle (“LNP”) comprising the composition as provided herein. For example, the LNP may encapsulate an mRNA that encodes for the Cas12b protein and a gRNA that is comprised of a spacer that is complementary to a target polynucleotide of a target gene of interest.

In another aspect, provided herein is a method for modifying a targeted polynucleotide sequence in a gene of interest in a cell using the structure guided chemically modified single guide RNA and a Cas12b protein complex called ribonucleoprotein complex (RNP), the composition as provided herein, wherein the single guide RNA directs the Cas12b protein to effect a modification in the target polynucleotide sequence in the cell.

In some embodiments, the targeted polynucleotide sequence is present in a gene of interest. In some embodiments, the targeted polynucleotide sequence is present in a PCSK9 gene. In some embodiments, the targeted polynucleotide sequence is present in an ANGPTL3 gene.

In some embodiments, the cell of interest is a mammalian cell.

In some embodiments, the modification to the gene produced by the modified single guide comprising Cas12b RNP complex results in less off-target effect in the cell as compared to an unmodified single guide RNA comprising Cas12b RNP complex. In some embodiments, the single guide RNA exhibits increased stability in the cell compared to an unmodified single guide RNA, wherein the stability is measured by half-life of the single guide RNA in the cell.

In some embodiments, the modification to the target polynucleotide sequence or target gene is a double stranded break, a non-sense mutation, a frameshift mutation, a splice site alteration, or an inversion.

In some embodiments, the modification to the gene results in reducing or abolishing expression of functional protein encoded by the gene. In some embodiments, the modified gene is PCSK9 which results in reducing or abolishing expression of functional PCSK9 protein in the cell. In some embodiments, the modified gene is ANGPTL3 which result in reducing or abolishing expression of functional ANGPTL3 protein in the cell.

In another aspect, provided herein is a method for treating or preventing a condition in a subject in need thereof by administering the RNP complex comprising chemically modified single guide RNA and Cas12b protein, wherein the spacer of single guide RNA that is designed to be complementary to the targeted polynucleotide sequence of the gene hybridize with the target to facilitate the Cas12b protein to effect a modification to the target polynucleotide sequence of the gene, thereby producing a therapeutic effect for treating or preventing certain disease condition.

In some embodiments, the disease condition is atherosclerotic vascular disease such as hypertriglyceridemia and diabetes.

In some embodiments, the target polynucleotide sequence is in an ANGPTL3 gene. In some embodiments, the modification reduces expression of functional ANGPTL3 protein encoded by the ANGPTL3 gene in the subject. In some embodiments, the condition is a atherosclerotic vascular disease, hypertriglyceridemia, or diabetes.

In some embodiments, the subject post-treatment/post-administration exhibits a reduced blood LDL cholesterol level, and/or a reduced blood triglycerides level as compared to pre-administration.

In another aspect, provided herein is a single guide RNA that comprises (i) a spacer sequence and (ii) a tracr sequence, wherein the spacer sequence hybridizes with a target polynucleotide sequence in a PCSK9 gene or an ANGPTL3 gene when contacted with the target polynucleotide, wherein the scaffold sequence binds a Cas12b protein when contacted with the Cas12b protein, and wherein the single guide RNA comprises a chemical modification.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a Sanger sequencing chromatogram demonstrating Cas12b editing in cells. HEK293 cells were transfected 1:1 with a plasmid encoding Cas12b and a plasmid encoding the hANG_Cas12b_3 gRNA. The gRNA protospacer is aligned to the sanger sequencing chromatogram. FIG. 1 discloses SEQ ID NOS 111 and 114, respectively, in order of appearance.

FIG. 2 depicts a Sanger sequencing chromatogram demonstrating Cas12b editing in cells. HEK293 cells were transfected 1:1 with a plasmid encoding Cas12b and a plasmid encoding the hANG_Cas12b_5 gRNA. The gRNA protospacer is aligned to the sanger sequencing chromatogram. FIG. 2 discloses SEQ ID NOS 121 and 116, respectively, in order of appearance.

FIG. 3 depicts a Sanger sequencing chromatogram demonstrating Cas12b editing in cells. HEK293 cells were transfected 1:1 with a plasmid encoding Cas12b and a plasmid encoding the hANG_Cas12b_7 gRNA. The gRNA protospacer is aligned to the sanger sequencing chromatogram. FIG. 3 discloses SEQ ID NOS 123 and 118, respectively, in order of appearance.

FIG. 4 depicts a Sanger sequencing chromatogram demonstrating Cas12b editing in cells. HEK293 cells were transfected 1:1 with a plasmid encoding Cas12b and a plasmid encoding the hANG_Cas12b_14 gRNA. The gRNA protospacer is aligned to the sanger sequencing chromatogram. FIG. 4 discloses SEQ ID NOS 124 and 94, respectively, in order of appearance.

FIG. 5 depicts a Sanger sequencing chromatogram demonstrating Cas12b editing in cells. HEK293 cells were transfected 1:1 with a plasmid encoding Cas12b and a plasmid encoding the hANG_Cas12b_33 gRNA. The gRNA protospacer is aligned to the sanger sequencing chromatogram. FIG. 5 discloses SEQ ID NOS 126 and 96, respectively, in order of appearance.

FIG. 6 depicts a Sanger sequencing chromatogram demonstrating Cas12b editing in cells. HEK293 cells were transfected 1:1 with a plasmid encoding Cas12b and a plasmid encoding the hANG_Cas12b_34 gRNA. The gRNA protospacer is aligned to the sanger sequencing chromatogram. FIG. 6 discloses SEQ ID NOS 137-138, respectively, in order of appearance.

FIG. 7 depicts a Sanger sequencing chromatogram demonstrating Cas12b editing in cells. HEK293 cells were transfected 1:1 with a plasmid encoding Cas12b and a plasmid encoding the hANG_Cas12b_42 gRNA. The gRNA protospacer is aligned to the sanger sequencing chromatogram. FIG. 7 discloses SEQ ID NOS 144 and 90, respectively, in order of appearance.

FIG. 8 depicts a Sanger sequencing chromatogram demonstrating Cas12b editing in cells. HEK293 cells were transfected 1:1 with a plasmid encoding Cas12b and a plasmid encoding the hANG_Cas12b_64 gRNA. The gRNA protospacer is aligned to the sanger sequencing chromatogram. FIG. 8 discloses SEQ ID NOS 158 and 91, respectively, in order of appearance.

FIG. 9 depicts a Sanger sequencing chromatogram demonstrating Cas12b editing in cells. HEK293 cells were transfected 1:1 with a plasmid encoding Cas12b and a plasmid encoding the hANG_Cas12b_82 gRNA. The gRNA protospacer is aligned to the sanger sequencing chromatogram. FIG. 9 discloses SEQ ID NOS 170 and 97, respectively, in order of appearance.

FIG. 10 depicts a Sanger sequencing chromatogram demonstrating Cas12b editing in cells. HEK293 cells were transfected 1:1 with a plasmid encoding Cas12b and a plasmid encoding the hANG_Cas12b_84 gRNA. The gRNA protospacer is aligned to the sanger sequencing chromatogram. FIG. 10 discloses SEQ ID NOS 171 and 174, respectively, in order of appearance.

FIG. 11 depicts a Sanger sequencing chromatogram demonstrating Cas12b editing in cells. HEK293 cells were transfected 1:1 with a plasmid encoding Cas12b and a plasmid encoding the hANG_Cas12b_87 gRNA. The gRNA protospacer is aligned to the sanger sequencing chromatogram. FIG. 11 discloses SEQ ID NOS 175 and 188, respectively, in order of appearance.

FIG. 12 depicts a Sanger sequencing chromatogram demonstrating Cas12b editing in cells. HEK293 cells were transfected 1:1 with a plasmid encoding Cas12b and a plasmid encoding the hANG_Cas12b_88 gRNA. The gRNA protospacer is aligned to the sanger sequencing chromatogram. FIG. 12 discloses SEQ ID NOS 190-191, respectively, in order of appearance.

FIG. 13 depicts a Sanger sequencing chromatogram demonstrating Cas12b editing in cells. HEK293 cells were transfected 1:1 with a plasmid encoding Cas12b and a plasmid encoding the hANG_Cas12b_95 gRNA. The gRNA protospacer is aligned to the sanger sequencing chromatogram. FIG. 13 discloses SEQ ID NOS 192-193, respectively, in order of appearance.

FIG. 14 depicts a Sanger sequencing chromatogram demonstrating Cas12b editing in cells. HEK293 cells were transfected 1:1 with a plasmid encoding Cas12b and a plasmid encoding the hANG_Cas12b_117 gRNA. The gRNA protospacer is aligned to the sanger sequencing chromatogram. FIG. 14 discloses SEQ ID NOS 194 and 101, respectively, in order of appearance.

FIG. 15 shows the results from a CEL I enzymatic mutation detection assay (surveyor nuclease assay). HEK293 cells were transfected 1:1 with a plasmid encoding Cas12b and a plasmid encoding the gRNA depicted. The genomic DNA was harvested and PCR amplified at the target region. After performing the Surveyor nuclease assay per manufacturer's instructions, the PCR amplicons were separated and visualized on an agarose gel. Editing of target sites is identified by cleavage bands below the top amplicon band.

FIG. 16 shows the results from a CEL I enzymatic mutation detection assay (surveyor nuclease assay). HEK293 cells were transfected 1:1 with a plasmid encoding Cas12b and a plasmid encoding the gRNA depicted. The genomic DNA was harvested and PCR amplified at the target region. After performing the Surveyor nuclease assay per manufacturer's instructions, the PCR amplicons were separated and visualized on an agarose gel. Editing of target sites is identified by cleavage bands below the top amplicon band.

FIG. 17 shows the results from a CEL I enzymatic mutation detection assay (surveyor nuclease assay). HEK293 cells were transfected 1:1 with a plasmid encoding Cas12b and a plasmid encoding the gRNA depicted. The genomic DNA was harvested and PCR amplified at the target region. After performing the Surveyor nuclease assay per manufacturer's instructions, the PCR amplicons were separated and visualized on an agarose gel. Editing of target sites is identified by cleavage bands below the top amplicon band.

FIG. 18 illustrates key details from a structure-based approach to: 1) identify bases where 2′-O-methyl could be substituted in lieu of 2′-hydroxyl groups to offer stability to an exemplary Cas12b gRNA, while not causing steric hindrance with either the protein or gRNA; 2) introduce changes to the gRNA sequence to improve stability and/or improve nuclease activity. FIG. 18 discloses SEQ ID NO: 1.

FIG. 19A-19B show exemplary sgRNAs for Alicyclobacillus acidoterrestris Cas12b (FIG. 19A) and Bacillus thermoamylovorans Cas12b (FIG. 19B). FIG. 19A discloses SEQ ID NO: 195 and FIG. 19B discloses SEQ ID NO: 196.

FIG. 20 is an alignment of a reference unmodified guide RNA for ANGPTL3 (SEQ ID NO: 1) along with corresponding chemically modified guides, GB0002, GB0003, GB0007, and GB0008. Underlined nucleotides depict the spacer sequence for the unmodified and modified guides (positions 98 to 120). Letters indicate: A: Adensine, C: Cytidine; G: Guanosine, U: Uridine; T: thymidine; a: 2′-O-methyladenosine (2′-OMe-A); c: 2′-O-methylcytidine (2′-OMe-C); g: 2′-O-methylguanosine (2′-OMe-G); u: 2′-O-methyluridine (2′-OMe-U); and s: phosphorothioate (PS) linkage. FIG. 20 discloses SEQ ID NOS 1, 197-198 AND 202-203

### DETAILED DESCRIPTION OF THE DISCLOSURE

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Further, headings provided herein are for convenience only and do not interpret the scope or meaning of the claimed disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Certain Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. Furthermore, use of the term “including” as well as other forms, such as “include”, “includes,” and “included,” is not limiting.

The terms “modified” and “chemically modified” are interchangeably use and/or represent chemically modified or chemical modification in a given context, for example, “modified polynucleotide” and “chemically modified polynucleotide” indicates chemical modification on the polynucleotide.

Reference in the specification to “embodiments,” “some embodiments,” “an embodiment,” “one embodiment” or “other embodiments” means that a particular feature, structure, or characteristic described in connection with the embodiments is included in at least some embodiments, but not necessarily all embodiments, of the disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 comprises 1 to 10, 1 to 20, 1 to 30, and 1 to 40 in one direction, or 50 to 40, 50 to 30, 50 to 20, and 50 to 10 in the other direction.

The terms “nucleic acid,” “nucleotides,” and “polynucleotides,” as used herein, are used interchangeably and refer to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone.

Examples of modified sugar moieties include, but are not limited to, 2′-O-methyl, 2′-O-methoxyethyl, 2′-O-aminoethyl, 2′-Flouro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. Backbone modifications can also include having peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in some embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The term “protospacer” “or “target polynucleotide sequence” and its grammatical equivalents as used herein can refer to a PAM-adjacent nucleic acid sequence upon which the “spacer sequence” of the guide RNA (gRNA) is adapted from protospacer as an RNA version thereof. A protospacer can be a nucleotide sequence within gene, genome, or chromosome that is targeted by a gRNA. In the native state, a protospacer is adjacent to a PAM (protospacer adjacent motif). The site of cleavage by an RNA-guided nuclease is within a protospacer sequence. For example, when a gRNA targets a specific protospacer, the Cas protein will generate a double strand break within the protospacer sequence, thereby cleaving the protospacer. Following cleavage, disruption of the protospacer can result though non-homologous end joining (NHEJ) or homology-directed repair (HDR). Disruption of the protospacer can result in the deletion of the protospacer. Additionally or alternatively, disruption of the protospacer can result in an exogenous nucleic acid sequence being inserted into or replacing the protospacer.

The terms “gene editing,” or “gene modification” and its grammatical equivalents as used herein can refer to genetic engineering in which one or more nucleotides are inserted, replaced, or removed from a genome. Gene editing can be performed using a nuclease (e.g., a natural-existing nuclease or an artificially engineered nuclease). Gene modification can include introducing a double stranded break, a non-sense mutation, a frameshift mutation, a splice site alteration, or an inversion in a polynucleotide sequence, e.g., a target polynucleotide sequence.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

### CRISPR/CAS System

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. A CRISPR/Cas system comprises a non-coding RNA molecule (e.g., guide RNA) that binds to DNA (e.g., target DNA sequence) and Cas proteins (e.g., Cas9, Cas12b) with nuclease functionality (e.g., two nuclease domains). See, e.g., Sander, et al., Nature Biotechnology, 32:347-355 (2014); see also e.g., Hsu, et al., Cell 157(6):1262-1278 (2014). The general mechanism and recent advances of CRISPR system is discussed in Cong, et al., Science, 339(6121): 819-823 (2013); Fu, et al., Nature Biotechnology, 31, 822-826 (2013); Chu, et al., Nature Biotechnology 33, 543-548 (2015); Shmakov, et al., Molecular Cell, 60, 1-13 (2015); Makarova, et al., Nature Reviews Microbiology, 13, 1-15 (2015). CRISPR/Cas systems can be used to introduce site-specific cleavage of a target DNA. The locations for site-specific cleavage are determined by both 1) base-pairing complementarity between the guide RNA (gRNA) and the target DNA sequence that is complementary to a protospacer sequence and 2) a short motif in the target DNA referred to as the protospacer adjacent motif (PAM). CRISPR/Cas systems (e.g., Type II CRISPR/Cas system) can be used to generate, e.g., an engineered cell in which a target gene is disrupted or mutated. A Cas enzyme (e.g., Cas9, Cas12b) can be used to catalyze DNA cleavage. A Cas9 protein (e.g., a Streptococcus pyogenes Cas9 or any closely related Cas9), for example, can derive an enzymatic action to generate double stranded breaks at target site sequences which hybridize to about 20 nucleotides of a guide sequence (e.g., gRNA) and that have a protospacer-adjacent motif (PAM) following the target sequence.

CRISPR/Cas system comprises Class 1 or Class 2 system components, including ribonucleic acid protein complexes. The Class 2 Cas nuclease families of proteins are enzymes with DNA endonuclease activity, and they can be directed to cleave a desired nucleic acid target by designing an appropriate guide RNA, as described further herein. A Class 2 CRISPR/Cas system component may be from a Type II, Type IIA, Type IIB, Type IIC, Type V, or Type VI system. Class 2 Cas nucleases include, for example, Cas9 (also known as Csn1 or Csx12), Csn2, Cas4, Cas12a (Cpf1), Cas12b (C2c1), Cas12c (C2c3), Cas13a (C2c2), Cas13b, Cas13c, and Cas13d proteins.

Other non-limiting examples of Cas proteins include Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, Cas9HiFi, homologues thereof, or modified versions thereof.

CRISPR clusters (e.g., spacers, sequences complementary to antecedent mobile elements, and target invading nucleic acids) are transcribed and processed into CRISPR RNA (crRNA). In Type II CRISPR systems correct processing of pre-crRNA requires a trans-activating crRNA (tracrRNA), endogenous ribonuclease 3 (rnc) and a Class 2 Cas nuclease. The tracrRNA serves as a guide for ribonuclease 3-aided processing of pre-crRNA. Subsequently, Cas nuclease/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (sgRNA) can be engineered so as to incorporate aspects of both the crRNA and tracrRNA into a single RNA species (Jinek M., et al., Science (2012) 337:816-821). Cas nucleases recognize a short motif in the CRISPR repeat sequences (the PAM or protospacer adjacent motif) to help distinguish self versus non-self. For example, Cas9 nuclease sequences and structures are well known to those of skill in the art (see, e.g., Ferretti, et al., Proc. Natl. Acad. Sci. U.S.A. (2001) 98:4658-4663; Deltcheva, et al., Nature (2011) 471:602-607; and Jinek, et al., Science (2012) 337:816-821, the entire contents of each of which are incorporated herein by reference).

### CAS12B (C2C1)

The term “Cas12b (C2C1),” as used herein, refers to a type V-B CRISPR-Cas system RNA-guided DNA endonuclease that has a double-stranded DNA cleavage activity. For example, a crRNA in combination with a minimal 78 nt (nucleotides) tracrRNA or a fused sgRNA is sufficient for Cas12b-mediated DNA cleavage. For instance, in some embodiments, a 14 nt direct repeat (DR) hybridizes with tracrRNA to form a crRNA: tracrRNA duplex, which is then loaded onto Cas12b to guide DNA recognition and cleavage. Cas12b contains a RuvC-like nuclease domain and a putative Nuc domain, generating a staggered double-stranded break at the target locus, with a 5′ overhang, or a “sticky end” at the PAM distal side of the target sequence (See, e.g., Garneau et al, Nature. 2010; 468:67-71; Gasiunas et al., Proc Natl Acad Sci USA. 2012; 109: E2579-2586, the entire contents of which are incorporated herein by reference). In some embodiments, the 5′ overhang is 7 nt. (See, e.g., Lewis and Ke, Mol Cell. 2017, 65(3):377-379 and Liu et al., Molecular cell, 2017, 65(2):310-32, the entire contents of which are incorporated herein by reference). Cas12b is sometimes also referred to as Cpf2.

In some embodiments, Cas12b originates from a bacterium selected from the group consisting of: Alicyclobacillus kakegawensis, Alicyclobacillus acidoterrestris, Alicyclobacillus contaminans, Alicyclobacillus macrosporangiidus, Bacillus sp. V3-13, Bacillus hisashii, Candidatus Lindowbacteria bacterium RIFCSPLOW02, Desulfovibrio inopinatus, Desulfonatronum thiodismutans, Elusimicrobia bacterium RIFOXYA12, Lentisphaeria bacterium, Omnitrophica WOR 2 bacterium RIFCSPHIGH02, Opitutaceae bacterium TAV5, Phycisphaerae bacterium ST-NAGAB-D1, Planctomycetes bacterium RBG_13_46_1O, Spirochaetes bacterium GWB1 27 13, Verrucomicrobiaceae bacterium UBA2429, Tuberibacillus calidus, Bacillus thermoamylovorans, Brevibacillus sp. CF112, Bacillus sp. NSP2.1, Desulfatirhabdium butyrativorans, Alicyclobacillus herbarius, Citrobacter freundii, Brevibacillus agri, Methylobacterium nodulans and Laceyella sediminis. In some embodiments, Cas12b originates from a bacterium selected from the group consisting of: Alicyclobacillus, Desulfovibrio, Desulfonatronum, Opitutaceae, Tuberibacillus, Bacillus, Brevibacillus, Candidatus, Desulfatirhabdium, Citrobacter, Desulfatirhabdium butyrativorans, Desulfonatronum thiodismutans, Elusimicrobia, Methylobacterium, Methylobacterium nodulans, Omnitrophica, Phycisphaerae, Planctomycetes, Spirochaetes, Verrucomicrobiaceae, Lentisphaeria, and Laceyella, Laceyella sediminis.

In some embodiment, Cas12b recognizes the T-rich PAM at the 5′ end of the protospacer sequence to mediate DNA interference. In some embodiments, Cas12b PAM sequences may be T-rich sequences. In some embodiments, the PAM sequence is 5′ TTN 3′ or 5′ ATTN 3′, wherein Nis any nucleotide. In some embodiments, the PAM sequence is 5′ TTC 3′. In some embodiments, the Alicyclobacillus acidoterrestris ATCC 49025 C2cl protein (AacC2cl) cleaves target sites preceded by a 5′ TTN PAM, where N is A, C, G, or T, more preferably where N is A, G, or T. In some embodiments, the Bacillus thermoamylovorans strain B4166 C2cl protein (BthC2cl) cleaves sites preceded by a ATTN, where N is A/C/G or T.

An exemplary Bacillus hisashii Cas12b has an amino acid sequence of

(SEQ ID NO: 102)
MATRSFILKIEPNEEVKKGLWKTHEVLNHGIAYYMNILKLIRQEAIY
EHHEQDPKNPKKVSKAEIQAELWDFVLKMQKCNSFTHEVDKDEVFNI
LRELYEELVPSSVEKKGEANQLSNKFLYPLVDPNSQSGKGTASSGRK
PRWYNLKIAGDPSWEEEKKKWEEDKKKDPLAKILGKLAEYGLIPLFI
PYTDSNEPIVKEIKWMEKSRNQSVRRLDKDMFIQALERFLSWESWNL
KVKEEYEKVEKEYKTLEERIKEDIQALKALEQYEKERQEQLLRDTLN
TNEYRLSKRGLRGWREIIQKWLKMDENEPSEKYLEVFKDYQRKHPRE
AGDYSVYEFLSKKENHFIWRNHPEYPYLYATFCEIDKKKKDAKQQAT
FTLADPINHPLWVRFEERSGSNLNKYRILTEQLHTEKLKKKLTVQLD
RLIYPTESGGWEEKGKVDIVLLPSRQFYNQIFLDIEEKGKHAFTYKD
ESIKFPLKGTLGGARVQFDRDHLRRYPHKVESGNVGRIYFNMTVNIE
PTESPVSKSLKIHRDDFPKVVNFKPKELTEWIKDSKGKKLKSGIESL
EIGLRVMSIDLGQRQAAAASIFEVVDQKPDIEGKLFFPIKGTELYAV
HRASFNIKLPGETLVKSREVLRKAREDNLKLMNQKLNFLRNVLHFQQ
FEDITEREKRVTKWISRQENSDVPLVYQDELIQIRELMYKPYKDWVA
FLKQLHKRLEVEIGKEVKHWRKSLSDGRKGLYGISLKNIDEIDRTRK
FLLRWSLRPTEPGEVRRLEPGQRFAIDQLNHLNALKEDRLKKMANTI
IMHALGYCYDVRKKKWQAKNPACQIILFEDLSNYNPYEERSRFENSK
LMKWSRREIPRQVALQGEIYGLQVGEVGAQFSSRFHAKTGSPGIRCS
VVTKEKLQDNRFFKNLQREGRLTLDKIAVLKEGDLYPDKGGEKFISL
SKDRKCVTTHADINAAQNLQKRFWTRTHGFYKVYCKAYQVDGQTVYI
PESKDQKQKIIEEFGEGYFILKDGVYEWVNAGKLKIKKGSSKQSSSE
LVDSDILKDSFDLASELKGEKLMLYRDPSGNVFPSDKWMAAGVFFGK
LERILISKLTNQYSISTIEDDSSKQSM

In some embodiments, Cas12b is Bacillus hisashii Cas12b. In some embodiments, Cas12b has a mutation. In some embodiments, Cas12b is Bacillus hisashii Cas12b that has a mutation. In some embodiments, Cas12b is Bacillus hisashii Cas12b that has K846R mutation. In some embodiments, Cas12b is Bacillus hisashii Cas12b that has S893R mutation. In some embodiments, Cas12b is Bacillus hisashii Cas12b that has E837G mutation. In some embodiments, Cas12b is Bacillus hisashii Cas12b that has K846R mutation, S893R mutation, E837G mutation, and any combination thereof. In some embodiments, Cas12b is Bacillus hisashii Cas12b that has K846R/S893R/E837G mutations.

In some embodiments, the recombinant Cas12b protein comprises one or more nuclear localization signals. In some embodiments, the recombinant Cas12b protein is catalytically inactive. In some embodiments, the recombinant Cas12b protein is associated with one or more functional domains.

### Guide RNAs

The guide RNA (gRNA) is capable of guiding the Class 2 Cas nuclease to a target sequence on a target nucleic acid molecule where the target sequence is complementary to the protospacer sequence, where the gRNA hybridizes with and the Cas nuclease cleaves or modulates the target sequence. In some embodiments, a gRNA binds with and provides specificity of cleavage by a Class 2 nuclease. In some embodiments, the gRNA and the Cas protein may form a ribonucleoprotein (RNP), e.g., a CRISPR/Cas complex. In some embodiments, the CRISPR complex may be a Type II CRISPR/Class 2 Cas nuclease. In some embodiments, the CRISPR/Cas complex may be a Type V CRISPR/Cas complex, such as a Cas12b/guide RNA complex. In some embodiments, the Cas nuclease may be a single-protein Cas nuclease, e.g., a Cas12b protein. In some embodiments, the gRNA targets cleavage by a Cas12b protein.

A gRNA for Cas12b editing can comprise at least two distinct regions: a first distinct region at the 5′ end called the scaffold sequence comprises of stem loops and single strands that recognize and bind to the Cas12b protein, and the second distinct regions at the 3′ end of the scaffold sequence is designed to be complementary to a target site in a chromosomal sequence (i.e., spacer region) that facilitate binding of the Cas12b protein-gRNA complex to the targeted section of the gene of interest to effect Cas12b/guide RNA-mediated gene modification. Further, scaffold regions of each gRNA can be identical in all gRNAs as it is the protein binding domain of the gRNA. The spacer region of each guide RNA can also be different such that each gRNA guides a protein, e.g., Cas12b, to a specific target site. A scaffold region of a gRNA may form a secondary structure. In some embodiments, a secondary structure formed by a gRNA can comprise a stem (or hairpin) and a loop. The length of a loop and/or a stem can vary. In some embodiments, a loop can range from about 3 to about 10 nucleotides in length. In some embodiments, a stem can range from about 6 to about 20 nucleotides in length. A stem can comprise one or more bulges of 1 to 10 nucleotides or about 10 nucleotides. In some embodiments, the overall length of a second region can range from about 16 to 60 nucleotides in length. In some embodiments, a loop can be about 4 nucleotides in length. In some embodiments, a stem can be about 12 in length. A third region of scaffold sequence can be essentially single-stranded. In addition, the length of the spacer at the 3′-end of the Cas12b gRNA can vary. In some embodiments, the spacer sequence can be more than 3 or more than 4 nucleotides in length. For example, the length of the spacer at the 3′ end of the gRNA can range from about 20 to 40 nucleotides in length.

A gRNA for a CRISPR/Cas12b system can comprise a CRISPR RNA (crRNA) and a trans-activating crRNA (tracrRNA, tracr sequence, or scaffold sequence). As used herein, tracrRNA, tracr sequence, or scaffold sequence are used interchangeably. In some embodiments, the crRNA comprises a targeting or spacer sequence that is complementary to and capable of hybridizing with the target sequence on the target nucleic acid molecule. The crRNA may also comprise a portion that is complementary to and capable of hybridizing with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas12b system, and a portion of a repeat sequence flanking the spacers on the CRISPR locus. The gRNA may target any sequence of interest via the targeting sequence of the crRNA. In some embodiments, the degree of complementarity between the targeting sequence of the gRNA and the target sequence on the target nucleic acid molecule is at least about 60%, 65%, 70%, 75%, 80%, 85%, 90%, 95%, 97%, 98%, 99%, or 100%. In some embodiments, the targeting sequence of the gRNA and the target sequence on the target nucleic acid molecule may be 100% complementary. In other embodiments, the targeting sequence of the gRNA and the target sequence on the target nucleic acid molecule may contain at least one mismatch. For example, the targeting sequence of the gRNA and the target sequence on the target nucleic acid molecule may contain 1, 2, 3, 4, 5, 6, 7, 8, 9, or 10 mismatches.

In some embodiments, the length of the targeting sequence depends on the CRISPR/Cas system and components used. For example, different Cas proteins from different bacterial species have varying optimal targeting sequence lengths. Accordingly, the targeting sequence comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the targeting sequence comprised 20-40 nucleotides in length. In some embodiments, the targeting sequence comprises 20-25 nucleotides in length. In some embodiments, the targeting sequence comprises 24 nucleotides in length. In some embodiments, the targeting sequence comprises 23 nucleotides in length.

In some embodiments, the guide RNA is a “dual guide RNA” or “dgRNA”. In some embodiments, the dgRNA comprises a first RNA molecule comprising a crRNA, and a second RNA molecule comprising a tracrRNA. The first and second RNA molecules may form a RNA duplex via the base pairing between the crRNA and the tracrRNA (e.g. repeat and anti-repeat). In some embodiments, the guide RNA is a “single guide RNA” or “sgRNA.” In some embodiments, the sgRNA comprises a crRNA covalently linked to a tracrRNA. In some embodiments, the crRNA and the tracrRNA may be covalently linked via a linker. In some embodiments, the single-molecule guide RNA comprises a stem-loop structure via the base pairing between the crRNA and the tracrRNA. In some embodiments, the sgRNA is a “Cas12b sgRNA” capable of mediating RNA-guided DNA cleavage by a Cas12b protein. In certain embodiments, the guide RNA comprises a crRNA and tracrRNA sufficient for forming an active complex with a Cas12b protein and mediating RNA-guided DNA cleavage. The terms “guide RNA,” “single guide RNA,” “gRNA,” and “sgRNA” are used interchangeably throughout this application. In some embodiments, more than one guide RNA can be used; each guide RNA contains a different targeting sequence, such that the CRISPR/Cas12b system cleaves more than one target sequence. In some embodiments, one or more guide RNAs may have the same or differing properties such as activity or stability within a CRISPR/Cas12b complex. Where more than one guide RNA is used, each guide RNA can be encoded on the same or on different expression cassettes. The promoters used to drive expression of the more than one guide RNA may be the same or different.

In some embodiments, the gRNA or mRNA encoding Cas12b nuclease is modified. The modifications can comprise chemical alterations, synthetic modifications, nucleotide additions, and/or nucleotide subtractions and modified nucleosides or nucleotides can be present in a gRNA. A gRNA or Cas nuclease-encoding mRNA comprising one or more modified nucleosides or nucleotides is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide. Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, addition, modification, or replacement of a terminal phosphate group or conjugation of a moiety, cap, or linker (such 3′ or 5′ cap modifications comprises a sugar and/or backbone modification); and (vii) modification or replacement of the sugar (an exemplary sugar modification). The modifications can enhance genome editing by CRISPR/Cas12b. A modification can alter chirality of a gRNA. In some cases, chirality may be uniform or stereopure after a modification. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides.

Methods for selecting, designing, and validating gRNAs and targeting sequences (or spacer sequences) are described herein and known to those skilled in the art. Software tools can be used to optimize the gRNAs corresponding to a target nucleic acid sequence, e.g., to minimize total off-target activity across the genome. For example, for each possible targeting domain choice using B. hisashii Cas12b, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. In some embodiments, a DNA sequence searching algorithm can be used to identify a target sequence in crRNAs of a gRNA for use with Cas12b. A custom gRNA design software based on the public tool cas-offinder, which scores guides after calculating their genome-wide off-target propensity, can be also used to design a gRNA (e.g., Bae, et al., Cas-OFFinder: A fast and versatile algorithm that searches for potential off-target sites of Cas9 RNA-guided endonucleases. Bioinformatics 30, 1473-1475 (2014)). In some embodiments, RepeatMasker program can be used to screen repeat elements and regions of low complexity in the input DNA sequences. In addition, the number of residues that could unintentionally be targeted (e.g., off-target residues that could potentially reside on ssDNA within the target nucleic acid locus) may be minimized to reduce the impact of potential substrate promiscuity of the Cas12b editing system. Candidate gRNAs can be functionally evaluated by using methods known in the art and/or as set forth herein.

The gRNAs described herein can be synthesized chemically, enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include, but are not limited to, T7, T3, SP6 promoter sequences, or variations thereof. In some embodiments, gRNA comprises two separate molecules (e.g., crRNA and tracrRNA) and one molecule (e.g., crRNA) can be chemically synthesized and the other molecule (e.g., tracrRNA) can be enzymatically synthesized.

### Cas12b Guide RNAs

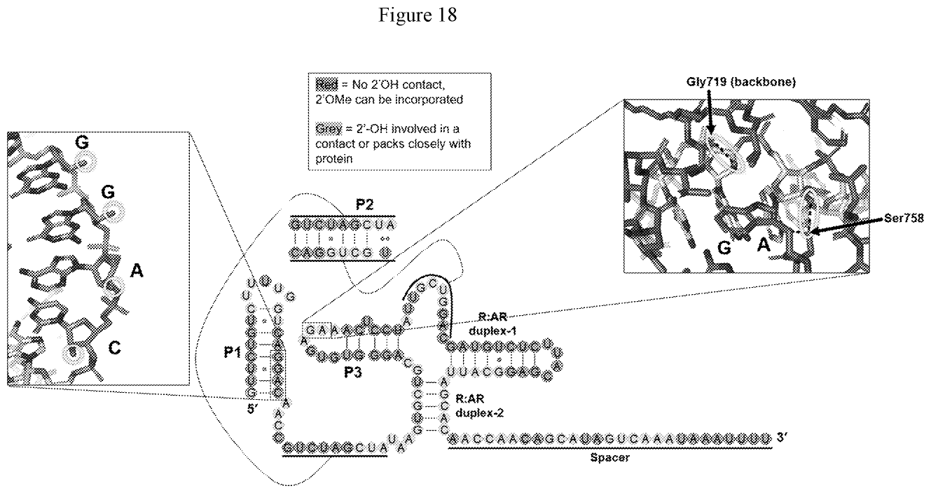

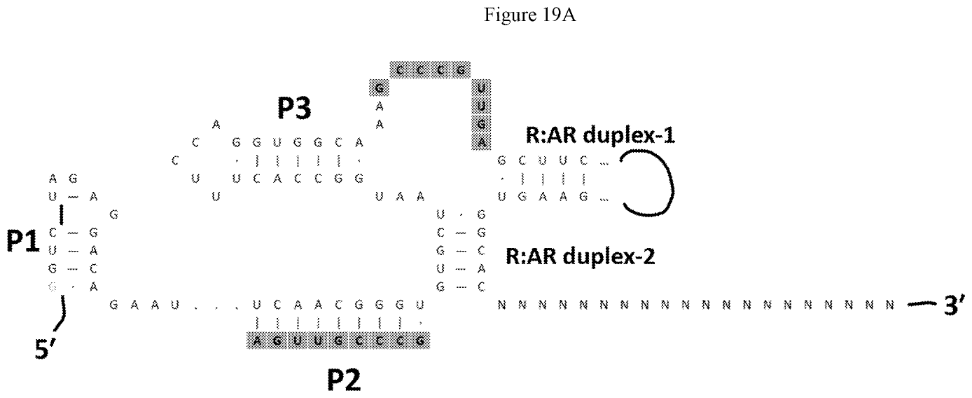

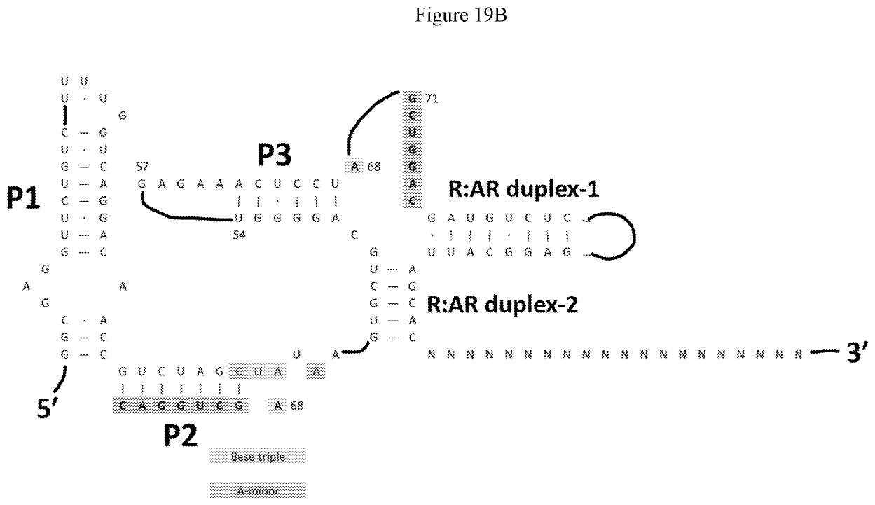

Described herein are guide RNAs that have improved properties or functionalities for a a CRISPR/Cas12b gene editing system. In some embodiments, the guide RNA comprises two RNA molecules, i.e., a crRNA, and a tracrRNA. In some embodiments, the two molecules are connected by a linker. In some embodiments, the two molecules are connected by a non-nucleic acid linker. In some embodiments, the two molecules are connected by a peptide linker or a chemical linker. In some embodiments, the guide RNA is a single molecule or single guide RNA. Illustrative Cas12b single guide RNAs and their interactions with Cas12b protein are depicted in FIGS. 18-19.

In some embodiments, a guide RNA comprises a total of 50-150 nucleotides in length. In some embodiments, a guide RNA comprises a total of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides in length. In some embodiments, a guide RNA comprises a total of about 50 to about 60, about 50 to about 70, about 50 to about 80, about 50 to about 90, about 50 to about 100, about 50 to about 110, about 50 to about 120, about 50 to about 130, about 50 to about 140, about 60 to about 70, about 60 to about 80, about 60 to about 90, about 60 to about 100, about 60 to about 110, about 60 to about 120, about 60 to about 130, about 60 to about 140, about 70 to about 80, about 70 to about 90, about 70 to about 100, about 70 to about 110, about 70 to about 120, about 70 to about 130, about 70 to about 140, about 80 to about 90, about 80 to about 100, about 80 to about 110, about 80 to about 120, about 80 to about 130, about 80 to about 140, about 90 to about 100, about 90 to about 110, about 90 to about 120, about 90 to about 130, about 90 to about 140, about 100 to about 110, about 100 to about 120, about 100 to about 130, about 100 to about 140, about 110 to about 120, about 110 to about 130, about 110 to about 140, about 120 to about 130, about 120 to about 140, or about 130 to about 140 nucleotides in length. In some embodiments, a guide RNA comprises a total of about 50, about 60, about 70, about 80, about 90, about 100, about 110, about 120, about 130, or about 140 nucleotides in length. In some embodiments, a guide RNA comprises a total of at least about 50, about 60, about 70, about 80, about 90, about 100, about 110, about 120, or about 130 nucleotides in length. In some embodiments, a guide RNA comprises a total of at most about 60, about 70, about 80, about 90, about 100, about 110, about 120, about 130, or about 140 nucleotides in length. In some embodiment, a guide RNA comprises a total of 121, 122, 123, 124, 125, 126, 127, 128, or 129 nucleotides in length. In another embodiment, a guide RNA comprises a total of 124 nucleotides in length.

In some embodiments, a guide RNA comprises a spacer region or sequence (i.e., the target sequence) of 10 to 40 nucleotides in length. Illustrative spacer sequences are depicted in the single guide RNAs shown in FIGS. 18-19. In some embodiments, a guide RNA comprises a spacer sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a guide RNA comprises a spacer sequence of about 10 to about 40 nucleotides in length. In some embodiments, a guide RNA comprises a spacer sequence of about 10 to about 15, about 10 to about 20, about 10 to about 25, about 10 to about 30, about 15 to about 20, about 15 to about 25, about 15 to about 30, about 20 to about 25, about 20 to about 30, about 25 to about 30 nucleotides, about 30 to about 35 nucleotides, or about 35 to about 40 nucleotides in length. In some embodiments, a guide RNA comprises a spacer sequence of about 10, about 15, about 20, about 25, about 30, about 35, or about 40 nucleotides in length. In some embodiments, a guide RNA comprises a spacer sequence of at least about 10, about 15, about 20, about 25, or about 30 nucleotides in length. In some embodiments, a guide RNA comprises a spacer sequence of at most about 15, about 20, about 25, about 30, or about 35 nucleotides in length. In some embodiment, a guide RNA comprises a spacer sequence of 10, 21, 22, 23, 24, 25, 26, 27, 28, 29, 30, 31, 32, 33, 34, or 35 nucleotides in length. In another embodiment, a guide RNA comprises a spacer sequence of 27 nucleotides in length. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

In some embodiments, a guide RNA comprises a scaffold sequence. The scaffold sequence acts as a substrate for a Cas12b protein to facilitate the formation of Cas12b protein-guide RNA complex. Illustrative scaffold sequences are depicted in the single guide RNAs shown in FIGS. 18-19. In some embodiments, the scaffold sequence comprises a tracrRNA sequence, or a stem loop structure. In some embodiments, the scaffold sequence comprises a tracrRNA sequence, and a stem loop structure. In some embodiments, a guide RNA comprises a scaffold sequence of from 30 to 130 nucleotides in length. In some embodiments, a guide RNA comprises a scaffold sequence of 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 nucleotides in length. In some embodiments, a guide RNA comprises a scaffold sequence of from about 30 to about 130 nucleotides in length. In some embodiments, a guide RNA comprises a scaffold sequence of from about 30 to about 40, about 30 to about 50, about 30 to about 60, about 30 to about 70, about 30 to about 75, about 30 to about 80, about 30 to about 90, about 30 to about 100, about 30 to about 110, about 30 to about 120, about 30 to about 130, about 40 to about 50, about 40 to about 60, about 40 to about 70, about 40 to about 75, about 40 to about 80, about 40 to about 90, about 40 to about 100, about 40 to about 110, about 40 to about 120, about 40 to about 130, about 50 to about 60, about 50 to about 70, about 50 to about 75, about 50 to about 80, about 50 to about 90, about 50 to about 100, about 50 to about 110, about 50 to about 120, about 50 to about 130, about 60 to about 70, about 60 to about 75, about 60 to about 80, about 60 to about 90, about 60 to about 100, about 60 to about 110, about 60 to about 120, about 60 to about 130, about 70 to about 75, about 70 to about 80, about 70 to about 90, about 70 to about 100, about 70 to about 110, about 70 to about 120, about 70 to about 130, about 75 to about 80, about 75 to about 90, about 75 to about 100, about 75 to about 110, about 75 to about 120, about 75 to about 130, about 80 to about 90, about 80 to about 100, about 80 to about 110, about 80 to about 120, about 80 to about 130, about 90 to about 100, about 90 to about 110, about 90 to about 120, about 90 to about 130, about 100 to about 110, about 100 to about 120, about 100 to about 130, about 110 to about 120, about 110 to about 130, or about 120 to about 130 nucleotides in length. In some embodiments, a guide RNA comprises a scaffold sequence of from about 30, about 40, about 50, about 60, about 70, about 75, about 80, about 90, about 100, about 110, about 120, or about 130 nucleotides in length. In some embodiments, a guide RNA comprises a scaffold sequence of from at least about 30, about 40, about 50, about 60, about 70, about 75, about 80, about 90, about 100, about 110, or about 120 nucleotides in length. In some embodiments, a guide RNA comprises a scaffold sequence of from at most about 40, about 50, about 60, about 70, about 75, about 80, about 90, about 100, about 110, about 120, or about 130 nucleotides in length.

In some embodiments, a guide RNA comprises a sequence comprising a stem loop structure. In some embodiments, a guide RNA comprises a sequence comprising a stem loop structure of from 8 to 35 nucleotides in length. In some embodiments, a guide RNA comprises a sequence comprising a stem loop structure that is 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length. In some embodiments, a guide RNA comprises a sequence comprising a stem structure of from 2 to 16 nucleotides in length. In some embodiments, a guide RNA comprises a sequence comprising a stem structure of 2, 3, 4, 5, 6, 7, 8, 9, 10, 11, 12, 13, 14, 15, or 16 nucleotides in length. In some embodiments, a guide RNA comprises a sequence comprising a loop structure of from 3 to 31 nucleotides in length. In some embodiments, a guide RNA comprises a sequence comprising a loop structure of 3, 4, 5, 6, 7, 8, 9, 10, 11, 12, 13, 14, 15, 16, 17, 18, 19, 20, 21, 22, 23, 24, 25, 26, 27, 28, 29, 30, or 31 nucleotides in length.

In some embodiments, the guide RNA comprises a crRNA. In some embodiments, the crRNA comprises one or more chemically modified nucleotides. In some embodiments, the chemically modified crRNA may be 30 to 50 nucleotides in length. In some embodiments, the chemically modified crRNA may be of 30, 31, 32, 33, 34, 35, 36, 37, 38, 39, 40, 41, 42, 43, 44, 45, 46, 47, 48, 49, or 50 nucleotides in length. In some embodiments, the chemically modified crRNA may be of from about 30 to about 50 nucleotides in length. In some embodiments, the chemically modified crRNA may be of from about 30 to about 32, about 30 to about 34, about 30 to about 36, about 30 to about 38, about 30 to about 40, about 30 to about 42, about 30 to about 44, about 30 to about 46, about 30 to about 48, about 30 to about 50, about 32 to about 34, about 32 to about 36, about 32 to about 38, about 32 to about 40, about 32 to about 42, about 32 to about 44, about 32 to about 46, about 32 to about 48, about 32 to about 50, about 34 to about 36, about 34 to about 38, about 34 to about 40, about 34 to about 42, about 34 to about 44, about 34 to about 46, about 34 to about 48, about 34 to about 50, about 36 to about 38, about 36 to about 40, about 36 to about 42, about 36 to about 44, about 36 to about 46, about 36 to about 48, about 36 to about 50, about 38 to about 40, about 38 to about 42, about 38 to about 44, about 38 to about 46, about 38 to about 48, about 38 to about 50, about 40 to about 42, about 40 to about 44, about 40 to about 46, about 40 to about 48, about 40 to about 50, about 42 to about 44, about 42 to about 46, about 42 to about 48, about 42 to about 50, about 44 to about 46, about 44 to about 48, about 44 to about 50, about 46 to about 48, about 46 to about 50, or about 48 to about 50 nucleotides in length. In some embodiments, the chemically modified crRNA may be of from about 30, about 32, about 34, about 36, about 38, about 40, about 42, about 44, about 46, about 48, or about 50 nucleotides in length. In some embodiments, the chemically modified crRNA may be of from at least about 30, about 32, about 34, about 36, about 38, about 40, about 42, about 44, about 46, or about 48 nucleotides in length. In some embodiments, the chemically modified crRNA may be of from at most about 32, about 34, about 36, about 38, about 40, about 42, about 44, about 46, about 48, or about 50 nucleotides in length.

In some embodiments, the guide RNA comprises a tracrRNA. In some embodiments, the tracrRNA comprises one or more chemically modified nucleotides. In some embodiments, the chemically modified tracrRNA comprises a total of from 50 to 130 nucleotides in length. In some embodiments, the chemically modified tracrRNA comprises a total of 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 nucleotides in length. In some embodiments, the chemically modified tracrRNA comprises a total of about 30 to about 130 nucleotides in length. In some embodiments, the chemically modified tracrRNA comprises a total of about 30 to about 40, about 30 to about 50, about 30 to about 60, about 30 to about 70, about 30 to about 75, about 30 to about 80, about 30 to about 90, about 30 to about 100, about 30 to about 110, about 30 to about 120, about 30 to about 130, about 40 to about 50, about 40 to about 60, about 40 to about 70, about 40 to about 75, about 40 to about 80, about 40 to about 90, about 40 to about 100, about 40 to about 110, about 40 to about 120, about 40 to about 130, about 50 to about 60, about 50 to about 70, about 50 to about 75, about 50 to about 80, about 50 to about 90, about 50 to about 100, about 50 to about 110, about 50 to about 120, about 50 to about 130, about 60 to about 70, about 60 to about 75, about 60 to about 80, about 60 to about 90, about 60 to about 100, about 60 to about 110, about 60 to about 120, about 60 to about 130, about 70 to about 75, about 70 to about 80, about 70 to about 90, about 70 to about 100, about 70 to about 110, about 70 to about 120, about 70 to about 130, about 75 to about 80, about 75 to about 90, about 75 to about 100, about 75 to about 110, about 75 to about 120, about 75 to about 130, about 80 to about 90, about 80 to about 100, about 80 to about 110, about 80 to about 120, about 80 to about 130, about 90 to about 100, about 90 to about 110, about 90 to about 120, about 90 to about 130, about 100 to about 110, about 100 to about 120, about 100 to about 130, about 110 to about 120, about 110 to about 130, or about 120 to about 130 nucleotides in length. In some embodiments, the chemically modified tracrRNA comprises a total of about 30, about 40, about 50, about 60, about 70, about 75, about 80, about 90, about 100, about 110, about 120, or about 130 nucleotides in length. In some embodiments, the chemically modified tracrRNA comprises a total of at least about 30, about 40, about 50, about 60, about 70, about 75, about 80, about 90, about 100, about 110, or about 120 nucleotides in length. In some embodiments, the chemically modified tracrRNA comprises a total of at most about 40, about 50, about 60, about 70, about 75, about 80, about 90, about 100, about 110, about 120, or about 130 nucleotides in length.

In one aspect, provided herein is a single guide RNA that comprises (i) a spacer sequence and (ii) a scaffold sequence, wherein the spacer sequence is designed to be complementary and hybridize with a targeted polynucleotide sequence in a gene of interest, and wherein the scaffold sequence is a substrate for a Cas12b protein to facilitate the formation of Cas12b protein-guide RNA complex, the ribonucleoprotein (RNP) complex. In some embodiments, the scaffold sequence is of about 95 to 105 nucleotide long and constitute the 5′-end of the single guide RNA, and the spacer sequence that constitute the 3′-end of the single guide RNA is about 20 to 40 nucleotide long. In some embodiments, the scaffold sequence is of about 97 nucleotide long and constitute the 5′-end of the single guide RNA, and the spacer sequence that constitute the 3′-end of the single guide RNA is about 20 to 40 nucleotide long. In some embodiments, the scaffold sequence is of about 97 nucleotide long and constitute the 5′-end of the single guide RNA, and the spacer sequence that constitute the 3′-end of the single guide RNA is 23 nucleotide long.

### Structure-Based Modification of Cas12b Guide RNAs

Also provided herein are chemical modifications to the guide RNAs described herein. In one aspect, the modifications are from a structurally based design that examines the interaction of the Cas12b protein and guide RNA, as exemplified FIG. 18, 19b and FIG. 19b. Nucleotide positions on the guide RNA are identified as targets for modification based on proximity to the nuclease and function.

In some embodiments, the structure of a guide RNA is maintained or modified based on the ribonucleoprotein (RNP)-sgRNA alignment. For example, in some embodiments, the guide RNA is designed to maintain or modify the stem-loop guide intramolecular interactions, e.g., the W-C base pairing at the stem. For another example, in some embodiments, the designed chemically modified guide RNA is designed to maintain or modify the loop nucleotide alignment with protein. In another example, in some embodiments, the designed chemically modified guide RNA is designed to maintain or modify the interaction of base (H-bond), 2′-hydroxyl(2′-OH), 4′-oxygen (ring-oxygen) of the sugar moiety, or phosphate linkage of each nucleotide to amino acid side chains of the protein. In another example, in some embodiments, the structure of the designed chemically modified guide RNA is maintained or modified based on the spatial arrangement of nucleotide within RNP, e.g., steric interaction, and room to accommodate bulky substitution like 2′-OMe and phosphorothioate (PS).

In some embodiments, an X-ray crystal approach is used for designing chemically modified Cas12b sgRNAs. For example, in some embodiments, selective 2′-hydroxyl substitution with 2′-O-methyl groups in Cas12b sgRNAs may improve the half-life of the sgRNA and sustain potency inside the cell. In some embodiments, modification of 2′-hydroxyl groups at sites where critical hydrogen bonds are formed between the sgRNA and the Cas12b protein is avoided, so as not to detriment RNP formation in the cytosol.

In some embodiments, a Cas12b protein is a Cas12b nuclease from Bacillus hisashii that contains 3 single mutations, e.g., K846R/S893R/E837G mutations that result in improved activity (see, e.g., Strecker et al. Nature communications 2019, 10(1): 212, of which entire contents are incorporated herein by reference).

In some embodiments, structure-based spacer and tracr designs are informed by the crystal structures of different Cas12b proteins in a ternary complex with gRNA and target or non-target DNAs. For example, in some embodiments, structure-based spacer and tracr designs are informed by the crystal structure of Bacillus thermoamylovorans Cas12b in a ternary complex with sgRNA and DNA (FIG. 19A) (see, e.g., Wu et al. Cell research 2017, 27(5):705 and Liang et al. Molecular cell 2017, 65(2):310-322, of which entire contents are incorporated herein by reference), which has 99% identity to the Bacillus hisashii Cas12b triple mutant construct having K846R/S893R/E837G mutations (FIG. 19B) (see, e.g., Wu et al. Cell research 2017, 27(5):705 and Strecker et al. Nature communications 2019, 10(1): 212, of which entire contents are incorporated herein by reference). By comparison, Alicyclobacillus acidoterrestris Cas12b has 33% to Bacillus hisashii Cas12b. In some embodiments, the crystal structure from B. thermoamylovorans is used as a model to determine where 2′ modifications would be tolerated in Bacillus hisashii Cas12b.

In some embodiments, any positions in the crystal structure where there appeared to be a hydrogen bond between the 2′OH of a given nucleotide and the Cas12b protein (or another part of the guide RNA) are left unmodified. In some embodiments, any positions where a clash was predicted to occur between a 2′-O-Me and the protein are left unmodified. In some embodiments, at sites where the 2′OH is solvent-exposed or otherwise distant from protein residues, a 2′-O-Me substitution is made. In some embodiments, the entire guide RNA is modified according to the aforementioned strategy. In some embodiments, only the tracr region of the guide RNA is modified according to the aforementioned strategy. In some embodiments, only the spacer region of the guide RNA is modified according to the aforementioned strategy. In some embodiments, specific stem loops of the tracr are left unmodified and it is identified whether the effect of incorporating 2′-O-Me substitutions in these structural regions is beneficial or not.

In some embodiments, the guide RNA is modified by the structure-based incorporation of 2′ modifications. In some embodiment, it is avoided to place 2′OMe modifications at sites that appear important for guide RNA structure or RNP formation. For example, in some embodiments, 2′-hydroxyls that do not form contacts may be converted to 2′-O-methyl groups to impart improved chemical stability. For another example, in some embodiments, 2′-hydroxyls that form contacts with protein or other regions of the gRNA may be left unmodified, since modification may have a negative impact on RNP formation and therefore nuclease activity. FIG. 18 shows exemplary structure-based incorporation of 2′ modifications.

In one aspect, the guide RNA comprises a chemical modification. In some embodiments, the chemical modifications include but not limited to sugar and nucleobase modifications and combinations thereof. In some embodiments, the guide RNA comprises a modification at a specific nucleotide position. In some embodiments, the guide RNA comprises one or more modifications at one or more specific positions.

In some embodiments, the chemical modification is in the spacer sequence of the RNA. In some embodiments, the chemical modification is in the scaffold sequence of the RNA. In some embodiments, the chemical modification is in a stem loop structure of the scaffold sequence. In some embodiments, the chemical modification is in the crRNA sequence of the guide RNA. In some embodiments, the chemical modification is in the tracrRNA sequence of the guide RNA.

In some embodiments, the chemical modification comprises a 2′-OMe modification. In some embodiments, the chemical modification comprises a nebularin. In some embodiments, the chemical modification comprises a deoxynebularin. In some embodiments, the chemical modification comprises a 2′-O-MOE modification. In some embodiments, the chemical modification comprises a 2′-F modification.

In some embodiments, the chemically modified guide RNA comprises modifications, such as, for example, 2′-O-methyl modifications, 2′-O-(2-methoxyethyl) modifications, 2′-fluoro modifications, phosphorothioate modifications, inverted abasic modifications, deoxyribonucleotides, bicylic ribose analog (e.g., locked nucleic acid (LNA), C-ethylene-bridged nucleic acid (ENA), bridged nucleic acid (BNA), unlocked nucleic acid (UNA)), base or nucleobase modifications, internucleoside linkage modifications, ribonebularine, 2′-O-methylnebularine, or 2′-deoxynebularine. Other examples of modifications include, but are not limited to, 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

In some embodiments, the chemically modified guide RNA comprises modification on the sugar group. In some embodiments, modified sugar group may control oligonucleotide binding affinity for complementary strands, duplex formation, or interaction with nucleases. Examples of chemical modifications to the sugar group include, but are not limited to, 2′-O-methyl(2′-OMe), 2′-fluoro (2′-F), 2′-deoxy, 2′-O-(2-methoxyethyl) (2′-MOE), 2′-NH2, 2′-O-Allyl, 2′-O-Ethylamine, 2′-O-Cyanoethyl, 2′-O-Acetalester, or a bicyclic nucleotide such as locked nucleic acid (LNA), 2′-(5-constrained ethyl (S-cEt)), constrained MOE, or 2′-0,4′-C-aminomethylene bridged nucleic acid (2′,4′-BNA^{NC}). In some embodiments, 2′-O-methyl modification can increase binding affinity of oligonucleotides. In some embodiments, 2′-O-methyl modification can enhance nuclease stability of oligonucleotides. In some embodiments, 2′-fluoro modification can increase oligonucleotide binding affinity and nuclease stability.

In some embodiments, the chemically modified guide RNA binds the Cas12b protein with increased binding affinity as compared to an unmodified single guide RNA. In some embodiments, the chemically modified single guide RNA binds the Cas12b protein with increased binding affinity by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified single guide RNA. In some embodiments, the chemically modified guide RNA binds the Cas12b protein with increased binding affinity by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the editing efficiency as compared to an unmodified guide RNA. In some embodiments, the chemically modified single guide RNA increases the editing efficiency by at least 5%, at least 10%, at least 20%, at least 30%, at least 40%, at least 50%, at least 60%, at least 70%, at least 80%, at least 90%, at least 100% or >100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified guide RNA. In some embodiments, the chemically modified single guide RNA increases editing efficiency by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the stability of the Cas12b-gRNA complex as compared to an unmodified single guide RNA. In some embodiments, the single guide RNA increases the stability of the Cas12b-gRNA complex by at least 5%, at least 10%, at least 20%, at least 30%, at least 40%, at least 50%, at least 60%, at least 70%, at least 80%, at least 90%, at least 100%, or higher at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified single guide RNA. In some embodiments, the chemically modified guide RNA increases the stability of the Cas12b-gRNA complex by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the stability of the guide RNA in vitro as compared to an unmodified guide RNA. In some embodiments, the chemically modified single guide RNA increases the stability of the guide RNA in vitro by at least 5%, at least 10%, at least 20%, at least 30%, at least 40%, at least 50%, at least 60%, at least 70%, at least 80%, at least 90%, at least 100% or >100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified single guide RNA. In some embodiments, the chemically modified guide RNA increases the stability of the guide RNA in vitro by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the stability of the guide RNA in vivo as compared to an unmodified guide RNA. In some embodiments, the chemically modified single guide RNA increases the stability of the guide RNA in vivo by at least 5%, at least 10%, at least 20%, at least 30%, at least 40%, at least 50%, at least 60%, at least 70%, at least 80%, at least 90%, at least 100% or >100% at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified guide RNA. In some embodiments, the chemically modified guide RNA increases the stability of the guide RNA in vivo by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the stability of the guide RNA ex vivo as compared to an unmodified guide RNA. In some embodiments, the chemically modified single RNA increases the stability of the guide RNA ex vivo by at least 5%, at least 10%, at least 20%, at least 30%, at least 40%, at least 50%, at least 60%, at least 70%, at least 80%, at least 90%, at least 100% or >100% at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified guide RNA. In some embodiments, the chemically modified guide RNA increases the stability of the guide RNA ex vivo by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold or >100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA increases the resistance to degradation in a cell as compared to an unmodified guide RNA. In some embodiments, the chemically modified guide RNA increases the resistance to degradation in a cell by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an unmodified guide RNA. In some embodiments, the modified guide RNA increases the resistance to degradation in a cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemical modification on the guide RNA results in less off-target effect in the cell as compared to an unmodified guide RNA. In some embodiments, the modification results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% less off-target effect in the cell as compared to an unmodified guide RNA. In some embodiments, the modification results in at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold less off-target effect in the cell as compared to an unmodified guide RNA.

In some embodiments, the chemically modified guide RNA exhibits increased stability in the cell compared to an unmodified guide RNA, wherein the stability is measured by half-life of the guide RNA in the cell. In some embodiments, the guide RNA exhibits increased stability in the cell by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% compared to an unmodified single guide RNA. In some embodiments, the single guide RNA exhibits increased stability in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, or higher compared to an unmodified single guide RNA.

In some embodiments, the chemically modified guide RNA exhibits increased half-life in the cell compared to an unmodified guide RNA. In some embodiments, the chemically modified guide RNA exhibits increased half-life in the cell by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% compared to an unmodified guide RNA. In some embodiments, the chemically modified guide RNA exhibits increased half-life in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an unmodified guide RNA.

In some embodiments, the chemically modified guide RNA comprises modification on the phosphate group. Examples of chemical modifications to the phosphate group includes, but are not limited to, a phosphorothioate (PS), phosphonoacetate (PACE), thiophosphonoacetate (thioPACE), amide, triazole, phosphonate, or phosphotriester modification. In some embodiments, PS linkage can refer to a bond where a sulfur is substituted for one bridging phosphate oxygen in a phosphodiester linkage, e.g., between nucleotides.

In some embodiments, the chemically modified guide RNA comprises a phosphorothioate (PS) linkage at a 5′ end or at a 3′ end. In some embodiments, a guide RNA comprises a phosphorothioate (PS) linkage at a 5′ end. In some embodiments, the chemically modified guide RNA comprises a phosphorothioate (PS) linkage at a 3′ end. In some embodiments, the chemically modified guide RNA comprises a phosphorothioate (PS) linkage at a 5′ end and at a 3′ end. In some embodiments, the chemically modified guide RNA comprises one, two, three, four or more than four phosphorothioate linkages at the 5′ end or at the 3′ end. In some embodiments, the chemically modified guide RNA comprises three phosphorothioate (PS) linkages at the 5′ end or at the 3′ end. In some embodiments, the chemically modified guide RNA comprises three phosphorothioate linkages at the 3′ end. In some embodiments, the chemically modified guide RNA comprises two and no more than two (i.e., only two) phosphorothioate (PS) linkages at the 5′ end or at the 3′ end. In some embodiments, the chemically modified guide RNA comprises two and no more than two (i.e., only two) contiguous phosphorothioate (PS) linkages at the 5′ end or at the 3′ end. In some embodiments, the chemically modified guide RNA comprises three contiguous phosphorothioate (PS) linkages at the 5′ end or at the 3′ end. In some embodiments, the chemically modified guide RNA comprises two and no more than two (i.e., only two) contiguous phosphorothioate (PS) linkages at the 5′ end and three contiguous phosphorothioate (PS) linkages at the 3′ end. In some embodiments, the chemically modified guide RNA comprises two and no more than two (i.e., only two) contiguous phosphorothioate (PS) linkages at the 3′ end and three contiguous phosphorothioate (PS) linkages at the 5′ end. In some embodiments, the chemically modified guide RNA comprises two or three phosphorothioate (PS) linkages after the first 3 nucleotides on the 5′ end or before the last three nucleotides on the 3′ end. In some embodiments, the chemically modified guide RNA comprises two or three contiguous phosphorothioate (PS) linkages after the first 3 nucleotides on the 5′ end or before the last three nucleotides on the 3′ end.

In some embodiments, the chemically modified guide RNA comprises modifications on the nucleobase. Examples of chemical modifications to the nucleobase include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

In some embodiments, chemically modified guide RNA comprises nebularine. In some embodiments, nebularine is a purine ribonucleoside that is derived from a beta-D-ribose and is 9H-purine attached to a beta-D-ribofuranosyl residue at position 9 via a glycosidic (N-glycosyl) linkage. In some embodiments, nebularine is a purine ribonucleoside with no exocyclic functional moiety or substitution. In some embodiments, nebularine is a purine ribonucleoside. In some embodiments, nebularine is a purine D-ribonucleoside. In some embodiments, nebularine is further modified chemically. In this application, “X,” “x,” and “dX” may be used to depict a ribonebularine modification, a 2′-O-methylnebularine modification, and a 2′-deoxynebularine modification, respectively, in guide RNA sequences (e.g., Table 1). In some embodiments, substitution of a nucleotide (e.g., A) of a guide RNA sequence (e.g., spacer region or tracr region) with nebularine may reduce off-target effects without affecting the guide RNA activity. In some embodiments, the nebularine, the deoxynebularine, or 2′-O-methylnebularine replaces an adenine in an unmodified guide RNA. In some embodiments, the nebularine, the deoxynebularine, or 2′-O-methylnebularine is in the spacer sequence. In some embodiments, the nebularine, the deoxynebularine, or 2′-O-methylnebularine is in the scaffold sequence. In some embodiments, the nebularine, the deoxynebularine, or 2′-O-methylnebularine is in a tracrRNA sequence in the scaffold sequence. In some embodiments, the nebularine, the deoxynebularine, or 2′-O-methylnebularine is in a crRNA sequence in the scaffold sequence. In some embodiments, the nebularine, the deoxynebularine, or 2′-O-methylnebularine is in a stem loop structure in the scaffold sequence.

In some embodiments, the modified guide RNA comprises from 1 to 150 chemically modified nucleotides. In some embodiments, a guide RNA comprises a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 chemically modified nucleotides. In some embodiments, the chemically modified guide RNA comprises a total of about 1 to about 45 chemically modified nucleotides. In some embodiments, the chemically modified guide RNA comprises a total of about 1 to about 3, about 1 to about 5, about 1 to about 10, about 1 to about 15, about 1 to about 20, about 1 to about 25, about 1 to about 30, about 1 to about 35, about 1 to about 40, about 1 to about 45, about 3 to about 5, about 3 to about 10, about 3 to about 15, about 3 to about 20, about 3 to about 25, about 3 to about 30, about 3 to about 35, about 3 to about 40, about 3 to about 45, about 5 to about 10, about 5 to about 15, about 5 to about 20, about 5 to about 25, about 5 to about 30, about 5 to about 35, about 5 to about 40, about 5 to about 45, about 10 to about 15, about 10 to about 20, about 10 to about 25, about 10 to about 30, about 10 to about 35, about 10 to about 40, about 10 to about 45, about 15 to about 20, about 15 to about 25, about 15 to about 30, about 15 to about 35, about 15 to about 40, about 15 to about 45, about 20 to about 25, about 20 to about 30, about 20 to about 35, about 20 to about 40, about 20 to about 45, about 25 to about 30, about 25 to about 35, about 25 to about 40, about 25 to about 45, about 30 to about 35, about 30 to about 40, about 30 to about 45, about 35 to about 40, about 35 to about 45, about 40 to about 45, or about 45 to about 50 chemically modified nucleotides. In some embodiments, the chemically modified guide RNA comprises a total of about 1, about 3, about 5, about 10, about 15, about 20, about 25, about 30, about 35, about 40, about 45, or about 50 chemically modified nucleotides. In some embodiments, the chemically modified guide RNA comprises a total of at least about 1, about 3, about 5, about 10, about 15, about 20, about 25, about 30, about 35, about 40, about 45, or about 50 chemically modified nucleotides. In some embodiments, the chemically modified guide RNA comprises a total of at most about 3, about 5, about 10, about 15, about 20, about 25, about 30, about 35, about 40, about 45, or about 50 chemically modified nucleotides. In some embodiments, the chemically modified guide RNA comprises a total of about 50 to about 140 chemically modified nucleotides. In some embodiments, the chemically modified guide RNA comprises a total of about 50 to about 60, about 50 to about 70, about 50 to about 80, about 50 to about 90, about 50 to about 100, about 50 to about 110, about 50 to about 120, about 50 to about 130, about 50 to about 140, about 60 to about 70, about 60 to about 80, about 60 to about 90, about 60 to about 100, about 60 to about 110, about 60 to about 120, about 60 to about 130, about 60 to about 140, about 70 to about 80, about 70 to about 90, about 70 to about 100, about 70 to about 110, about 70 to about 120, about 70 to about 130, about 70 to about 140, about 80 to about 90, about 80 to about 100, about 80 to about 110, about 80 to about 120, about 80 to about 130, about 80 to about 140, about 90 to about 100, about 90 to about 110, about 90 to about 120, about 90 to about 130, about 90 to about 140, about 100 to about 110, about 100 to about 120, about 100 to about 130, about 100 to about 140, about 110 to about 120, about 110 to about 130, about 110 to about 140, about 120 to about 130, about 120 to about 140, or about 130 to about 140 chemically modified nucleotides. In some embodiments, the chemically modified guide RNA comprises a total of about 50, about 60, about 70, about 80, about 90, about 100, about 110, about 120, about 130, or about 140 chemically modified nucleotides. In some embodiments, the chemically modified guide RNA comprises a total of at least about 50, about 60, about 70, about 80, about 90, about 100, about 110, about 120, or about 130 chemically modified nucleotides. In some embodiments, the chemically modified guide RNA comprises a total of at most about 60, about 70, about 80, about 90, about 100, about 110, about 120, about 130, or about 140 chemically modified nucleotides. In some embodiment, the chemically modified guide RNA comprises a total of 121, 122, 123, 124, 125, 126, 127, 128, or 129 chemically modified nucleotides. In another embodiment, the modified guide RNA comprises a total of 124 chemically modified nucleotides.

In some embodiments, the modified guide RNA comprises about 1% to about 100% chemically modified nucleotides. In some embodiments, the chemically modified guide RNA comprises about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 100%, or about 95% to about 100% chemically modified nucleotides. In some embodiments, the chemically modified nucleotide comprises about 1%, about 10%, about 20%, about 30%, about 40%, about 50%, about 60%, about 70%, about 80%, about 90%, about 95%, or about 100% chemically modified nucleotides. In some embodiments, the chemically modified guide RNA comprises at least about 1%, about 10%, about 20%, about 30%, about 40%, about 50%, about 60%, about 70%, about 80%, about 90%, or about 95% chemically modified nucleotides. In some embodiments, the chemically modified guide RNA comprises at most about 10%, about 20%, about 30%, about 40%, about 50%, about 60%, about 70%, about 80%, about 90%, about 95%, or about 100% chemically modified nucleotides.

In some embodiments, the chemically modified guide RNA has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 in the 5′-3′ direction in the guide RNA sequence.

In some embodiments, the chemically modified guide RNA has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149 nucleotides from the 5′ end of the guide RNA sequence.

In some embodiments, the chemically modified guide RNA has a chemically modified nucleotide on one or more the positions where are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149 nucleotides from the 3′ end of the guide RNA sequence.

In some embodiments, the chemically modified guide RNA has a one or more chemical modifications in the spacer region, i.e., the targeting or crRNA sequence. In some embodiments, the spacer sequence comprises a total of from 1 to 40 chemically modified nucleotides. In some embodiments, the spacer region comprises a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 chemically modified nucleotides. In some embodiments, the spacer sequence comprises a total of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 23, about 25, about 26, about 27, about 28, about 29 or about 30 chemically modified nucleotides. In some embodiments, the spacer sequence comprises a total of at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 23, about 25, about 26, about 27, about 28, about 29 or about 30 chemically modified nucleotides. In some embodiments, the spacer sequence comprises a total of at most about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 23, about 25, about 26, about 27, about 28, about 29 or about 30 chemically modified nucleotides. In some embodiments, the spacer sequence comprises a total of about 10 to about 30 chemically modified nucleotides. In some embodiments, the spacer sequence comprises a total of about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 chemically modified nucleotides. In some embodiments, the spacer sequence comprises a total of about 10, about 15, about 20, about 25, or about 30 chemically modified nucleotides. In some embodiments, the spacer sequence comprises a total of at least about 10, about 15, about 20, or about 25 chemically modified nucleotides. In some embodiments, the spacer sequence comprises a total of at most about 15, about 20, about 25, or about 30 chemically modified nucleotides. In one embodiment, the spacer sequence comprises a total of 27 chemically modified nucleotides. In another embodiment, the spacer sequence comprises a total of 13 chemically modified nucleotides.

In some embodiments, the spacer sequence comprises from 1% to 100% chemically modified nucleotides. In some embodiments, the spacer sequence comprises about 1% to about 100% chemically modified nucleotides. In some embodiments, the spacer sequence comprises about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 100%, or about 95% to about 100% chemically modified nucleotides. In some embodiments, the spacer sequence comprises about 1%, about 10%, about 20%, about 30%, about 40%, about 50%, about 60%, about 70%, about 80%, about 90%, about 95%, or about 100% chemically modified nucleotides. In some embodiments, the spacer sequence comprises at least about 1%, about 10%, about 20%, about 30%, about 40%, about 50%, about 60%, about 70%, about 80%, about 90%, or about 95% chemically modified nucleotides. In some embodiments, the spacer sequence comprises at most about 10%, about 20%, about 30%, about 40%, about 50%, about 60%, about 70%, about 80%, about 90%, about 95%, or about 100% chemically modified nucleotides.

In some embodiments, the chemically modified spacer sequence has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 in the 5′-3′ direction in the spacer sequence.

In some embodiments, the chemically modified spacer region has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 nucleotides from the 5′ end of the spacer sequence.

In some embodiments, the chemically modified spacer region has a chemically modified nucleotide on one or more the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 nucleotides from the 3′ end of the spacer sequence.

In some embodiments, the chemically modified guide RNA has a one or more chemical modifications in the scaffold sequence. In some embodiments, the scaffold sequence comprises a total of from 1 to 130 chemically modified nucleotides. In some embodiments, the chemically modified guide RNA comprises a scaffold sequence of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 chemically modified nucleotides. In some embodiments, the scaffold sequence comprises a total of from about 1 to about 50 chemically modified nucleotides. In some embodiments, the scaffold sequence comprises a total of from about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 1 to about 40, about 1 to about 35, about 1 to about 30, about 1 to about 25, about 1 to about 20, about 1 to about 15, about 1 to about 10, about 1 to about 5 chemically modified nucleotides. In some embodiments, the scaffold sequence comprises a total of from about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50 chemically modified nucleotides. In some embodiments, the scaffold sequence comprises a total of from at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50 chemically modified nucleotides. In some embodiments, the scaffold sequence comprises a total of from at most about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50 chemically modified nucleotides. In some embodiments, the scaffold sequence comprises a total of from about 10 to about 30 chemically modified nucleotides. In some embodiments, the scaffold sequence comprises a total of from about 10 to about 15, about 10 to about 20, about 10 to about 25, about 10 to about 30, about 10 to about 35, about 10 to about 40, about 15 to about 45, about 15 to about 20, about 15 to about 25, about 15 to about 30, about 15 to about 35, about 15 to about 40, about 15 to about 45, about 20 to about 25, about 20 to about 30, about 25 to about 30, about 25 to about 35, about 25 to about 40, about 25 to about 45, about 30 to about 35, about 30 to about 40, about 30 to about 45, about 35 to about 40, or about 35 to about 45 chemically modified nucleotides. In some embodiments, the scaffold sequence comprises a total of from about 10, about 15, about 20, about 25, about 30, about 35, about 40, or about 45 chemically modified nucleotides. In some embodiments, the scaffold sequence comprises a total of from at least about 10, about 15, about 20, or about 25 chemically modified nucleotides. In some embodiments, the spacer region comprises a total of at most about 15, about 20, about 25, about 30, about 35, about 40, or about 45 chemically modified nucleotides. In some embodiments, the scaffold sequence comprises a total of from about 30 to about 130 chemically modified nucleotides. In some embodiments, the scaffold sequence comprises a total of from about 30 to about 40, about 30 to about 50, about 30 to about 60, about 30 to about 70, about 30 to about 75, about 30 to about 80, about 30 to about 90, about 30 to about 100, about 30 to about 110, about 30 to about 120, about 30 to about 130, about 40 to about 50, about 40 to about 60, about 40 to about 70, about 40 to about 75, about 40 to about 80, about 40 to about 90, about 40 to about 100, about 40 to about 110, about 40 to about 120, about 40 to about 130, about 50 to about 60, about 50 to about 70, about 50 to about 75, about 50 to about 80, about 50 to about 90, about 50 to about 100, about 50 to about 110, about 50 to about 120, about 50 to about 130, about 60 to about 70, about 60 to about 75, about 60 to about 80, about 60 to about 90, about 60 to about 100, about 60 to about 110, about 60 to about 120, about 60 to about 130, about 70 to about 75, about 70 to about 80, about 70 to about 90, about 70 to about 100, about 70 to about 110, about 70 to about 120, about 70 to about 130, about 75 to about 80, about 75 to about 90, about 75 to about 100, about 75 to about 110, about 75 to about 120, about 75 to about 130, about 80 to about 90, about 80 to about 100, about 80 to about 110, about 80 to about 120, about 80 to about 130, about 90 to about 100, about 90 to about 110, about 90 to about 120, about 90 to about 130, about 100 to about 110, about 100 to about 120, about 100 to about 130, about 110 to about 120, about 110 to about 130, or about 120 to about 130 chemically modified nucleotides. In some embodiments, the scaffold sequence comprises a total of from about 30, about 40, about 50, about 60, about 70, about 75, about 80, about 90, about 100, about 110, about 120, or about 130 chemically modified nucleotides. In some embodiments, the scaffold sequence comprises a total of from at least about 30, about 40, about 50, about 60, about 70, about 75, about 80, about 90, about 100, about 110, or about 120 chemically modified nucleotides. In some embodiments, the scaffold sequence comprises a total of from at most about 40, about 50, about 60, about 70, about 75, about 80, about 90, about 100, about 110, about 120, or about 130 chemically modified nucleotides. In one embodiment, the scaffold sequence comprises a total of 60 chemically modified nucleotides. In another embodiment, the scaffold sequence comprises a total of 36 chemically modified nucleotides.

In some embodiments, the scaffold sequence comprises from 1% to 100% chemically modified nucleotides. In some embodiments, the scaffold sequence comprises about 1% to about 100% chemically modified nucleotides. In some embodiments, the scaffold sequence comprises about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 100%, or about 95% to about 100% chemically modified nucleotides. In some embodiments, the scaffold sequence comprises about 1%, about 10%, about 20%, about 30%, about 40%, about 50%, about 60%, about 70%, about 80%, about 90%, about 95%, or about 100% chemically modified nucleotides. In some embodiments, the scaffold sequence comprises at least about 1%, about 10%, about 20%, about 30%, about 40%, about 50%, about 60%, about 70%, about 80%, about 90%, or about 95% chemically modified nucleotides. In some embodiments, the scaffold sequence comprises at most about 10%, about 20%, about 30%, about 40%, about 50%, about 60%, about 70%, about 80%, about 90%, about 95%, or about 100% chemically modified nucleotides.

In some embodiments, the chemically modified scaffold sequence has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 in the 5′-3′ direction in the scaffold sequence.

In some embodiments, the chemically modified scaffold region has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 nucleotides from the 5′ end of the scaffold sequence.

In some embodiments, the chemically modified scaffold region has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 nucleotides from the 3′ end of the scaffold sequence.

In some embodiments, the modified crRNA comprises a total of from 1 to 50 chemically modified nucleotides. In some embodiments, the chemically modified crRNA comprises a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 chemically modified nucleotides. In some embodiments, the chemically modified crRNA comprises a total of about 1 to about 10 chemically modified nucleotides. In some embodiments, the chemically modified crRNA comprises a total of about 1 to about 2, about 1 to about 3, about 1 to about 4, about 1 to about 5, about 1 to about 6, about 1 to about 7, about 1 to about 8, about 1 to about 9, about 1 to about 10, about 2 to about 3, about 2 to about 4, about 2 to about 5, about 2 to about 6, about 2 to about 7, about 2 to about 8, about 2 to about 9, about 2 to about 10, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10, or about 9 to about 10 chemically modified nucleotides. In some embodiments, the chemically modified crRNA comprises a total of about 1, about 2, about 3, about 4, about 5, about 6, about 7, about 8, about 9, or about 10 chemically modified nucleotides. In some embodiments, the chemically modified crRNA comprises a total of at least about 1, about 2, about 3, about 4, about 5, about 6, about 7, about 8, or about 9 chemically modified nucleotides. In some embodiments, the chemically modified crRNA comprises a total of at most about 2, about 3, about 4, about 5, about 6, about 7, about 8, about 9, or about 10 chemically modified nucleotides. In some embodiments, the chemically modified crRNA comprises a total of about 10 to about 30 chemically modified nucleotides. In some embodiments, the chemically modified crRNA comprises a total of about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 chemically modified nucleotides. In some embodiments, the chemically modified crRNA comprises a total of about 10, about 15, about 20, about 25, or about 30 chemically modified nucleotides. In some embodiments, the chemically modified crRNA comprises a total of at least about 10, about 15, about 20, or about 25 chemically modified nucleotides. In some embodiments, the chemically modified crRNA comprises a total of at most about 15, about 20, about 25, or about 30 chemically modified nucleotides. In some embodiments, the chemically modified crRNA comprises a total of from about 30 to about 50 chemically modified nucleotides. In some embodiments, the chemically modified crRNA comprises a total of from about 30 to about 32, about 30 to about 34, about 30 to about 36, about 30 to about 38, about 30 to about 40, about 30 to about 42, about 30 to about 44, about 30 to about 46, about 30 to about 48, about 30 to about 50, about 32 to about 34, about 32 to about 36, about 32 to about 38, about 32 to about 40, about 32 to about 42, about 32 to about 44, about 32 to about 46, about 32 to about 48, about 32 to about 50, about 34 to about 36, about 34 to about 38, about 34 to about 40, about 34 to about 42, about 34 to about 44, about 34 to about 46, about 34 to about 48, about 34 to about 50, about 36 to about 38, about 36 to about 40, about 36 to about 42, about 36 to about 44, about 36 to about 46, about 36 to about 48, about 36 to about 50, about 38 to about 40, about 38 to about 42, about 38 to about 44, about 38 to about 46, about 38 to about 48, about 38 to about 50, about 40 to about 42, about 40 to about 44, about 40 to about 46, about 40 to about 48, about 40 to about 50, about 42 to about 44, about 42 to about 46, about 42 to about 48, about 42 to about 50, about 44 to about 46, about 44 to about 48, about 44 to about 50, about 46 to about 48, about 46 to about 50, or about 48 to about 50 chemically modified nucleotides. In some embodiments, the chemically modified crRNA comprises a total of from about 30, about 32, about 34, about 36, about 38, about 40, about 42, about 44, about 46, about 48, or about 50 chemically modified nucleotides. In some embodiments, the chemically modified crRNA comprises a total of from at least about 30, about 32, about 34, about 36, about 38, about 40, about 42, about 44, about 46, or about 48 chemically modified nucleotides. In some embodiments, the chemically modified crRNA comprises a total of from at most about 32, about 34, about 36, about 38, about 40, about 42, about 44, about 46, about 48, or about 50 chemically modified nucleotides.

In some embodiments, the modified crRNA comprises from 1% to 100% chemically modified nucleotides. In some embodiments, the crRNA comprises about 1% to about 100% chemically modified nucleotides. In some embodiments, the crRNA comprises about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 100%, or about 95% to about 100% chemically modified nucleotides. In some embodiments, the crRNA comprises about 1%, about 10%, about 20%, about 30%, about 40%, about 50%, about 60%, about 70%, about 80%, about 90%, about 95%, or about 100% chemically modified nucleotides. In some embodiments, the crRNA comprises at least about 1%, about 10%, about 20%, about 30%, about 40%, about 50%, about 60%, about 70%, about 80%, about 90%, or about 95% chemically modified nucleotides. In some embodiments, the crRNA comprises at most about 10%, about 20%, about 30%, about 40%, about 50%, about 60%, about 70%, about 80%, about 90%, about 95%, or about 100% chemically modified nucleotides.

In some embodiments, the chemically modified crRNA has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 in the 5′-3′ direction in the crRNA sequence.

In some embodiments, the chemically modified crRNA has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides from the 5′ end of the crRNA sequence.

In some embodiments, the chemically modified crRNA has a chemically modified nucleotide on one or more of the positions which are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides from the 3′ end of the crRNA sequence.

In some embodiments, the chemically modified tracrRNA comprises a total of from 1 to 130 chemically modified nucleotides. In some embodiments, the chemically modified tracrRNA comprises a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 chemically modified nucleotides. In some embodiments, the chemically modified tracrRNA comprises a total of about 1 to about 10 chemically modified nucleotides. In some embodiments, the chemically modified tracrRNA comprises a total of about 1 to about 2, about 1 to about 3, about 1 to about 4, about 1 to about 5, about 1 to about 6, about 1 to about 7, about 1 to about 8, about 1 to about 9, about 1 to about 10, about 2 to about 3, about 2 to about 4, about 2 to about 5, about 2 to about 6, about 2 to about 7, about 2 to about 8, about 2 to about 9, about 2 to about 10, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10, or about 9 to about 10 chemically modified nucleotides. In some embodiments, the chemically modified tracrRNA comprises a total of about 1, about 2, about 3, about 4, about 5, about 6, about 7, about 8, about 9, or about 10 chemically modified nucleotides. In some embodiments, the chemically modified tracrRNA comprises a total of at least about 1, about 2, about 3, about 4, about 5, about 6, about 7, about 8, or about 9 chemically modified nucleotides. In some embodiments, the chemically modified tracrRNA comprises a total of at most about 2, about 3, about 4, about 5, about 6, about 7, about 8, about 9, or about 10 chemically modified nucleotides. In some embodiments, the chemically modified tracrRNA comprises a total of about 10 to about 30 chemically modified nucleotides. In some embodiments, the chemically modified tracrRNA comprises a total of about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 chemically modified nucleotides. In some embodiments, the chemically modified tracrRNA comprises a total of about 10, about 15, about 20, about 25, or about 30 chemically modified nucleotides. In some embodiments, the chemically modified tracrRNA comprises a total of at least about 10, about 15, about 20, or about 25 chemically modified nucleotides. In some embodiments, the spacer region comprises a total of at most about 15, about 20, about 25, or about 30 chemically modified nucleotides. In some embodiments, the chemically modified tracrRNA comprises a total of about 30 to about 130 chemically modified nucleotides. In some embodiments, the chemically modified tracrRNA comprises a total of about 30 to about 40, about 30 to about 50, about 30 to about 60, about 30 to about 70, about 30 to about 75, about 30 to about 80, about 30 to about 90, about 30 to about 100, about 30 to about 110, about 30 to about 120, about 30 to about 130, about 40 to about 50, about 40 to about 60, about 40 to about 70, about 40 to about 75, about 40 to about 80, about 40 to about 90, about 40 to about 100, about 40 to about 110, about 40 to about 120, about 40 to about 130, about 50 to about 60, about 50 to about 70, about 50 to about 75, about 50 to about 80, about 50 to about 90, about 50 to about 100, about 50 to about 110, about 50 to about 120, about 50 to about 130, about 60 to about 70, about 60 to about 75, about 60 to about 80, about 60 to about 90, about 60 to about 100, about 60 to about 110, about 60 to about 120, about 60 to about 130, about 70 to about 75, about 70 to about 80, about 70 to about 90, about 70 to about 100, about 70 to about 110, about 70 to about 120, about 70 to about 130, about 75 to about 80, about 75 to about 90, about 75 to about 100, about 75 to about 110, about 75 to about 120, about 75 to about 130, about 80 to about 90, about 80 to about 100, about 80 to about 110, about 80 to about 120, about 80 to about 130, about 90 to about 100, about 90 to about 110, about 90 to about 120, about 90 to about 130, about 100 to about 110, about 100 to about 120, about 100 to about 130, about 110 to about 120, about 110 to about 130, or about 120 to about 130 chemically modified nucleotides. In some embodiments, the chemically modified tracrRNA comprises a total of about 30, about 40, about 50, about 60, about 70, about 75, about 80, about 90, about 100, about 110, about 120, or about 130 chemically modified nucleotides. In some embodiments, the chemically modified tracrRNA comprises a total of at least about 30, about 40, about 50, about 60, about 70, about 75, about 80, about 90, about 100, about 110, or about 120 chemically modified nucleotides. In some embodiments, the chemically modified tracrRNA comprises a total of at most about 40, about 50, about 60, about 70, about 75, about 80, about 90, about 100, about 110, about 120, or about 130 chemically modified nucleotides.

In some embodiments, the chemically modified tracrRNA comprises about 1% to about 100% chemically modified nucleotides. In some embodiments, the chemically modified tracrRNA comprises about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 100%, or about 95% to about 100% chemically modified nucleotides. In some embodiments, the chemically modified tracrRNA comprises about 1%, about 10%, about 20%, about 30%, about 40%, about 50%, about 60%, about 70%, about 80%, about 90%, about 95%, or about 100% chemically modified nucleotides. In some embodiments, the chemically modified tracrRNA comprises at least about 1%, about 10%, about 20%, about 30%, about 40%, about 50%, about 60%, about 70%, about 80%, about 90%, or about 95% chemically modified nucleotides. In some embodiments, the chemically modified tracrRNA comprises at most about 10%, about 20%, about 30%, about 40%, about 50%, about 60%, about 70%, about 80%, about 90%, about 95%, or about 100% chemically modified nucleotides.

In some embodiments, the chemically modified tracrRNA has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 in the 5′-3′ direction in the tracrRNA sequence.

In some embodiments, the chemically modified tracrRNA has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 nucleotides from the 5′ end of the tracrRNA sequence.

In some embodiments, the chemically modified tracrRNA has a chemically modified nucleotide on one or more of the positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 nucleotides from the 3′ end of the tracrRNA sequence.

In some embodiments, the chemically modified guide RNA comprises phosphorothioate (PS) linkages at the 5′ or 3′ end of the gRNA sequence. In some embodiments, the chemically modified guide RNA comprises 0, 1, 2, 3, 4, or 5 PS linkages at the 5′ end of the guide RNA sequence. In some embodiments, the chemically modified guide RNA comprises 0, 1, 2, 3, 4, or 5 PS linkages at the 3′ end of the guide RNA sequence. In some embodiments, the chemically modified guide RNA comprises 0, 1, 2, 3, 4, or 5 PS linkages, or any combinations thereof at each of 5′ and 3′ end of the guide RNA sequence. In some embodiments, the chemically modified guide RNA comprises 0, 1, 2 or 3 PS linkages, or any combinations thereof at each of 5′ and 3′ end of the guide RNA sequence. In some embodiments, the chemically modified guide RNA comprises 1 PS linkage at each of 5′ and 3′ end of the guide RNA sequence. In some embodiments, the chemically modified guide RNA comprises 2 PS linkages at each of 5′ and 3′ end of the guide RNA sequence. In some embodiments, the chemically modified guide RNA comprises 3 PS linkages at each of 5′ and 3′ end of the guide RNA sequence. In some embodiments, the chemically modified guide RNA comprises 4 PS linkages at each of 5′ and 3′ end of the guide RNA sequence. In some embodiments, the chemically modified guide RNA comprises 5 PS linkages at each of 5′ and 3′ end of the guide RNA sequence. In some embodiments, the chemically modified guide RNA comprises 3 PS linkages at the 5′ end and 0 PS linkage (i.e., no modification) at the 3′ end of the guide RNA sequence. In some embodiments, the chemically modified guide RNA comprises 3 PS linkages at the 5′ end and 1 PS linkage at the 3′ end of the guide RNA sequence. In some embodiments, the chemically modified guide RNA comprises 3 PS linkages at the 5′ end and 2 PS linkages at the 3′ end of the guide RNA sequence. In some embodiments, the chemically modified guide RNA comprises 3 PS linkages at the 5′ end and 4 PS linkages at the 3′ end of the guide RNA sequence. In some embodiments, the chemically modified guide RNA comprises 3 PS linkages at the 5′ end and 5 PS linkages at the 3′ end of the guide RNA sequence. In some embodiments, the chemically modified guide RNA comprises 2 PS linkages at the 5′ end and 0 PS linkage (i.e., no modification) at the 3′ end of the guide RNA sequence. In some embodiments, the chemically modified guide RNA comprises 2 PS linkages at the 5′ end and 1 PS linkage at the 3′ end of the guide RNA sequence. In some embodiments, the chemically modified guide RNA comprises 2 PS linkages at the 5′ end and 3 PS linkages at the 3′ end of the guide RNA sequence. In some embodiments, the chemically modified guide RNA comprises 2 PS linkages at the 5′ end and 4 PS linkages at the 3′ end of the guide RNA sequence. In some embodiments, the chemically modified guide RNA comprises 2 PS linkages at the 5′ end and 5 PS linkages at the 3′ end of the guide RNA sequence.

In some embodiments, the chemically modified guide RNA comprises 2 and no more than 2 contiguous phosphorothioate (PS) linkages at the 5′ end. In some embodiments, the chemically modified guide RNA comprises 2 and no more than 2 contiguous phosphorothioate (PS) linkages at the 3′ end. In some embodiments, the chemically modified guide RNA comprises 3 contiguous phosphorothioate (PS) linkages at the 5′ end. In some embodiments, the chemically modified guide RNA comprises 3 contiguous phosphorothioate (PS) linkages at the 3′ end. In some embodiments, the chemically modified guide RNA comprises the sequence 5′-UsUsU-3′ at the 3′end or at the 5′ end, wherein U indicates a uridine and wherein s indicates a phosphorothioate (PS) linkage. In some embodiments, the chemically modified guide RNA comprises 3 phosphorothioate (PS) linkages at the 5′ end. In some embodiments, the chemically modified guide RNA comprises 3 phosphorothioate (PS) linkages at the 3′ end. In some embodiments, the chemically modified guide RNA comprises the two phosphorothioate linkages at the 5′ end, wherein the two phosphorothioate (PS) linkages are two contiguous phosphorothioate (PS) linkages at the first two nucleotide positions of the 5′ end. In some embodiments, the chemically modified guide RNA comprises the two phosphorothioate linkages at the 5′ end, wherein the two phosphorothioate (PS) linkages are within the first 3-10 nucleotides of the 5′ end. In some embodiments, the chemically modified guide RNA comprises the two phosphorothioate (PS) linkages at the 3′ end, wherein the two phosphorothioate (PS) linkages are two contiguous phosphorothioate (PS) linkages at the first two nucleotide positions of the 3′ end. In some embodiments, the chemically modified guide RNA comprises the two phosphorothioate (PS) linkages at the 3′ end, wherein the two phosphorothioate (PS) linkages are within the first 3-10 nucleotides of the 3′ end. In some embodiments, the chemically modified guide RNA comprises the sequence 5′-UsUsUs-3′ at the 3′ end, wherein U indicates a uridine and s indicates a phosphorothioate (PS) linkage. In some embodiments, the chemically modified guide RNA comprises the sequence 5′-UsUsU-3′ at the 3′ end, wherein U indicates a uridine and s indicates a phosphorothioate (PS) linkage.

In some embodiments, the chemically modified guide RNA comprises PS linkages at the internal positions of the guide RNA sequence. In some embodiments, the chemically modified guide RNA comprises 1, 2, 3, 4, 5, 6, 7, 8, 9, or 10 PS linkages at the internal positions of the guide RNA sequence. In some embodiments, the chemically modified guide RNA comprises 1 PS linkage at the internal position of the guide RNA sequence. In some embodiments, the chemically modified guide RNA comprises 2 PS linkages at the internal positions of the guide RNA sequence. In some embodiments, the chemically modified guide RNA comprises 3 PS linkages at the internal positions of the guide RNA sequence. In some embodiments, the chemically modified guide RNA comprises 4 PS linkages at the internal positions of the guide RNA sequence. In some embodiments, the chemically modified guide RNA comprises 5 PS linkages at the internal positions of the guide RNA sequence. In some embodiments, the chemically modified guide RNA comprises 6 PS linkages at the internal positions of the guide RNA sequence. In some chemically modified embodiments, the guide RNA comprises 7 PS linkages at the internal positions of the guide RNA sequence. In some embodiments, the chemically modified guide RNA comprises 8 PS linkages at the internal positions of the guide RNA sequence. In some embodiments, the chemically modified guide RNA comprises 9 PS linkages at the internal positions of the guide RNA sequence. In some embodiments, the chemically modified the guide RNA comprises 10 PS linkages at the internal positions of the guide RNA sequence.

In some embodiments, the chemically modified guide RNA comprises PS linkages at the 5′ end, the 3′ end, or at the internal positions, or any combination thereof, of the guide RNA sequence. In some embodiments, the chemically modified guide RNA comprises 0, 1, 2, 3, 4, 5, 6, 7, 8, 9, or 10 PS linkages at the 5′end, the 3′ end, or at the internal positions, or any combination thereof, of the guide RNA sequence.

In some embodiments, the chemically modified guide RNA comprises 2 PS linkages at the 5′ end, 3 PS linkages at the 3′ end, and 0 PS linkage (i.e., no modification) at the internal position of the guide RNA sequence. In some embodiments, the chemically modified guide RNA comprises 2 PS linkages at the 5′ end, 3 PS linkages at the 3′ end, and 1 PS linkage at the internal position of the guide RNA sequence. In some embodiments, the chemically modified guide RNA comprises 2 PS linkages at the 5′ end, 3 PS linkages at the 3′ end, and 2 PS linkages at the internal positions of the guide RNA sequence. In some embodiments, the chemically modified guide RNA comprises 2 PS linkages at the 5′ end, 3 PS linkages at the 3′ end, and 3 PS linkages at the internal positions of the guide RNA sequence. In some embodiments, the chemically modified guide RNA comprises 2 PS linkages at the 5′ end, 3 PS linkages at the 3′ end, and 4 PS linkages at the internal positions of the guide RNA sequence. In some embodiments, the chemically modified guide RNA comprises 2 PS linkages at the 5′ end, 3 PS linkages at the 3′ end, and 5 PS linkages at the internal positions of the guide RNA sequence. In some embodiments, the chemically modified guide RNA comprises 2 PS linkages at the 5′ end, 3 PS linkages at the 3′ end, and 6 PS linkages at the internal positions of the guide RNA sequence.

In some embodiments, the chemically modified guide RNA comprises 3 PS linkages at the 5′ end, 2 PS linkages at the 3′ end, and 0 PS linkage (i.e., no modification) at the internal position of the guide RNA sequence. In some embodiments, the chemically modified guide RNA comprises 3 PS linkages at the 5′ end, 2 PS linkages at the 3′ end, and 1 PS linkage at the internal position of the guide RNA sequence. In some embodiments, the chemically modified guide RNA comprises 3 PS linkages at the 5′ end, 2 PS linkages at the 3′ end, and 2 PS linkages at the internal positions of the guide RNA sequence. In some embodiments, the chemically modified guide RNA comprises 3 PS linkages at the 5′ end, 2 PS linkages at the 3′ end, and 3 PS linkages at the internal positions of the guide RNA sequence. In some embodiments, the chemically modified guide RNA comprises 3 PS linkages at the 5′ end, 2 PS linkages at the 3′ end, and 4 PS linkages at the internal positions of the guide RNA sequence. In some embodiments, the chemically modified guide RNA comprises 3 PS linkages at the 5′ end, 2 PS linkages at the 3′ end, and 5 PS linkages at the internal positions of the guide RNA sequence. In some embodiments, the chemically modified guide RNA comprises 3 PS linkages at the 5′ end, 2 PS linkages at the 3′ end, and 6 PS linkages at the internal positions of the guide RNA sequence.

In some embodiments, the chemically modified guide RNA comprises 2 PS linkages at the 5′ end, 2 PS linkages at the 3′ end, and 0 PS linkage (i.e., no PS backbone modification) at the internal position of the guide RNA sequence. In some embodiments, the chemically modified guide RNA comprises 2 PS linkages at the 5′ end and 2 PS linkages at the 3′ end and 1 PS linkage at the internal position of the guide RNA sequence. In some embodiments, the chemically modified guide RNA comprises 2 PS linkages at the 5′ end and 2 PS linkages at the 3′ end and 2 PS linkages at the internal positions of the guide RNA sequence. In some embodiments, the chemically modified guide RNA comprises 2 PS linkages at the 5′ end and 2 PS linkages at the 3′ end and 3 PS linkages at the internal positions of the guide RNA sequence. In some embodiments, the chemically modified guide RNA comprises 2 PS linkages at the 5′ end and 2 PS linkages at the 3′ end and 4 PS linkages at the internal positions of the guide RNA sequence. In some embodiments, the chemically modified guide RNA comprises 2 PS linkages at the 5′ end and 2 PS linkages at the 3′ end and 5 PS linkages at the internal positions of the guide RNA sequence. In some embodiments, the chemically modified guide RNA comprises 2 PS linkages at the 5′ end and 2 PS linkages at the 3′ end and 6 PS linkages at the internal positions of the guide RNA sequence.

In some embodiments, the chemically modified guide RNA comprises 3 PS linkages at the 5′ end, 3 PS linkages at the 3′ end, and 0 PS linkage (i.e., no modification) at the internal position of the guide RNA sequence. In some embodiments, the chemically modified guide RNA comprises 3 PS linkages at the 5′ end and 3 PS linkages at the 3′ end and 1 PS linkage at the internal position of the guide RNA sequence. In some embodiments, the chemically modified guide RNA comprises 3 PS linkages at the 5′ end and 3 PS linkages at the 3′ end and 2 PS linkages at the internal positions of the guide RNA sequence. In some embodiments, the chemically modified guide RNA comprises 3 PS linkages at the 5′ end and 3 PS linkages at the 3′ end and 3 PS linkages at the internal positions of the guide RNA sequence. In some embodiments, the chemically modified guide RNA comprises 3 PS linkages at the 5′ end and 3 PS linkages at the 3′ end and 4 PS linkages at the internal positions of the guide RNA sequence. In some embodiments, the chemically modified guide RNA comprises 3 PS linkages at the 5′ end and 3 PS linkages at the 3′ end and 5 PS linkages at the internal positions of the guide RNA sequence. In some embodiments, the chemically modified guide RNA comprises 3 PS linkages at the 5′ end and 3 PS linkages at the 3′ end and 6 PS linkages at the internal positions of the guide RNA sequence.

In some embodiments, the chemically modified guide RNA comprises additional modified or unmodified nucleotide (N) with phosphodiester linkage, wherein N is an A, C, G, U, dA (deoxyA), dC (deoxyC), dG (deoxyG), or T, and any combinations thereof. In some embodiments, the guide RNA comprises 1, 2, 3, 4, or 5 additional N with phosphodiester linkage, wherein Nis an A, G, U, dA, dG, dC, or T, and any combinations thereof. In some embodiments, the chemically modified guide RNA comprises 1, 2, 3, 4, or 5 additional N with phosphodiester linkage at the 5′ or 3′ end. In one embodiment, the chemically modified guide RNA comprises 1, 2, 3, 4, or 5 additional N with phosphodiester linkage at the 5′ end. In a preferred embodiment, the guide RNA comprises 1, 2, 3, 4, or 5 additional N with phosphodiester linkage at the 3′ end. In some embodiments, the chemically modified guide RNA comprises 1, 2, 3, 4, or 5 additional N with phosphodiester linkage and each N is the same modified or unmodified nucleotide. For example, the chemically modified guide RNA comprises 4 additional N with phosphodiester linkage and each N of the 4 additional N is A, C, G, U, dA, dC, dG, or T. For example, the guide RNA comprises 4 additional N with phosphodiester linkage and each N of the 4 additional Nis A. For example, the chemically modified guide RNA comprises 3 additional N with phosphodiester linkage and each N of the 3 additional N is A, C, G, U, dA, dC, dG, or T. For example, the chemically modified guide RNA comprises 3 additional N with phosphodiester linkage and each N of the 3 additional N is A. For example, the chemically modified guide RNA comprises 3 additional N with phosphodiester linkage and each N of the 3 additional Nis C. For example, the chemically modified guide RNA comprises 3 additional N with phosphodiester linkage and each N of the 3 additional N is G. For example, the chemically modified guide RNA comprises 3 additional N with phosphodiester linkage and each N of the 3 additional Nis U. For example, the guide RNA comprises 3 additional N with phosphodiester linkage and each N of the 3 additional N is dA. For example, the chemically modified guide RNA comprises 3 additional N with phosphodiester linkage and each N of the 3 additional N is dC. For example, the chemically modified guide RNA comprises 3 additional N with phosphodiester linkage and each N of the 3 additional N is dG. For example, the guide RNA comprises 3 additional N with phosphodiester linkage and each N of the 3 additional N is T.

In some embodiments, the chemically modified guide RNA comprises additional Uracil (U) with phosphodiester linkage at the 5′ or 3′ end. In one embodiment, the chemically modified guide RNA comprises additional U with phosphodiester linkage at the 5′ end. In a preferred embodiment, the chemically modified guide RNA comprises additional U with phosphodiester linkage at the 3′ end. In some embodiments, the chemically modified guide RNA comprises 1, 2, 3, 4, or 5 additional U with phosphodiester linkage at the 5′ or 3′ end. In some embodiments, the chemically modified guide RNA comprises 1 additional U with phosphodiester linkage at the 5′ end. In some embodiments, the chemically modified guide RNA comprises 1 additional U with phosphodiester linkage at the 3′ end. In some embodiments, the guide RNA comprises 2 additional U with phosphodiester linkage at the 5′ end. In some embodiments, the chemically modified guide RNA comprises 2 additional U with phosphodiester linkage at the 3′ end. In some embodiments, the chemically modified guide RNA comprises 3 additional U with phosphodiester linkage at the 5′ end. In a preferred embodiment, the guide RNA comprises 3 additional U with phosphodiester linkage at the 3′ end. In some embodiments, the chemically modified guide RNA comprises 4 additional U with phosphodiester linkage at the 5′ end. In some embodiments, the guide RNA comprises 4 additional U with phosphodiester linkage at the 3′ end. In some embodiments, the chemically modified guide RNA comprises 5 additional U with phosphodiester linkage at the 5′ end. In some embodiments, the chemically modified guide RNA comprises 5 additional U with phosphodiester linkage at the 3′ end.

In some embodiments, the chemically modified guide RNA comprises a ribonebularine (depicted as “X” in this application, e.g., in Table 1). In some embodiments, the chemically modified guide RNA comprises 0, 1, 2, 3, 4, 5, 6, 7, 8, 9, or 10 ribonebularines. In some embodiments, the chemically modified guide RNA does not comprise ribonebularine. In some embodiments, the chemically modified guide RNA comprises 1 ribonebularine. In some embodiments, the chemically modified guide RNA comprises 2 ribonebularines. In some embodiments, the guide RNA comprises 3 ribonebularines. In some embodiments, the chemically modified guide RNA comprises 4 ribonebularines. In some embodiments, the chemically modified guide RNA comprises 5 ribonebularines. In some embodiments, the g chemically modified guide polynucleotide comprises 6 ribonebularines. In some embodiments, the chemically modified guide RNA comprises 7 ribonebularines. In some embodiments, the chemically modified guide RNA comprises 8 ribonebularines. In some embodiments, the chemically modified guide RNA comprises 9 ribonebularines. In some embodiments, the chemically modified guide RNA comprises 10 ribonebularines. In some embodiments, the nebularine replaces an adenine in an unmodified guide RNA. In some embodiments, the nebularine is in the spacer sequence. In some embodiments, the nebularine is in a tracrRNA sequence in the scaffold sequence. In some embodiments, the nebularine is in a crRNA sequence in the scaffold sequence. In some embodiments, the nebularine is in a stem loop structure in the scaffold sequence.

In some embodiments, the chemically modified guide RNA comprises a 2′-O-methylnebularine (depicted as “x” in this application, e.g., in Table 1). In some embodiments, the chemically modified guide RNA comprises 0, 1, 2, 3, 4, 5, 6, 7, 8, 9, or 10 2′-O-methylnebularines. In some embodiments, the chemically modified guide RNA does not comprise 2′-O-methylnebularine. In some embodiments, the chemically modified guide RNA comprises 1 2′-O-methylnebularine. In some embodiments, the chemically modified guide RNA comprises 2 2′-O-methylnebularines. In some embodiments, the chemically modified guide RNA comprises 3 2′-O-methylnebularines. In some embodiments, the chemically modified guide RNA comprises 4 2′-O-methylnebularines. In some embodiments, the chemically modified guide RNA comprises 5 2′-O-methylnebularines. In some embodiments, the chemically modified guide RNA comprises 6 2′-O-methylnebularines. In some embodiments, the chemically modified guide RNA comprises 7 2′-O-methylnebularines. In some embodiments, the chemically modified guide RNA comprises 8 2′-O-methylnebularines. In some embodiments, the chemically modified guide RNA comprises 9 2′-O-methylnebularines. In some embodiments, the chemically modified guide RNA comprises 10 2′-O-methylnebularines. In some embodiments, the 2′-O-methylnebularine replaces an adenine in an unmodified guide RNA. In some embodiments, the 2′-O-methylnebularine is in the spacer sequence. In some embodiments, the 2′-O-methylnebularine is in a tracrRNA sequence in the scaffold sequence. In some embodiments, the 2′-O-methylnebularine is in a crRNA sequence in the scaffold sequence. In some embodiments, the 2′-O-methylnebularine is in a stem loop structure in the scaffold sequence.

In some embodiments, the chemically modified guide RNA comprises a 2′-deoxynebularine (depicted as “dX” in this application, e.g., in Table 1). In some embodiments, the chemically modified guide RNA comprises 0, 1, 2, 3, 4, 5, 6, 7, 8, 9, or 10 2′-deoxynebularine. In some embodiments, the chemically modified guide RNA does not comprise 2′-deoxynebularine. In some embodiments, the chemically modified guide RNA comprises 1 2′-deoxynebularine. In some embodiments, the chemically modified guide RNA comprises 2 2′-deoxynebularine. In some embodiments, the chemically modified guide RNA comprises 3 2′-deoxynebularines. In some embodiments, the chemically modified guide RNA comprises 4 2′-deoxynebularines. In some embodiments, the chemically modified guide RNA comprises 5 2′-deoxynebularines. In some embodiments, the chemically modified guide RNA comprises 6 2′-deoxynebularines. In some embodiments, the chemically modified guide RNA comprises 7 2′-deoxynebularines. In some embodiments, the chemically modified guide RNA comprises 8 2′-deoxynebularines. In some embodiments, the chemically modified guide RNA comprises 9 2′-deoxynebularines. In some embodiments, the chemically modified guide RNA comprises 10 2′-deoxynebularines. In some embodiments, the 2′-deoxynebularine replaces an adenine in an unmodified guide RNA. In some embodiments, the 2′-deoxynebularine is in the spacer sequence. In some embodiments, the 2′-deoxynebularine is in a tracrRNA sequence in the scaffold sequence. In some embodiments, the 2′-deoxynebularine is in a crRNA sequence in the scaffold sequence. In some embodiments, the 2′-deoxynebularine is in a stem loop structure in the scaffold sequence.

In some embodiments, the chemically modified guide RNA comprises a 2′-O-methylribonucleotide (2′-OMe). In some embodiments, the guide RNA comprises from about 0 to about 70 2′-OMe. In some embodiments, the chemically modified guide RNA comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 2′-OMe. In some embodiments, the chemically modified guide RNA comprises from about 0 to about 70 2′-OMe. In some embodiments, the chemically modified guide RNA comprises from about 0 to about 10, about 0 to about 20, about 0 to about 30, about 0 to about 35, about 0 to about 40, about 0 to about 45, about 0 to about 50, about 0 to about 55, about 0 to about 60, about 0 to about 65, about 0 to about 70, about 10 to about 20, about 10 to about 30, about 10 to about 35, about 10 to about 40, about 10 to about 45, about 10 to about 50, about 10 to about 55, about 10 to about 60, about 10 to about 65, about 10 to about 70, about 20 to about 30, about 20 to about 35, about 20 to about 40, about 20 to about 45, about 20 to about 50, about 20 to about 55, about 20 to about 60, about 20 to about 65, about 20 to about 70, about 30 to about 35, about 30 to about 40, about 30 to about 45, about 30 to about 50, about 30 to about 55, about 30 to about 60, about 30 to about 65, about 30 to about 70, about 35 to about 40, about 35 to about 45, about 35 to about 50, about 35 to about 55, about 35 to about 60, about 35 to about 65, about 35 to about 70, about 40 to about 45, about 40 to about 50, about 40 to about 55, about 40 to about 60, about 40 to about 65, about 40 to about 70, about 45 to about 50, about 45 to about 55, about 45 to about 60, about 45 to about 65, about 45 to about 70, about 50 to about 55, about 50 to about 60, about 50 to about 65, about 50 to about 70, about 55 to about 60, about 55 to about 65, about 55 to about 70, about 60 to about 65, about 60 to about 70, or about 65 to about 70 2′-OMe. In some embodiments, the chemically modified guide RNA comprises from about 0, about 10, about 20, about 30, about 35, about 40, about 45, about 50, about 55, about 60, about 65, or about 70 2′-OMe. In some embodiments, the chemically modified guide RNA comprises from at least about 0, about 10, about 20, about 30, about 35, about 40, about 45, about 50, about 55, about 60, or about 65 2′-OMe. In some embodiments, the chemically modified guide RNA comprises from at most about 10, about 20, about 30, about 35, about 40, about 45, about 50, about 55, about 60, about 65, or about 70 2′-OMe. In some embodiments, the chemically modified guide RNA does not comprise 2′-OMe. In some embodiments, the chemically modified guide RNA comprises 62 2′-OMe. In some embodiments, the chemically modified guide RNA comprises 54 2′-OMe.

In some embodiments, the chemically modified guide RNA comprises 2′-OMe at the 5′ end of the guide RNA sequence. In some embodiments, the chemically modified guide RNA comprises 2′-OMe at the 3′ end of the guide RNA sequence. In some embodiments, the chemically modified guide RNA comprises 2′-OMe at the internal positions of the guide RNA sequence. In some embodiments, the chemically modified guide RNA comprises 2′-OMe at the 5′ end, 3′ end, or internal positions of the guide RNA sequence. In some embodiments, the chemically modified guide RNA comprises 2′-OMe at the 5′ end, 3′ end, and internal positions of the guide RNA sequence.

In some embodiments, each one of the last nucleotides at the 3′end of the chemically modified single guide RNA comprises a 2′-OMe modification. In some embodiments, each one of the last two nucleotides at the 3′end of the chemically modified single guide RNA comprises a 2′-OMe modification. In some embodiments, each one of the last three nucleotides at the 3′end of the chemically modified single guide RNA comprises a 2′-OMe modification. In some embodiments, each one of the last four nucleotides at the 3′end of the chemically modified single guide RNA comprises a 2′-OMe modification. In some embodiments, each one of the last five nucleotides at the 3′end of the chemically modified single guide RNA comprises a 2′-OMe modification. In some embodiments, each one of the last six nucleotides at the 3′end of the chemically modified single guide RNA comprises a 2′-OMe modification. In some embodiments, each one of the last seven nucleotides at the 3′end of the chemically modified single guide RNA comprises a 2′-OMe modification. In some embodiments, each one of the last eight nucleotides at the 3′end of the chemically modified single guide RNA comprises a 2′-OMe modification. In some embodiments, each one of the last nine nucleotides at the 3′end of the chemically modified single guide RNA comprises a 2′-OMe modification. In some embodiments, each one of the last ten nucleotides at the 3′end of the chemically modified single guide RNA comprises a 2′-OMe modification.

In some embodiments, the chemically modified guide RNA has a one or more chemical modifications in the 5′ end of the guide RNA sequence. In some embodiments, the chemically modified guide RNA comprises one or more chemically modified nucleotides in the 5′ end of the guide RNA sequence. In some embodiments, the chemically modified guide RNA has a one or more chemical modifications in the 3′ end of the guide RNA sequence. In some embodiments, the chemically modified guide RNA comprises one or more chemically modified nucleotides in the 3′ end of the guide RNA sequence.

In some embodiments, the chemically modified guide RNA comprises 1 additional Us at the 3′ end, with phosphodiester linkage (3′ UUU). In some embodiments, the chemically modified guide RNA comprises 2 additional Us at the 3′ end, with phosphodiester linkage (3′ UUU). In some embodiments, the chemically modified guide RNA comprises 3 additional Us at the 3′ end, with phosphodiester linkage (3′ UUU). In some embodiments, the chemically modified guide RNA comprises 4 additional Us at the 3′ end, with phosphodiester linkage (3′ UUU). In some embodiments, the chemically modified guide RNA comprises 5 additional Us at the 3′ end, with phosphodiester linkage (3′ UUU). In some embodiments, the chemically modified guide RNA comprises 6 additional Us at the 3′ end, with phosphodiester linkage (3′ UUU). In some embodiments, the chemically modified guide RNA comprises 7 additional Us at the 3′ end, with phosphodiester linkage (3′ UUU). In some embodiments, the chemically modified guide RNA comprises have 3 additional Us at the 3′ end, with phosphodiester linkage (3′ UUU). In some embodiments, the chemically modified guide RNA comprises 8 additional Us at the 3′ end, with phosphodiester linkage (3′ UUU). In some embodiments, the chemically modified guide RNA comprises 9 additional Us at the 3′ end, with phosphodiester linkage (3′ UUU). In some embodiments, the chemically modified guide RNA comprises 10 additional Us at the 3′ end, with phosphodiester linkage (3′ UUU).

In some embodiments, the chemically modified guide RNA comprises one or more combination(s) of all the aforementioned nucleobase, sugar and backbone modifications at select position(s). In some embodiments, the chemically modified guide RNA comprises a 2′-O-methylribonucleotide (2′-OMe) and PS linkages. In some embodiments, the chemically modified single guide RNA comprises the sequence 5′-NsNsN-3′, 5′-NsNsNsS-3′, 5′-nsnsnsn-3′, or 5′-nsnsn-3′ at the 3′ end, wherein, each uppercase N independently indicates unmodified nucleotide adenosine, cytidine, guanosine and/or uridine; and lowercase letters indicates modified nucleotides including but not limited to 2′-H, 2′-OMe and base modification; and each s independently indicates phosphorothioate backbone modification. In some embodiments, each one of the last four nucleotides at the 3′end of the single guide RNA comprises a 2′-OMe modification.

In some embodiments, the chemically modified guide RNA comprises 2′-OMe modified Us at the 3′ end. In some embodiments, the chemically modified guide RNA comprises 2′-OMe modified Us at the 3′ end with PS linkages (3′ UsUsUsU). In some embodiments, the chemically modified guide RNA comprises 2′-OMe modified Us at the 3′ end without PS linkages (3′usususu). In some embodiments, the chemically modified single guide RNA comprises the sequence 5′-UsUsU, 5′-UsUsUsU-3′, 5′-usususu-3′, or 5′-ususu-3′ at the 3′ end. In some embodiments, the chemically modified single guide RNA comprises the sequence 5′-UsUsU at the 3′ end. In some embodiments, the chemically modified single guide RNA comprises the sequence 5′-UsUsUsU-3′ at the 3′ end. In some embodiments, the chemically modified single guide RNA comprises the sequence 5′-usususu-3′ at the 3′ end. In some embodiments, the chemically modified single guide RNA comprises the sequence 5′-ususu-3′ at the 3′ end.

In some embodiments, the guide RNA comprises an unmodified nucleotide. In some embodiments, the unmodified nucleotide comprises a 2′hydroxyl(2′OH) that is in close proximity to the Cas12b protein when contacted with the Cas12b protein. “Close proximity” as used herein in some embodiments, refers to a hydrogen bond (real or predicted) between the 2′hydroxyl of a given nucleotide and the Cas12b protein in a three-dimensional complex. In other embodiments, “close proximity” refers to physical distance between the 2′hydroxyl and the Cas12b protein, such as 1, 2, 3, 4, 5, 6, 7, 8, 9, or 10 angstroms (Å) distance. In yet other embodiments, “close proximity” refers to other electrostatic or dipole-dipole interactions between the atoms of the 2′hydroxyl of a given nucleotide and the Cas12b protein. In some embodiments, the unmodified nucleotide comprises a 2′hydroxyl that is in contact with the Cas12b protein when contacted with the Cas12b protein. In some embodiments, the unmodified nucleotide comprises a 2′hydroxyl that is in close proximity with a second nucleotide in the guide RNA.

In some embodiments, the single guide RNA comprises an unmodified nucleotide. In some embodiments, the unmodified nucleotide comprises a 2′hydroxyl that is in close proximity to the Cas12b protein when contacted with the Cas12b protein. In some embodiments, the unmodified nucleotide comprises a 2′hydroxyl that is in contact with the Cas12b protein when contacted with the Cas12b protein. In some embodiments, the unmodified nucleotide comprises a 2′hydroxyl that is in close proximity with a second nucleotide in the single guide RNA.

### Exemplary Modified Guide RNAs

Reference Guide Sequence
SEQ ID NO: 1
GUUCUGUCUUUUGGUCAGGACAACCGUCUAGCUAUAAGUGCUGCAGG
GUGUGAGAAACUCCUAUUGCUGGACGAUGUCUCUUACGAGGCAUUAG
CACAACCAACAGCAUAGUCAAAUAAAUUUU,
gRNA scaffold sequence: positions 1 to 97 of
SEQ ID NO: 1.
Underlined sequence: ANGPTL3 spacer (positions
98 to 120 of SEQ ID NO: 1)

SEQ ID NO: 1 is an exemplary Cas12b unmodified guide RNA sequence of 124 nucleotides in length which can be modified according to the modifications described herein. In some embodiments, a chemically modified guide RNA comprises SEQ ID NO: 1 with one or more modifications. In some embodiments, a chemically modified guide RNA comprises SEQ ID NO: 1 as a single guide RNA. In some embodiments, a chemically modified guide RNA comprises SEQ ID NO:1 as a dual guide RNA. In some embodiments, a chemically modified guide RNA comprises a scaffold sequence and a spacer sequence, wherein the scaffold sequence comprises a sequence at positions 1 to 97 of SEQ ID NO: 1 or a chemically modified version thereof. In these embodiments, the spacer sequence is contemplated to target a polynucleotide in a gene of interest, e.g., ANGPTL3, and can also be chemically modified.

In some embodiments, a guide RNA comprises the sequence of SEQ ID NO: 1. In embodiments, a guide RNA comprises a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to the sequence of SEQ ID NO: 1. In some embodiments, a guide RNA is SEQ ID NO: 1. In embodiments, a guide RNA is a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to the sequence of SEQ ID NO: 1.

In some embodiments, a single guide RNA comprises one or more unmodified nucleotide independently at a nucleotide position selected from positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 as numbered in SEQ ID NO: 1 or a corresponding position thereof. In some embodiments, a single guide RNA comprises an unmodified nucleotide independently at a nucleotide position selected from positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 as numbered in SEQ ID NO: 1 or a corresponding position thereof. In some embodiments, a single guide RNA comprises one or more unmodified nucleotide at a nucleotide position selected from positions 8-10, 12-15, 22-24, 32-38, 40, 41, 43, 44, 53-56, 63, 66-69, 88-97, 99-103, 106-108, 111-116 as numbered in SEQ ID NO: 1 or a corresponding position thereof. In some embodiments, the unmodified nucleotide is at a nucleotide position selected from positions 1, 8-10, 12-15, 22-24, 32-38, 40, 41, 43, 44, 53-56, 63, 66-69, 88-97, 99-103, 106-108, 111-116 as numbered in SEQ ID NO: 1 or a corresponding position thereof.

In some embodiments, a single guide RNA comprises one or more chemical modification independently at a nucleotide position selected from positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 as numbered in SEQ ID NO: 1 or a corresponding position thereof. In some embodiments, a single guide RNA comprises a chemical modification independently at a nucleotide position selected from positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 as numbered in SEQ ID NO: 1 or a corresponding position thereof. In some embodiments, a single guide RNA comprises one or more chemical modification at a nucleotide position selected from positions 2-7, 11, 16-21, 25-31, 39, 42, 45-52, 57-62, 64, 65, 70-87, 98, 104, 105, 109, and 110 as numbered in SEQ ID NO: 1 or a corresponding position thereof. In some embodiments, the chemical modification is at a nucleotide position selected from positions 2-7, 11, 16-21, 25-31, 39, 42, 45-52, 57-62, 64, 65, 70-87, 98, 104, 105, 109, and 110 as numbered in SEQ ID NO: 1 or a corresponding position thereof. In some embodiments, the chemical modification comprises a 2′-OMe modification. In some embodiments, the chemical modification comprises a nebularin. In some embodiments, the chemical modification comprises a deoxynebularin.

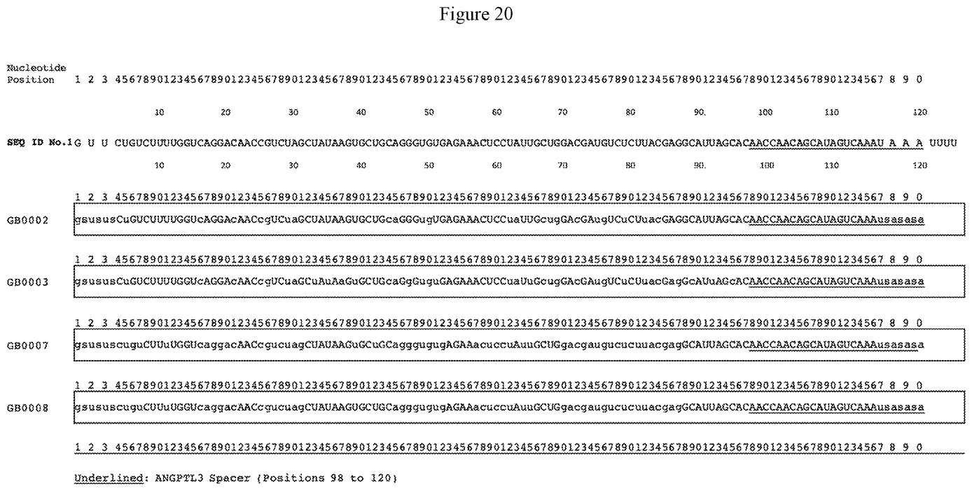

In some embodiments, the guide RNA comprises one or more structure based site-specific chemical modification(s) as illustrated in Table 1. Table 1 as shown below depicts exemplary guide RNAs which incorporate one or more modifications described herein and comprises an RNA spacer sequence which is analogous to a protospacer polynucleotide in the ANGPTL3 gene. FIG. 20 depicts an alignment of the reference guide RNA (SEQ ID NO:1) along with exemplary chemically modified guide RNAs, GB0002, GB0003, GB0007, GB0008. FIG. 20 further shows where which the modifications at certain positions as well as the spacer (positions 98 to 120) and scaffold sequence (positions 1 to 97) of these guide RNAs. As shown in FIG. 20, GB002 has modified nucleotides at positions 1-3, 5, 16, 21, 25, 29, 30, 44, 45, 49, 50, 62, 63, 67-69, 72, 75, 76, 79, 82-84 and 117-120, GB003 has modified nucleotides at positions 1, 2, 3, 5, 16, 21, 25, 29, 30, 33, 35, 37, 39, 44, 45, 47, 49-51, 62, 63, 65, 67-69, 72, 75, 76, 79, 82-84, 86, 87, 89, 92, 95 and 117-120. GB007 has modified nucleotides at positions 1-7, 11, 16-21, 25-31, 39, 42, 45-52, 57-62, 64, 65, 70-87, and 117-120, and GB008 has modified nucleotides at positions 1-7, 11, 16-21, 25-31, 45-52, 57-62, 64, 65, 70-87 and 117-120. Conversely, GB002 has unmodified nucleotides at positions 4, 6-15, 17-20, 22-24, 27, 28, 31-43, 46-48, 51-61, 64-66, 70, 71, 73, 74, 77, 78, 80, 81, 85-116, GB003 has unmodified nucleotides at positions 4, 6-15, 17-20, 22-24, 27, 28, 31, 32, 34, 36, 38, 40-43, 46, 48, 52-61, 64, 66, 70, 71, 73, 74, 77, 78, 80, 81, 85, 88, 90, 91, 93, 94 and 96-116. GB007 has unmodified nucleotides at positions 8-10, 12-15, 22-24, 32-38, 40, 41, 43, 44, 53-56, 63, 66-69 and 88-116, and GB008 has unmodified nucleotides at positions 8-10, 12-15, 22-24, 32-44, 53-56, 63, 66-69 and 88-116.

It is further contemplated, in some embodiments, that the exemplary guide RNAs of Table 1 can have the ANGPTL3 spacer sequence swapped with different spacer sequences to target other genes of interest, such as PCSK9, APOC3, and the like. Furthermore, the modifications on spacer sequences of other genes are also contemplated, in some embodiments, to correspond to the modifications used in the exemplary ANGPTL3 spacer sequences. Thus, in some embodiments, are guide RNAs that comprise a scaffold sequence in one of the gRNAs in Table 1 and a different spacer sequence than the spacer sequence in one of the gRNAs in Table 1.

In another aspect, provided herein is a guide RNA, including single guides thereof, comprising a gRNA sequence of Table 1, wherein a, u, g, and c indicate 2′-OMe modified adenine, uridine, guanine, and cytidine, wherein s indicates a phosphorothioate linkage, wherein X indicates a nebularine, and wherein dX indicates a 2′-deoxynebularine. In some embodiments, a guide RNA comprises a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to any one of the gRNA sequences of Table 1. In some embodiments, a guide RNA is a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to any one of the gRNA sequences of Table 1. In some embodiments, a guide RNA is any one of the sequences of Table 1.

One aspect provided herein is a single guide RNA comprising a sequence selected from any one of SEQ ID NOs: 2-89, wherein a, u, g, and c indicate 2′-OMe modified adenine, uridine, guanine, and cytidine, and wherein s indicates a phosphorothioate linkage. In some embodiments, a single guide RNA comprises a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to any one of SEQ ID NOs: 2-89. In some embodiments, a single guide RNA is a sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, and 100% identity to any one of SEQ ID NOs: 2-89.

In some embodiments, a single guide RNA is any one of SEQ ID NOs: 2-89 having one or more chemical modifications.

In some embodiments, the single guide RNA comprises a chemically modified nucleotide selected from positions 1-3, 5, 16, 21, 25, 29, 30, 44, 45, 49, 50, 62, 63, 67-69, 72, 75, 76, 79, 82-84 and 117-120 as numbered in SEQ ID NO: 1 or a corresponding position thereof.

In some embodiments, the single guide RNA comprises a chemically modified nucleotide selected from positions 1, 2, 3, 5, 16, 21, 25, 29, 30, 33, 35, 37, 39, 44, 45, 47, 49-51, 62, 63, 65, 67-69, 72, 75, 76, 79, 82-84, 86, 87, 89, 92, 95 and 117-120 as numbered in SEQ ID NO: 1 or a corresponding position thereof.

In some embodiments, the single guide RNA comprises a chemically modified nucleotide selected from positions 1-7, 11, 16-21, 25-31, 39, 42, 45-52, 57-62, 64, 65, 70-87, and 117-120 as numbered in SEQ ID NO: 1 or a corresponding position thereof.

In some embodiments, the single guide RNA comprises a chemically modified nucleotide selected from positions 1-7, 11, 16-21, 25-31, 45-52, 57-62, 64, 65, 70-87 and 117-120 as numbered in SEQ ID NO: 1 or a corresponding position thereof.

In some embodiments, the single guide RNA comprises an unmodified nucleotide at a nucleotide position selected from positions 4, 6-15, 17-20, 22-24, 27, 28, 31-43, 46-48, 51-61, 64-66, 70, 71, 73, 74, 77, 78, 80, 81, and 85-116 as numbered in in SEQ ID NO: 1 or a corresponding position thereof.

In some other embodiments, the single guide RNA comprises an unmodified nucleotide at a nucleotide position selected from positions 4, 6-15, 17-20, 22-24, 27, 28, 31, 32, 34, 36, 38, 40-43, 46, 48, 52-61, 64, 66, 70, 71, 73, 74, 77, 78, 80, 81, 85, 88, 90, 91, 93, 94 and 96-116 as numbered in SEQ ID NO: 1 or a corresponding position thereof.

In some other embodiments, the single guide RNA comprises an unmodified nucleotide at a nucleotide position selected from positions 8-10, 12-15, 22-24, 32-38, 40, 41, 43, 44, 53-56, 63, 66-69 and 88-116 as numbered in SEQ ID NO: 1 or a corresponding position thereof.

In some other embodiments, the single guide RNA comprises an unmodified nucleotide at a nucleotide position selected from positions 8-10, 12-15, 22-24, 32-44, 53-56, 63, 66-69 and 88-116 as numbered in SEQ ID NO: 1 or a corresponding position thereof.

TABLE 1
Exemplary Cas12b gRNAs targeting human ANGPTL3 gene

| | Proto- | Exemplary | | |
|---|---|---|---|---|
| | Spacer | ANGPTL3 | gRNA | |
| gRNA | SEQ ID | Protospacer | SEQ ID | gRNA (5′-3′) with |
| ID | NO: | (5′-3′) | NO: | Modified Exemplary Spacer |
| GB0001 | 90 | AACCAACAGCAT | 2 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAusasasa |
| GB0002 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB0003 | 90 | AACCAACAGCAT | 198 | gsususCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Ausasasa |
| GB0004 | 90 | AACCAACAGCAT | 199 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAuAGUcAAAusasasa |
| GB0005 | 90 | AACCAACAGCAT | 200 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAusasasa |
| GB0006 | 90 | AACCAACAGCAT | 201 | gsususCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Ausasasa |
| GB0007 | 90 | AACCAACAGCAT | 202 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAusasasa |
| GB0008 | 90 | AACCAACAGCAT | 203 | gsususcuguCUUuUGGUcaggacAACcgucuag |
| | | AGTCAAATAAA | | CUAUAAGUGCUGCagggugugAGAAacucc |
| | | | | uAuuGCUGgacgaugucucuuacgagGCAUUAG |
| | | | | CACAACCAACAGCAUAGUCAAAusasasa |
| GB0009 | 90 | AACCAACAGCAT | 204 | gsususCUGUCUUUUGGUCAGGACAACcg |
| | | AGTCAAATAAA | | ucuagCUAUAAGuGCuGCagggugugAGAAa |
| | | | | cuccuAuuGCUGgacgaugucucuuacgagGCAU |
| | | | | UAGCACAACCAACAGCAUAGUCAAAus |
| | | | | asasa |
| GB0010 | 90 | AACCAACAGCAT | 205 | gsususcuguCUUuUGGUcaggacAACcgucuag |
| | | AGTCAAATAAA | | CUAUAAGuGCuGCAGGGUGUGAGAAA |
| | | | | CUCCUAuuGCUGgacgaugucucuuacgagGC |
| | | | | AUUAGCACAACCAACAGCAUAGUCAA |
| | | | | Ausasasa |
| GB0011 | 90 | AACCAACAGCAT | 206 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAusasasa |
| GB0012 | 90 | AACCAACAGCAT | 207 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAusasasa |
| GB0013 | 90 | AACCAACAGCAT | 208 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACcAAc |
| | | | | aGcAuaGUCAAAusasasa |
| GB0014 | 90 | AACCAACAGCAT | 209 | gsususcuguCUUuUGGUcaggacAACcgucuag |
| | | AGTCAAATAAA | | CuAUAAGuGCuGCagggugugAGAAacuccu |
| | | | | AuuGCUGgacgaugucucuuacgagGcAUUAGC |
| | | | | ACaACcAAcaGcAuaGUCAAAusasasa |
| GB0015 | 90 | AACCAACAGCAT | 3 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAusususu |
| GB0016 | 90 | AACCAACAGCAT | 4 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUGUCAAAuaaausususu |
| GB0017 | 90 | AACCAACAGCAT | 210 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuAAA |
| | | | | usususu |
| GB0018 | 90 | AACCAACAGCAT | 211 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuaaausususu |
| GB0019 | 90 | AACCAACAGCAT | 212 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAUAAA |
| | | | | usususu |
| GB0020 | 90 | AACCAACAGCAT | 213 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuaaaus |
| | | | | ususu |
| GB0021 | 90 | AACCAACAGCAT | 214 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuaaaususu |
| | | | | su |
| GB0022 | 91 | AGGTAGTCCATG | 5 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | UCCAUGGACAUUAasususc |
| GB0023 | 91 | AGGTAGTCCATG | 215 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGuAGu |
| | | | | cCAuggACAUuAasususc |
| GB0024 | 91 | AGGTAGTCCATG | 216 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | ucCAUggACAUUAasususc |
| GB0025 | 91 | AGGTAGTCCATG | 217 | gsususCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAasususc |
| GB0026 | 91 | AGGTAGTCCATG | 218 | gsususCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aasususc |
| GB0027 | 91 | AGGTAGTCCATG | 219 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAasusus |
| | | | | c |
| GB0028 | 91 | AGGTAGTCCATG | 220 | gsususcuguCUUuUGGUcaggacAACcgucuag |
| | | GACATTAATTC | | CUAUAAGUGCUGCagggugugAGAAacucc |
| | | | | uAuuGCUGgacgaugucucuuacgagGCAUUAG |
| | | | | CACAGGUAGUCCAUGGACAUUAasusus |
| | | | | c |
| GB0029 | 91 | AGGTAGTCCATG | 221 | gsususCUGUCUUUUGGUCAGGACAACcg |
| | | GACATTAATTC | | ucuagCUAUAAGuGCuGCagggugugAGAAa |
| | | | | cuccuAuuGCUGgacgaugucucuuacgagGCAU |
| | | | | UAGCACAGGUAGUCCAUGGACAUUAas |
| | | | | ususc |
| GB0030 | 91 | AGGTAGTCCATG | 222 | gsususcuguCUUuUGGUcaggacAACcgucuag |
| | | GACATTAATTC | | CUAUAAGuGCuGCAGGGUGUGAGAAA |
| | | | | CUCCUAuuGCUGgacgaugucucuuacgagGC |
| | | | | AUUAGCACAGGUAGUCCAUGGACAUU |
| | | | | Aasususc |
| GB0031 | 91 | AGGTAGTCCATG | 223 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGUAG |
| | | | | ucCAUggACAUUAasususc |
| GB0032 | 91 | AGGTAGTCCATG | 224 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAasususc |
| GB0033 | 91 | AGGTAGTCCATG | 225 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGuAGucCAuggACAUuAasususc |
| GB0034 | 91 | AGGTAGTCCATG | 226 | gsususcuguCUUuUGGUcaggacAACcgucuag |
| | | GACATTAATTC | | CuAUAAGuGCuGCagggugugAGAAacuccu |
| | | | | AuuGCUGgacgaugucucuuacgagGcAUUAGC |
| | | | | ACaGGuAGucCAuggACAUuAasususc |
| GB0035 | 91 | AGGTAGTCCATG | 6 | gsususcuguCUUuUGGUcaggacAACcgucuag |
| | | GACATTAATTC | | CUAUAAGuCuGCagggugugAGAAacuccuA |
| | | | | uuGCUGgacgaugucucuuacgagGCAUUAGC |
| | | | | ACAGGUAGUCCAUGGACAUUAAUUCu |
| | | | | sususu |
| GB0036 | 91 | AGGTAGTCCATG | 7 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAauucus |
| | | | | ususu |
| GB0037 | 91 | AGGTAGTCCATG | 227 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAauucususu |
| | | | | su |
| GB0038 | 92 | CAAAAACTCAAC | 8 | gsusUCUGUCUUUUGGUCAGGACAACC |
| | | ATATTTGATCA | | GUCUAGCUAUAAGUGCUGCAGGGUGU |
| | | | | GAGAAACUCCUAUUGCUGGACGAUGU |
| | | | | CUCUUACGAGGCAUUAGCACCAAAAA |
| | | | | CUCAACAUAUUUGAUCAUsususu |
| GB0039 | 93 | TACTTCAACAAA | 9 | gsusUCUGUCUUUUGGUCAGGACAACC |
| | | AAGTGAAATAT | | GUCUAGCUAUAAGUGCUGCAGGGUGU |
| | | | | GAGAAACUCCUAUUGCUGGACGAUGU |
| | | | | CUCUUACGAGGCAUUAGCACUACUUC |
| | | | | AACAAAAAGUGAAAUAUUsususu |
| GB0040 | 94 | AGAAGAGCAAC | 10 | gsusUCUGUCUUUUGGUCAGGACAACC |
| | | TAACTAACTTAA | | GUCUAGCUAUAAGUGCUGCAGGGUGU |
| | | | | GAGAAACUCCUAUUGCUGGACGAUGU |
| | | | | CUCUUACGAGGCAUUAGCACAGAAGA |
| | | | | GCAACUAACUAACUUAAUsususu |
| GB0041 | 95 | TGGAGGAAATA | 11 | gsusUCUGUCUUUUGGUCAGGACAACC |
| | | ACTAGAGGAAC | | GUCUAGCUAUAAGUGCUGCAGGGUGU |
| | | A | | GAGAAACUCCUAUUGCUGGACGAUGU |
| | | | | CUCUUACGAGGCAUUAGCACUGGAGG |
| | | | | AAAUAACUAGAGGAACAUsususu |
| GB0042 | 96 | AAGAACCCACA | 12 | gsusUCUGUCUUUUGGUCAGGACAACC |
| | | GAAATTTCTCTA | | GUCUAGCUAUAAGUGCUGCAGGGUGU |
| | | | | GAGAAACUCCUAUUGCUGGACGAUGU |
| | | | | CUCUUACGAGGCAUUAGCACAAGAAC |
| | | | | CCACAGAAAUUUCUCUAUsususu |
| GB0043 | 90 | AACCAACAGCAT | 13 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAUsususu |
| GB0044 | 91 | aGGTAGTCCATG | 14 | gsusUCUGUCUUUUGGUCAGGACAACC |
| | | GACATTAATTC | | GUCUAGCUAUAAGUGCUGCAGGGUGU |
| | | | | GAGAAACUC |
| | | | | CUAUUGCUGGACGAUGUCUCUUACGA |
| | | | | GGCAUUAGCACAGGUAGUCCAUGGAC |
| | | | | AUUAAUUCUsususu |
| GB0045 | 97 | CTGGCAATGTCC | 15 | gsusUCUGUCUUUUGGUCAGGACAACC |
| | | CCAATGCAATC | | GUCUAGCUAUAAGUGCUGCAGGGUGU |
| | | | | GAGAAACUCCUAUUGCUGGACGAUGU |
| | | | | CUCUUACGAGGCAUUAGCACCUGGCA |
| | | | | AUGUCCCCAAUGCAAUCUsususu |
| GB0046 | 98 | CATTGGGGACAT | 16 | gsusUCUGUCUUUUGGUCAGGACAACC |
| | | TGCCAGTAATC | | GUCUAGCUAUAAGUGCUGCAGGGUGU |
| | | | | GAGAAACUCCUAUUGCUGGACGAUGU |
| | | | | CUCUUACGAGGCAUUAGCACCAUUGG |
| | | | | GGACAUUGCCAGUAAUCUsususu |
| GB0047 | 99 | CCCAAGTAAAAA | 17 | gsusUCUGUCUUUUGGUCAGGACAACC |
| | | GAATATTCAAT | | GUCUAGCUAUAAGUGCUGCAGGGUGU |
| | | | | GAGAAACUCCUAUUGCUGGACGAUGU |
| | | | | CUCUUACGAGGCAUUAGCACCCCAAG |
| | | | | UAAAAAGAAUAUUCAAUUsususu |
| GB0048 | 100 | AATATAATGTTT | 18 | gsusUCUGUCUUUUGGUCAGGACAACC |
| | | GTTGTCTTTCC | | GUCUAGCUAUAAGUGCUGCAGGGUGU |
| | | | | GAGAAACUCCUAUUGCUGGACGAUGU |
| | | | | CUCUUACGAGGCAUUAGCACAAUAUA |
| | | | | AUGUUUGUUGUCUUUCCUsususu |
| GB0049 | 101 | GAAGAGCAACT | 19 | gsusUCUGUCUUUUGGUCAGGACAACC |
| | | AACTAACTTAAT | | GUCUAGCUAUAAGUGCUGCAGGGUGU |
| | | | | GAGAAACUCCUAUUGCUGGACGAUGU |
| | | | | CUCUUACGAGGCAUUAGCACGAAGAG |
| | | | | CAACUAACUAACUUAAUUsususu |
| GB00001 | 90 | AACCAACAGCAT | 20 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAusasasa |
| GB00002 | 90 | AACCAACAGCAT | 244 | gsusUCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00003 | 90 | AACCAACAGCAT | 245 | gsusUCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Ausasasa |
| GB00004 | 90 | AACCAACAGCAT | 246 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAuAGUcAAAusasasa |
| GB00005 | 90 | AACCAACAGCAT | 247 | gsusUCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAusasasa |
| GB00006 | 90 | AACCAACAGCAT | 248 | gsusUCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Ausasasa |
| GB00007 | 90 | AACCAACAGCAT | 249 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAusasasa |
| GB00008 | 90 | AACCAACAGCAT | 250 | gsusUcuguCUUuUGGUcaggacAACcgucuag |
| | | AGTCAAATAAA | | CUAUAAGUGCUGCagggugugAGAAacucc |
| | | | | uAuuGCUGgacgaugucucuuacgagGCAUUAG |
| | | | | CACAACCAACAGCAUAGUCAAAusasasa |
| GB00009 | 90 | AACCAACAGCAT | 251 | gsusUCUGUCUUUUGGUCAGGACAACcg |
| | | AGTCAAATAAA | | ucuagCUAUAAGuGCuGCagggugugAGAAa |
| | | | | cuccuAuuGCUGgacgaugucucuuacgagGCAU |
| | | | | UAGCACAACCAACAGCAUAGUCAAAus |
| | | | | asasa |
| GB00010 | 90 | AACCAACAGCAT | 252 | gsusUcuguCUUuUGGUcaggacAACcgucuag |
| | | AGTCAAATAAA | | CUAUAAGuGCuGCAGGGUGUGAGAAA |
| | | | | CUCCUAuuGCUGgacgaugucucuuacgagGC |
| | | | | AUUAGCACAACCAACAGCAUAGUCAA |
| | | | | Ausasasa |
| GB00011 | 90 | AACCAACAGCAT | 253 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAusasasa |
| GB00012 | 90 | AACCAACAGCAT | 254 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAusasasa |
| GB00013 | 90 | AACCAACAGCAT | 255 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACcAAc |
| | | | | aGcAuaGUCAAAusasasa |
| GB00014 | 90 | AACCAACAGCAT | 256 | gsusUcuguCUUuUGGUcaggacAACcgucuag |
| | | AGTCAAATAAA | | CuAUAAGuGCuGCagggugugAGAAacuccu |
| | | | | AuuGCUGgacgaugucucuuacgagGcAUUAGC |
| | | | | ACaACcAAcaGcAuaGUCAAAusasasa |
| GB00015 | 90 | AACCAACAGCAT | 228 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAusususu |
| GB00016 | 90 | AACCAACAGCAT | 21 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUGUCAAAuaaausususu |
| GB00017 | 90 | AACCAACAGCAT | 229 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuAAA |
| | | | | usususu |
| GB00018 | 90 | AACCAACAGCAT | 230 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuaaausususu |
| GB00019 | 90 | AACCAACAGCAT | 231 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAUAAA |
| | | | | usususu |
| GB00020 | 90 | AACCAACAGCAT | 232 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuaaaus |
| | | | | ususu |
| GB00021 | 90 | AACCAACAGCAT | 233 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuaaaususu |
| | | | | su |
| GB00022 | 91 | AGGTAGTCCATG | 22 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | UCCAUGGACAUUAasususc |
| GB00023 | 91 | AGGTAGTCCATG | 272 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGuAGu |
| | | | | cCAuggACAUuAasususc |
| GB00024 | 91 | AGGTAGTCCATG | 273 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | ucCAUggACAUUAasususc |
| GB00025 | 91 | AGGTAGTCCATG | 274 | gsusUCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAasususc |
| GB00026 | 91 | AGGTAGTCCATG | 275 | gsusUCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aasususc |
| GB00027 | 91 | AGGTAGTCCATG | 276 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAasusus |
| | | | | c |
| GB00028 | 91 | AGGTAGTCCATG | 277 | gsusUcuguCUUuUGGUcaggacAACcgucuag |
| | | GACATTAATTC | | CUAUAAGUGCUGCagggugugAGAAacucc |
| | | | | uAuuGCUGgacgaugucucuuacgagGCAUUAG |
| | | | | CACAGGUAGUCCAUGGACAUUAasusus |
| | | | | c |
| GB00029 | 91 | AGGTAGTCCATG | 278 | gsusUCUGUCUUUUGGUCAGGACAACcg |
| | | GACATTAATTC | | ucuagCUAUAAGuGCuGCagggugugAGAAa |
| | | | | cuccuAuuGCUGgacgaugucucuuacgagGCAU |
| | | | | UAGCACAGGUAGUCCAUGGACAUUAas |
| | | | | ususc |
| GB00030 | 91 | AGGTAGTCCATG | 279 | gsusUcuguCUUuUGGUcaggacAACcgucuag |
| | | GACATTAATTC | | CUAUAAGuGCuGCAGGGUGUGAGAAA |
| | | | | CUCCUAuuGCUGgacgaugucucuuacgagGC |
| | | | | AUUAGCACAGGUAGUCCAUGGACAUU |
| | | | | Aasususc |
| GB00031 | 91 | AGGTAGTCCATG | 280 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGUAG |
| | | | | ucCAUggACAUUAasususc |
| GB00032 | 91 | AGGTAGTCCATG | 281 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAasususc |
| GB00033 | 91 | AGGTAGTCCATG | 282 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGuAGucCAuggACAUuAasususc |
| GB00034 | 91 | AGGTAGTCCATG | 283 | gsusUcuguCUUuUGGUcaggacAACcgucuag |
| | | GACATTAATTC | | CuAUAAGuGCuGCagggugugAGAAacuccu |
| | | | | AuuGCUGgacgaugucucuuacgagGcAUUAGC |
| | | | | ACaGGuAGucCAuggACAUuAasususc |
| GB00035 | 91 | AGGTAGTCCATG | 23 | gsusUcuguCUUuUGGUcaggacAACcgucuag |
| | | GACATTAATTC | | CUAUAAGuCuGCagggugugAGAAacuccuA |
| | | | | uuGCUGgacgaugucucuuacgagGCAUUAGC |
| | | | | ACAGGUAGUCCAUGGACAUUAAUUCu |
| | | | | sususu |
| GB00036 | 91 | AGGTAGTCCATG | 240 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAauucus |
| | | | | ususu |
| GB00037 | 91 | AGGTAGTCCATG | 241 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAauucususu |
| | | | | su |
| GB00038 | 90 | AACCAACAGCAT | 257 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUAGUCAAAusasasa |
| GB00039 | 90 | AACCAACAGCAT | 258 | gsusuCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00040 | 90 | AACCAACAGCAT | 259 | gsusuCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Ausasasa |
| GB00041 | 90 | AACCAACAGCAT | 260 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAuAGUcAAAusasasa |
| GB00042 | 90 | AACCAACAGCAT | 261 | gsusuCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAusasasa |
| GB00043 | 90 | AACCAACAGCAT | 262 | gsusuCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Ausasasa |
| GB00044 | 90 | AACCAACAGCAT | 263 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAusasasa |
| GB00045 | 90 | AACCAACAGCAT | 264 | gsusucuguCUUuUGGUcaggacAACcgucuagC |
| | | AGTCAAATAAA | | UAUAAGUGCUGCagggugugAGAAacuccu |
| | | | | AuuGCUGgacgaugucucuuacgagGCAUUAG |
| | | | | CACAACCAACAGCAUAGUCAAAusasasa |
| GB00046 | 90 | AACCAACAGCAT | 265 | gsusuCUGUCUUUUGGUCAGGACAACcgu |
| | | AGTCAAATAAA | | cuagCUAUAAGuGCuGCagggugugAGAAac |
| | | | | uccuAuuGCUGgacgaugucucuuacgagGCAUU |
| | | | | AGCACAACCAACAGCAUAGUCAAAusa |
| | | | | sasa |
| GB00047 | 90 | AACCAACAGCAT | 266 | gsusucuguCUUuUGGUcaggacAACcgucuagC |
| | | AGTCAAATAAA | | UAUAAGuGCuGCAGGGUGUGAGAAAC |
| | | | | UCCUAuuGCUGgacgaugucucuuacgagGCA |
| | | | | UUAGCACAACCAACAGCAUAGUCAAA |
| | | | | usasasa |
| GB00048 | 90 | AACCAACAGCAT | 267 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACCAAca |
| | | | | GCAuaGUCAAAusasasa |
| GB00049 | 90 | AACCAACAGCAT | 268 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACaACCAAcaGCAuaGUCAAAusasasa |
| GB00050 | 90 | AACCAACAGCAT | 269 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACcAAca |
| | | | | GcAuaGUCAAAusasasa |
| GB00051 | 90 | AACCAACAGCAT | 270 | gsusucuguCUUuUGGUcaggacAACcgucuagC |
| | | AGTCAAATAAA | | uAUAAGuGCuGCagggugugAGAAacuccuA |
| | | | | uuGCUGgacgaugucucuuacgagGcAUUAGCA |
| | | | | CaACcAAcaGcAuaGUCAAAusasasa |
| GB00052 | 90 | AACCAACAGCAT | 234 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUAGUCAAAUAAAusususu |
| GB00053 | 90 | AACCAACAGCAT | 271 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUGUCAAAuaaausususu |
| GB00054 | 90 | AACCAACAGCAT | 235 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuAAAus |
| | | | | ususu |
| GB00055 | 90 | AACCAACAGCAT | 236 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACcAAca |
| | | | | GCAuaGUCAAAuaaausususu |
| GB00056 | 90 | AACCAACAGCAT | 237 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuAAAus |
| | | | | ususu |
| GB00057 | 90 | AACCAACAGCAT | 238 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuaaausus |
| | | | | usu |
| GB00058 | 90 | AACCAACAGCAT | 239 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACaACCAAcaGCAuaGUCAAAuaaausususu |
| GB00059 | 91 | AGGTAGTCCATG | 284 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACAGGUAGU |
| | | | | CCAUGGACAUUAasususc |
| GB00060 | 91 | AGGTAGTCCATG | 285 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACaGGuAGuc |
| | | | | CAuggACAUuAasususc |
| GB00061 | 91 | AGGTAGTCCATG | 286 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACAGGUAGu |
| | | | | cCAUggACAUUAasususc |
| GB00062 | 91 | AGGTAGTCCATG | 287 | gsusuCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAasususc |
| GB00063 | 91 | AGGTAGTCCATG | 288 | gsusuCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aasususc |
| GB00064 | 91 | AGGTAGTCCATG | 289 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACAGGUAGUCCAUGGACAUUAasususc |
| GB00065 | 91 | AGGTAGTCCATG | 290 | gsusucuguCUUuUGGUcaggacAACcgucuagC |
| | | GACATTAATTC | | UAUAAGUGCUGCagggugugAGAAacuccu |
| | | | | AuuGCUGgacgaugucucuuacgagGCAUUAG |
| | | | | CACAGGUAGUCCAUGGACAUUAasusus |
| | | | | c |
| GB00066 | 91 | AGGTAGTCCATG | 291 | gsusuCUGUCUUUUGGUCAGGACAACcgu |
| | | GACATTAATTC | | cuagCUAUAAGuGCuGCagggugugAGAAac |
| | | | | uccuAuuGCUGgacgaugucucuuacgagGCAUU |
| | | | | AGCACAGGUAGUCCAUGGACAUUAasu |
| | | | | susc |
| GB00067 | 91 | AGGTAGTCCATG | 292 | gsusucuguCUUuUGGUcaggacAACcgucuagC |
| | | GACATTAATTC | | UAUAAGuGCuGCAGGGUGUGAGAAAC |
| | | | | UCCUAuuGCUGgacgaugucucuuacgagGCA |
| | | | | UUAGCACAGGUAGUCCAUGGACAUUA |
| | | | | asususc |
| GB00068 | 91 | AGGTAGTCCATG | 293 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACaGGuAGuc |
| | | | | CAUggACAUUAasususc |
| GB00069 | 91 | AGGTAGTCCATG | 294 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGUAGucCAUggACAUUAasususc |
| GB00070 | 91 | AGGTAGTCCATG | 295 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGuAGucCAuggACAUuAasususc |
| GB00071 | 91 | AGGTAGTCCATG | 296 | gsusucuguCUUuUGGUcaggacAACcgucuagC |
| | | GACATTAATTC | | uAUAAGuGCuGCagggugugAGAAacuccuA |
| | | | | uuGCUGgacgaugucucuuacgagGcAUUAGCA |
| | | | | CaGGuAGucCAuggACAUuAasususc |
| GB00072 | 91 | AGGTAGTCCATG | 297 | gsusucuguCUUuUGGUcaggacAACcgucuagC |
| | | GACATTAATTC | | UAUAAGuCuGCagggugugAGAAacuccuAu |
| | | | | uGCUGgacgaugucucuuacgagGCAUUAGCA |
| | | | | CAGGUAGUCCAUGGACAUUAAUUCusu |
| | | | | susu |
| GB00073 | 91 | AGGTAGTCCATG | 242 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACAGGUAGUCCAUGGACAUUAauucusus |
| | | | | usu |
| GB00074 | 91 | AGGTAGTCCATG | 243 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGUAGucCAUggACAUUAauucususus |
| | | | | u |
| GB00075 | 90 | AACCAACAGCAT | 24 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAuasasa |
| GB00076 | 90 | AACCAACAGCAT | 298 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00077 | 90 | AACCAACAGCAT | 299 | gsususCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Auasasa |
| GB00078 | 90 | AACCAACAGCAT | 300 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAuAGUcAAAuasasa |
| GB00079 | 90 | AACCAACAGCAT | 301 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAuasasa |
| GB00080 | 90 | AACCAACAGCAT | 302 | gsususCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Auasasa |
| GB00081 | 90 | AACCAACAGCAT | 303 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuasasa |
| GB00082 | 90 | AACCAACAGCAT | 304 | gsususcuguCUUuUGGUcaggacAACcgucuag |
| | | AGTCAAATAAA | | CUAUAAGUGCUGCagggugugAGAAacucc |
| | | | | uAuuGCUGgacgaugucucuuacgagGCAUUAG |
| | | | | CACAACCAACAGCAUAGUCAAAuasasa |
| GB00083 | 90 | AACCAACAGCAT | 305 | gsususCUGUCUUUUGGUCAGGACAACcg |
| | | AGTCAAATAAA | | ucuagCUAUAAGuGCuGCagggugugAGAAa |
| | | | | cuccuAuuGCUGgacgaugucucuuacgagGCAU |
| | | | | UAGCACAACCAACAGCAUAGUCAAAua |
| | | | | sasa |
| GB00084 | 90 | AACCAACAGCAT | 306 | gsususcuguCUUuUGGUcaggacAACcgucuag |
| | | AGTCAAATAAA | | CUAUAAGuGCuGCAGGGUGUGAGAAA |
| | | | | CUCCUAuuGCUGgacgaugucucuuacgagGC |
| | | | | AUUAGCACAACCAACAGCAUAGUCAA |
| | | | | Auasasa |
| GB00085 | 90 | AACCAACAGCAT | 307 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuasasa |
| GB00086 | 90 | AACCAACAGCAT | 308 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuasasa |
| GB00087 | 90 | AACCAACAGCAT | 309 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACcAAc |
| | | | | aGcAuaGUCAAAuasasa |
| GB00088 | 90 | AACCAACAGCAT | 310 | gsususcuguCUUuUGGUcaggacAACcgucuag |
| | | AGTCAAATAAA | | CuAUAAGuGCuGCagggugugAGAAacuccu |
| | | | | AuuGCUGgacgaugucucuuacgagGcAUUAGC |
| | | | | ACaACcAAcaGcAuaGUCAAAuasasa |
| GB00089 | 90 | AACCAACAGCAT | 25 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAuususu |
| GB00090 | 90 | AACCAACAGCAT | 26 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUGUCAAAuaaauususu |
| GB00091 | 90 | AACCAACAGCAT | 311 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuAAA |
| | | | | uususu |
| GB00092 | 90 | AACCAACAGCAT | 312 | gsususCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuaaauususu |
| GB00093 | 90 | AACCAACAGCAT | 313 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAUAAA |
| | | | | uususu |
| GB00094 | 90 | AACCAACAGCAT | 314 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuaaauu |
| | | | | susu |
| GB00095 | 90 | AACCAACAGCAT | 315 | gsususcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuaaauusus |
| | | | | u |
| GB00096 | 91 | AGGTAGTCCATG | 27 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | UCCAUGGACAUUAaususc |
| GB00097 | 91 | AGGTAGTCCATG | 316 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGuAGu |
| | | | | cCAuggACAUuAaususc |
| GB00098 | 91 | AGGTAGTCCATG | 317 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | ucCAUggACAUUAaususc |
| GB00099 | 91 | AGGTAGTCCATG | 318 | gsususCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAaususc |
| GB00100 | 91 | AGGTAGTCCATG | 319 | gsususCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aaususc |
| GB00101 | 91 | AGGTAGTCCATG | 320 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAaususc |
| GB00102 | 91 | AGGTAGTCCATG | 321 | gsususcuguCUUuUGGUcaggacAACcgucuag |
| | | GACATTAATTC | | CUAUAAGUGCUGCagggugugAGAAacucc |
| | | | | uAuuGCUGgacgaugucucuuacgagGCAUUAG |
| | | | | CACAGGUAGUCCAUGGACAUUAaususc |
| GB00103 | 91 | AGGTAGTCCATG | 322 | gsususCUGUCUUUUGGUCAGGACAACcg |
| | | GACATTAATTC | | ucuagCUAUAAGuGCuGCagggugugAGAAa |
| | | | | cuccuAuuGCUGgacgaugucucuuacgagGCAU |
| | | | | UAGCACAGGUAGUCCAUGGACAUUAa |
| | | | | ususc |
| GB00104 | 91 | AGGTAGTCCATG | 323 | gsususcuguCUUuUGGUcaggacAACcgucuag |
| | | GACATTAATTC | | CUAUAAGuGCuGCAGGGUGUGAGAAA |
| | | | | CUCCUAuuGCUGgacgaugucucuuacgagGC |
| | | | | AUUAGCACAGGUAGUCCAUGGACAUU |
| | | | | Aaususc |
| GB00105 | 91 | AGGTAGTCCATG | 324 | gsususCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGUAG |
| | | | | ucCAUggACAUUAaususc |
| GB00106 | 91 | AGGTAGTCCATG | 325 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAaususc |
| GB00107 | 91 | AGGTAGTCCATG | 27 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGuAGucCAuggACAUuAaususc |
| GB00108 | 91 | AGGTAGTCCATG | 27 | gsususcuguCUUuUGGUcaggacAACcgucuag |
| | | GACATTAATTC | | CuAUAAGuGCuGCagggugugAGAAacuccu |
| | | | | AuuGCUGgacgaugucucuuacgagGcAUUAGC |
| | | | | ACaGGuAGucCAuggACAUuAaususc |
| GB00109 | 91 | AGGTAGTCCATG | 28 | gsususcuguCUUuUGGUcaggacAACcgucuag |
| | | GACATTAATTC | | CUAUAAGuCuGCagggugugAGAAacuccuA |
| | | | | uuGCUGgacgaugucucuuacgagGCAUUAGC |
| | | | | ACAGGUAGUCCAUGGACAUUAAUUCu |
| | | | | ususu |
| GB00110 | 91 | AGGTAGTCCATG | 29 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAauucuu |
| | | | | susu |
| GB00111 | 91 | AGGTAGTCCATG | 328 | gsususcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAauucuusus |
| | | | | u |
| GB00112 | 92 | CAAAAACTCAAC | 30 | gsusUCUGUCUUUUGGUCAGGACAACC |
| | | ATATTTGATCA | | GUCUAGCUAUAAGUGCUGCAGGGUGU |
| | | | | GAGAAACUCCUAUUGCUGGACGAUGU |
| | | | | CUCUUACGAGGCAUUAGCACCAAAAA |
| | | | | CUCAACAUAUUUGAUCAUususu |
| GB00113 | 93 | TACTTCAACAAA | 31 | gsusUCUGUCUUUUGGUCAGGACAACC |
| | | AAGTGAAATAT | | GUCUAGCUAUAAGUGCUGCAGGGUGU |
| | | | | GAGAAACUCCUAUUGCUGGACGAUGU |
| | | | | CUCUUACGAGGCAUUAGCACUACUUC |
| | | | | AACAAAAAGUGAAAUAUUususu |
| GB00114 | 94 | AGAAGAGCAACT | 32 | gsusUCUGUCUUUUGGUCAGGACAACC |
| | | AACTAACTTAA | | GUCUAGCUAUAAGUGCUGCAGGGUGU |
| | | | | GAGAAACUCCUAUUGCUGGACGAUGU |
| | | | | CUCUUACGAGGCAUUAGCACAGAAGA |
| | | | | GCAACUAACUAACUUAAUususu |
| GB00115 | 95 | TGGAGGAAATAA | 33 | gsusUCUGUCUUUUGGUCAGGACAACC |
| | | CTAGAGGAACA | | GUCUAGCUAUAAGUGCUGCAGGGUGU |
| | | | | GAGAAACUCCUAUUGCUGGACGAUGU |
| | | | | CUCUUACGAGGCAUUAGCACUGGAGG |
| | | | | AAAUAACUAGAGGAACAUususu |
| GB00116 | 96 | AAGAACCCACA | 34 | gsusUCUGUCUUUUGGUCAGGACAACC |
| | | GAAATTTCTCTA | | GUCUAGCUAUAAGUGCUGCAGGGUGU |
| | | | | GAGAAACUCCUAUUGCUGGACGAUGU |
| | | | | CUCUUACGAGGCAUUAGCACAAGAAC |
| | | | | CCACAGAAAUUUCUCUAUususu |
| GB00117 | 90 | AACCAACAGCAT | 35 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAUususu |
| GB00118 | 91 | aGGTAGTCCATG | 36 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | UCCAUGGACAUUAAUUCUususu |
| GB00119 | 97 | CTGGCAATGTCC | 37 | gsusUCUGUCUUUUGGUCAGGACAACC |
| | | CCAATGCAATC | | GUCUAGCUAUAAGUGCUGCAGGGUGU |
| | | | | GAGAAACUCCUAUUGCUGGACGAUGU |
| | | | | CUCUUACGAGGCAUUAGCACCUGGCA |
| | | | | AUGUCCCCAAUGCAAUCUususu |
| GB00120 | 98 | CATTGGGGACAT | 38 | gsusUCUGUCUUUUGGUCAGGACAACC |
| | | TGCCAGTAATC | | GUCUAGCUAUAAGUGCUGCAGGGUGU |
| | | | | GAGAAACUCCUAUUGCUGGACGAUGU |
| | | | | CUCUUACGAGGCAUUAGCACCAUUGG |
| | | | | GGACAUUGCCAGUAAUCUususu |
| GB00121 | 99 | CCCAAGTAAAAA | 39 | gsusUCUGUCUUUUGGUCAGGACAACC |
| | | GAATATTCAAT | | GUCUAGCUAUAAGUGCUGCAGGGUGU |
| | | | | GAGAAACUCCUAUUGCUGGACGAUGU |
| | | | | CUCUUACGAGGCAUUAGCACCCCAAG |
| | | | | UAAAAAGAAUAUUCAAUUususu |
| GB00122 | 100 | AATATAATGTTT | 40 | gsusUCUGUCUUUUGGUCAGGACAACC |
| | | GTTGTCTTTCC | | GUCUAGCUAUAAGUGCUGCAGGGUGU |
| | | | | GAGAAACUCCUAUUGCUGGACGAUGU |
| | | | | CUCUUACGAGGCAUUAGCACAAUAUA |
| | | | | AUGUUUGUUGUCUUUCCUususu |
| GB00123 | 101 | GAAGAGCAACT | 41 | gsusUCUGUCUUUUGGUCAGGACAACC |
| | | AACTAACTTAAT | | GUCUAGCUAUAAGUGCUGCAGGGUGU |
| | | | | GAGAAACUCCUAUUGCUGGACGAUGU |
| | | | | CUCUUACGAGGCAUUAGCACGAAGAG |
| | | | | CAACUAACUAACUUAAUUususu |
| GB00124 | 90 | AACCAACAGCAT | 42 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAuasasa |
| GB00125 | 90 | AACCAACAGCAT | 42 | gsusUCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00126 | 90 | AACCAACAGCAT | 346 | gsusUCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Auasasa |
| GB00127 | 90 | AACCAACAGCAT | 347 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAuAGUcAAAuasasa |
| GB00128 | 90 | AACCAACAGCAT | 348 | gsusUCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAuasasa |
| GB00129 | 90 | AACCAACAGCAT | 349 | gsusUCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Auasasa |
| GB00130 | 90 | AACCAACAGCAT | 350 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuasasa |
| GB00131 | 90 | AACCAACAGCAT | 351 | gsusUcuguCUUuUGGUcaggacAACcgucuag |
| | | AGTCAAATAAA | | CUAUAAGUGCUGCagggugugAGAAacucc |
| | | | | uAuuGCUGgacgaugucucuuacgagGCAUUAG |
| | | | | CACAACCAACAGCAUAGUCAAAuasasa |
| GB00132 | 90 | AACCAACAGCAT | 352 | gsusUCUGUCUUUUGGUCAGGACAACcg |
| | | AGTCAAATAAA | | ucuagCUAUAAGuGCuGCagggugugAGAAa |
| | | | | cuccuAuuGCUGgacgaugucucuuacgagGCAU |
| | | | | UAGCACAACCAACAGCAUAGUCAAAua |
| | | | | sasa |
| GB00133 | 90 | AACCAACAGCAT | 353 | gsusUcuguCUUuUGGUcaggacAACcgucuag |
| | | AGTCAAATAAA | | CUAUAAGuGCuGCAGGGUGUGAGAAA |
| | | | | CUCCUAuuGCUGgacgaugucucuuacgagGC |
| | | | | AUUAGCACAACCAACAGCAUAGUCAA |
| | | | | Auasasa |
| GB00134 | 90 | AACCAACAGCAT | 354 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuasasa |
| GB00135 | 90 | AACCAACAGCAT | 355 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuasasa |
| GB00136 | 90 | AACCAACAGCAT | 356 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACcAAc |
| | | | | aGcAuaGUCAAAuasasa |
| GB00137 | 90 | AACCAACAGCAT | 357 | gsusUcuguCUUuUGGUcaggacAACcgucuag |
| | | AGTCAAATAAA | | CuAUAAGuGCuGCagggugugAGAAacuccu |
| | | | | AuuGCUGgacgaugucucuuacgagGcAUUAGC |
| | | | | ACaACcAAcaGcAuaGUCAAAuasasa |
| GB00138 | 90 | AACCAACAGCAT | 329 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUAGUCAAAUAAAuususu |
| GB00139 | 90 | AACCAACAGCAT | 43 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACAACCAA |
| | | | | CAGCAUGUCAAAuaaauususu |
| GB00140 | 90 | AACCAACAGCAT | 330 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuAAA |
| | | | | uususu |
| GB00141 | 90 | AACCAACAGCAT | 331 | gsusUCUGUCUUUUGGUCAGGACAACC |
| | | AGTCAAATAAA | | GUCUAGCUAUAAGUGCUGCAGGGUGU |
| | | | | GAGAAACUCCUAUUGCUGGACGAUGU |
| | | | | CUCUUACGAGGCAUUAGCACaACCAAc |
| | | | | aGCAuaGUCAAAuaaauususu |
| GB00142 | 90 | AACCAACAGCAT | 332 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAUAAA |
| | | | | uususu |
| GB00143 | 90 | AACCAACAGCAT | 333 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACAACCAACAGCAUAGUCAAAuaaauu |
| | | | | susu |
| GB00144 | 90 | AACCAACAGCAT | 334 | gsusUcuguCUUuUGGUcaggacAACcgucuag |
| | | AGTCAAATAAA | | CUAUAAGuGCuGCagggugugAGAAacuccu |
| | | | | AuuGCUGgacgaugucucuuacgagGCAUUAG |
| | | | | CACaACCAAcaGCAuaGUCAAAuaaauusus |
| | | | | u |
| GB00145 | 91 | AGGTAGTCCATG | 44 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | UCCAUGGACAUUAaususc |
| GB00146 | 91 | AGGTAGTCCATG | 372 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGuAGu |
| | | | | cCAuggACAUuAaususc |
| GB00147 | 91 | AGGTAGTCCATG | 373 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACAGGUAG |
| | | | | ucCAUggACAUUAaususc |
| GB00148 | 91 | AGGTAGTCCATG | 374 | gsusUCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAaususc |
| GB00149 | 91 | AGGTAGTCCATG | 375 | gsusUCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aaususc |
| GB00150 | 91 | AGGTAGTCCATG | 376 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAaususc |
| GB00151 | 91 | AGGTAGTCCATG | 377 | gsusUcuguCUUuUGGUcaggacAACcgucuag |
| | | GACATTAATTC | | CUAUAAGUGCUGCagggugugAGAAacucc |
| | | | | uAuuGCUGgacgaugucucuuacgagGCAUUAG |
| | | | | CACAGGUAGUCCAUGGACAUUAaususc |
| GB00152 | 91 | AGGTAGTCCATG | 378 | gsusUCUGUCUUUUGGUCAGGACAACcg |
| | | GACATTAATTC | | ucuagCUAUAAGuGCuGCagggugugAGAAa |
| | | | | cuccuAuuGCUGgacgaugucucuuacgagGCAU |
| | | | | UAGCACAGGUAGUCCAUGGACAUUAa |
| | | | | ususc |
| GB00153 | 91 | AGGTAGTCCATG | 379 | gsusUcuguCUUuUGGUcaggacAACcgucuag |
| | | GACATTAATTC | | CUAUAAGuGCuGCAGGGUGUGAGAAA |
| | | | | CUCCUAuuGCUGgacgaugucucuuacgagGC |
| | | | | AUUAGCACAGGUAGUCCAUGGACAUU |
| | | | | Aaususc |
| GB00154 | 91 | AGGTAGTCCATG | 380 | gsusUCUGUCUUUUGGUCAGGACAACC |
| | | GACATTAATTC | | GUCUAGCUAUAAGUGCUGCAGGGUGU |
| | | | | GAGAAACUCCUAUUGCUGGACGAUGU |
| | | | | CUCUUACGAGGCAUUAGCACaGGUAG |
| | | | | ucCAUggACAUUAaususc |
| GB00155 | 91 | AGGTAGTCCATG | 381 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAaususc |
| GB00156 | 91 | AGGTAGTCCATG | 382 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGuAGucCAuggACAUuAaususc |
| GB00157 | 91 | AGGTAGTCCATG | 383 | gsusUcuguCUUuUGGUcaggacAACcgucuag |
| | | GACATTAATTC | | CuAUAAGuGCuGCagggugugAGAAacuccu |
| | | | | AuuGCUGgacgaugucucuuacgagGcAUUAGC |
| | | | | ACaGGuAGucCAuggACAUuAaususc |
| GB00158 | 91 | AGGTAGTCCATG | 45 | gsusUcuguCUUuUGGUcaggacAACcgucuag |
| | | GACATTAATTC | | CUAUAAGuCuGCagggugugAGAAacuccuA |
| | | | | uuGCUGgacgaugucucuuacgagGCAUUAGC |
| | | | | ACAGGUAGUCCAUGGACAUUAAUUCu |
| | | | | ususu |
| GB00159 | 91 | AGGTAGTCCATG | 341 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACAGGUAGUCCAUGGACAUUAauucuu |
| | | | | susu |
| GB00160 | 91 | AGGTAGTCCATG | 342 | gsusUcuguCUUuUGGUcaggacAACcgucuag |
| | | GACATTAATTC | | CUAUAAGuGCuGCagggugugAGAAacuccu |
| | | | | AuuGCUGgacgaugucucuuacgagGCAUUAG |
| | | | | CACaGGUAGucCAUggACAUUAauucuusus |
| | | | | u |
| GB00161 | 90 | AACCAACAGCAT | 358 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUAGUCAAAuasasa |
| GB00162 | 90 | AACCAACAGCAT | 359 | gsusuCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00163 | 90 | AACCAACAGCAT | 360 | gsusuCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAUAGUCAA |
| | | | | Auasasa |
| GB00164 | 90 | AACCAACAGCAT | 361 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAuAGUcAAAuasasa |
| GB00165 | 90 | AACCAACAGCAT | 362 | gsusuCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAuAG |
| | | | | UcAAAuasasa |
| GB00166 | 90 | AACCAACAGCAT | 363 | gsusuCuGUCUUUUGGUcAGGAcAACcgU |
| | | AGTCAAATAAA | | CuaGCuAuAaGuGCUGcaGgGuguGAGAAA |
| | | | | CUCCuaUuGcugGAcGAugUCuCUuacGagG |
| | | | | cAUuAGcACAACCAACAGCAuAGUcAA |
| | | | | Auasasa |
| GB00167 | 90 | AACCAACAGCAT | 364 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuasasa |
| GB00168 | 90 | AACCAACAGCAT | 365 | gsusucuguCUUuUGGUcaggacAACcgucuagC |
| | | AGTCAAATAAA | | UAUAAGUGCUGCagggugugAGAAacuccu |
| | | | | AuuGCUGgacgaugucucuuacgagGCAUUAG |
| | | | | CACAACCAACAGCAUAGUCAAAuasasa |
| GB00169 | 90 | AACCAACAGCAT | 366 | gsusuCUGUCUUUUGGUCAGGACAACcgu |
| | | AGTCAAATAAA | | cuagCUAUAAGuGCuGCagggugugAGAAac |
| | | | | uccuAuuGCUGgacgaugucucuuacgagGCAUU |
| | | | | AGCACAACCAACAGCAUAGUCAAAuas |
| | | | | asa |
| GB00170 | 90 | AACCAACAGCAT | 367 | gsusucuguCUUuUGGUcaggacAACcgucuagC |
| | | AGTCAAATAAA | | UAUAAGuGCuGCAGGGUGUGAGAAAC |
| | | | | UCCUAuuGCUGgacgaugucucuuacgagGCA |
| | | | | UUAGCACAACCAACAGCAUAGUCAAA |
| | | | | uasasa |
| GB00171 | 90 | AACCAACAGCAT | 368 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACCAAca |
| | | | | GCAuaGUCAAAuasasa |
| GB00172 | 90 | AACCAACAGCAT | 369 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACaACCAAcaGCAuaGUCAAAuasasa |
| GB00173 | 90 | AACCAACAGCAT | 370 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACcAAca |
| | | | | GcAuaGUCAAAuasasa |
| GB00174 | 90 | AACCAACAGCAT | 371 | gsusucuguCUUuUGGUcaggacAACcgucuagC |
| | | AGTCAAATAAA | | uAUAAGuGCuGCagggugugAGAAacuccuA |
| | | | | uuGCUGgacgaugucucuuacgagGcAUUAGCA |
| | | | | CaACcAAcaGcAuaGUCAAAuasasa |
| GB00175 | 90 | AACCAACAGCAT | 335 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUAGUCAAAUAAAuususu |
| GB00176 | 90 | AACCAACAGCAT | 271 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACAACCAAC |
| | | | | AGCAUGUCAAAuaaausususu |
| GB00177 | 90 | AACCAACAGCAT | 336 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuAAAuu |
| | | | | susu |
| GB00178 | 90 | AACCAACAGCAT | 337 | gsusuCUGUCUUUUGGUCAGGACAACCG |
| | | AGTCAAATAAA | | UCUAGCUAUAAGUGCUGCAGGGUGUG |
| | | | | AGAAACUCCUAUUGCUGGACGAUGUC |
| | | | | UCUUACGAGGCAUUAGCACaACCAAca |
| | | | | GCAuaGUCAAAuaaauususu |
| GB00179 | 90 | AACCAACAGCAT | 338 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAUAAAu |
| | | | | ususu |
| GB00180 | 90 | AACCAACAGCAT | 339 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACAACCAACAGCAUAGUCAAAuaaauusu |
| | | | | su |
| GB00181 | 90 | AACCAACAGCAT | 340 | gsusucuguCUUuUGGUcaggacAACcgucuagC |
| | | AGTCAAATAAA | | UAUAAGuGCuGCagggugugAGAAacuccuA |
| | | | | uuGCUGgacgaugucucuuacgagGCAUUAGC |
| | | | | ACaACCAAcaGCAuaGUCAAAuaaauususu |
| GB00182 | 91 | AGGTAGTCCATG | 384 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACAGGUAGU |
| | | | | CCAUGGACAUUAaususc |
| GB00183 | 91 | AGGTAGTCCATG | 385 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACaGGuAGuc |
| | | | | CAuggACAUuAaususc |
| GB00184 | 91 | AGGTAGTCCATG | 386 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACAGGUAGu |
| | | | | cCAUggACAUUAaususc |
| GB00185 | 91 | AGGTAGTCCATG | 387 | gsusuCuGUCUUUUGGUcAGGAcAACcgU |
| | | GACATTAATTC | | CuaGCUAUAAGUGCUGcaGGGugUGAGA |
| | | | | AACUCCuaUUGcugGAcGAugUCuCUuacG |
| | | | | AGGCAUUAGCACAGGUAGUCCAUGGA |
| | | | | CAUUAaususc |
| GB00186 | 91 | AGGTAGTCCATG | 388 | gsusuCuGUCUUUUGGUcAGGAcAACcgU |
| | | GACATTAATTC | | CuaGCuAuAaGuGCUGcaGgGuguGAGAAA |
| | | | | CUCCuaUuGcugGAcGAugUCuCUuacGagG |
| | | | | cAUuAGcACAGGUAGUCCAUGGACAUU |
| | | | | Aaususc |
| GB00187 | 91 | AGGTAGTCCATG | 389 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACAGGUAGUCCAUGGACAUUAaususc |
| GB00188 | 91 | AGGTAGTCCATG | 390 | gsusucuguCUUuUGGUcaggacAACcgucuagC |
| | | GACATTAATTC | | UAUAAGUGCUGCagggugugAGAAacuccu |
| | | | | AuuGCUGgacgaugucucuuacgagGCAUUAG |
| | | | | CACAGGUAGUCCAUGGACAUUAaususc |
| GB00189 | 91 | AGGTAGTCCATG | 391 | gsusuCUGUCUUUUGGUCAGGACAACcgu |
| | | GACATTAATTC | | cuagCUAUAAGuGCuGCagggugugAGAAac |
| | | | | uccuAuuGCUGgacgaugucucuuacgagGCAUU |
| | | | | AGCACAGGUAGUCCAUGGACAUUAaus |
| | | | | usc |
| GB00190 | 91 | AGGTAGTCCATG | 392 | gsusucuguCUUuUGGUcaggacAACcgucuagC |
| | | GACATTAATTC | | UAUAAGuGCuGCAGGGUGUGAGAAAC |
| | | | | UCCUAuuGCUGgacgaugucucuuacgagGCA |
| | | | | UUAGCACAGGUAGUCCAUGGACAUUA |
| | | | | aususc |
| GB00191 | 91 | AGGTAGTCCATG | 393 | gsusuCUGUCUUUUGGUCAGGACAACCG |
| | | GACATTAATTC | | UCUAGCUAUAAGUGCUGCAGGGUGUG |
| | | | | AGAAACUCCUAUUGCUGGACGAUGUC |
| | | | | UCUUACGAGGCAUUAGCACaGGUAGuc |
| | | | | CAUggACAUUAaususc |
| GB00192 | 91 | AGGTAGTCCATG | 394 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGUAGucCAUggACAUUAaususc |
| GB00193 | 91 | AGGTAGTCCATG | 395 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGuAGucCAuggACAUuAaususc |
| GB00194 | 91 | AGGTAGTCCATG | 396 | gsusucuguCUUuUGGUcaggacAACcgucuagC |
| | | GACATTAATTC | | uAUAAGuGCuGCagggugugAGAAacuccuA |
| | | | | uuGCUGgacgaugucucuuacgagGcAUUAGCA |
| | | | | CaGGuAGucCAuggACAUuAaususc |
| GB00195 | 91 | AGGTAGTCCATG | 397 | gsusucuguCUUuUGGUcaggacAACcgucuagC |
| | | GACATTAATTC | | UAUAAGuCuGCagggugugAGAAacuccuAu |
| | | | | uGCUGgacgaugucucuuacgagGCAUUAGCA |
| | | | | CAGGUAGUCCAUGGACAUUAAUUCuus |
| | | | | usu |
| GB00196 | 91 | AGGTAGTCCATG | 343 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACAGGUAGUCCAUGGACAUUAauucuus |
| | | | | usu |
| GB00197 | 91 | AGGTAGTCCATG | 344 | gsusucuguCUUuUGGUcaggacAACcgucuagC |
| | | GACATTAATTC | | UAUAAGuGCuGCagggugugAGAAacuccuA |
| | | | | uuGCUGgacgaugucucuuacgagGCAUUAGC |
| | | | | ACaGGUAGucCAUggACAUUAauucuususu |
| GB00198 | 90 | AACCAACAGCAT | 46 | gsususCuGUCUUUUGGUcAGGAcXACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00199 | 90 | AACCAACAGCAT | 47 | gsususCuGUCUUUUGGUcAGGAcAXCcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00200 | 90 | AACCAACAGCAT | 48 | gsususCuGUCUUUUGGUcAGGAcAACcgU |
| | | AGTCAAATAAA | | CuaGCUXUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00201 | 90 | AACCAACAGCAT | 49 | gsususCuGUCUUUUGGUcAGGAcAACcgU |
| | | AGTCAAATAAA | | CuaGCUAUXAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00202 | 90 | AACCAACAGCAT | 50 | gsususCuGUCUUUUGGUcAGGAcAACcgU |
| | | AGTCAAATAAA | | CuaGCUAUAXGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00203 | 90 | AACCAACAGCAT | 51 | gsususCuGUCUUUUGGUcAGGAcAACcgU |
| | | AGTCAAATAAA | | CuaGCUAUAAGUGCUGcaGGGugUGXGA |
| | | | | AACUCCuaUUGcugGAcGAugUCuCUuacG |
| | | | | AGGCAUUAGCACAACCAACAGCAUAG |
| | | | | UCAAAusasasa |
| GB00204 | 90 | AACCAACAGCAT | 52 | gsususCuGUCUUUUGGUcAGGAcAACcgU |
| | | AGTCAAATAAA | | CuaGCUAUAAGUGCUGcaGGGugUGAGX |
| | | | | AACUCCuaUUGcugGAcGAugUCuCUuacG |
| | | | | AGGCAUUAGCACAACCAACAGCAUAG |
| | | | | UCAAAusasasa |
| GB00205 | 90 | AACCAACAGCAT | 53 | gsususCuGUCUUUUGGUcAGGAcAACcgU |
| | | AGTCAAATAAA | | CuaGCUAUAAGUGCUGcaGGGugUGAGA |
| | | | | XACUCCuaUUGcugGAcGAugUCuCUuacG |
| | | | | AGGCAUUAGCACAACCAACAGCAUAG |
| | | | | UCAAAusasasa |
| GB00206 | 90 | AACCAACAGCAT | 54 | gsususCuGUCUUUUGGUcAGGAcAACcgU |
| | | AGTCAAATAAA | | CuaGCUAUAAGUGCUGcaGGGugUGAGA |
| | | | | AXCUCCuaUUGcugGAcGAugUCuCUuacG |
| | | | | AGGCAUUAGCACAACCAACAGCAUAG |
| | | | | UCAAAusasasa |
| GB00207 | 90 | AACCAACAGCAT | 55 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGXcGAugUCuCUuacG |
| | | | | AGGCAUUAGCACAACCAACAGCAUAG |
| | | | | UCAAAusasasa |
| GB00208 | 90 | AACCAACAGCAT | 56 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGXugUCuCUuacG |
| | | | | AGGCAUUAGCACAACCAACAGCAUAG |
| | | | | UCAAAusasasa |
| GB00209 | 90 | AACCAACAGCAT | 57 | gsususCuGUCUUUUGGUcAGGAcAACcgU |
| | | AGTCAAATAAA | | CuxGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00210 | 90 | AACCAACAGCAT | 58 | gsususCuGUCUUUUGGUcAGGAcAACcgU |
| | | AGTCAAATAAA | | CuaGCUAUAAGUGCUGcxGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00211 | 90 | AACCAACAGCAT | 59 | gsususCuGUCUUUUGGUcAGGAcAACcgU |
| | | AGTCAAATAAA | | CuaGCUAUAAGUGCUGcaGGGugUGAGA |
| | | | | AACUCCuxUUGcugGAcGAugUCuCUuacG |
| | | | | AGGCAUUAGCACAACCAACAGCAUAG |
| | | | | UCAAAusasasa |
| GB00212 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00213 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00214 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00215 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00216 | 90 | AACCAACAGCAT | 197 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00217 | 90 | AACCAACAGCAT | 60 | gsususCuGUCUUUUGGUcAGGAcXACcgU |
| | | AGTCAAATAAA | | CuaGCUAUAAGUGCUGcaGGGugUGAGA |
| | | | | XACUCCuaUUGcugGAcGAugUCuCUuacG |
| | | | | AGGCAUUAGCACAACCAACAGCAUAG |
| | | | | UCAAAusasasa |
| GB00218 | 90 | AACCAACAGCAT | 61 | gsususCuGUCUUUUGGUcAGGAcAXCcgU |
| | | AGTCAAATAAA | | CuxGCUAUAAGUGCUGcxGGGugUGAG |
| | | | | AAACUCCuaUUGcugGAcGAugUCuCUuac |
| | | | | GAGGCAUUAGCACAACCAACAGCAUA |
| | | | | GUCAAAusasasa |
| GB00219 | 90 | AACCAACAGCAT | 62 | gsususCuGUCUUUUGGUcAGGAcAACcgU |
| | | AGTCAAATAAA | | CuaGCUXUAAGUGCUGcaGGGugUGAGA |
| | | | | AACUCCuxUUGcugGAcGAugUCuCUuacG |
| | | | | AGGCAUUAGCACAACCAACAGCAUAG |
| | | | | UCAAAusasasa |
| GB00220 | 90 | AACCAACAGCAT | 49 | gsususCuGUCUUUUGGUcAGGAcAACcgU |
| | | AGTCAAATAAA | | CuaGCUAUXAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusasasa |
| GB00221 | 90 | AACCAACAGCAT | 63 | gsususCuGUCUUUUGGUcAGGAcAACcgU |
| | | AGTCAAATAAA | | CuaGCUAUAXGUGCUGcaGGGugUGAGA |
| | | | | AACUCCuaUUGcugGXcGAugUCuCUuacG |
| | | | | AGGCAUUAGCACAACCAACAGCAUAG |
| | | | | UCAAAusasasa |
| GB00222 | 90 | AACCAACAGCAT | 51 | gsususCuGUCUUUUGGUcAGGAcAACcgU |
| | | AGTCAAATAAA | | CuaGCUAUAAGUGCUGcaGGGugUGXGA |
| | | | | AACUCCuaUUGcugGAcGAugUCuCUuacG |
| | | | | AGGCAUUAGCACAACCAACAGCAUAG |
| | | | | UCAAAusasasa |
| GB00223 | 90 | AACCAACAGCAT | 64 | gsususCuGUCUUUUGGUcAGGAcAACcgU |
| | | AGTCAAATAAA | | CuxGCUAUAAGUGCUGcaGGGugUGAGX |
| | | | | AACUCCuaUUGcugGAcGAugUCuCUuacG |
| | | | | AGGCAUUAGCACAACCAACAGCAUAG |
| | | | | UCAAAusasasa |
| GB00224 | 90 | AACCAACAGCAT | 65 | gsususCuGUCUUUUGGUcAGGAcAACcgU |
| | | AGTCAAATAAA | | CuxGCUAUAAGUGCUGcxGGGugUGAG |
| | | | | AXACUCCuaUUGcugGAcGAugUCuCUuac |
| | | | | GAGGCAUUAGCACAACCAACAGCAUA |
| | | | | GUCAAAusasasa |
| GB00225 | 90 | AACCAACAGCAT | 66 | gsususCuGUCUUUUGGUcAGGAcAXCcgU |
| | | AGTCAAATAAA | | CuaGCUAUAAGUGCUGcaGGGugUGAGA |
| | | | | AXCUCCuaUUGcugGAcGAugUCuCUuacG |
| | | | | AGGCAUUAGCACAACCAACAGCAUAG |
| | | | | UCAAAusasasa |
| GB00226 | 90 | AACCAACAGCAT | 67 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGXcGAugUCuCUuxcG |
| | | | | AGGCAUUAGCACAACCAACAGCAUAG |
| | | | | UCAAAusasasa |
| GB00227 | 90 | AACCAACAGCAT | 68 | gsususCuGUCUUUUGGUcAGGAcAACcgU |
| | | AGTCAAATAAA | | CuxGCUAUAAGUGCUGcxGGGugUGAG |
| | | | | AAACUCCuxUUGcugGAcGAugUCuCUuxc |
| | | | | GAGGCAUUAGCACAACCAACAGCAUA |
| | | | | GUCAAAusasasa |
| GB00228 | 90 | AACCAACAGCAT | 69 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuxcG |
| | | | | AGGCAUUAGCACAACCAACAGCAUAG |
| | | | | UCAAAusasasa |
| GB00229 | 90 | AACCAACAGCAT | 70 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | XGGCAUUAGCACAACCAACAGCAUAG |
| | | | | UCAAAusasasa |
| GB00230 | 90 | AACCAACAGCAT | 298 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00231 | 90 | AACCAACAGCAT | 298 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00232 | 90 | AACCAACAGCAT | 298 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00233 | 90 | AACCAACAGCAT | 298 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00234 | 90 | AACCAACAGCAT | 71 | gsususCuGUCUUUUGGUcAGGAcXACcgU |
| | | AGTCAAATAAA | | CuaGCUAUAAGUGCUGcaGGGugUGAGA |
| | | | | XACUCCuaUUGcugGAcGAugUCuCUuacG |
| | | | | AGGCAUUAGCACAACCAACAGCAUAG |
| | | | | UCAAAuasasa |
| GB00235 | 90 | AACCAACAGCAT | 72 | gsususCuGUCUUUUGGUcAGGAcAXCcgU |
| | | AGTCAAATAAA | | CuxGCUAUAAGUGCUGcxGGGugUGAG |
| | | | | AAACUCCuaUUGcugGAcGAugUCuCUuac |
| | | | | GAGGCAUUAGCACAACCAACAGCAUA |
| | | | | GUCAAAuasasa |
| GB00236 | 90 | AACCAACAGCAT | 73 | gsususCuGUCUUUUGGUcAGGAcAACcgU |
| | | AGTCAAATAAA | | CuaGCUXUAAGUGCUGcaGGGugUGAGA |
| | | | | AACUCCuxUUGcugGAcGAugUCuCUuacG |
| | | | | AGGCAUUAGCACAACCAACAGCAUAG |
| | | | | UCAAAuasasa |
| GB00237 | 90 | AACCAACAGCAT | 74 | gsususCuGUCUUUUGGUcAGGAcAACcgU |
| | | AGTCAAATAAA | | CuaGCUAUXAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAuasasa |
| GB00238 | 90 | AACCAACAGCAT | 75 | gsususCuGUCUUUUGGUcAGGAcAACcgU |
| | | AGTCAAATAAA | | CuaGCUAUAXGUGCUGcaGGGugUGAGA |
| | | | | AACUCCuaUUGcugGXcGAugUCuCUuacG |
| | | | | AGGCAUUAGCACAACCAACAGCAUAG |
| | | | | UCAAAuasasa |
| GB00239 | 90 | AACCAACAGCAT | 76 | gsususCuGUCUUUUGGUcAGGAcAACcgU |
| | | AGTCAAATAAA | | CuaGCUAUAAGUGCUGcaGGGugUGXGA |
| | | | | AACUCCuaUUGcugGAcGAugUCuCUuacG |
| | | | | AGGCAUUAGCACAACCAACAGCAUAG |
| | | | | UCAAAuasasa |
| GB00240 | 90 | AACCAACAGCAT | 77 | gsususCuGUCUUUUGGUcAGGAcAACcgU |
| | | AGTCAAATAAA | | CuxGCUAUAAGUGCUGcaGGGugUGAGX |
| | | | | AACUCCuaUUGcugGAcGAugUCuCUuacG |
| | | | | AGGCAUUAGCACAACCAACAGCAUAG |
| | | | | UCAAAuasasa |
| GB00241 | 90 | AACCAACAGCAT | 78 | gsususCuGUCUUUUGGUcAGGAcAACcgU |
| | | AGTCAAATAAA | | CuxGCUAUAAGUGCUGcxGGGugUGAG |
| | | | | AXACUCCuaUUGcugGAcGAugUCuCUuac |
| | | | | GAGGCAUUAGCACAACCAACAGCAUA |
| | | | | GUCAAAuasasa |
| GB00242 | 90 | AACCAACAGCAT | 79 | gsususCuGUCUUUUGGUcAGGAcAXCcgU |
| | | AGTCAAATAAA | | CuaGCUAUAAGUGCUGcaGGGugUGAGA |
| | | | | AXCUCCuaUUGcugGAcGAugUCuCUuacG |
| | | | | AGGCAUUAGCACAACCAACAGCAUAG |
| | | | | UCAAAuasasa |
| GB00243 | 90 | AACCAACAGCAT | 80 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGXcGAugUCuCUuxcG |
| | | | | AGGCAUUAGCACAACCAACAGCAUAG |
| | | | | UCAAAuasasa |
| GB00244 | 90 | AACCAACAGCAT | 81 | gsususCuGUCUUUUGGUcAGGAcAACcgU |
| | | AGTCAAATAAA | | CuxGCUAUAAGUGCUGcxGGGugUGAG |
| | | | | AAACUCCuxUUGcugGAcGAugUCuCUuxc |
| | | | | GAGGCAUUAGCACAACCAACAGCAUA |
| | | | | GUCAAAuasasa |
| GB00245 | 90 | AACCAACAGCAT | 82 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuxcG |
| | | | | AGGCAUUAGCACAACCAACAGCAUAG |
| | | | | UCAAAuasasa |
| GB00246 | 90 | AACCAACAGCAT | 83 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | XGGCAUUAGCACAACCAACAGCAUAG |
| | | | | UCAAAuasasa |
| GB00247 | 90 | AACCAACAGCAT | 84 | gsususCUGUCUUUUGGUCAGGACXXCC |
| | | AGTCAAATAAA | | GUCUXGCUXUXXGUGCUGCXGGGUGU |
| | | | | GXGXXXCUCCUXUUGCUGGXCGXUGU |
| | | | | CUCUUXCGXGGCXUUXGCXCXXCCXX |
| | | | | CXGCXUXGUCXXXusXsXsX |
| GB00248 | 90 | AACCAACAGCAT | 85 | gsususCUGUCUUUUGGUCAGGACXXCC |
| | | AGTCAAATAAA | | GUCUAGCUAUXXGUGCUGCAGGGUGU |
| | | | | GXGXXACUCCUAUUGCUGGACGAUGU |
| | | | | CUCUUXCGAGGCAUUAGCACAACCAA |
| | | | | CAGCAUAGUCAAAusasasa |
| GB00249 | 90 | AACCAACAGCAT | 399 | gsususCUGUCUUUUGGUCAGGACXXCC |
| | | AGTCAAATAAA | | GUCUAGCUAUXXGUGCUGCAGGGUGU |
| | | | | GXGXxACUCCUAUUGCUGGACGAUGU |
| | | | | CUCUUxCGAGGCAUUAGCACAACCAA |
| | | | | CAGCAUAGUCAAAusasasa |
| GB00250 | 90 | AACCAACAGCAT | 86 | gsususCUGUCUUUUGGUCxGGxCxxCCG |
| | | AGTCAAATAAA | | UCUxGCUxUxxGUGCUGCxGGGUGUGx |
| | | | | GxxxCUCCUxUUGCUGGxCGxUGUCUCU |
| | | | | UxCGxGGCxUUxGCxCxxCCxxCxGCxUxG |
| | | | | UCxxxusxsxsx |
| GB00251 | 90 | AACCAACAGCAT | 398 | gsususCuGUCUUUUGGUcAGGAcXXCcgU |
| | | AGTCAAATAAA | | CuXGCUXUXXGUGCUGcXGGGugUGXG |
| | | | | XXXCUCCuXUUGcugGXcGXugUCuCUuX |
| | | | | cGXGGCXUUXGCXCXXCCXXCXGCXU |
| | | | | XGUCXXXusXsXsX |
| GB00252 | 90 | AACCAACAGCAT | 87 | gsususCuGUCUUUUGGUcAGGAcAACcgU |
| | | AGTCAAATAAA | | CuxGCUAUAAGUGCUGcxGGGugUGAG |
| | | | | AAACUCCuxUUGcugGAcGAugUCuCUuxc |
| | | | | GAGGCAUUAGCACAACCAACAGCAUA |
| | | | | GUCAAAusxsxsx |
| GB00253 | 90 | AACCAACAGCAT | 88 | gsususCuGUCUUUUGGUcXGGAcAACcgU |
| | | AGTCAAATAAA | | CuaGCUAUAAGUGCUGcxGGGugUGAGA |
| | | | | AACUCCuaUUGcugGAcGAugUCuCUuacG |
| | | | | AGGCAUUAGCACAACCAACAGCAUAG |
| | | | | UCAAAusxsxsx |
| GB00254 | 90 | AACCAACAGCAT | 89 | gsususCuGUCUUUUGGUcAGGAcAACcgU |
| | | AGTCAAATAAA | | CuaGCUAUAAGUGCUGcaGGGugUGAGA |
| | | | | AACUCCuaUUGcugGAcGAugUCuCUuacG |
| | | | | AGGCAUUAGCACAACCAACAGCAUAG |
| | | | | UCAAAusxsxsx |

Letters indicate:
For Protospacer-A: 2′-deoxyadenosine, C: 2′-deoxycytidine, G: 2′-deoxyguanosine and T: thymidine or 2′-deoxythymidine
For sgRNA/gRNA-A: Adensine, C: Cytidine; G: Guanosine, U: Uridine; T: thymidine; X: nebularine (Purine riboside) a: 2′-O-methyladenosine (2′-OMe-A); c: 2′-O-methylcytidine (2′-OMe-C); g: 2′-O-methylguanosine (2′-OMe-G); u: 2′-O-methyluridine (2′-OMe-U); x: 2′-O-methylnebularine; dX: 2′-deoxynebularine and s: phosphorothioate (PS) linkage
Underlined positions in the gRNA: ANGPTL3 RNA spacer sequence analogous to the protospacer (modified and adapted from the exemplary protospacer in the third column of Table 1)

### Target Genes

It is contemplated that the chemically modified guide RNAs described herein target a polynucleotide sequence of a particular gene of interest. To edit a target gene, the compositions disclosed herein comprising a single gRNA and, e.g., a Cas12b protein, wherein the single gRNA comprises a spacer sequence and a scaffold sequence, wherein the spacer sequence hybridizes with a target polynucleotide sequence in a target gene and the scaffold sequence binds, e.g., a Cas12b protein, contact a target gene polynucleotide. The single gRNA therefore directs, e.g., a Cas12b protein to the target polynucleotide sequence to result in a modification in the target gene. In some embodiments, the target gene is selected from a gene encoding PCSK9, APOC3 and ANGPTL3. In some embodiments, the target polynucleotide sequence is in a PCSK9 gene. In some embodiments, the target polynucleotide sequence is in a ANTPLT3 gene.

In some embodiments, the disclosure provides genome/base-editing systems, compositions and methods for editing a polynucleotide encoding an Apolipoprotein C3 (APOC3) protein and variants thereof. In some embodiments, provided herein are genome/base-editing systems, compositions and methods for editing a polynucleotide encoding Proprotein convertase subtilisin/kexin type 9 (PCSK9) and variants thereof. In some embodiments, provided herein are genome/base-editing systems, compositions and methods for editing a polynucleotide encoding Angiopoietin-like 3 (ANGPTL3) and variants thereof.

For example, where editing of a gene target is desired (e.g., a liver cell), the gRNA in a ribonucleoprotein (RNP) complex of the gRNA and a Cas12b protein in a cell facilitate binding to the target gene with the composition disclosed herein to elicit Cas12b-mediated gene editing. In some embodiments, the binding of, e.g., a Cas12b-gRNA complex to its target polynucleotide sequence in the target gene is directed by a single guide RNA disclosed herein, e.g., a single guide RNA comprising (i) a spacer sequence and (ii) a scaffold sequence, wherein the spacer sequence hybridizes with a targeted complementary polynucleotide sequence in a target gene. Thus, by designing the guide RNA sequence for any gene of interest that binds to a Cas12b protein can guide the gRNA-protein complex to the desired target polynucleotide sequence in the target gene (e.g., target gene encoding PCSK9, APOC3 and ANGPTL3) to elicit Cas12b-mediated gene editing. In some embodiments, the guide RNA sequence is co-delivered or -transfected with, e.g., a Cas12b protein in a cell where editing is desired.

In some embodiments, a target gene comprising more than one mutation described herein are contemplated. For example, a target gene encoding a variant protein can be produced using the methods described herein that includes 1, 2, 3, 4, 5, 6, 7, 8, 9, 10, or more mutations. To make multiple mutations in the target gene, a plurality of guide RNA sequences can be used, each guide RNA sequence targeting one target polynucleotide sequence in the target gene. For example, a Cas12b protein is capable of editing each and every target polynucleotide sequence dictated by the guide RNA sequence. For instance, 1, 2, 3, 4, 5, 6, 7, 8, 9, 10, or more guide RNA sequences can be used in a gene editing reaction. In some embodiments, the guide RNA sequences as used (e.g., gRNA). In some embodiments, DNA molecule encoding the guide RNA sequences can also be used.

In some embodiments, simultaneous modifications of more than one target genes (e.g., more than one target gene in the LDL-mediated cholesterol clearance pathway) are also contemplated herein. For example, in some embodiments, a modification may be simultaneously introduced into PCSK9 and APOC3 gene. In some embodiments, a modification may be simultaneously introduced into PCSK9 and ANGPTL3 gene. In some embodiments, a modification may be simultaneously introduced into APOC3 and ANGPTL3 gene. In some embodiments, a modification may be simultaneously introduced into PCSK9, APOC3 and/or ANGPTL3 gene. To simultaneously introduce modifications into more than one target genes, multiple guide nucleotide sequences are used.

To edit a gene encoding the PCSK9 protein, the gene is contacted with the composition described herein. In some embodiments, a target polynucleotide sequence in a target gene is hybridized with the designed spacer complementary single guide RNA of the Cas12b protein-gRNA RNP complex disclosed herein and, e.g., a Cas12b protein or a nucleic acid sequence encoding, e.g., a Cas12b mRNA, wherein the mRNA translate to protein in the cell and the protein thus produced first forming RNP complex with single guide RNA to effect gRNA-directed Cas12b protein-mediated modification to the target gene (e.g., target gene encoding PCSK9, APOC3 and ANGPTL3). In some embodiments, the target polynucleotide sequence is the gene locus in the genomic DNA of a cell. In some embodiments, the cell is a cultured cell. In some embodiments, the cell is in vivo. In some embodiments, the cell is in vitro. In some embodiments, the cell is ex vivo.

In some embodiments, the cell is from a mammal. In some embodiments, the mammal is a human. In some embodiments, the mammal is a rodent. In some embodiments, the rodent is a mouse. In some embodiments, the rodent is a rat. As would be understood be those skilled in the art, a target polynucleotide sequence may be a DNA molecule comprising a coding strand and a complementary strand, e.g., the PCSK9 gene locus in a genome. As such, the target polynucleotide sequence may also include coding regions (e.g., exons) and non-coding regions (e.g., introns or splicing sites). In some embodiments, the target polynucleotide sequence is located in the coding region (e.g., an exon) of the target gene (e.g., the PCSK9 gene locus). As such, the modification in the coding region may result in an amino acid change in the protein encoded by the target gene, i.e., a mutation. In some embodiments, the mutation is a loss of function mutation. In some embodiments, the loss-of-function mutation is a naturally occurring loss-of-function mutation. In some embodiments, the target polynucleotide sequence is located in a non-coding region of the target gene, e.g., in an intron or a splicing site.

In some embodiments, a target polynucleotide sequence is located in a splicing site and the editing of such sequence causes alternative splicing of the mRNA of a target gene. In some embodiments, the alternative splicing leads to leading to loss-of-function mutants. In some embodiments, the alternative splicing leads to the introduction of a premature stop codon in a mRNA encoded by the target gene, resulting in truncated and unstable proteins. In some embodiments, mutants that are defective in folding are produced. A loss-of-function variant generated by a gene that is modified using the compositions and methods disclosed herein, may have reduced activity compared to a wild-type protein encoded by an unmodified target gene. Activity refers to any known biological activity of the wild-type protein in the art.

In some embodiments, the activity of a loss-of-function variant may be reduced by at least 20%, at least 30%, at least 40%, at least 50%, at least 60%, at least 70%, at least 80%, at least 90%, at least 99%, or more. In some embodiments, the loss-of-function variant has no more than 50%, no more than 40%, no more than 30%, no more than 20%, no more than 10%, no more than 5%, no more than 1% or less activity compared to a wild-type protein.

In some embodiments, cellular activity of a protein encoded by a target gene may be reduced by reducing the level of properly folded and active protein. Introducing destabilizing mutations into the wild-type protein may cause misfolding or deactivation of the protein. A variant generated by modifying a target gene using the compositions and methods disclosed herein comprises one or more destabilizing mutations may have reduced activity compared to the wild-type protein encoded by an unmodified target gene. For example, the activity of a variant comprising one or more destabilizing mutations may be reduced by at least about 20%, at least about 30%, at least about 40%, at least about 50%, at least about 60%, at least about 70%, at least about 80%, at least about 90%, at least about 95%, at least about 99%, or more.

In some embodiments, the methods and composition disclosed herein reduces or abolishes expression and/or function of protein encoded by a target gene. For example, the methods and composition disclosed herein reduces expression and/or function of protein encoded by the target gene by at least 3-fold, at least 4-fold, at least 5-fold, at least 6-fold, at least 7-fold, at least 8-fold, at least 9-fold, or at least 10-fold relative to a control.

In some embodiments, the methods and composition disclosed herein reduces or abolishes expression and/or function of the protein encoded by a target gene by at least 2-fold relative to a control. For example, the methods and composition disclosed herein reduces or abolishes expression and/or function of the protein encoded by a target gene by at least 3-fold, at least 4-fold, at least 5-fold, at least 6-fold, at least 7-fold, at least 8-fold, at least 9-fold, or at least 10-fold relative to a control.

Some aspects of the present disclosure provide strategies of editing target gene to reduce the amount of full-length, functional protein being produced. In some embodiments, stop codons may be introduced into the coding sequence of target gene upstream of the normal stop codon (referred to as a “premature stop codon”). Premature stop codons cause premature translation termination, in turn resulting in truncated and nonfunctional proteins and induces rapid degradation of the mRNA via the non-sense mediated mRNA decay pathway. See, e.g., Baker et al., Current Opinion in Cell Biology 16 (3): 293-299, 2004; Chang et al, Annual Review of Biochemistry 76:51-74, 2007; and Behm-Ansmant et al, Genes & Development 20 (4): 391-398, 2006, each of which is incorporated herein by reference.

The methods and compositions described herein may be used to convert several amino acid codons to a stop codon (e.g., TAA, TAG, or TGA). Thus, it is envisioned that, for amino acid codons containing a C base, the C base may be converted to T. For example, a CAG (Gln/Q) codon may be changed to a TAG (amber) codon via the deamination of the first C on the coding strand. For sense codons that contain a guanine (G) base, a C base is present on the complementary strand; and the G base may be converted to an adenosine (A) via the deamination of the C on the complementary strand. For example, a TGG (Trp/W) codon may be converted to a TAG (amber) codon via the deamination of the second C on the complementary strand. In some embodiments, two C to T changes are required to convert a codon to a nonsense codon. For example, a CGG (R) codon is converted to a TAG (amber) codon via the deamination of the first C on the coding strand and the deamination of the second C on the complementary strand.

In some embodiments, the introduction of stop codons may be efficacious in generating truncations when the target polynucleotide sequence is located in a flexible loop. In some embodiments, two codons adjacent to each other may both be converted to stop codons, resulting in two stop codons adjacent to each other (also referred to as “tandem stop codons”). “Adjacent” means there are no more than 5 amino acids between the two stop codons. For example, the two stop codons may be immediately adjacent to each other (0 amino acids in between) or have 1, 2, 3, 4, or 5 amino acids in between.

Some aspects of the present disclosure provide strategies of reducing cellular activity of the protein encoded by a target gene via preventing maturation and production of mRNA encoded by the target gene. In some embodiments, such strategies involve alterations of splicing sites in the target gene. Altered splicing site may lead to altered splicing and maturation of the mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an in frame stop codon is encountered by the translating ribosome in the intron. In some embodiments, a start codon is edited and protein translation initiates at the next ATG codon, which may not be in the correct coding frame. The splicing sites typically comprises an intron donor site, a Lariat branch point, and an intron acceptor site. The mechanism of splicing is familiar to those skilled in the art.

### Proprotein Convertase Subtilisin-Kexin Type 9 (PCSK9)

In some embodiments, the target gene for modification using the compositions and methods disclosed herein is gene encoding PCSK9. Proprotein convertase subtilisin-kexin type 9 (PCSK9), also known as neural apoptosis-regulated convertase 1 (“NARC-I”), is a proteinase K-like subtilase identified as the 9th member of the secretory subtilase family. “Proprotein convertase subtilisin/kexin type 9 (PCSK9)” refers to an enzyme encoded by the PCSK9 gene. PCSK9 binds to the receptor for low-density lipoprotein (LDL) particles. In the liver, the LDL receptor removes LDL particles from the blood through the endocytosis pathway. When PCSK9 binds to the LDL receptor, the receptor is channeled towards the lysosomal pathway and broken down by proteolytic enzymes, limiting the number of times that a given LDL receptor is able to uptake LDL particles from the blood. Thus, blocking PCSK9 activity may lead to more LDL receptors being recycled and present on the surface of the liver cells, and will remove more LDL cholesterol from the blood.

Therefore, blocking PCSK9 can lower blood cholesterol levels. PCSK9 orthologs are found across many species. PCSK9 is inactive when first synthesized, a pre-pro enzyme, because a section of the peptide chain blocks its activity; proprotein convertases remove that section to activate the enzyme. Pro-PCSK9 is a secreted, globular, serine protease capable of proteolytic auto-processing of its N-terminal pro-domain into a potent endogenous inhibitor of PCSK9, which blocks its catalytic site. PCSK9's role in cholesterol homeostasis has been exploited medically. Drugs that block PCSK9 can lower the blood level of low-density lipoprotein cholesterol (LDL-C). The first two PCSK9 inhibitors, alirocumab and evolocumab, were approved by the U.S. Food and Drug Administration in 2015 for lowering cholesterol where statins and other drugs were insufficient.

The human gene for PCSK9 localizes to human chromosome Ip33-p34.3. PCSK9 is expressed in cells capable of proliferation and differentiation including, for example, hepatocytes, kidney mesenchymal cells, intestinal ileum, and colon epithelia as well as embryonic brain telencephalon neurons. See, e.g., Seidah et al., 2003 PNAS 100:928-933, which is incorporated herein by reference.

Original synthesis of PCSK9 is in the form of an inactive enzyme precursor, or zymogen, of 72-kDa, which undergoes autocatalytic, intramolecular processing in the endoplasmic reticulum (“ER”) to activate its functionality. This internal processing event has been reported to occur at the SSVFAQ jSIP motif (SEQ ID NO: 103), and has been reported as a requirement of exit from the ER. “j” indicates cleavage site. See, Benjannet et al., 2004 J. Biol. Chem. 279:48865-48875, and Seidah et al, 2003 PNAS 100:928-933, each of which are incorporated herein by reference. The cleaved protein is then secreted. The cleaved peptide remains associated with the activated and secreted enzyme.

The gene sequence for human PCSK9 is ~22-kb long with 12 exons encoding a 692 amino acid protein. The protein sequence of human PCSK9 can be found, for example, at Deposit No. NP_777596.2, which sequence is incorporated herein in its entirety. Human, mouse and rat PCSK9 nucleic acid sequences have been deposited; see, e.g., GenBank Accession Nos.: AX127530 (also AX207686), AX207688, and AX207690, respectively, each of which sequence is incorporated herein in its entirety. The gene sequence of Macaca fascicularis can be found publicly, for example, NCBI Gene ID: 102142788, which sequence is incorporated herein in their entirety. Macaca fascicularis proprotein convertase subtilisin/kexin type 9 isoform X2 sequence can be found publicly, for example, at NCBI Reference Sequence: XP_005543317.1, which sequence is incorporated herein in its entirety.

The translated protein contains a signal peptide in the NH2-terminus, and in cells and tissues an about 74 kDa zymogen (precursor) form of the full-length protein is found in the endoplasmic reticulum. During initial processing in the cell, the about 14 kDa prodomain peptide is autocatalytically cleaved to yield a mature about 60 kDa protein containing the catalytic domain and a C-terminal domain often referred to as the cysteine-histidine rich domain (CHRD). This about 60 kDa form of PCSK9 is secreted from liver cells. The secreted form of PCSK9 appears to be the physiologically active species, although an intracellular functional role of the about 60 kDa form has not been ruled out.

Numerous PCSK9 variants are disclosed and/or claimed in several patent publications including, but not limited to the following: PCT Publication Nos. WO2001031007, WO2001057081, WO2002014358, WO2001098468, WO2002102993, WO2002102994, WO2002046383, WO2002090526, WO2001077137, and WO2001034768; US Publication Nos. US 2004/0009553 and US 2003/0119038, and European Publication Nos. EP 1 440 981, EP 1 067 182, and EP 1 471 152, each of which are incorporated herein by reference.

Several mutant forms of PCSK9 are well characterized, including S 127R, N157K, F216L, R218S, and D374Y, with S 127R, F216L, and D374Y being linked to autosomal dominant hypercholesterolemia (ADH). Benjannet et al. (J. Biol. Chem., 279(47):48865-48875 (2004)) demonstrated that the S 127R and D374Y mutations result in a significant decrease in the level of pro-PCSK9 processed in the ER to form the active secreted zymogen. As a consequence, it is believed that wild-type PCSK9 increases the turnover rate of the LDL receptor causing inhibition of LDL clearance (Maxwell et al, PNAS, 102(6):2069-2074 (2005); Benjannet et al, and Lalanne et al), while PCSK9 autosomal dominant mutations result in increased levels of LDLR, increased clearance of circulating LDL, and a corresponding decrease in plasma cholesterol levels. See, Rashid et al, PNAS, 102(15):5374-5379 (2005); Abifadel et al, 2003 Nature Genetics 34:154-156; Timms et al, 2004 Hum. Genet. 114:349-353; and Leren, 2004 Clin. Genet. 65:419-422, each of which are incorporated herein by reference.

A later-published study on the S 127R mutation of Abifadel et al, reported that patients carrying such a mutation exhibited higher total cholesterol and apoB 100 in the plasma attributed to (1) an overproduction of apoB 100-containing lipoproteins, such as low-density lipoprotein (“LDL”), very low-density lipoprotein (“VLDL”) and intermediate-density lipoprotein (“IDL”), and (2) an associated reduction in clearance or conversion of said lipoproteins. Together, the studies referenced above evidence the fact that PCSK9 plays a role in the regulation of LDL production. Expression or upregulation of PCSK9 is associated with increased plasma levels of LDL cholesterol, and inhibition or the lack of expression of PCSK9 is associated with low LDL cholesterol plasma levels. Significantly, lower levels of LDL cholesterol associated with sequence variations in PCSK9 have conferred protection against coronary heart disease; Cohen et al, 2006 N. Engl. J. Med. 354:1264-1272.

Lalanne et al. demonstrated that LDL catabolismwas impaired and apolipoprotein B-containing lipoprotein synthesis was enhanced in two patients harboring S 127R mutations in PCSK9 (J. Lipid Research, 46:1312-1319 (2005)). Sun et al. also provided evidence that mutant forms of PCSK9 are also the cause of unusually severe dominant hypercholesterolaemia as a consequence of its effect of increasing apolipoprotein B secretion (Sun et al, Hum. Mol. Genet, 14(9): 1161-1169 (2005)). These results were consistent with earlier results which demonstrated adenovirus-mediated overexpression of PCSK9 in mice results in severe hypercholesteromia due to drastic decreases in the amount of LDL receptor Dubuc et al., Thromb. Vase. Biol., 24:1454-1459 (2004), in addition to results demonstrating mutant forms of PCSK9 also reduce the level of LDL receptor (Park et al., J. Biol. Chem., 279:50630-50638 (2004). The overexpression of PCSK9 in cell lines, including liver-derived cells, and in livers of mice in vivo, results in a pronounced reduction in LDLR protein levels and LDLR functional activity without changes in LDLR mRNA level (Maxwell et al., Proc. Nat. Amer. Set, 101:7100-7105 (2004); Benjannet S. et al, J. Bio. Chem. 279:48865-48875 (2004)).

Various therapeutic approaches to the inhibition of PSCK9 have been proposed, including: inhibition of PSCK9 synthesis by gene silencing agents, e.g., RNAi; inhibition of PCSK9 binding to LDLR by monoclonal antibodies, small peptides or adnectins; and inhibition of PCSK9 autocatalytic processing by small molecule inhibitors. These strategies have been described in Hedrick et al., Curr Opin Investig Drugs 2009; 10:938-46; Hooper et al, Expert Opin Biol Ther, 2013; 13:429-35; Rhainds et al, Clin Lipid, 2012; 7:621-40; Seidah et al, Expert Opin Ther Targets 2009; 13:19-28; and Seidah et al, Nat Rev Drug Discov 2012; 11:367-83, each of which are incorporated herein by reference.

In some embodiments, the loss of function mutation induced in PCSK9 e.g., G106R, L253F, A443T, R93C, etc. In some embodiments, the loss-of-function mutation is engineered (i.e., not naturally occurring), e.g., G24D, S47F, R46H, S 153N, H193Y, etc.

PCSK9 variants that can be useful in the present disclosure are loss-of-function variants that may boost LDL receptor-mediated clearance of LDL cholesterol, alone or in combination with other genes involved in the pathway, e.g., APOC3, LDL-R, or Idol. In some embodiments, the PCSK9 loss-of-function variants produced using the methods of the present disclosure express efficiently in a cell. In some embodiments, the PCKS9 loss-of-function variants produced using the methods of the present disclosure is activated and exported to engage the clathrin-coated pits from unmodified cells in a paracrine mechanism, thus competing with the wild-type PCSK9 protein. In some embodiments, the PCSK9 loss-of-function variant comprises mutations in residues in the LDL-R bonding region that make direct contact with the LDL-R protein. In some embodiments, the residues in the LDL-R bonding region that make direct contact with the LDL-R protein are selected from the group consisting of R194, R237, F379, S372, D374, D375, D378, R46, R237, and A443.

As described herein, a loss-of-function PCSK9 variant, may have reduced activity compared to a wild-type PCSK9 protein. PCSK9 activity refers to any known biological activity of the PCSK9 protein in the art. For example, in some embodiments, PCSK9 activity refers to its protease activity. In some embodiments, PCSK9 activity refers to its ability to be secreted through the cellular secretory pathway. In some embodiments, PCSK9 activity refers to its ability to act as a protein-binding adaptor in clathrin-coated vesicles. In some embodiments, PCSK9 activity refers to its ability to interact with LDL receptor. In some embodiments, PCSK9 activity refers to its ability to prevent LDL receptor recycling. These examples are not meant to be limiting.

In some embodiments, the activity of a loss-of-function PCSK9 variant may be reduced by at least 20%, at least 30%, at least 40%, at least 50%, at least 60%, at least 70%, at least 80%, at least 90%, at least 99%, or more. In some embodiments, the loss-of-function PCSK9 variant has no more than 50%, no more than 40%, no more than 30%, no more than 20%, no more than 10%, no more than 5%, no more than 1% or less activity compared to a wild-type PCSK9 protein. Non-limiting, exemplary assays for determining PCSK9 activity have been described in the art, e.g., in US Patent Application Publication US20120082680, which are incorporated herein by reference.

In some embodiments, cellular PCSK9 activity may be reduced by reducing the level of properly folded and active PCSK9 protein. Introducing destabilizing mutations into the wild-type PCSK9 protein may cause misfolding or deactivation of the protein. A PCSK9 variant comprising one or more destabilizing mutations described herein may have reduced activity compared to the wild-type PCSK9 protein. For example, the activity of a PCSK9 variant comprising one or more destabilizing mutations described herein may be reduced by at least about 20%, at least about 30%, at least about 40%, at least about 50%, at least about 60%, at least about 70%, at least about 80%, at least about 90%, at least about 95%, at least about 99%, or more.

In some embodiments, the methods and composition disclosed herein reduces or abolishes expression of protein encoded by a target gene and/or function thereof. For example, the methods and composition disclosed herein reduces expression and/or function of PCSK9 protein encoded by the PCSK9 gene by at least 3-fold, at least 4-fold, at least 5-fold, at least 6-fold, at least 7-fold, at least 8-fold, at least 9-fold, or at least 10-fold relative to a control. For example, the methods and composition disclosed herein reduces expression and/or function of APOC3 protein encoded by the APOC3 gene by at least 3-fold, at least 4-fold, at least 5-fold, at least 6-fold, at least 7-fold, at least 8-fold, at least 9-fold, or at least 10-fold relative to a control. For example, the methods and composition disclosed herein reduces expression and/or function of ANGPTL3 protein encoded by the ANGPTL3 gene by at least 3-fold, at least 4-fold, at least 5-fold, at least 6-fold, at least 7-fold, at least 8-fold, at least 9-fold, or at least 10-fold relative to a control.

In some embodiments, the loss of function PCSK9 variant produced using the methods described herein comprises a R46C mutation (CGT to TGT), mimicking the natural protective variant R46L. The PCSK9 R46L variant has been characterized to possess cholesterol-lowering effect and to reduce the risk of early-onset myocardial infraction. See, e.g., in Strom et al., Clinica Chimica Acta, Volume 411, Issues 3-4, 2, Pages 229-233, 2010; Saavedra et al., Arterioscler Thromb Vase Biol., 34(12):2700-5, 2014; Cameron et al., Hum. Mol. Genet, 15 (9): 1551-1558, 2006; and Bonnefond et al., Diabetologia, Volume 58, Issue 9, pp 2051-2055, 2015, each of which is incorporated herein by reference.

In some embodiments, the loss-of-function PCSK9 variant produced using the method described herein comprises a L253F mutation (CTC to TTC). PCSK9 L253F variant has been shown to reduce plasma LDL-Cholesterol levels. See, e.g., in Kotowski et al., Am J Hum Genet, 78(3): 410-422, 2006; Zhao et al., Am J Hum Genet, 79(3): 514-523, 2006; Huang et al., Circ Cardiovasc Genet, 2(4): 354-361, 2009; and Hampton et al., PNAS, vol 104, No. 37, 14604-14609, 2007, each of which are incorporated herein by reference.

In some embodiments, the loss-of-function PCSK9 variant produced using the method described herein comprises a A443T mutation (GCC to ACC). PCSK9 A443T mutant has been shown to be associated with reduced plasma LDL-Cholesterol levels. See, e.g., in Mayne et al., Lipids in Health and Disease, 2013-12:70, 2013; Allard et al., Hum Mutat, 26(5):497, 2005; Huang et al, Circ Cardiovasc Genet, 2(4): 354-361, 2009; and Benjannet et al., Journal of Biological Chemistry, Vol. 281, No. 41, 2006, each of which are incorporated herein by reference.

In some embodiments, the loss-of-function PCSK9 variant produced using the method described herein comprises a R93C mutation (CGC to TGC). PCSK9 R93C variant has been shown to be associated with reduced plasma LDL-Cholesterol levels. See, e.g., in Mayne et al., Lipids in Health and Disease, 2013-12:70, 2013; Miyake et al., Atherosclerosis, 196(1):29-36, 2008; and Tang et al., Nature Communications, 6, Article number: 10206, 2015, each of which are incorporated herein by reference.

Further, the present disclosure also contemplates the use of destabilizing mutations to counteract the effect of gain-of-function PCSK9 variant. Gain-of-function PCSK9 variants (e.g., the gain-of-function variants have been described in the art and are found to be associated with hypercholesterolemia (e.g., in Peterson et al., J Lipid Res. 2008 June; 49(6): 1152-1156; Benjannet et al., J Biol Chem. 2012 Sep. 28; 287(40):33745-55; Abifadel et al, Atherosclerosis. 2012 August; 223(2):394-400; and Cameron et al, Hum. Mol. Genet. (1 May 2006) 15(9): 1551-1558, each of which is incorporated herein by reference). Introducing destabilizing mutations into these gain-of-function PCSK9 variants may cause misfolding and deactivation of these gain-of-function variants, thereby counteracting the hyper-activity caused by the gain-of-function mutation. Further, gain-of-function mutations in several other key factors in the LDL-R mediated cholesterol clearance pathway, e.g., LDL-R, APOB, or APOC, have also been described in the art. Thus, making destabilizing mutations in these factors to counteract the deleterious effect of the gain-of-function mutation using the compositions and methods described herein, is also within the scope of the present disclosure. As such, the present disclosure further provides mutations that cause misfolding of PCSK9 protein or structurally destabilization of PCSK9 protein.

The introduction of tandem stop codons may be especially efficacious in generating truncation and nonfunctional PCSK9 mutations. Non-limiting examples of tandem stop codons that may be introduced include: W10X-W11X, Q99X-Q101X, Q342X-Q344X, and Q554X-Q555X, wherein X indicates the stop codon. In some embodiments, a stop codon may be introduced after a structurally destabilizing mutation to effectively produce truncation PCSK9 proteins. Non-limiting examples of a structurally destabilizing mutation followed by a stop codon include: P530S/L-Q531X, P581S/L-R582X, and P618S/L-Q619X, wherein X indicates the stop codon.

Some aspects of the present disclosure provide strategies of reducing cellular PCSK9 activity via preventing PCSK9 mRNA maturation and production. In some embodiments, such strategies involve alterations of splicing sites in the PCSK9 gene. Altered splicing site may lead to altered splicing and maturation of the PCSK9 mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an in frame stop codon is encountered by the translating ribosome in the intron. In some embodiments, a start codon is edited and protein translation initiates at the next ATG codon, which may not be in the correct coding frame.

The splicing sites typically comprises an intron donor site, a Lariat branch point, and an intron acceptor site. The mechanism of splicing is familiar to those skilled in the art. As a non-limiting example, the intron donor site has a consensus sequence of GGGTRAGT, and the C bases paired with the G bases in the intron donor site consensus sequence may be targeted by the methods and compositions described herein, thereby altering the intron donor site. The Lariat branch point also has consensus sequences, e.g., YTRAC, wherein Y is a pyrimidine and R is a purine. The intron acceptor site has a consensus sequence of YNCAGG, wherein Y is a pyrimidine and N is any nucleotide. As described herein, gene sequence for human PCSK9 is-22-kb long and contains 12 exons and 11 introns. Each of the exon-intron junction may be altered to disrupt the processing and maturation of the PCSK9 mRNA.

Wild-type PCSK9 Gene (NG_009061.1), Homo sapiens proprotein
convertase subtilisin/kexin type 9 (PCSK9), RefSeqGene
(LRG_275) on chromosome 1:
(SEQ ID NO: 104)
GTCCGATGGGGCTCTGGTGGCGTGATCTGCGCGCCCCAGGCGTCAAGCACCCACAC
CCTAGAAGGTTTCCGCAGCGACGTCGAGGCGCTCATGGTTGCAGGCGGGCGCCGCC
GTTCAGTTCAGGGTCTGAGCCTGGAGGAGTGAGCCAGGCAGTGAGACTGGCTCGGG
CGGGCCGGGACGCGTCGTTGCAGCAGCGGCTCCCAGCTCCCAGCCAGGATTCCGCG
CGCCCCTTCACGCGCCCTGCTCCTGAACTTCAGCTCCTGCACAGTCCTCCCCACCGC
AAGGCTCAAGGCGCCGCCGGCGTGGACCGCGCACGGCCTCTAGGTCTCCTCGCCAG
GACAGCAACCTCTCCCCTGGCCCTCATGGGCACCGTCAGCTCCAGGCGGTCCTGGTG
GCCGCTGCCACTGCTGCTGCTGCTGCTGCTGCTCCTGGGTCCCGCGGGCGCCCGTGC
GCAGGAGGACGAGGACGGCGACTACGAGGAGCTGGTGCTAGCCTTGCGTTCCGAG
GAGGACGGCCTGGCCGAAGCACCCGAGCACGGAACCACAGCCACCTTCCACCGCTG
CGCCAAGGTGCGGGTGTAGGGATGGGAGGCCGGGGCGAACCCGCAGCCGGGACGG
TGCGGTGCTGTTTCCTCTCGGGCCTCAGTTTCCCCCCATGTAAGAGAGGAAGTGGAG
TGCAGGTCGCCGAGGGCTCTTCGCTTGGCACGATCTTGGGGACTGCAGGCAAGGCG
GCGGGGGAGGACGGGTAGTGGGGAGCACGGTGGAGAGCGGGGACGGCCGGCTCTT
TGGGGACTTGCTGGGGCGTGCGGCTGCGCTATTCAGTGGGAAGGTTCGCGGGGTTG
GGAGACCCGGAGGCCGAGGAAGGGCGAGCAGAGCACTGCCAGGATATCCTGCCCA
GATTTCCCAGTTTCTGCCTCGCCGCGGCACAGGTGGGTGAAGGAGTGAATGCCTGG
AACGTACTGGGAACTGCACCAGGCACAGAGAAAGCGGGCTTGCCATTATAGTGGGT
TCCGATTTGGTTTGGAAAACATGGGCAGCGGAGGGTGGAGGGCCTGGAGAGAAGG
CCCTACCCGAGACAGGGGCGGGGTGGGAAGGACGGCAGATGCTGGGAGCACGAGG
CAATTTCTTTATGACACAGAACTCATGCTCTAGTATTCCATCTGTTTCAGCCGAAGA
AAAGAACCAGCTGAAGGGGCAGGGGAGAAGGGGCGGAGGTATTCTCGAGGCCCAT
TGGCGTCCTTTAGGACTCAGGCAGGGAAGGGCCCTTGGTGCTCTGGAGCCGGAGGT
GGTGCGCCTGGTACTGGGACCCCGGAGCTGAGCCCGGCGCCTCAGCCCACCTGGCT
GTCTGCCGACCGTGTGCGGGGCGAGTTTGCTCAACAACTCTGCCAGCTTCTGGCCCT
CAGGCTGTGGGAAGCTTCTTCCCGGGGCGAGACCACTAGCTTTTTCTAAGTATTACC
AGCCCAGGACTTGGCTGAGGTTCTGTGTCCCCCAGCTTGGAGTCAGATGTGGGGTTG
AATCTTGGCTTCCTCTCACTAGCTGTGGTGCTTGACAAGTCACTTATCCTTGAGCCTC
CATTGCCTAATCTTTAAAAGGGAGGTGACAATCGTCCCTACGGCTCAGTGGCAGCA
GATGGGGAGATGAAGGGAAAGTTCTGTTGACCATGAGTGAACTTACAATGCAAGCC
CCGGGGGGATCACTTGCAGTTTTGTCCCTGTCTGCAGTGTGACCTGTTGGTGACATT
GTCTTTGCTCCAAACCACAGCTCCTGGGGCAGAGGGGAAAATTCTGCCACTCACAG
CTGCCTGCCCACGCTTCTGTCTGAGTGTGCTGGGTGGCAGGATGGCAAGTCCTTACT
CAGCTCAGTATAGCCCTCTTCCTTGTTCCCTGAGCCTTTGACTTTCTCGAGGGATGTT
GTGGGGTTGTGGCCAGGATAAGAAAGGGCATTTCAAGTTACCACTGCTCCAAAACA
ACTGTTCTGGAAATAGTGAGTACCCCATCCTGAGAGGTGAGTAAGCAGAGGCTGTA
TGACCACCTGAACCAAGCCCTTGAGGATGTTTCTTCTCTGGTGGAAGTTTGGAACAG
GAGCCTCCTCAAGTTCATTTATTCATTCATTCAATGGTTATTTTGTGGGAATCGAATT
TAGAATGAAAATATTTTTTGGCAAGCAGAAAATAATTTTTAGACCAATCCTTTTCTT
TTAGTCATGAGAAACTGAGGCCCAGAGAGAGGAGGTCACCCCAGGTGCATTAGAAC
TGGGTTTCCAGAACTGACACTCCACTGCACAGAGTACTCTCCCAATTCATTCAATTT
TTATTTAGCGGAAGGCATTTTCAGATGGGTCTTTGAAGCATTAGTAGGAGTTCAGCG
ATGATGGTGTCATGAGAATTTTATTCTAGGATTAGGAGGTACCATGAACAAAGATA
CAGAGCTGGGAAAACCAGAGGTGGAAGATAAGGAGCACATGTCCACAGTTCTTTTT
CTTTTTTTTTTGAGATGGAGTTTCGCTCTTGTTGCCCAGGCTGGAGTGCAATGGTGCA
GTCTCAGCTCACTGCAACATCTGTCTCCCGGGTTCAAGTGGTTCTCCTGCCTCAGCC
TCCCAAGAAGCTGGGATTACAGGTACCTGCCACCACGCCCGGCTAATTTTTGTATTT
TTAGTAGAGAAGGGGTTTCACCACGTTGGCCAGGCTAGTCGCAAACTCCTGACCTC
CTCAGTGGATCCGAGGAGGTGATCCTCCCGCCTCAGCCTCCCAAAGTGCTCGAATTA
CAGGTGTGAGCCACCACGCCTGGCCTCCACAGTTCTTTATCCACCGTCTGAAATGTA
AAATGTTACGAAAACCAAAAGTTTTTTTTGTGATTTATTTGATGGTAGCACCTGACG
TGAACTGACATGAGATTATTTTTAATTTAGTTGTGTGAATATGCATATTCATATATTT
TGCTGCATAGATTACAGTATGCAGCTCCAGATTCTTCCAAGCAGACTCTGATTGCCC
ATTACTGCCTTTCTAAAATCCAAACAAGTTCTGAGGTTCAAAACCGTTTTGGCCCTA
AGGCTTTGGGTAAAGGGGGTGGACTCTGTTCTACTCTGACTGGAGTCCAAGATGCA
TATATACAGAGATATGGGTGATGGGGCTGCAAGGTAGGTTGAGGTAGGGGCCAAG
GAGGAGCATGGAGTTTGGACTTGATTCATGAGGCTGTGGGGAGCCAGTGAAGGTTC
TTAAGCAGGTATGTCTGCCTGAGAGCAGTTGGAGCAGACAAGAGCTAAAAACCAAA
CAAATCACCATAGATAGTGGCTGCTATAATTTGTTTGTCCCCTCCAAATCTCATGTG
GAAATTTGGTCCTCAGTGTTGGAAGTGGGGCCTAATGGGAGGTGTTTGGGTCATGG
GGGAGGAACCCCTGTGAAAGGCTTGGTGCCGTCCTTGTGATAATGAGTAAGTTCTC
CCGCTATGATTTCCCTTGAAGGCTGATTATTAAAAAGAGCTTGGCACCTCCCTCTCT
TCTCTCTTGCTTCTTCTCTTGCCATGTGATTGATCTCTGCACATGTAGGCTCCCCTTC
ACCTTCTGCCATCAGTGAAAGCAGCTTAAGGCCCTCACCAGAAGCAGATGCTGGTG
CCATGCTTCCTGGAGAGCTTGCAGAATCATGAGCTGAATAAATCCCTTTTCCTTGTA
AATTACTCACCTTCAGGTATTCCTTTATATAGCAACACAAAAGGACTAAGACAGTG
GCCTTGACTTTTCTCTCTCTTTAAGAAGTGTTGCCTTTGCTCACTTAGTCATCCCTTCT
GCCTGCATTTGTAGAGCATCTGGATGGGAGATTTATATAACCGTCACTCTTGACTTT
CCCAGCAGGCCTATGTCATAGGTACTGTGGTCTCTACAATACAGCAGAGGTATCTG
AGGCTCCGAGAGGTTGAGTGACTTGCTCATGGCTGCACAACCAGTAAATATTGGAG
CTGGAATTCAGGTCCACGGTTTCCTGGCTCCAAAGCCCATGATTTTTTCCCTCAATTT
ATTCTGACTGGGGCATGGGGGAGGGGGTGGCCTTTGGGCAGGGCCACCAGGAGCG
ACCAGGCCCGTAGAGAGCTGGGTGCAGGTACAGAGGAAAACCTGTTGTCGAGTGTG
GCCCGTAGTTCCCATTTTTGCCTGAATGGCACATTTGAAAGTGTTATATAACCATGT
GAATAATAATAGTTGGCCTATATGAGTTCTTTAATTTGCTTTTTGGTCCGCATTTGGT
AACTTCTTTATCATCTACTATACTCTGTTGTGTCTCTTTTGTTGTAATTTGTAAGTAG
GGGTGAGATAAAGTACACCTAGGGTTTGCTGGGTTTCTTCCATGTCATCATGTTCCT
CCTTGCATGGGGCCAGGATCCGTGGAGGTTGCCTGGCACCTACGTGGTGGTGCTGA
AGGAGGAGACCCACCTCTCGCAGTCAGAGCGCACTGCCCGCCGCCTGCAGGCCCAG
GCTGCCCGCCGGGGATACCTCACCAAGATCCTGCATGTCTTCCATGGCCTTCTTCCT
GGCTTCCTGGTGAAGATGAGTGGCGACCTGCTGGAGCTGGTGAGCCACCCTTTTTGG
GAATGGCACTTCCTGATAGGGCTGGGCCACTGCATATACACTGGGGACTGTGCTTA
GTAGGCCCATTGCTGAAAATCAGAAGGGGACAGCAAGTATGTATTGAGCACTTATC
GGGTACCAAGCACAGTAACTACTGGCTTTCTGTATAGAATTCCCTTTAAGCCTGGCC
ATGCCCCAGTGGTACGTCTATCTTCATTTGAAAGACGAGGAGACTGAAGTTCAGAG
GGGACCACACAGACAGCTAGGGGTAGAGCCTGGATCAAACCCATTGGTCTGCCTGC
CAGCCATTCTTGTGCCAATGCATCTGCTGCCTACGGAAACCTGTAGGGACAAGGCC
CTGGGATGTTCAGTGGAGCCTGAGTCATTTTATAAAAAAGCATGACTCTAGGGTCC
AAAATTCCTTTGAAGCTGTTGCTATCCAGAGTGAAGTCCCTTCTTTAGGACAGGGTG
GCCCTCCTCCCTCCTGGATGTCACATCTTCGGTGGAGGGGCAGAAAGGGGACTGGG
TATTCTCCTCACCCTGGCCCTAGTGCTTCAAATCTTAAAAAAACGTTTTTATTTGTGC
TTCTGCACCACCTTCTAGCCCACCTCGTTTCCTGGCCTCTAACTTGATGAGAGCGTGT
GTCATTTTCACACTGATTCTCCACATGGCAGGCGGTGCTTCTTAGCCTCCTGCAGAC
AGTGAGGCCCCACGGTCTTGTCCAAGGTCACACAGCGTGTAATGGGCAGGGTCAGA
GTCTGGAGTCTGGACCTGGGTCTCCTAGCTGCACTGCACTGCTGCCCCATGGGTTAA
TCAGCTCAGCATACCGTGGCTGAACAGCTACCTCATACCAAGGCCTGTGGCGCCAT
GACAGGGATTGACAGGGTCCCTGCCTTGGAAACCCGTAGTCTAAGTAGAGGAGACT
GACAAGTCAATGCCTTCCATCAGTCTGCTCAACACACGTTTACCAAGTGCCTACTGT
GTGCTGCAGAGGCGAAGATGACACAGCTCAGGCCTTTCCCTTGAGCTTACAGTTCA
GGAGGAGAGACTGACCAGTGACTGCCAGTACAGTTGACTATGGGACAATGTGCTCA
GCCTTGGGGAGAGACGAAGAAGGTACCCGTATAGCACCAGATGACAGGCACGAGC
CCCACAGGCCAGGGCAGCTGCTCAGAGGAGAGTAGGCCAAGCAGAAGGCAAACAG
AAGGCTGCAGGCATTTGCCATCGAGAGCTGGACTTCAAACTGGGCATCATACCAGC
CTGGGTTCGAGTCCTGCCCAGCCCCTTATTGGCTGTCTAACCCTGAGCAAATCCCTT
CACCTCTCTGAGCCTCATTCCTCTATCTGTAAACCAGTTATAATAATTGGAACATTC
ATTTAAGGACTAAATGAGGTCGTGAAGCATTCAGCAGATGCTAGGTACGGAAACTC
GCTGAAGTGGGGGCAGGTTAAGAAGCCTCTGGGGATACGAAGGCATCCAGGGACT
AGTTGTGGCAGGAGGCTGTTACCACTTAGGTCTGAAGGGTAAGGAGAGGGAATAGC
TTTCCCTCTGCCCAGTTGGAGCCGGTGGCATGGAGGAGAGGCTGCCTGTGGGGAAT
CACCCGAGGGTTCACCGCTGCCATGCGCAGGGAGTCAGGAGGTAGGGAGGGAGTG
GGGCAGATGCACACCATTTTTTTTTTTTTTTGAGACTCTGTTGCCCAGACTGGAGTGC
AGTGGTGCCATATCTGCACCTCTGCCTCCCGGGTTCAAGCTCACTGCAACCTCTGCC
TCCCGGGTTCAAGCGATTCTCCTGCCTCAGCCTCCCGAGTAGCTGGGACTACAGGTG
TGTGCCACCATGCCTGGCTAATTTTTGTATTTTTAATAGAGATGGGGTTTCACCATGT
TGGCCAGGCTGGTCTCGAACTCTCGACCTCAGGTGATCCCCCACCTCGGCCTCCCAA
AGTGCTGGGATTACAGGCGTGAGTCACCGCTCCCAGCTGCTGATGCACTCTTGTCCT
TCTAACTCCTGCTAGTGCCTCCCATTGGCTGAGCCCAACTGGAAGCTTTGCAAGGGA
GCTGGTGCTGCAGTTTGCACTGAGCAGGCTGGAGAAGGCTGGAGAATAGACTAGGG
GACAAACCGAATTGCCAGTGCTGTTATGTCATGATTTAGGCATGGAGTCCAGGGCC
TGAGCTTCACTCCATGTCCATCCTGCCCAGAGCCTTGGCACAGCCTGGCTCCCAGAC
AAGATGTCAAGTTCAGAATCCTTCCTAAAAGGAATCCTCTATGCCAGACCGTGTTGC
AGGGATATGGGAGTGCTGGGCTCCCAGCCTGATCAAGGAGCGAGAAAACTCAGGCT
CCTAGTCTGTCCTCCGGGGCACTAGCAGGGACAAGGTGGGAGGCTGCTGGGCTGGG
ATGTGGGGACAGGTTTGATCAGGTAAGGCCAGGCTGTGGCTGTGTTTGCTGCTGTCC
AAATGGCTTAAGCAGAGTCCCCCGGCCTCTCTGGCTTCTGCAGGCCTTGAAGTTGCC
CCATGTCGACTACATCGAGGAGGACTCCTCTGTCTTTGCCCAGAGCATCCCGTGGAA
CCTGGAGCGGATTACCCCTCCACGGTACCGGGCGGATGAATACCAGCCCCCCGGTA
AGACCCCCATCTGTGCCCTGCCCCACCCCATCTGAGCTGAATCCATTTGCTCTGCCC
TGGCCTGGCCTCCCTGCTGGTGGTTTCCACTTCTCGGGGGGCTTTGGGACTCAGCAC
CTCCACTGACCCCTTTTTTTCTGTCCCATCCCCATCCCCTGCAGCCCCCACTGCCTGC
CTTCCTGTTGCCCCACAAATGCAAAAGTCTTGCCTTAAATGATCCTCTTTTCCTTCTT
TTCTCTTGTTTTCCTTTTCTCACCATTTGGAATGGCCCAGCAGGCTGCACTTACCTTG
GAAGGAGGGTTCATCTGATGGTGACTCTACCTAGGGCCCCCAGGCCTCTATAACTCC
CAGTGCCCTGCAGACTGGACCAGATCCTTTAATGGGATAGACACAACCCTGTCTGG
GATGCCTCTGCCTACCTTCCTGTTTTGCTGCTCCACCTGCCTCCAGCTCCGTTTGGCT
TCCTGGGGCTCCCTGCCTGGGCCACTTTGTGTCTTCCCTCTAGGCCTTTCTTTCCACT
GTTCCCTCTGCCTGGTGTGGCCTGGCTATGGAAGGGAGGGAGGAGGAGCGGCCATG
GAAAACGGTCTGCATTCTAGCAGGGACTTGCAGGTGGCAATTCAGTCGGGGAAGAC
TCTAGATGCACCTGGCCTGAGGAGAGAATGAAGGGTTCTAGTTGGACTGTGTTAAG
TTTGAGGTGCCCATGGTGTGAGGTCTGGAGCTCAGCGCAGAGATGATGCAATGTGG
TGGGTCCATGCAACATGGTGCCAGGACGCAGAGCTTGGGGTGAACTCAGCTTTCAC
CCCTTACCGGTTCTCGTGGGATCTTGGGAAGCCACTTTCTTCTATGAGCTTTGTCGTT
CTTGTCTGTAAAATGGGCACATAACCCTGTCCCTGTCCTTCTCACAGGTTGCTGTGA
GACTCCAATGAGTTGAAGGATGTGCAGATGCTTTTGGAAGTGAAAAGTTGGGGGGC
TACTGTGTGACTTTGCATACACCCAAACTGTGTGACCTTGCATATGTCTGAGTTGCT
GCCATTGCAACAGATCAGAGCTGGTGGGCTGGGTGTGGAGAAAGGGTTTGTGTGGG
GGACATCCTCTGGCAAGGGTGGCAGCAGCAGAAGTGAGGGGCCTGGTCGGTCATGT
GTGCTGACCCGGCCTGGGCAGCCTGTGGCCAGGGAGAGGACAGCTCCTCTGTAGGA
AGAGCCTGTTCCTTTCCAACCAGGTGAGACCTCTTCAGTGGAGCCCTGGAGCCCCCT
GTACTCCACATCAGTGCCTCAGGGACCTCCCGGAGCAGGCTAATATCAGAGACCAA
GAGGGACACTGGCAGAGGATCACAGAGACCCCAGTCCAGGCAGGGACTGAGAAGA
TCTTGCCCCCTAAGTTAGTTTCCTAGCACTGCTGTGACAAATTACCACCCCCTCGGTT
GGAACAAGTTGATTCTCTGCAGTCCTGGAGGCCAGAAGCCTGAATCAGTGTCGGCA
GGACCACTTTCTCCCGGGGGGCTCCAGGGAGAAGCTTCTCTTGCCTCTTCCGTGTCC
CAACAGCGGCAGCACACCAATCCCAGCCTCTGTCTTCACACAGCCTTCTCTGTGTCT
CTCTCCTCTTCATTGTCTCATAAGGACACTTGTCATTGGATTTAGGGCCCACTGGATC
CTCCAGGATGATCTCATGTGGGGAACCTTAACCACATCTGCAAGGACCCTTTTTCCA
AATAAGGTCACAGCCACAGGTTGTGGGGGTTAGGATGTGAGTGTATCTCTTTGGCA
GCCACTGTTCCCTCCTCTCCCTTGGGCCAGAAGCAGACGTGGGGCCCTTTCTTCCCC
ATAGGATGCCCATGGATTGCCCCCCTTCCCGCTTCCCCCGAGTGTCTGTGGGAGGTG
GCAGGAATGGCAGGCAGGGGTGTGGAACCCCTTCTGGAGTCATATCAAGGGCTTGG
CTGGAGGAAGTCCTCCTGGAGCTGTTGGGCTGGCATGGGGCAGGCTGGCTGGGCCC
AGCAGCAGCTTCTTCATTCATGGGGAGGCCACAAGCATGGGCCCTAGAGCTGGCTG
CCGCCCTCAAACCCAGACCCTGCACTCTTAACTGTGTGACCTTGCATACGTCACTCA
CCCTCTCTGATCTTCAGGTTCCTCTGCAAAAGGGAGGTAATGATAACCCTCACTCTG
GGGGGCTGTTTGGAGGGTTAAATCAGTTATTGCTGTAGCATGCATTTCTCTGTCAGG
TATTGAGTGAGGTGCTGTGATTTTAGCCCTGCATTTTTCTTTTCTTACCATTCAATAA
TAACGTTTTGAGCACCCACTGTGCGCCAGGCACCATATTAGGTGCTGGGGATACAA
ATGTGAATGAAATGAATGTGGTCTCTTCCCCCAACAGTGTATCCAGAAGATTAATCC
ATTCCTTAAACAAATGCTACTTGACACAGATTAGTTCTGGATAGGCTGAGAGCTCTG
AAGGAGTGCAGGCAGCTGCGAGCCTGTGTATCCAGCAGAAGGATCAGGAAAGGAT
TCCTGGAGGAAGCGCTGTTCTAGCCAAGACCTACGGGGGCATTATTAACCAGGCAA
AGGGGACGGTGTCCAAGCAGTGGAATGAACGTGGATTGAAGCTGTGAGGCAGGAG
GGAGTGTGGCCTGTGCAGAAGGGACCGAGGCTGGTGAGACCAGGAGGGCCTGGGT
GGCCTCCAGGTCAGATGTGAAAGGAAGAACTTGGCCACAGTCTGAGCTTCTCAGGC
GTATGGCAGGGCTGCCTGGTGAGAGGGAATGAGCTCCCTGCTCTGGAGGTATGCAA
GCAGGACTGGGCTCTCACCTGCCAGAGGCCACAGAGCTTTCCAGAGGCTGGAAGAG
GCCACTCCAAGGCCTCTTTGCCCCTGAGAGTGGTGGCTCTTCTTGAGGCCACCTTGC
CACGCTGTCACAGGGAACTAGCAGCCCCTGCCTCACCCGGGGGTTTGGAAGATAGA
GGGAGGCCTAGGAAGGGCCCTGTGTCTCATCCGAGCTGGGCCCCTTTCCAGCCTCTC
ACTGGAAGGAAGCCCAAGGATGTTCCTGTGGGGGCTTTTACCAGGCCCACCTGCCC
TCTGCTGGCCATGCTTGCAGCCTCCTGACCCTGTCCCAGCAGGACAGTGGGCTGGTG
TGAGCGGGCAGGAACCGCCTGCACTTAGAAGGTGTGGGGCTGCCTCCCCGAGCTTC
CATCTGCCGCTGGGGCCACACCCCAGGCCCAGGGATGGGACCCCACAGTGGTCACA
TCATCTTGCAGCAGAACCCAGGTACAGCTCCTGGAGCAGATGGTGGTCCCAAGCAC
GGGTGGGACCAGAAAGGACTCTCACCTGGGCTAACTCAGCTGCAGCCTCAGTTCCC
TCCTCACACACGACGAGGAACATGGACTGGAAGCCTGCCCAGCAGGCCTTCTGCTC
GATGTGCGTTGTGTGGCTTACGTCCAGGGAGGGAAGCAGCCTCTGTGCTGTCTTCTA
GATAAGCCTGTATTCCCCGGGCTGTCTGCCAATGTATCCAGTTGTCCCGTCAGCCTG
GAAGCTCTGAGGGAAAACCTTGGGCTGCTTCCTGAGCACCTGTATCCCCTGCAGCC
AGCCCGGGGCCTCTGCTAGGAGCAGACTGAGCATGGCTTATGGGCCTGGCACCATC
TGGCCTCTGCCCACCTTGCTGGCCTTGTCTTGTGTCTGCCCCTTCGACATTCCATAGC
CCAGCTCAATATCTAGTGGTTCCTCTAGGGTGGCGAGCACTGTTTGGTCTCCAGATG
TCTTCAGGTCGGAGCTCACAGCGCTCTCAGCCACCCCTTCCCAGTGTAGCACCGGGC
ACATGGTAGATGCCTATTGATGAGTGAAAGCTCCTAACACACTCAGAGAGCAAGGA
CTCCGCCTCATCCCACAGCCTGGGAGGAGAGGCAGACTGCCAAGGACCTGCTCAGC
ATGCTACAGAAGAAACCAAAGTGCCCACGGGACTGATCAGTGGAGCTTCCTGCCGA
GACTGGAGGCCTTAGGGCAGGGTAGACAGTGTGTGTGCAGGCTGGGGACTCACAGT
TCGGACTGTGCCCAGACCTACTAGCATAGTGGGTGGGTGGGAGGATGCGGGACTGG
GGGCCGACCTTGCCTGAAATTCATGTGGGATCTCAGAGCAGCCACTGAATTGCTCTG
TAGGGGGCTAAATAGTGGCCCCCACAGATACACACACCCAGACAGAGCCTGTGAGC
CAGACCTTATTTGGAGAAAAGGTCTTTGTAGATGTAATTAAGCATCTCAAGATGGC
ATCATCTGGATTATGCGGTGGGCTGTAAGTCCTGTGATGTGTCTTTATGAGAGAAAG
GCAGAGGGAGATTTGACACACACAGGAGGGGCCACGTGGAGACAGAGGTGGAGAT
TGGAGAAATGTGGCCACAAGCCAGGGAACACCAGCAGCCACCAGAAGCCGGAAGA
CGTGAGGCAGGGTTCTTCCCAGAGCCTTCGCTGCTGAGTCTGGGAATTTGTGACCGA
AGCCATAAGAAGTGGGTACACGCCCTGAGCCTCCCACACTTGCTCACCTGTCCTGA
GATGAGAATCTCTACTCTGCAGCATATTTGGAGGATCACTGCGGGGGCCACAGAGG
TGCTGTTCAGATGGCACTTCAGAAGACTCAGGAGACCCTGGGGCAGGAGCAGTTTG
ACTGACAGCCCAGAGGGCTGCCCTCTGATTCCACCTGAGGCCCTGCTTTTCCTGGCT
GCAGGGGTTCCAGGGCCAGGCCATTTCCGCTGGCGCAGGACTCTGCTAGCAGCAAC
CTGCCTGAAGTCTTCCTTTGGCCTGGCTGAGAGTTTCTGAGACCTGCGCTGGAGCGG
AGGTGCTTCCTTCCTTGCTTCCTTTCTTCCTCTCTCCCTTCTCCATCCAGCAGGCTGG
ACCTGCCTGGCATCTGTGAGCTCTCCCTACTTTCTCCTATACCCTAACCTTTGTCCTG
CATGGGCGACTCCCCCAGTGAGTCTCTTGCAGCTTTTACCCCAGTGCCTGCTTCTTG
GAGAATCCAAACTGATCCAGTTAGGGATGATAAAGTGTAGGGTAGGCGCTCGGTGA
CTGTTTTCTCTGAGGTTGTGACTCGTGTGAGGCAGAAGCAGTCCCCGTGAGCCCTCC
TGGTATCTTGTGGAGTGGAGAACGCTTGGACCTGGAGCCAGGAGGCCCAGACATAC
ATCCTGTCCGAGCTGCAGCTTCCTGTCTCTAAAATGAGCCGGCCAGCGCAGGTGGCC
AGACATCACTGTTATTCTCCTTTGAGTCTTTAAATCTTGTTGTCTTTCTTGCAGACTC
GGTGAGCTGTGAAAGGCTATAATAGGGGCTTTATTTTACACTTTGATACTATTTTTT
GAACATTCATATTATTGTTAGATATTGATATTCATATGAAGGAGCAGGATGACTTGG
GTCCTTCTTGGCAGTAGCATTGCCAGCTGATGGCCTTGGACAGTTACCTGCCCTCTC
TAGGCCTCCCTTTCCTTGTCTATGAAATACATTATAGAATAGGATGTAGTGTGTGAG
GATTTTTTGGAGGTTAAACGAGTGAATATATTTAAGGCGCTTTCACCAGTGCCTGGG
ATGTGCTCTGTAGTTTCTGTGTGTTAACTATAAGGTTGACTTTATGCTCATTCCCTCC
TCTCCCACAAATGTCGCCTTGGAAAGACGGAGGCAGCCTGGTGGAGGTGTATCTCC
TAGACACCAGCATACAGAGTGACCACCGGGAAATCGAGGGCAGGGTCATGGTCAC
CGACTTCGAGAATGTGCCCGAGGAGGACGGGACCCGCTTCCACAGACAGGTAAGCA
CGGCCGTCTGATGGGAGGGCTGCCTCTGCCCATATCCCCATCCTGGAGGTGGGTGG
GGACTGCCACCCCAGAGCGTTGCAGCTGTACTCCTGGGTTGCACCCCCCCCAGCTGT
CACTGTCCCCTCCCTGCCATCAGTTGTGGGAAGGGCGTTCATCCATCCAGCCACCTG
CTGATTTGTTATAGGGTGGAGGGGGGGTCTTTCTCATGTGGTCCTTGTGTTCGTCGA
GCAGGCCAGCAAGTGTGACAGTCATGGCACCCACCTGGCAGGGGTGGTCAGCGGCC
GGGATGCCGGCGTGGCCAAGGGTGCCAGCATGCGCAGCCTGCGCGTGCTCAACTGC
CAAGGGAAGGGCACGGTTAGCGGCACCCTCATAGGTAAGTGATGGCCCCAGACGCT
GGTCTCTCTCCATCTGGACCTGGCCTGGGAGGTGGCTTGGGCTGGGCCCAGGGAGA
GCTAATGTCTCCTAACCAAGAATGCTGTGGCAGCCTCTGCCGCAGAGCCAGAGAAC
CAGAGTGCCAAGGCTGGCAGGGTTCCCAGTGGCCACGAGTGCAGATGAAGAAACC
CAGGCCCCAAGAGGGTCATGCAGGTAGCCCAGGGAGTTCAGCCTTGACCCTGGGTC
AATGACCTTTCCACAGTTCCACACTGCTCCCCTTTTAAAATCCGGTGATGTCTTTATG
TCTTTTGTTATGTTATCTTCAATGTGGAGGGACTCGAGGTGATCTAAGCAAACTTTTT
CTATCTTCTGCTTGCATACCTCTGAGACCAGGGGACTCACTCACTTGCATGACTGGG
CCCTGCAGGTCACACTGGCCAGGCAGATGTGGTGGAGGAACTGGCAGAGGACTTTT
TCTAGACTGTGACTACATTTAGTCCACCCAGCGGCCCCCCTATGAAGTCCAGTTGAG
AACTAGGACTCTGGGGGCCGGTGGACAGAGAAGAGGGAGGGTTCTCTCCCTTACTG
ACTTCCTTCTGTGGCCAGACATTGAGCAAGGCCTCTGTACAGCATGTCCTGGGGCTG
GCCTTGCCGTAGCTGCTAAATAGTTGACGAAACCAGTCCAGAGAGGGGAGGTGACT
GCCAGGGTCGCACAGCTCAAGCTGGGGAACTCGCTGGGAAAACTGTCAGCTCTGGG
CAGCAGCTTGACTTCCACTGTAAGCCCCAGCCCCCAGGGTCAAACACTGGCTCTGGT
GCTGGCAGAGGCAGCCCACTAGCCTGTTTCAAAGGCTGAGAAGGCCCAGGAGTCTG
CCCTGTGCTCCACCAGTTCTGCCCGAGACTTTCCTACAGAGTACAGGTTTTGATGTT
CAGTTTTAAAGGCAAGAATCAATAACCTTCTGCCCCATCAGGTGACCCCTTGTGCCT
GTCCCACCCCTTTATTGACTGACCTCGGCTCAGTCAGGTCAGTTCCTGAAGGTCAGT
GTGTGGAGGGGAGGCTGTTCTTTCCCAGAAAGGCCTTCCCCAGGCCTGGTGCTCTGG
CCTCTGGAGGACTTCCTGGAGAAGTCCCTTCTTTGGGGTCCCAGTCAGTGTATGGGA
AGCCCTTATTGCATGACCTGGCACGGGGCAGGGGCTCAACAGTCACTATTGCCTTCC
TTGCCACTGCCATTTCCTCCTCTGTAAGCAGGTGATTGTGTGTCCAGTCTGAGCACA
GAGATAAGCACACAGCAGGTGCTTAATAACTAGCAGCTGTAGGCTGGGCGCGGTGG
CTCATGCCTGTAATCCCAGCACTTTGGGAGGCCGAGGTGGGCAGATCACCTGAGGT
CAGGAGTTCGAGACCAGCCTGTTCAACATGGTGAAACCCCGTCTCTACTAAAAATA
CAAAAATTAGCCAGGCATGGTGGTGGGTGTCTGTATCCCAGCTACTTGGGAGGCTA
AGGCAGGAGAATCGCTTGAACCCAGGAGGTGGAGGTTGCAGTGAGCTGAGATCGT
GCCACTGCAATCCAGCCTGAGTGATAGAGCGAGATTCCATCTCAAAAATAAATAAG
TAAATAACTAGCAGCTGTAAATGTGGCTGTTGTTCTTCACCTCCACACTCAGTGCCA
CTCCACTCCCTCCCTCCGTGGTGTGAGGGGCCTCACTAGCTGTCTCCTAGGAGGAGC
ATGGCTGTGAGATTCCAGCTCCATCCTTGGCCACGGCTCCTGGAGACATCTTAGAGG
CCAGGATCCAGAAGGCTCCCACACCTCATTTGACAGGGGAGAAGCTGTCAGTTCCA
GGTCCCCTTGCACATCAGGGCCAGAGCTGCGTTAGGCCTCCAGTCTCCAGGCCACTG
GGCCAGAGCTCACAGGCTGGCAGAGGGTTAGAACTGTTACTGGTGGCTGGGTGCAG
TGGCTCACGCCTGTAATCTTAGCACTTTGGGAGGGCAAGGCGGGAGGATCATGAGG
TCAGGACATCGAGACCATCCTTGCTAACACGGTGAAGCCCCGTCTCTACTAAAACT
ACAAAAAATTAGCCGGGCGTGGTGGCAGGCGCCTGTAGTCCCAGCTACTCAGGAGG
CTGAGGCAGGAGAATGGCGTGAACCCGGGAGGCGGAGCTTGCAGTGAGCCGAGAT
TGCGCCACTGCACTCCAGCCTGGGCAATAGAGCGAGACTCCGTCTGGAAAGAAAAA
AAAAAAAAAGAGCTGTTACTGTTGACAGTAGCATGAGGTAGACCATGGCCTGCACC
AAAATGGGGGAGTGGAGTGCCACTGAGGCCAGAAGGAACCACACCCTCAAGGGTG
GGGAGTTATGGTATGGGGGGTCCTAGGCATGGAGTCTTTTAATTCTTTAGACAATCC
TGGGAGCAACTGTCCCTGTTTCACAGAGGGCGGGGCCACACAGCTGGTGAGTGGGC
AGCCAAGACTCTGTTCAAGTTTGTGTGGGTCCAACACTTGCGGCCACGGTGGAGGG
GCATCTGAGCCAGGCCTCAGAGAGTGGCGGGGGAAGTTGGGTGGGGAAGTGTGCC
CTTCTCATTCCTCTGAGGCTCATCCTCTTGGTGCCTCTCTTTCATGGAAAGGGATAAT
AAGGTTATTGTGAGGATCCCCTGAGTTCGTATATTCAGACGCTTAGACAGAGCCAG
GCACAGAGAAGGGCCCGGGGTTGGCTAGTTTGATTGCTGGTGTAATTGCTAATATCT
TCCAGTTTGTATTGGTCAAGGTTCTGCAGAGAAGCAGAACCAGTAGGATGTATATA
TTAAGAGTTTCAAGCTCATGTGACCGTGCGGGCTGGCAAGTCTGAAATCCGCAGGG
CAGGCCAGGCAGGCTGGCAATTCCTGCAGAATTTGATGTTGCAATACTGAGTCCTA
AGGCAGTCCTGGGGCAGAATTCCTTCTTCCCTGGGAGGCCTCAGTCTGTTCTCTTAA
GGCCTTCAACTGATTAAATGAGGCCTGCCCAAGTTATAGAGAGTAACCTGCCTTACT
CCGTCTTCTGATTTAAATGTTAGTCACATCTAAAAAATATTTTCGCAGCAGCATTTC
CACTGGCTTTTGACCAAACATCAGGCCACAAAGTTGATCCCCAAAATTAACCATCA
CTCTGTGCCTGTAAGGGAGGGGCTGGGAAAGGGGAGCAGGTCTCCCCAAGGGGTG
ACCTTGGCTTTGTTCCTCCCAGGCCTGGAGTTTATTCGGAAAAGCCAGCTGGTCCAG
CCTGTGGGGCCACTGGTGGTGCTGCTGCCCCTGGCGGGTGGGTACAGCCGCGTCCTC
AACGCCGCCTGCCAGCGCCTGGCGAGGGCTGGGGTCGTGCTGGTCACCGCTGCCGG
CAACTTCCGGGACGATGCCTGCCTCTACTCCCCAGCCTCAGCTCCCGAGGTAGGTGC
TGGGGCTGCTGCCCCAAGGCGCGGGTAGGGGGCGGAGGGCGGAGGGCGGAGGGAG
GGCGGGCGGGCAGGCGGGCTTCTTGTGGCACGTGGGCTTCTTGTGGCACGTTCCTG
GAGGCCGAACCCTTCTGGCTTTGGAAGGAGTCGTCAGAGACCCCCGCCATGCGGGA
GGCTGGGGAGGAAGGGGCTCGAAACCTCCATCATCGCAGAGTCTGAATAGCAGTGG
CCCCGCCATGCGCCCACGTAGCGGCGCCTACGTAGCCACGCCCCCACACCCCGTCC
TGGCCACTCTCCCTCCTGAAGGTCTTCTGGTACCCGCCCCCTCCCCATCTCCATCCCC
AGGCCCTGCGTCCTCTGCCCAATACTCTTTGGGCCTCCCTGTTGTCCAGCTCTCTCCG
CGGCTCCATGACTGACAACTTGAGCAAGGCTAATGTGAATGGGAGCGGTTGAGGGC
TCAGACCTCTCACCCGAGGAACATCCACAGAGTGTGCCGCATGCCCGGTGCAGTGT
GGCTGCGGGGACACAGACACGGAGCCTCGGCCCTGAGGAGCTGGGGGGCAGTGAC
CGTCCCTCCTCTGACCCACCACTCCTCCAGTGTCAGGACACTGCGGGTATCTAGGGG
AAGGAATCTTGTTCCACTTCAAGTCTGGAACTTCAAGTCTGTGTGTGTGCGTGCGCG
CGCGCGCGTTGGGGGTGGGGGTTGCAGAGCAGATGCGTACCTGACAGCGGTAACCT
AGGTCCCCCCTGGCCTATCAAGGCTTCCCTGGCGGCCGAATTTAAAGGCATCAAGC
AAACAAAGCCCAACACATCTCTGCCTTGTCCTCTCAGTTTCCCCCCGTGGCACTTAG
AACCACTTGATACACCGAATAGTTTCCTATCTCCCCCACTAGGATGTAAACTCCACA
GGGGCATTGGGAATGCTGCCTGGCTATGGTAGGGACAGAGGGGAGCACCAGGGCG
GGGCAGGGGTGCCAGAGTTCTGCCTGGGCAGTCAGATTTTCCTTAGGAGGGGACAT
TTGAGTGGGACCCAAACAGGTGTATAGCAGTTGTCCAGCCCAGCTGGCAAGGCCTG
AGTCTGCCTCTGCAACCCCTCTCTTGGGCTCCTTTCTCTGCCACCCACCTCCTCACCT
TTCCAGGTCATCACAGTTGGGGCCACCAATGCCCAAGACCAGCCGGTGACCCTGGG
GACTTTGGGGACCAACTTTGGCCGCTGTGTGGACCTCTTTGCCCCAGGGGAGGACAT
CATTGGTGCCTCCAGCGACTGCAGCACCTGCTTTGTGTCACAGAGTGGGACATCACA
GGCTGCTGCCCACGTGGCTGGTAAGTCACCACCCCACTGCCTCGGCCACCGTGATGC
TAACAGCCCCTTTGGCAGTCAGGGTCTGTGCCGGGACCTCCAGTGCCAGGCTCTGTG
CAGGGGGACCAGAGATGAAGTAGGCCTGATGGTGCCTTCAAGGACACTCAGTCTGA
TGAGGGAGGCGAGTGCACAGAGGAAACACGAGGTCAGGGCTGTATTAGAGGGAGC
CCAGAGGAGGCACCTGCCCAGCCCGAGGGTCAGAGAAGGCATCTTGGAGGAGGGA
CATTTGATCGGGAGCTTGATGGATGAATAGGAGTTCACCTGGCCGATAAGACAGCA
ACTACCAAGGCTTAGAGGTGTGAGAGGAGGCTGTCTTACCTCACTGAGTAAGGACT
GCAGGCGGCTTACCTTCGAGAAGAGAGCTTAGTGTCTGTGTGCACGTGTGTTTGTGT
GTATGTGTGTGCGTGTGTGCACTGGCAGGAGTCCCCTGCTGGGGCAGGAGGGCCGG
GCCATCACCATCTTTCACCATTCACCCCTGCACCAGGCATTGCAGCCATGATGCTGT
CTGCCGAGCCGGAGCTCACCCTGGCCGAGTTGAGGCAGAGACTGATCCACTTCTCT
GCCAAAGATGTCATCAATGAGGCCTGGTTCCCTGAGGACCAGCGGGTACTGACCCC
CAACCTGGTGGCCGCCCTGCCCCCCAGCACCCATGGGGCAGGTAAGCAGGATGGCA
GGGTGGGCAAGTCCAGGCTGGGGCTTGGGAGGTCTGTGTGACCTTGACAGTCTCTC
CCTTCTCCCTTGTCTGTGTAAGGAGGATGACGCCACCTTAAATAGGATTAAATGAGA
ATGGGGCTCTGAAAGGGCTGTGCAATATTTTCATAACGTGTTTTTATAGAGACAGTT
GAGTATGTTCTTTAAGCCCTCCTCTCTCCTACCATGAACTAAAGATTTCTGTGGAGG
TCCCCTCACTCCCAGCACCCCCTCCTCATCCCAGGCCCTTTTTGCAGGTTGGCAGCT
GTTTTGCAGGACTGTATGGTCAGCACACTCGGGGCCTACACGGATGGCCACAGCCG
TCGCCCGCTGCGCCCCAGATGAGGAGCTGCTGAGCTGCTCCAGTTTCTCCAGGAGTG
GGAAGCGGCGGGGCGAGCGCATGGAGGTGACTGTACCCCTCCTTCGTGTGTGTGTG
TGTGTGTGTGTGTGTGTGTGTGTGTGTGTGTGTGTGTGTGTCAGTGCTGGGCCCTCA
GGGACCCCCAGCAAGCCCCTCCATCCTCCAGACTCCAGCTCTTCTGTAAGCTTACAG
GGCTGGCCAGACCAGGAGTGGGGCACTCCTCACTTCACGCGGCTGGGGGCTGCTGG
AGAGAGCCACAGCGGGAAGGGTTTCCTAGAGGCTGCAGGACAGTGCTGGATGGATT
TTCAATGCTCACCTGGGTGTGAGCGTGCGGCAGGGCCGCGTGAGGGTCAGCGATCT
GCTACTCTGGACTCAGCCATCTCTAGGCCCCTCTCACTCAGGTGCTCCATGGTTCTG
GGAGCTGAGAAATCTCAAACCAGCAAAAAAGTGGAATTGATGTTGATGCTACAGGA
TAGTGCACAGATGCCATCTGGTTGCAGCATTTTGGTGGAAGGGCAGTGCCCAGCTA
GGAGAGTGAGGAGGGGCAGGCATTTCTGGCTTGAGGAGATGGGGTCTTAATGCTCG
TGTGAGAGGCAGAGTGGGTGGAGTGGAGCTGGCTGGATCCTTGCTTTGGCCTCCTG
GATTTCTCTCTATCTCCATTTTGAAACCACTCTGTGTTTGGAAGAACTTTTGAGTATT
CAGAGCTGCCCACTGGCAGAACAGTCTTCCTTGGGCAGGAGTGAGCTCCTTGTCCCC
AGAAGGCTGGGTCTGGCTGGCCCCTGGCAGGGACACTGATGAGGGTGCTTGAGTTG
ATCCTGTCTAGTCCCTTTCTGTGTTTTCAAAGCCCATTCTAAAGCAGATTCCCATTTC
CGTCTTTGACTCTAAGGCCCAAGGGGGCAAGCTGGTCTGCCGGGCCCACAACGCTT
TTGGGGGTGAGGGTGTCTACGCCATTGCCAGGTGCTGCCTGCTACCCCAGGCCAACT
GCAGCGTCCACACAGCTCCACCAGCTGAGGCCAGCATGGGGACCCGTGTCCACTGC
CACCAACAGGGCCACGTCCTCACAGGTAGGAGGCTGGGCTTGCCCTGGGGTGAGGA
GGGGTCTCTTTCTCCTTATGCACCCACTGCCCGCGAGGCTTGGTCCTCACAAGTGTG
ATCCATGAGACTCAAGCCTGACTTGCAGTTCCATACTCTGGTTCTGCCACTTCCATG
CCCTTTGAGCCTGGGCAGGTGACCTTACTTCTCCTCATCTCAGCTTCCTCCTCCATAA
GAGGGAAAAAGGTATTACCTGCCTCATTGTGTTGCAAGGAGATGGGCAGCATCTAG
GGCACTGGCCTGGAGTATCGCAGGTGCTTTGCCTAAGGTGGTGCAGTCCAGGAGAG
GCAGCTCCAGAGAGAGGCCCCCGGCTGGGGCTGAAAGGAGGGCAGACCTCGGTTT
GAATTTCACCCTGCCGCTCTATAGCTGTGTGACTTGGGCAAATTACTTAACATCTCT
GTATGAGGAAATGATGAGTGCTAAGCACTTAGCTTAGTGCCGGGACAATATAAATT
CTAGCTATCGTTACTATTGTTTTCATCACCCGTTGCTTTAAAATCCAGCCTCTGGTAT
AGGCAACTATTGACGGGCTACCCTGTGTCGAAAACATGCCCAGGCAGGTAGCAGGA
AGTCACAGATGGGGACCTCTTGGGGCATCAAGGGATGGTGCCCTGAGGCTGAGCTG
TTCTGGTTGGGTGGAGCATGAGAGGTCTGGGAAGACAGTGGGACTCCAGCCTGGAA
TAAGAGGCTCAGAGTTGATTCTCGTCTGAGCACGTCCAGGGGAACCACTGAGGGTT
TGGGAACAGGAGAGTGAGGGTGAGAACCTGGTTCTGGGCACAGCAGGCTGGCATG
TAGGATGGATGTTCAGGAAAGATGAGCATAGTCAGGTGGCTGGTGCCCTTGTCCAG
GGGAGAGGCTCCGTCAGGTTCAGGGGTCCTGGCTTGGAGGGAAGTCCGCCATGCTC
TAATCACGCTCCCCTTTGGAAGTGCTCAGCCGATGAGCTCACAGGCACATGTCAGTT
TGAAGTCATGGAATCTGACTCCATGAAGCGCACCTCAAAGAGCACCATTTTGCAGC
TAAGGGAACTGCAGGCTGGACATGCTGAGTGGCTGCCCCGAGCCCTTGCAGCTAGG
ACATAGAGAATGCTAGTAACCACAACCCTACCATGTTCAGAGCACATGCCAGGCTC
CATGCTGGGGCTTCGCACGTGTCATCTTCACAGTGTCCCTGTGAGTAGGTGTGGTTT
CTCTTTCCATCTTACAAATGAGTAAACAGAGCCTCAGTGTAGCTAAGTAACCACTAT
TTTAGGTTTCTTAGCCAATGGGTGTGTCTGACTCCTAAGCCCATGGAGGGCATTCTG
AGGTGGTTCAGACAGACCCCGGCTTACCCTTGAACTTCTGCCTGCTGGCTGCATAGG
GAGGGGCTGGGGGGAGTTTGAGCATCTCAGGCCATAGAGCCCCTGCCTCACTGTCT
CCATCTCTGGGTGGAAAGATGGTGTTTTCCCTGAGAAACTAAGGCTCAGAGAGGTT
GAATGGCTCTCCCAAGGTCACACAGCTGGTCAGCTGCAGAGTTGAGAACACAGGAG
TCCTGGTGCTCAGGCCAGCATCTCTTTTTTTCTTTGAGTTGTTTCTAGGTTTCCTAGC
TCTTGCCTCAGACCTTAAAGAGAGAGGGTCTGATGGGGATGGGCACTGGAGACGGA
GCATCCCAGCATTTCACATCTGAGCTGGCTTTCCTCTGCCCCAGGCTGCAGCTCCCA
CTGGGAGGTGGAGGACCTTGGCACCCACAAGCCGCCTGTGCTGAGGCCACGAGGTC
AGCCCAACCAGTGCGTGGGCCACAGGGAGGCCAGCATCCACGCTTCCTGCTGCCAT
GCCCCAGGTCTGGAATGCAAAGTCAAGGAGCATGGAATCCCGGCCCCTCAGGAGCA
GGTGAAGAGGCCCGTGAGGCCGGGTGGGTGGGGTGCTGCGTGTCTCTCCTGCACAG
CTTTTCTGTGTCAGTTTGTGCCACCACCATACCGCCATGCATCAGGGTGGCGGTTTG
CCAGGTAGATGCTGTGGGCAGCTTCCGCCATTGTGTGGACAGCATGTATATGTGTCT
CTGTGTGGCTGGGTCTGTTTTTGCTTTTGTCCAGATCAGTAAGGTTTGCTACCTGGGT
ACCCCACTCCACTTGGAGTAGAATGTGCATAAATATGGCATAAAGAAATGCAATAT
GCATGCATTTATTGATTGATCTATTTTTTTCTGAGATGGGGTCTTGCTGTGTTGCCCA
GGCTGGTCTCAAATTCCTGGGCTCAAGCAATCCTCTGGTCTCAGCCTCCCCAAGTGT
TGGGATTATAGGCATGAGCCGCTGCACCTGGCCTCTCTGATCTATTTAACAAACCTG
CTGGGAGGGTCTCAGGGTCAGGAGCAGCACTGGGCTCTGAGGACACAGAGCTCACT
CAGCCGTGACCCAGAGGGGGTGCCTGAGCTGCATGCTGAAGGTTGTTAGCATGACC
AGCAAGGCAAGAAAAGGCCCTGCCGAGATTAGCAAGGCATGTGCCAAGCCCTGGA
ATGTGACAGCCGGGCCTTCTAGAAACCTGAGTGTATAACTCTCCTTAAAAGCCAGT
AGGAGCTCCTCAAAAGGCAGCCCTAAGGAGTCCACTCTTAAATGAACTCAGAGTCA
GTTTTAAAATGCAAGTCTGTGTTGATTCTGGTCTGGATGGTGCATTCCTCGAGAGCA
AAAGACAGTCTTGGTCTTGGATCCACTTGCCCTGGGTACACTGAGGGCTGCTAGGTT
CCAGGTGCTCTTCCTGGCACTGGGGAGGGATACAGGCCCAAGAGACATGCTGTTCT
CCCTCCTGGAGCATCTATTTTAGTGGAGGAAGACAGAAAACAAACCATTAATATAG
AGTACTGAAAAGATGCGATGGAGAAAACTATAGCAAGGAAGGGAATGGGGTGGGA
GAGAGGTCAGGAGAGGTCTCGCTGACAAGGTGGACGAAACAGGCCATGAGGCAGA
GAACATGTTCCAGGCAAAGCAAAGGCCCCCAGGTGGGGATGTGCAGGGAGTACCA
GGAAACCAGAGAGGTGGGAATAGTTATGAGATGGGGGGTGCCTCAGAGGGGACAG
GGCCAAGTCAGGTGAGACCTGAGGGTCACAGTCAGCAGTGAGCTGGGGCCATGCA
GGGGTCTGGCCTCAGAGGAGTGTGGTCTGGCCTGGATCTGAACCTCTCACTGTGGCC
TAGCTGCTGAGCTGAGAAGAGATGACAAGGACCTTGGGCAGAAGCAGGGAGACTG
GAGGGAGGCGGTGGAGGGTCCAGGCGTTGGGGCGGGGCTCAGGCTGGAGTCTGAA
GGGAGCCTGCAGGCCTGGTGGGTGGATGTGGGTGGGAGAGGGGGAGGATGGCACC
AAGGCTCGGGCCCCTGGACAGATGGAGTTGCCATTAAGTGGGATGGGGCAGGCTAT
GGGGCCATCAGTTTCAGAGGGATGAGTTTGGCACTGGCATGGTAGGCATCTGTCTA
TCTCCACGGCCCTCAAACCAGGCATGAAGCAGGAGCTCACGTGTTTGGTCAGCCAT
GGTGCAGAACCGCCTGGGTGGGAGGTGCGGGGTGGGAGATACACGGTTGTGTCCCA
AATGGGCTCTGAGCCAGCGAGGGCCGTCTGCACTTTGGCCTCACAGAAGGATGTCG
GAGGGAGAAATGAAGTGTGGGTGGGGGTCCCGGGCCACGCTAGACATGTGCTTTCT
TTTCCTCGGGCTCTGGCAGGTGACCGTGGCCTGCGAGGAGGGCTGGACCCTGACTG
GCTGCAGTGCCCTCCCTGGGACCTCCCACGTCCTGGGGGCCTACGCCGTAGACAAC
ACGTGTGTAGTCAGGAGCCGGGACGTCAGCACTACAGGCAGCACCAGCGAAGGGG
CCGTGACAGCCGTTGCCATCTGCTGCCGGAGCCGGCACCTGGCGCAGGCCTCCCAG
GAGCTCCAGTGACAGCCCCATCCCAGGATGGGTGTCTGGGGAGGGTCAAGGGCTGG
GGCTGAGCTTTAAAATGGTTCCGACTTGTCCCTCTCTCAGCCCTCCATGGCCTGGCA
CGAGGGGATGGGGATGCTTCCGCCTTTCCGGGGCTGCTGGCCTGGCCCTTGAGTGG
GGCAGCCTCCTTGCCTGGAACTCACTCACTCTGGGTGCCTCCTCCCCAGGTGGAGGT
GCCAGGAAGCTCCCTCCCTCACTGTGGGGCATTTCACCATTCAAACAGGTCGAGCTG
TGCTCGGGTGCTGCCAGCTGCTCCCAATGTGCCGATGTCCGTGGGCAGAATGACTTT
TATTGAGCTCTTGTTCCGTGCCAGGCATTCAATCCTCAGGTCTCCACCAAGGAGGCA
GGATTCTTCCCATGGATAGGGGAGGGGGCGGTAGGGGCTGCAGGGACAAACATCGT
TGGGGGGTGAGTGTGAAAGGTGCTGATGGCCCTCATCTCCAGCTAACTGTGGAGAA
GCCCCTGGGGGCTCCCTGATTAATGGAGGCTTAGCTTTCTGGATGGCATCTAGCCAG
AGGCTGGAGACAGGTGCGCCCCTGGTGGTCACAGGCTGTGCCTTGGTTTCCTGAGC
CACCTTTACTCTGCTCTATGCCAGGCTGTGCTAGCAACACCCAAAGGTGGCCTGCGG
GGAGCCATCACCTAGGACTGACTCGGCAGTGTGCAGTGGTGCATGCACTGTCTCAG
CCAACCCGCTCCACTACCCGGCAGGGTACACATTCGCACCCCTACTTCACAGAGGA
AGAAACCTGGAACCAGAGGGGGCGTGCCTGCCAAGCTCACACAGCAGGAACTGAG
CCAGAAACGCAGATTGGGCTGGCTCTGAAGCCAAGCCTCTTCTTACTTCACCCGGCT
GGGCTCCTCATTTTTACGGGTAACAGTGAGGCTGGGAAGGGGAACACAGACCAGGA
AGCTCGGTGAGTGATGGCAGAACGATGCCTGCAGGCATGGAACTTTTTCCGTTATC
ACCCAGGCCTGATTCACTGGCCTGGCGGAGATGCTTCTAAGGCATGGTCGGGGGAG
AGGGCCAACAACTGTCCCTCCTTGAGCACCAGCCCCACCCAAGCAAGCAGACATTT
ATCTTTTGGGTCTGTCCTCTCTGTTGCCTTTTTACAGCCAACTTTTCTAGACCTGTTTT
GCTTTTGTAACTTGAAGATATTTATTCTGGGTTTTGTAGCATTTTTATTAATATGGTG
ACTTTTTAAAATAAAAACAAACAAACGTTGTCCTAAC
Human PCSK9 Amino Acid Sequence (NP_777596.2):
(SEQ ID NO: 105)
MGTVSSRRSWWPLPLLLLLLLLLGPAGARAQEDEDGDYEELVLALRSEEDGLAEAPEH
GTTATFHRCAKDPWRLPGTYVVVLKEETHLSQSERTARRLQAQAARRGYLTKILHVFH
GLLPGFLVKMSGDLLELALKLPHVDYIEEDSSVFAQSIPWNLERITPPRYRADEYQPPDG
GSLVEVYLLDTSIQSDHREIEGRVMVTDFENVPEEDGTRFHRQASKCDSHGTHLAGVVS
GRDAGVAKGASMRSLRVLNCQGKGTVSGTLIGLEFIRKSQLVQPVGPLVVLLPLAGGY
SRVLNAACQRLARAGVVLVTAAGNFRDDACLYSPASAPEVITVGATNAQDQPVTLGTL
GTNFGRCVDLFAPGEDIIGASSDCSTCFVSQSGTSQAAAHVAGIAAMMLSAEPELTLAE
LRQRLIHFSAKDVINEAWFPEDQRVLTPNLVAALPPSTHGAGWQLFCRTVWSAHSGPT
RMATAVARCAPDEELLSCSSFSRSGKRRGERMEAQGGKLVCRAHNAFGGEGVYAIAR
CCLLPQANCSVHTAPPAEASMGTRVHCHQQGHVLTGCSSHWEVEDLGTHKPPVLRPR
GQPNQCVGHREASIHASCCHAPGLECKVKEHGIPAPQEQVTVACEEGWTLTGCSALPG
TSHVLGAYAVDNTCVVRSRDVSTTGSTSEGAVTAVAICCRSRHLAQASQELQ

### Apolipoprotein C3 (APOC3)

In some embodiments, the target gene for modification using the compositions and methods disclosed herein is gene encoding APOC3. The LDL-R mediated cholesterol clearance pathway involves multiple players. Non-limiting examples of protein factors involved in this pathway include: Apolipoprotein C3 (APOC3), LDL receptor (LDL-R), and Increased Degradation of LDL Receptor Protein (IDOL). These protein factors and their respective function are described in the art. Further, loss-of-function variants of these factors have been identified and characterized, and are determined to have cardio protective functions. See, e.g., J0rgensen et al., N Engl J Med 2014; 371:32-41 Jul. 3, 2014; Scholtzl et al, Hum. Mol. Genet. (1999) 8 (11): 2025-2030; De Castro-Oros et al., BMC Medical Genomics, 20147:17; and Gu et al., J Lipid Res. 2013, 54(12):3345-57, each of which are incorporated herein by reference. Thus, some aspects of the present disclosure provide the generation of loss-of-function variants of APOC3 {e.g., A43T and R19X), LDL-R, and IDOL {e.g., R266X) using the methods and compositions disclosed herein.

“Apolipoprotein C-III (APOC3)” is a protein that in humans is encoded by the APOC3 gene. APOC3 is a component of very low density lipoproteins (VLDL). APOC3 inhibits lipoprotein lipase and hepatic lipase. It is also thought to inhibit hepatic uptake of triglyceride-rich particles. An increase in APOC3 levels induces the development of hypertriglyceridemia. Recent evidence suggests an intracellular role for APOC3 in promoting the assembly and secretion of triglyceride-rich VLDL particles from hepatic cells under lipid-rich conditions. However, two naturally occurring point mutations in human apoC3 coding sequence, A23T and K58E have been shown to abolish the intracellular assembly and secretion of triglyceride-rich VLDL particles from hepatic cells.

Loss-of-function mutations that may be made in APOC3 gene using the methods and compositions described herein are also provided. The strategies to generate loss-of-function mutation are similar to that used for PCSK9 (e.g., premature stop codons, destabilizing mutations, altering splicing, etc.).

The protein sequence of human APOC3 can be found, for example, at Deposit No. NP_000031.1, which reference is incorporated herein in its entirety. Human nucleic acid sequences can be found at e.g., GenBank Accession Nos.: NG_008949.1, which sequence is incorporated herein in its entirety. Mouse, rat and monkey APOC3 nucleic acid sequences have been deposited; see, e.g., Ensembl accession number ENSMUSG00000032081, ENSRNOG00000047503, and ENSMFAG00000001837 respectively, each of which sequences is incorporated herein in its entirety.

NG_008949.1:5000-8165 Homo sapiens apolipoprotein C3 (APOC3),
RefSeqGene on chromosome 11:
(SEQ ID NO: 106)
CTGCTCAGTTCATCCCTAGAGGCAGCTGCTCCAGGTAATGCCCTCTGGGGAGGGGA
AAGAGGAGGGGAGGAGGATGAAGAGGGGCAAGAGGAGCTCCCTGCCCAGCCCAGC
CAGCAAGCCTGGAGAAGCACTTGCTAGAGCTAAGGAAGCCTCGGAGCTGGACGGG
TGCCCCCCACCCCTCATCATAACCTGAAGAACATGGAGGCCCGGGAGGGGTGTCAC
TTGCCCAAAGCTACACAGGGGGTGGGGCTGGAAGTGGCTCCAAGTGCAGGTTCCCC
CCTCATTCTTCAGGCTTAGGGCTGGAGGAAGCCTTAGACAGCCCAGTCCTACCCCAG
ACAGGGAAACTGAGGCCTGGAGAGGGCCAGAAATCACCCAAAGACACACAGCATG
TTGGCTGGACTGGACGGAGATCAGTCCAGACCGCAGGTGCCTTGATGTTCAGTCTG
GTGGGTTTTCTGCTCCATCCCACCCACCTCCCTTTGGGCCTCGATCCCTCGCCCCTCA
CCAGTCCCCCTTCTGAGAGCCCGTATTAGCAGGGAGCCGGCCCCTACTCCTTCTGGC
AGACCCAGCTAAGGTTCTACCTTAGGGGCCACGCCACCTCCCCAGGGAGGGGTCCA
GAGGCATGGGGACCTGGGGTGCCCCTCACAGGACACTTCCTTGCAGGAACAGAGGT
GCCATGCAGCCCCGGGTACTCCTTGTTGTTGCCCTCCTGGCGCTCCTGGCCTCTGCC
CGTAAGCACTTGGTGGGACTGGGCTGGGGGCAGGGTGGAGGCAACTTGGGGATCCC
AGTCCCAATGGGTGGTCAAGCAGGAGCCCAGGGCTCGTCCAGAGGCCGATCCACCC
CACTCAGCCCTGCTCTTTCCTCAGGAGCTTCAGAGGCCGAGGATGCCTCCCTTCTCA
GCTTCATGCAGGGTTACATGAAGCACGCCACCAAGACCGCCAAGGATGCACTGAGC
AGCGTGCAGGAGTCCCAGGTGGCCCAGCAGGCCAGGTACACCCGCTGGCCTCCCTC
CCCATCCCCCCTGCCAGCTGCCTCCATTCCCACCCGCCCCTGCCCTGGTGAGATCCC
AACAATGGAATGGAGGTGCTCCAGCCTCCCCTGGGCCTGTGCCTCTTCAGCCTCCTC
TTTCCTCACAGGGCCTTTGTCAGGCTGCTGCGGGAGAGATGACAGAGTTGAGACTG
CATTCCTCCCAGGTCCCTCCTTTCTCCCCGGAGCAGTCCTAGGGCGTGCCGTTTTAG
CCCTCATTTCCATTTTCCTTTCCTTTCCCTTTCTTTCTCTTTCTATTTCTTTCTTTCTTTC
TTTCTTTCTTTCTTTCTTTCTTTCTTTCTTTCTTTCTTTCTTTCTTTCTTTCCTTTCTTTCT
TTCCTTTCTTTCTTTCCTTTCTTTCTTTCTTTCCTTTCTTTCTCTTTCTTTCTTTCTTTCC
TTTTTCTTTCTTTCCCTCTCTTCCTTTCTCTCTTTCTTTCTTCTTCTTTTTTTTTTAATGG
AGTCTCCCTCTGTCACCTAGGCTGGAGTGCAGTGGTGCCATCTCGGCTCACTGCAAC
CTCCGTCTCCCGGGTTCAACCCATTCTCCTGCCTCAGCCTCCCAAGTAGCTGGGATT
ACAGGCACGCGCCACCACACCCAGCTAATTTTTGTATTTTTAGCAGAGATGGGGTTT
CACCATGTTGGCCAGGTTGGTCTTGAATTCCTGACCTCAGGGGATCCTCCTGCCTCG
GCCTCCCAAAGTGCTGGGATTACAGGCATGAGCCACTGCGCCTGGCCCCATTTTCCT
TTTCTGAAGGTCTGGCTAGAGCAGTGGTCCTCAGCCTTTTTGGCACCAGGGACCAGT
TTTGTGGTGGACAATTTTTCCATGGGCCAGCGGGGATGGTTTTGGGATGAAGCTGTT
CCACCTCAGATCATCAGGCATTAGATTCTCATAAGGAGCCCTCCACCTAGATCCCTG
GCATGTGCAGTTCACAATAGGGTTCACACTCCTATGAGAATGTAAGGCCACTTGATC
TGACAGGAGGCGGAGCTCAGGCGGTATTGCTCACTCACCCACCACTCACTTCGTGCT
GTGCAGCCCGGCTCCTAACAGTCCATGGACCAGTACCTATCTATGACTTGGGGGTTG
GGGACCCCTGGGCTAGGGGTTTGCCTTGGGAGGCCCCACCTGACCCAATTCAAGCC
CGTGAGTGCTTCTGCTTTGTTCTAAGACCTGGGGCCAGTGTGAGCAGAAGTGTGTCC
TTCCTCTCCCATCCTGCCCCTGCCCATCAGTACTCTCCTCTCCCCTACTCCCTTCTCC
ACCTCACCCTGACTGGCATTAGCTGGCATAGCAGAGGTGTTCATAAACATTCTTAGT
CCCCAGAACCGGCTTTGGGGTAGGTGTTATTTTCTCACTTTGCAGATGAGAAAATTG
AGGCTCAGAGCGATTAGGTGACCTGCCCCAGATCACACAACTAATCAATCCTCCAA
TGACTTTCCAAATGAGAGGCTGCCTCCCTCTGTCCTACCCTGCTCAGAGCCACCAGG
TTGTGCAACTCCAGGCGGTGCTGTTTGCACAGAAAACAATGACAGCCTTGACCTTTC
ACATCTCCCCACCCTGTCACTTTGTGCCTCAGGCCCAGGGGCATAAACATCTGAGGT
GACCTGGAGATGGCAGGGTTTGACTTGTGCTGGGGTTCCTGCAAGGATATCTCTTCT
CCCAGGGTGGCAGCTGTGGGGGATTCCTGCCTGAGGTCTCAGGGCTGTCGTCCAGT
GAAGTTGAGAGGGTGGTGTGGTCCTGACTGGTGTCGTCCAGTGGGGACATGGGTGT
GGGTCCCATGGTTGCCTACAGAGGAGTTCTCATGCCCTGCTCTGTTGCTTCCCCTGA
CTGATTTAGGGGCTGGGTGACCGATGGCTTCAGTTCCCTGAAAGACTACTGGAGCA
CCGTTAAGGACAAGTTCTCTGAGTTCTGGGATTTGGACCCTGAGGTCAGACCAACTT
CAGCCGTGGCTGCCTGAGACCTCAATACCCCAAGTCCACCTGCCTATCCATCCTGCG
AGCTCCTTGGGTCCTGCAATCTCCAGGGCTGCCCCTGTAGGTTGCTTAAAAGGGACA
GTATTCTCAGTGCTCTCCTACCCCACCTCATGCCTGGCCCCCCTCCAGGCATGCTGG
CCTCCCAATAAAGCTGGACAAGAAGCTGCTATGA
NP_000031.1 Human apolipoprotein C-III precursor:
(SEQ ID NO: 107)
MQPRVLLVVALLALLASARASEAEDASLLSFMQGYMKHATKTAKDALSSVQESQVAQ
QARGWVTDGFSSLKDYWSTVKDKFSEFWDLDPEVRPTSAVAA

### Angiopoietin-Like 3 (ANGPTL3)

In some embodiments, the target gene for modification using the compositions and methods disclosed herein is gene encoding ANGPTL3. ANGPTL3 has been associated with diseases and disorders such as, but not limited to, Arteriosclerosis, Atherosclerosis, Cardiovascular Diseases, Coronary heart disease, Diabetes, Diabetes Mellitus, Non-Insulin-Dependent Diabetes Mellitus, Fatty Liver, Hyperinsulinism, Hyperlipidemia, Hypertriglyceridemia, Hypobetalipoproteinemias, Inflammation, Insulin Resistance, Metabolic Diseases, Obesity, Malignant neoplasm of mouth, Lipid Metabolism Disorders, Lip and Oral Cavity Carcinoma, Dyslipidemias, Metabolic Syndrome X, Hypotriglyceridemia, Opitz trigonocephaly syndrome, Ischemic stroke, Hypertriglyceridemia result, Hypobetalipoproteinemia Familial 2, Familial hypobetalipoproteinemia, and Ischemic Cerebrovascular Accident. Editing the ANGPTL3 gene using any of the methods described herein may be used to treat, prevent and/or mitigate the symptoms of the diseases and disorders described herein.

The ANGPTL3 gene encodes the Angiopoietin-Like 3 protein, which is a determinant factor of high-density lipoprotein (HDL) level in human. It positively correlates with plasma triglyceride and HDL cholesterol. The activity of ANGPTL3 is expressed predominantly in the liver. ANGPTL3 is associated with Dyslipidemias. Dyslipidemias is a genetic disease characterized by elevated level of lipids in the blood that contributes to the development of clogged arteries (atherosclerosis). These lipids include plasma cholesterol, triglycerides, or high-density lipoprotein. Dyslipidemia increases the risk of heart attacks, stroke, or other circulatory concerns. Current management includes lifestyle changes such as exercise and dietary modifications as well as use of lipid-lowering drugs such as statins. Non-statin lipid-lowering drugs include bile acid sequestrants, cholesterol absorption inhibitors, drugs for homozygous familial hypercholesteremia, fibrates, nicotinic acid, omega-3 fatty acids and/or combination products. Treatment options usually depend on the specific lipid abnormality, although different lipid abnormalities often coexist. Treatment of children is more challenging as dietary changes may be difficult to implement and lipid-lowering therapies have not been proven effective.

ANGPTL3 is also known to cause hypobetalipoproteinemia. Hypobetalipoproteinemia is an inherited disease (autosomal recessive) that affects between 1 in 1000 and 1 in 3000 people worldwide. Common symptoms of hypobetalipoproteinemia include plasma levels of LDL cholesterol or apolipoprotein B below the 5th percentile which impairs the body's ability to absorb and transport fats and can lead to retinal degeneration, neuropathy, coagulopathy, or abnormal buildup of fats in the liver called hepatic steatosis. In severely affected patients, hepatic steatosis may progress to chronic liver disease (cirrhosis). Current treatment of hypobetalipoproteinemia includes severe restriction of long-chain fatty acids to 15 grams per day to improve fat absorption. In infants with hypobetalipoproteinemia, brief supplementation with medium-chain triglycerides may be effective but amount must be closely monitored to avoid liver toxicity. Another option for treating hypobetalipoproteinemia is administration high doses of vitamin E to prevent neurologic complications. Alternatively, vitamin A (10,000-25,000 IU/d) supplementation may be effective if an elevated prothrombin time suggests vitamin K depletion.

In one example, the target tissue for the compositions and methods described herein is liver tissue. In one example, the gene is Angiopoietin-Like 3 (ANGPTL3) which may also be referred to as Angiopoietin 5, ANGPT5, ANG-5, Angiopoietin-Like Protein 3, Angiopoietin-5, FHBL2, and ANL3. ANGPTL3 has a cytogenetic location of lp31.3 and the genomic coordinate are on Chromosome 1 on the forward strand at position 62,597,487-62,606,159.

Loss-of-function mutations that may be made in ANGPTL3 gene using the methods and compositions described herein are also provided. The strategies to generate loss-of-function mutation are similar to that used for PCSK9 (e.g., premature stop codons, destabilizing mutations, altering splicing, etc.).

The nucleotide sequence of human ANGPTL3 is provided, for example, in NG_028169.1, which is incorporated herein in its entirety. The protein sequence of human ANGPTL3 is provided, for example, AAD34156.1, which is incorporated herein in its entirety.

Mouse, rat and monkey ANGPTL3 nucleic acid sequences have been deposited; see, e.g., Ensembl accession number ENSMUSG00000028553, ENSRNOG00000008638, and ENSMFAG00000007083 respectively, each of which sequences are incorporated herein its entirety.

The polypeptide and coding nucleic acid sequences of PCSK9, APOC3 and ANGPTL3 and of other members of the family of human origin and those of a number of animals are publically available, e.g., from the NCBI website or ENSEMBL website. Examples include, but are not limited to the following sequences, each of which sequences are incorporated herein in their entireties;

NG_028169.1 Human angiopoietin like 3 (ANGPTL3), RefSeqGene
on chromosome 1:
(SEQ ID NO: 108)
AATGACAAACTGAAAAAATCTATTGTTTGTTATATATATAACAAAGAATTAGTATCC
ACAATATGTAAATAATTCCTAAAATTAGTCAGAAAGAGACAAACTTAAAAAGAGGG
TAACAAGGAGGGGAGCAAATTATGTACATAACCAGATGATTCGCAAAGACGGCAA
CAGAGATGGCCAGCAAAACAAACTAGATATATACTTGTCTATTAGATTTATCAACA
TTTTTTGCCTTTTTCATTAAAAGCATTTGTAAAAGGATATAGGAAAAGAGGAACTCT
CATATACTCCTGGCAGGGATGTAAATTGGTACAACCCTTTTGAAGGACAATCTGAC
AAAAGCAATCGTAAGTTACAAGTCAACATCTATGAATGTATATGAAAATATTTATA
TACATACATCACCACCATAAAAGCATTTTCTATACATACTGTTTATAATTAGAAAAT
TGGAAACAAATGATTAAAAGGGGGCTGATTAAATTAAGGTTCATCTATATAACAGG
ATTATGCAGCTATTAAAAAGGACGTGGTAACTCTATAGACATTCATAGGAAAATAA
ATTTTAAAATACTAAGATCCTGAATGATATATATATCATGAGCTATTATACATAACA
AGATCCCACTTGTGTTATAAAAAATTATGTTTAGTCATTCAAAGGGTCTGGTATGAT
AGACCCAAAATGTTAATAGAGTCGAGATTTTTATTTTTTATAGGTTTTTGAAATACC
TGAATTTTCACAATAAGTACTTTGCACATTAAAAATCTTAGCTGGGCATGGTGGCTC
ACGCTTGTAATCCCAGCACTCTGGGAGGCCAAGGTAGGCAGATCACCTGAGGTCAG
GAGTTCGAGACCAGTCTGGCCAACATGGTGAAACCCCGTCTCTACTAAAAATACAA
AAATTAGCCAGGCTTGGTTGGGGGTGCCTGTAATTCCAGATACTCGGGAAGCTGAG
ACAGGAGAATCGCTTGAACCCAGGAGGGGGAAGTTGCAGTGAGCTGAGATCACGA
CGCTGCACTCCAGCCTGGGCAACAAAGAGCAAAACTCCGTCTCAAAAATAAATAAA
GAAAAAATCTTTACATGTCCAAAGATACGGCTGTTCAACTAAAAAATATATATGTA
TAAAACTTAACATGTTAATAGTGAACACACAAAACAGTAAGATAGATAAAATTATT
CCTTCAAAGCTCACTTAACCTCTGGATCTACACTGTCCAAAAAGACGGTCTAATGAG
ACAATTGAGCACTTGATAGGTGAGTGGTTCTAACTGAGATATGTTCTCAGTATAAAA
CATACAATAGGATCTTCCTATACAACATTAATTAAAAAACAAACTATTGTAGTTAAA
AAGGAAAAAATTAGAGATACTATGTAAAAAAGAGCCAAAATACCTTGTATTTTATT
TGAAAGACATATCTCCATAAGATTACACAACCTCGTGTAGGATAAAGGACTTTGCTT
TGCTTGGAATTTAAACAATTTAGGCTCTTAAATGTCCTAAAATTCTCTGTAGCTAAG
AAATTTTTATATTGGTTCCTAGGAACTAGGAATCCTTAAATTAGGCCCTACATTTGC
TTACAAGTTTATTTTCCTTGGCATAAAATTTTTTAGTTTTTACATTACTGGTTATATTT
GATCAGGGTTCTATTTAAATAGGCACAAGTTCAAGCAAAGATCAGATTCTGCTTTTA
GCAGTGTGTACTCAGACAGGAAGTATTAAAAGGCAGGCAGAAAATCCTTTATAAAA
TTACTACTTTCAATGCATTTTCCCACGTTGAAATGCTTCTGCAGTTTATAATTAGGCA
AATTACTTTAATTATAATCAATAATGCTGTTCAAATTACTATAAGAATTATACAGAT
ATTTATACCAAGAGACAATATACTAGAAACCAAGACTACGTGACCATTACCTCTAC
TCTGTCAGTGTTATTTGTGAGAAATTGCACAAATTTTGCAAAAAGTGTTAGTATCCT
ACTACAGTAGGATATAATATAGAAGGAAATAATTTCATAAAGCCTGTCTTTGGTACT
AGTGCTCAGTTACTTTCATTAACTAAAAAAGGGGCTACTCTTCAAATTCCCTTCTCT
AAAAAGAATGTACTATATCAAAAGGGGGTAAACACTACTACGTATACATTCTGCAC
TTAGAAATCCCTATATGTTGATTTTATCATTCTCTTATTCAATAAATATTGTTTCTAC
AATGTGTAAGGCATTACTGTACTAAAGCATTATAAGGAATATAAGTTAAAAACACA
TACAAATCTTGCCAATCAACAGCTTATAGTGTAATAGGGGAGAGAAGCTGGCCCAT
CTATATTCTCCCTCAACTAGCAAGTGGATGAAATATCAGGGTCAATAGTTATAAGCC
ACAAAAGCTGACAGCTTAATTAAGAGAAGTTTTGAAATATGTATTTCATGACCAAT
AATTACAACTGTAACTTTTCTATTTAAAGAAGGAGAAAATTTGAATTTCTTCTCTAG
CTCAACATACACTTCTATAATTCCATTACATGAACCAGAGTAAAGGGTAAGATGGA
AATGAAGAATATTTTCTTACCCTTTTGTGGTTCTATATTGGACACTTAAAAATCATA
CACAACCTAATCAAAAGATGTAATTCTTTAAAAAGGTACGAGACCAAAATTCAGAA
AATCTAGACTATAACAAAATTTCTCAATTTACATTATCTTAATATGCAATTAATTTTC
ACCAGTAAAATACTATAGTATGGGTACAAATGCATTGATTAGTTCTAATTACAAAA
ATGGCTAATATATAATACTGTGTAGTGTTTATGATACATCAGATAATGTTCTAAGTG
CTCTGAAAATATAAACTTTTAATCTTTTATACGACCCTATAAAATAGGTGTTATTCTC
ACTGGAGAGATGAGAAAACAGGGGTTCAGAGATGTGAAGTAATTTGACCAAAGGT
CACAAAGCTGAAGAATATGAAATCCGGGATTCTGATTCAGGCAGTCTTATTCCAGA
ATCATGCTCTTAACCACTATGGAATACTGCCTCTACTGTAACTATTATACCCAAAAC
CCTTAATCCTAAGTCATCAAAAGGAAGAGCCTCTATTTTACACAATGAAGAGGCAT
TTCTAAGAATAGAAATTTAGGGACGAGCACAGTGGCTTACTCCTTTAATATCAGCAC
TTTGAGAGGCTGATATGGGAGGTTCACTTGAAGTCAGGAGTTCAAGGTCAGCTTGG
GCAACATAGTGAAACACAGTCTCTACAAAATATTTAAAAATTAGCTGGGTGTGGTG
GCATGCATCTATAGTCTCAGCTACTTGGGAGACAGAGGGAGGAGGCTTGCTCGAGC
CCAGGAGTTCGTGGCTATAGTGAGCTATGATCATGCCACTGCACTCCAGCCTGGAC
AACAGAGCAAGACCCTGTCTCTAAAAAAGAAAAGAAATTTGGAAATGGTTTATTTT
GTATTAACAATTTATAATTTACACTGAAATTTATTATGATAAAACTTTTCCCTGTGTT
AAAAAGCTATTAACTTTATGAAAAATTTCTTTTAGGTAAGGTTGATTATATATACCC
ACACACATACACAGGTTAAAAGTTAGTTTCATGTGACATAATAACTAGCATTTTGAG
CACTACCTGTTTGCCCAGCACTGTTCTAAGTGCTCTACATGTATTATTGTTAAATTAT
CATAACACTATGAATTATGTACTATAATTACCCCAGCTTTACAGATGAGGAGACTAA
TCCATGGGGAGGTTAAGTAACTTGTCCAAGGCCAGACAGCTAGAGCCGGCTTTTGG
ACCCACACCACAGTCTGACTCCAGCACCCATATTCTTAACAATTTCACCATATTAAT
ATGTCAAGATTAAGCAGTTTTAAAGGATGCTATTTTCTCACAAATTTCTTAATATGA
ACACTCAATAAGAATAATCACTAATATAAGCATTTAGTATTTTTTTAACACTAAGTT
GGAAGCATAGTGGAACATTTATTTTTAGAAATATTATTAATTGGCTGGGCTCACGCT
TGTAATCGGCTGGGCTCATGCCTGTAAATTTTGGGAGGCCAAGGTAAAAGAATTGC
TTGAGCCCAGTATTTCCAGACCAGCATGGGCAATACATTAAGACATCATCTTTAAAA
AAAAAATGTTATTAATCTCCTCTTTTTGTTAAATGTATATTATCAAAATTGTTACTAA
GCTAACAAACTTCAGAAAAACTTATGATGGGCAAGCTGCTTGTGACATTGAAGGTA
TTTAAGATTCAATTCTAGTTTGGTCCTAGATGACCACATATCCATTGTTCCTTCAACG
AGCACATGGTAAAGAGCCTAGAACACAGAGACACAGAACACAGTGGAGAAAAGGG
AGTGAAATGTCTTTAATGACACTTACTATATATGGGATTTTGTGACAATATACAAGG
ATGGTTAAGACATATAAGGTGATGCAAAAAAACATATTAACAATTATAGTGACAAA
AAATGAGGAGCATATAATTATACATTGATTTATACAGAGTACCAGAGGAACACAGC
ATTGAGAGCCGTAACACCACCTGAGGGAGTGGAGAAAGGCTTCAGAGAGAAAGTG
TTTTTTGGAATGGATCACTGTTTCCAAAAGAACTAAAGTACAGTTTGAGAAATGCAT
ACTTAATTCATTACTTTTTTCCCCTCAACTTTAATAATAAATTTACCCAACAAAAAA
GTTTATTTTTGACTTGTAAATCTCTTAAAATCATAAAAAAGTAAAATTAGCTTTTAA
AAACAGGTAGTCACCATAGCATTGAATGTGTAGTTTATAATACAGCAAAGTTAAAT
ACAATTTCAAATTACCTATTAAGTTAGTTGCTCATTTCTTTGATTTCATTTAGCATTG
ATCTAACTCAATGTGGAAGAAGGTTACATTCGTGCAAGTTAACACGGCTTAATGATT
AACTATGTTCACCTACCAACCTTACCTTTTCTGGGCAAATATTGGTATATATAGAGT
TAAGAAGTCTAGGTCTGCTTCCAGAAGAAAACAGTTCCACGTTGCTTGAAATTGAA
AATCAAGATAAAAATGTTCACAATTAAGCTCCTTCTTTTTATTGTTCCTCTAGTTATT
TCCTCCAGAATTGATCAAGACAATTCATCATTTGATTCTCTATCTCCAGAGCCAAAA
TCAAGATTTGCTATGTTAGACGATGTAAAAATTTTAGCCAATGGCCTCCTTCAGTTG
GGACATGGTCTTAAAGACTTTGTCCATAAGACGAAGGGCCAAATTAATGACATATT
TCAAAAACTCAACATATTTGATCAGTCTTTTTATGATCTATCGCTGCAAACCAGTGA
AATCAAAGAAGAAGAAAAGGAACTGAGAAGAACTACATATAAACTACAAGTCAAA
AATGAAGAGGTAAAGAATATGTCACTTGAACTCAACTCAAAACTTGAAAGCCTCCT
AGAAGAAAAAATTCTACTTCAACAAAAAGTGAAATATTTAGAAGAGCAACTAACTA
ACTTAATTCAAAATCAACCTGAAACTCCAGAACACCCAGAAGTAACTTCACTTAAA
GTAAGTAGAAAATAAAGAGGGTTCATGTTTATGTTTTCAATGTGGATCTTTTAAAAA
AAATATTTCTAAGGCATGCCATTTGAAATACTTTGTTGCATTGTTGAAATACTTTTTT
TTCCAAGAAAAATAATCTCCAGAAAATAAAATTTCCTATTATAATTTCAAGTTAGTT
TTTTGTTTCCCTAATGTTATATATGAAAACACTGAAAATTTGCATTTTATATGAAAAT
TACAAATCGGTTAAATTATACAATCTAGAACACTATGTCATTACACTATTGTAAATT
ACTGAAGGTAAGTAAAAAGTTAAAAAAAATTTAAAACTATTCTCCAGTGTTTAAAA
CAGATTAAATAATACAGTAAATGGAAAAGATTTATTCATATGAAAATATGCTGGGC
TTTTTCTTTTAATTGAAGTTCAGAAAATCAAATTTTAGAGATAGTACAATTTAAATA
AAATGTTAAGGACAAAAATATGTGCTATTTGAAAGAAGCATACAAGGGGAAGGAA
TTGCCAATATTCATTTTTCAAATCCATTATTAGTTTAAAAATTTAGATTATGATAGTG
TTACAGGAAATTAATAGAAAAGAAAGAGGAAAGCAACTTATAACCAACCTACTCTC
TATATCCAGACTTTTGTAGAAAAACAAGATAATAGCATCAAAGACCTTCTCCAGAC
CGTGGAAGACCAATATAAACAATTAAACCAACAGCATAGTCAAATAAAAGAAATA
GAAAATCAGGTAAGTCAGTATTTTAATGGTATGTCCCATCTTTCACACAGGTCTGTA
AAAACACTGAATCCTAAAATTATTTACAAGCTTTAACTGGATCATGAGTAAAATTAT
CACATCAGCATAACTGTTAAAATTGCAGGCTCTGAAGCTAATAAACTACCTGCATTT
AAACCATGGCTCTAAAACTTTGTGTGACCTTGAATAAATTACTTCACCCCTTTATCT
CTCAGTTTCCTCACATATACTACAAAGATAATAACAGAACTTATAGGATTATTGTAA
GAAAAAAAATTAATTCATAGCAGCCAATGTCATCTTACTAAAATTCAAATTAGATC
ATGTTTCTCTTTGCTCAAAACCACACAATAGCTTTCCATTTCACTCATATTGGCTCTT
TAGACCAAGATTACCCAACCCTTCGTCATCTCACTGACTTCACCTCCTCTACTCTAGT
TATTCTGACCGCTTTACCAGTATTCAAACACATCAAACATACTGCCACCTCAAAGCC
TTTGCCCTTGTTGTTTCCTCTAACTGGAACGCTCTTCTGCCCTGGTATCTACGTGGCC
CACTCTCTGATTTCCCTTAGGGTCGTTATCAAACAAAAAATTCCCAATGAAGACTTA
CAAGGTCACTTAACCAAAAATCACAACCGCCTGGTCCCATCCCTGAAAACTTCTACT
TCCTTAGCTACTTTTCTCCTGCACACTCACCTTTATTTAACATAACATAAATTTTAGT
TATTTATCTCTTCTATTCCTGCACTAAAATGTAAGCTCTGTGAATACAGGGATTTTTT
CCATTATCTTCATATTTTCCATTATTTGTATATACTCCAGAATATAGAATACTGTATG
GCACACAGTAGGCATTTCTGTTGAATTAATAAATGTAATGTCATATTCACACAGAAG
CGTGTGCTATGATTATTATTACTTGGATTACTAGAAATAGTGTGCCTCATAATTAAA
GGTCAACATTCAACAATGTAATTAATCTACAATGTAAACATCTGGTGAAGTGACAG
AGGGAAGCACTTGTTTAGAAAAAAGCTATGTCAGAATCCATGTATTCTAATATGCA
GTACAATAGTTTAAAAATATTAATAATACTCTCAAACAGCTATTCAAGAGGATTCA
AAAAACATAATATAAACTCAGAGAAACTGGTAAACAAAATCATTTTCAAGAGATAT
AAAACAAATATTATTACCAATTTCCACTAAACAAACATAATGTTAGTAGTGCTGCTA
AAAGGTTTTTTATCAACTACTTTTGGTTTCCATACTTTCCTTCTTATGATGTTATTATT
CTAAATTCTTTTCAATTATATCTTTTACTATGATTAAATGAACCTGCTCCCCAAAGCA
AAATGTTACTATAGTAATATACATTGTGTCTAAAAATAAAAATGTGTGAAGAAACC
AAAACAATGAATTTCTGAGTTGGAAGAAGAGTTAGATCATTTAACTTTCTCATATTT
AAATTAAAAAAACAAAACTCTAAAAATTTAAGTAACTTTAAGATCACATAGTTACT
TAGTAGAAAAGAGTAATACCCAGCAAGCAAACTTTACAATAGATCCTTTTAAATAA
GGTCCTAGGAAATATCATTCATGCCAGCATCAAAAAACTAACACTAATAATGCAAG
ATATTATATATTCTGCTTTTCTTACTGTCAATGAGAAAAACTATCATTCAATAAATTG
CAAACCCAACACACTTAAATAAAAATAAAATGTTACTGCTAAACTAACGATAAACT
ACTGAATATATAGAAAGTAAGCAAACAAACTTGCCAACCTGCCAACATCTACAGAT
ATGTTTACAGGTCAAAAATTATCAAATTATCAAGAAAGCCTGGTTCAAATTATGTAT
TATGTCTTTATCACAGGTCTGAAGATCAGTAAGACCTAAAACTGAAAATTATTAAAC
TTAAAATCTGAACAGAATATCAAATATATTTTATTCATATAAATAAAAGAATACATT
ACAATATTCTAAGCAAAGCAGTCTCTACTTTTGGCCTTGCTCTGTTTTCCGACCAAT
GTCTGCTTTTTTGCCTTGCTTTATTTTTTTATCTTATTAAATAATGTCCCTGATTAAAT
ATTTTGAGAACAGGTAATCTGTACAATCTGAATAACACTGTTTATCTAAATATCAAA
CACCGTTATAACATTATGAACTGAAAGACAAACTGTACTTCTGACATCCTTACTCAG
ATTTCCCCTAATTGTATATTCAGTATCATTTTAAAAAACAGATTTATATTCTTTTATC
AGCTCAGAAGGACTAGTATTCAAGAACCCACAGAAATTTCTCTATCTTCCAAGCCA
AGAGCACCAAGAACTACTCCCTTTCTTCAGTTGAATGAAATAAGAAATGTAAAACA
TGATGGTAAGACACTTTGGTGGGTTTCCTTCTTGAAGCTATTATTATCAAATTCCCTA
TTCTTAGGACTTGTTCTAGACTAAAAGATAGTTAAGAGATATCCATCAAATACAATG
TATCAACCTAAACTGGATGCTGGGGTTCTTTTTACACCCTATAAAAGACATACCTAA
GACAATCAGAGAAATACAAATATGGACTTGATTATTAGATAATATAGAAGGTTTAT
TAATTTTCTTAGATGTGATCATGGTATTGCAGTTTTAAAGGAGAACAATCTCCTGTT
TAAGAGATACATGCTGAAATATTTACGGAGTTAAAGGTCACTGGACTCCAGACTGG
TGATAGAACAAGACTCTGTCTCTAAAAAATAATTAATTTTTTAAAAGAAAATAGTTT
GGTAAGATGATTCTTACATTCTTAAATAACACGCCATCTAAGAAAAATGCTTTAACA
TAAACATTACTGAAAAAATGCTACATTTGCCACAACTTCATAAAATGTCAAGTGAA
ATCTCAAGCTCCAAAGATATTATTCCTATTACTAAATCTGATGTAATAACATTTTATT
GATTCTAGGCATTCCTGCTGAATGTACCACCATTTATAACAGAGGTGAACATACAA
GTGGCATGTATGCCATCAGACCCAGCAACTCTCAAGTTTTTCATGTCTACTGTGATG
TTATATCAGGTAAAACCTGTCTAAGGAGAATAGACAGTAGTTAGTTCAACTTACTCA
TTACGTATTAGGAAGATTAACCTGGTTATCATTGTTTTATACATATATATATGAAAT
ATATATGAGTATTCGTATAAATATAATACTTTTACCTTGTTTATGTATTTACTCAATA
TTCTCCTTTTCCTCTAAAATAATCTGAAGTGACTATTATCAATAAGTTTACTATGCCA
AAATTCATTAATTGCCTTTCACTTAACTTTTGGGACCATAATAAATAATAAAATGTA
TTGCCATAACATTAATAAACTACCTTACAAAACCACCAATTAAAATCAAACAAACA
AAAAAGTGTTATTTACATCTGTCAACATAAATCTACTAAAAATACATGATTTCATTC
ATTATATTCAGGTAGTCCATGGACATTAATTCAACATCGAATAGATGGATCACAAA
ACTTCAATGAAACGTGGGAGAACTACAAATATGGTTTTGGGAGGCTTGATGGTAAG
GGGACTACATTCAATCATTCATTCACTTGCTAATCTACAAATATTTACTGAGAACCT
CTTATGGACCAGGTATTAGGAAAAGTAGTAACGAACGAGAAGCAGTCTCAGCCTTC
ATATAATTTATTATCAAACAATTACACATTTGTTAGTAAATTACACTTATTACAACT
GTTATTATTTGAATTATATTTATCACAATTACATGTCTGTTCTTAAATATACTTATCA
CAATTTAATTCCACGGCTTACAATGATCATAACTATAATTATTAAAGACAATTTTGA
TTAAATGTTATGTCATAAGTAGTAACTGTTACAAATAAGCTGTGAAAAGAACCACT
CCTAGCATTAGTCACTCTATTCTCTCATTAACGTTTTACATATCAATTAATTGGAAGT
TAAAAGGACCAGGAAACTCAGACATACAGTATACATTTTAAAATTTCAATTATTTA
AATATAATATATAGAATGTATGGCTTATAATGAATTAGTTAACTCAATGCAAATTAT
TCTATTTTGATTACAAATAGTAAAATAAGCAAGATAAAATAACAGATGTTTAAAAT
CCAAAAAGCACATACAAAAATCCATGAATGATGTCTAAGTACTCACTTATAAAGTA
GAAGACATTCATTATTATATCAAATTTTTAAATGCTCAGTACTATTTGACCATTTAA
AAATTTTGTATTCAAACTACCAGTGAAAGCCCTACCTAGAAGGTATACTCAGTGATA
AGTTTTGTAGCTCCAAATCTTCTAATAGTGAGTGTAACCCCAAAATAAAAGGCTGAC
AGGTAAGTCGAGAATACTCACTTAATTCTGGTAAGAAAGCAACCCATTTGTACTTGT
ATTTACCAGCAATCCTTAAAATGAAGCTTCCTACTAACTCAATAGCAATAAGACAAT
AGTGAATGTTTAATGAAAACAGTATTTTATAAATACTTTAATAAAAAGGATTGTGAT
GAAGAACAATCTATTTATATTTGTTATTTGTTTTTAATTCCAATAAAAATAATTTTTA
AAATTACAGAAAAAAGTTATTAAGAACCATGCTTTTAAATTTAAAATGATTTTTTAA
ATTTATTCCTGTCTTTTTCTACAAAGAAAGCATACATTAAGCAAATACCAAAGGCCA
GGTTTACATTTGAAGAAAGTGACATTATTATTACTCAAGTCTCTAGGAATACTTAAC
ACATCTCTTGACTGTATATGGATGTTAATAAATAGCTGACAGTAAAGTTTATCCATA
TAAAGACTTGCAAATATTCCTCTACCAATGACGAGACTTTAAAATATCTATAATAAT
GTAACACATTTCACTGGTGAAACATGTCTTGTCATATGCATTATAGAAAGGATAATC
AGACTTTCAGTTATATTAATATTTTTAACATTTTTGTGCACATAGCTATCTTCAATAA
AATTGTTTTAAAAGGTATTATTTTAAGATACACTAAAATGATCAAGGGATTCAAGAC
TAAACAACTCAATTAGTTGCACCAATAAAAAACACTTAAAAAAACTGTCAGTGTCC
AACCTGTACTTAATAACTCACAGATTTTTAAAACTTTTCTTTTCAGGAGAATTTTGGT
TGGGCCTAGAGAAGATATACTCCATAGTGAAGCAATCTAATTATGTTTTACGAATTG
AGTTGGAAGACTGGAAAGACAACAAACATTATATTGAATATTCTTTTTACTTGGGA
AATCACGAAACCAACTATACGCTACATCTAGTTGCGATTACTGGCAATGTCCCCAAT
GCAATCCCGGAAAACAAAGATTTGGTGTTTTCTACTTGGGATCACAAAGCAAAAGG
ACACTTCAACTGTCCAGAGGGTTATTCAGGTATCTTTTTCTGATACCAATACTTTATT
TTCATATCTTCAAAGTATCTTCCCACATTATTAGCTATTATCTGCAATGACAACTTTT
AAAAATCCGAATCCCAAATAAGCGTTTTCTCTCTAGACGAAAACCTCTTAACTATAA
TGAAAGTGTTCATTCTAGTTCAATCAGGTATTTTACCTCTAATCTTCCTCAGATTTTC
TATTTTTTGGTAGTGTATAGATTATTTATACAGATTATTTAAAATTGGGACTTATACA
GATTATTTAAAACTGGGATACATGCATCTAAAACACTGTAATATTTATAAGAAAGG
AAGATAAACTTACGGGGAAATACAGTAACAGTAACTACATACGAGTCTGTACCCAT
TAAATTGCATATCTATCTCCTTTAGGAGGCTGGTGGTGGCATGATGAGTGTGGAGAA
AACAACCTAAATGGTAAATATAACAAACCAAGAGCAAAATCTAAGCCAGAGAGGA
GAAGAGGATTATCTTGGAAGTCTCAAAATGGAAGGTTATACTCTATAAAATCAACC
AAAATGTTGATCCATCCAACAGATTCAGAAAGCTTTGAATGAACTGAGGCAAATTT
AAAAGGCAATAATTTAAACATTAACCTCATTCCAAGTTAATGTGGTCTAATAATCTG
GTATTAAATCCTTAAGAGAAAGCTTGAGAAATAGATTTTTTTTATCTTAAAGTCACT
GTCTATTTAAGATTAAACATACAATCACATAACCTTAAAGAATACCGTTTACATTTC
TCAATCAAAATTCTTATAATACTATTTGTTTTAAATTTTGTGATGTGGGAATCAATTT
TAGATGGTCACAATCTAGATTATAATCAATAGGTGAACTTATTAAATAACTTTTCTA
AATAAAAAATTTAGAGACTTTTATTTTAAAAGGCATCATATGAGCTAATATCACAAC
TTTCCCAGTTTAAAAAACTAGTACTCTTGTTAAAACTCTAAACTTGACTAAATACAG
AGGACTGGTAATTGTACAGTTCTTAAATGTTGTAGTATTAATTTCAAAACTAAAAAT
CGTCAGCACAGAGTATGTGTAAAAATCTGTAATACAAATTTTTAAACTGATGCTTCA
TTTTGCTACAAAATAATTTGGAGTAAATGTTTGATATGATTTATTTATGAAACCTAA
TGAAGCAGAATTAAATACTGTATTAAAATAAGTTCGCTGTCTTTAAACAAATGGAG
ATGACTACTAAGTCACATTGACTTTAACATGAGGTATCACTATACCTTATTTGTTAA
AATATATACTGTATACATTTTATATATTTTAACACTTAATACTATGAAAACAAATAA
TTGTAAAGGAATCTTGTCAGATTACAGTAAGAATGAACATATTTGTGGCATCGAGTT
AAAGTTTATATTTCCCCTAAATATGCTGTGATTCTAATACATTCGTGTAGGTTTTCAA
GTAGAAATAAACCTCGTAACAAGTTACTGAACGTTTAAACAGCCTGACAAGCATGT
ATATATGTTTAAAATTCAATAAACAAAGACCCAGTCCCTAAATTATAGAAATTTAA
ATTATTCTTGCATGTTTATCGACATCACAACAGATCCCTAAATCCCTAAATCCCTAA
AGATTAGATACAAATTTTTTACCACAGTATCACTTGTCAGAATTTATTTTTAAATAT
GATTTTTTAAAACTGCCAGTAAGAAATTTTAAATTAAACCCATTTGTTAAAGGATAT
AGTGCCCAAGTTATATGGTGACCTACCTTTGTCAATACTTAGCATTATGTATTTCAA
ATTATCCAATATACATGTCATATATATTTTTATATGTCACATATATAAAAGATATGT
ATGATCTATGTGAATCCTAAGTAAATATTTTGTTCCAGAAAAGTACAAAATAATAA
AGGTAAAAATAATCTATAATTTTCAGGACCACAGACTAAGCTGTCGAAATTAACGC
TGATTTTTTTAGGGCCAGAATACCAAAATGGCTCCTCTCTTCCCCCAAAATTGGACA
ATTTCAAATGCAAAATAATTCATTATTTAATATATGAGTTGCTTCCTCTATTTGGTTT
CCTTAAAAAAAAAAAAAACTCTCATAGGACATGTTTCATTTTGTTCCTTTCAGGAGT
AGTAAATTAGACGTTTTCCCCATATAAAGCTTTTTTCTACCAGAAAGATACTTCTGG
TAGAAGAAGAGAAAGGAGCTCTTTATGGTTCACACGACTGTCTCCTGTCCTAACTAC
TTTGCTTAAAGTGCTCAAATTCCATCACTACTCACAGTTGTCTAATCTAAGTCTAATC
CCCTTTGATCTCTCAGACTACCTTCCCTTTTATCTCTCTACTACTTAATAATAAGAAT
ATCTTTTTTTCAAACTTGACCTTCATTTTGCTTTCACAATACTATACTCTCCATGGAT
TATCCCTTATCTGAATCCATCTTTATAACCCTATTCCTTTCTCATATTTAGTACTGTG
GGCCAATGGACAACCTTCAATCATCTTTTCTACACTGACCCTCAGACATTCTATCTG
CTCTCACGGACTCCTTTATTTACCATGAATAAAGTTCCAAAATCTACATATTCATCC
CAAGTCTCTTTCCAGTTCCCCTTCTTACATTGCCTATTTGCCATTTCTCCCTTCAATAC
CCTATACTTCACTCAAATTCAACATACCAAAAATAAAAGGCCAGGCACGGTGGCTC
ACACCTGTAATCCCAGGACTTTGGGAGGCTGAGGCAGGTGGATCACCTGAGGTCAG
GAGTCTGACCAGCCTGACCAATATGGTGAAACCCCGTCTCTACCTAAAATACAAAA
ATTAGCCAGGCGTGGTGGCATGTGCCTACAGTCCCAGCTACTCAAGAGGCTGAGAC
AGGAGAATCGCTTGAACCCAGGAGGCGGAGGTTGCAGTGAGCTGAGATCACACCA
ATGCACTGGGTGACAGAACAAGACTGACTCAAAAAAAAATAAATAACAAATTCCCC
AGCCCCTTACTGCTACTGCTATCCCTTTCTACCCACCTTTCCCTCCTTTATACTCTTTC
ACACCATCTTCCTCACTTCTTTATATCCATTAATATGACCAGCATGTTCCCAGTCACA
GAAGCCTGGAACCCGGAAGACATCTCTGGCTTTTCACTCAACTTTGTAAACTACCTC
TTTTGTATCATAAGCCACCAAGTTCAATACAATCTTCTCTTGAAACGTCTCTTAATCT
TATAAGCTTTCTTCCCCAAAGACTGTCTTTAACTTCAGTGCTAGATTATATAAGT
AAD34156.1 human angiopoietin-related protein 3:
(SEQ ID NO: 109)
MFTIKLLLFIVPLVISSRIDQDNSSFDSLSPEPKSRFAMLDDVKILANGLLQLGHGLKDFV
HKTKGQINDIFQKLNIFDQSFYDLSLQTSEIKEEEKELRRTTYKLQVKNEEVKNMSLELN
SKLESLLEEKILLQQKVKYLEEQLTNLIQNQPETPEHPEVTSLKTFVEKQDNSIKDLLQTV
EDQYKQLNQQHSQIKEIENQLRRTSIQEPTEISLSSKPRAPRTTPFLQLNEIRNVKHDGIPA
ECTTIYNRGEHTSGMYAIRPSNSQVFHVYCDVISGSPWTLIQHRIDGSQNFNETWENYK
YGFGRLDGEFWLGLEKIYSIVKQSNYVLRIELEDWKDNKHYIEYSFYLGNHETNYTLHL
VAITGNVPNAIPENKDLVFSTWDHKAKGHFNCPEGYSGGWWWHDECGENNLNGKYN
KPRAKSKPERRRGLSWKSQNGRLYSIKSTKMLIHPTDSESFE

### ApoLipoprotein (a) (LPA)

In some embodiments, the target gene for modification using the compositions and methods disclosed herein is gene encoding lipoprotein a (LPA). LPA is a low-density lipoprotein variant. Genetic and epidemiologic studies have identified LPA as a risk factor for atherosclerosis and related diseases, such as coronary heart disease and stroke. LPA concentrations vary more than one thousand times between individuals: from <0.2 to >200 mg/dL. This range of concentrations is observed in all populations studied by scientists so far. The mean and median concentrations between different world populations show distinct particularities, the main being the two to threefold higher LPA plasma concentration of populations of African descent compared to Asian, Oceanic, or European populations. High LPA in blood correlates with coronary heart disease (CHD), cardiovascular disease (CVD), atherosclerosis, thrombosis, and stroke. Individuals without LPA or with very low LPA levels seem to be healthy. Thus, plasma LPA is not vital, at least under normal environmental conditions. Since apo (a)/LPA appeared rather recently in mammalian evolution-only old-world monkeys and humans have been shown to harbor LPA-its function might not be vital, but just evolutionarily advantageous under certain environmental conditions, e.g., in case of exposure to certain infectious diseases.

An exemplary LPA amino acid sequence encoded by Human reference sequence NG_016147.1 is provided below:

>sp|P08519|APOA_HUMAN Apolipoprotein(a) OS =
Homo sapiens OX = 9606 GN = LPA PE = 1 SV = 1
(SEQ ID NO: 110)
MEHKEVVLLLLLFLKSAAPEQSHVVQDCYHGDGQSYRGTYSTTVTGRTCQ
AWSSMTPHQHNRTTENYPNAGLIMNYCRNPDAVAAPYCYTRDPGVRWEYC
NLTQCSDAEGTAVAPPTVTPVPSLEAPSEQAPTEQRPGVQECYHGNGQSY
RGTYSTTVTGRTCQAWSSMTPHSHSRTPEYYPNAGLIMNYCRNPDAVAAP
YCYTRDPGVRWEYCNLTQCSDAEGTAVAPPTVTPVPSLEAPSEQAPTEQR
PGVQECYHGNGQSYRGTYSTTVTGRTCQAWSSMTPHSHSRTPEYYPNAGL
IMNYCRNPDAVAAPYCYTRDPGVRWEYCNLTQCSDAEGTAVAPPTVTPVP
SLEAPSEQAPTEQRPGVQECYHGNGQSYRGTYSTTVTGRTCQAWSSMTPH
SHSRTPEYYPNAGLIMNYCRNPDAVAAPYCYTRDPGVRWEYCNLTQCSDA
EGTAVAPPTVTPVPSLEAPSEQAPTEQRPGVQECYHGNGQSYRGTYSTTV
TGRTCQAWSSMTPHSHSRTPEYYPNAGLIMNYCRNPDAVAAPYCYTRDPG
VRWEYCNLTQCSDAEGTAVAPPTVTPVPSLEAPSEQAPTEQRPGVQECYH
GNGQSYRGTYSTTVTGRTCQAWSSMTPHSHSRTPEYYPNAGLIMNYCRNP
DAVAAPYCYTRDPGVRWEYCNLTQCSDAEGTAVAPPTVTPVPSLEAPSEQ
APTEQRPGVQECYHGNGQSYRGTYSTTVTGRTCQAWSSMTPHSHSRTPEY
YPNAGLIMNYCRNPDAVAAPYCYTRDPGVRWEYCNLTQCSDAEGTAVAPP
TVTPVPSLEAPSEQAPTEQRPGVQECYHGNGQSYRGTYSTTVTGRTCQAW
SSMTPHSHSRTPEYYPNAGLIMNYCRNPDAVAAPYCYTRDPGVRWEYCNL
TQCSDAEGTAVAPPTVTPVPSLEAPSEQAPTEQRPGVQECYHGNGQSYRG
TYSTTVTGRTCQAWSSMTPHSHSRTPEYYPNAGLIMNYCRNPDAVAAPYC
YTRDPGVRWEYCNLTQCSDAEGTAVAPPTVTPVPSLEAPSEQAPTEQRPG
VQECYHGNGQSYRGTYSTTVTGRTCQAWSSMTPHSHSRTPEYYPNAGLIM
NYCRNPDAVAAPYCYTRDPGVRWEYCNLTQCSDAEGTAVAPPTVTPVPSL
EAPSEQAPTEQRPGVQECYHGNGQSYRGTYSTTVTGRTCQAWSSMTPHSH
SRTPEYYPNAGLIMNYCRNPDAVAAPYCYTRDPGVRWEYCNLTQCSDAEG
TAVAPPTVTPVPSLEAPSEQAPTEQRPGVQECYHGNGQSYRGTYSTTVTG
RTCQAWSSMTPHSHSRTPEYYPNAGLIMNYCRNPDAVAAPYCYTRDPGVR
WEYCNLTQCSDAEGTAVAPPTVTPVPSLEAPSEQAPTEQRPGVQECYHGN
GQSYRGTYSTTVTGRTCQAWSSMTPHSHSRTPEYYPNAGLIMNYCRNPDA
VAAPYCYTRDPGVRWEYCNLTQCSDAEGTAVAPPTVTPVPSLEAPSEQAP
TEQRPGVQECYHGNGQSYRGTYSTTVTGRTCQAWSSMTPHSHSRTPEYYP
NAGLIMNYCRNPDAVAAPYCYTRDPGVRWEYCNLTQCSDAEGTAVAPPTV
TPVPSLEAPSEQAPTEQRPGVQECYHGNGQSYRGTYSTTVTGRTCQAWSS
MTPHSHSRTPEYYPNAGLIMNYCRNPDAVAAPYCYTRDPGVRWEYCNLTQ
CSDAEGTAVAPPTVTPVPSLEAPSEQAPTEQRPGVQECYHGNGQSYRGTY
STTVTGRTCQAWSSMTPHSHSRTPEYYPNAGLIMNYCRNPDAVAAPYCYT
RDPGVRWEYCNLTQCSDAEGTAVAPPTVTPVPSLEAPSEQAPTEQRPGVQ
ECYHGNGQSYRGTYSTTVTGRTCQAWSSMTPHSHSRTPEYYPNAGLIMNY
CRNPDAVAAPYCYTRDPGVRWEYCNLTQCSDAEGTAVAPPTVTPVPSLEA
PSEQAPTEQRPGVQECYHGNGQSYRGTYSTTVTGRTCQAWSSMTPHSHSR
TPEYYPNAGLIMNYCRNPDAVAAPYCYTRDPGVRWEYCNLTQCSDAEGTA
VAPPTVTPVPSLEAPSEQAPTEQRPGVQECYHGNGQSYRGTYSTTVTGRT
CQAWSSMTPHSHSRTPEYYPNAGLIMNYCRNPDAVAAPYCYTRDPGVRWE
YCNLTQCSDAEGTAVAPPTVTPVPSLEAPSEQAPTEQRPGVQECYHGNGQ
SYRGTYSTTVTGRTCQAWSSMTPHSHSRTPEYYPNAGLIMNYCRNPDAVA
APYCYTRDPGVRWEYCNLTQCSDAEGTAVAPPTVTPVPSLEAPSEQAPTE
QRPGVQECYHGNGQSYRGTYSTTVTGRTCQAWSSMTPHSHSRTPEYYPNA
GLIMNYCRNPDAVAAPYCYTRDPGVRWEYCNLTQCSDAEGTAVAPPTVTP
VPSLEAPSEQAPTEQRPGVQECYHGNGQSYRGTYSTTVTGRTCQAWSSMT
PHSHSRTPEYYPNAGLIMNYCRNPDAVAAPYCYTRDPGVRWEYCNLTQCS
DAEGTAVAPPTVTPVPSLEAPSEQAPTEQRPGVQECYHGNGQSYRGTYST
TVTGRTCQAWSSMTPHSHSRTPEYYPNAGLIMNYCRNPDAVAAPYCYTRD
PGVRWEYCNLTQCSDAEGTAVAPPTVTPVPSLEAPSEQAPTEQRPGVQEC
YHGNGQSYRGTYSTTVTGRTCQAWSSMTPHSHSRTPEYYPNAGLIMNYCR
NPDAVAAPYCYTRDPGVRWEYCNLTQCSDAEGTAVAPPTVTPVPSLEAPS
EQAPTEQRPGVQECYHGNGQSYRGTYSTTVTGRTCQAWSSMTPHSHSRTP
EYYPNAGLIMNYCRNPDAVAAPYCYTRDPGVRWEYCNLTQCSDAEGTAVA
PPTVTPVPSLEAPSEQAPTEQRPGVQECYHGNGQSYRGTYSTTVTGRTCQ
AWSSMTPHSHSRTPEYYPNAGLIMNYCRNPDAVAAPYCYTRDPGVRWEYC
NLTQCSDAEGTAVAPPTVTPVPSLEAPSEQAPTEQRPGVQECYHGNGQSY
RGTYSTTVTGRTCQAWSSMTPHSHSRTPEYYPNAGLIMNYCRNPDAVAAP
YCYTRDPGVRWEYCNLTQCSDAEGTAVAPPTVTPVPSLEAPSEQAPTEQR
PGVQECYHGNGQSYRGTYSTTVTGRTCQAWSSMTPHSHSRTPEYYPNAGL
IMNYCRNPDAVAAPYCYTRDPGVRWEYCNLTQCSDAEGTAVAPPTVTPVP
SLEAPSEQAPTEQRPGVQECYHGNGQSYRGTYSTTVTGRTCQAWSSMTPH
SHSRTPEYYPNAGLIMNYCRNPDAVAAPYCYTRDPGVRWEYCNLTQCSDA
EGTAVAPPTVTPVPSLEAPSEQAPTEQRPGVQECYHGNGQSYRGTYSTTV
TGRTCQAWSSMTPHSHSRTPEYYPNAGLIMNYCRNPDPVAAPYCYTRDPS
VRWEYCNLTQCSDAEGTAVAPPTITPIPSLEAPSEQAPTEQRPGVQECYH
GNGQSYQGTYFITVTGRTCQAWSSMTPHSHSRTPAYYPNAGLIKNYCRNP
DPVAAPWCYTTDPSVRWEYCNLTRCSDAEWTAFVPPNVILAPSLEAFFEQ
ALTEETPGVQDCYYHYGQSYRGTYSTTVTGRTCQAWSSMTPHQHSRTPEN
YPNAGLTRNYCRNPDAEIRPWCYTMDPSVRWEYCNLTQCLVTESSVLATL
TVVPDPSTEASSEEAPTEQSPGVQDCYHGDGQSYRGSFSTTVTGRTCQSW
SSMTPHWHQRTTEYYPNGGLTRNYCRNPDAEISPWCYTMDPNVRWEYCNL
TQCPVTESSVLATSTAVSEQAPTEQSPTVQDCYHGDGQSYRGSFSTTVTG
RTCQSWSSMTPHWHQRTTEYYPNGGLTRNYCRNPDAEIRPWCYTMDPSVR
WEYCNLTQCPVMESTLLTTPTVVPVPSTELPSEEAPTENSTGVQDCYRGD
GQSYRGTLSTTITGRTCQSWSSMTPHWHRRIPLYYPNAGLTRNYCRNPDA
EIRPWCYTMDPSVRWEYCNLTRCPVTESSVLTTPTVAPVPSTEAPSEQAP
PEKSPVVQDCYHGDGRSYRGISSTTVTGRTCQSWSSMIPHWHQRTPENYP
NAGLTENYCRNPDSGKQPWCYTTDPCVRWEYCNLTQCSETESGVLETPTV
VPVPSMEAHSEAAPTEQTPVVRQCYHGNGQSYRGTFSTTVTGRTCQSWSS
MTPHRHQRTPENYPNDGLTMNYCRNPDADTGPWCFTMDPSIRWEYCNLTR
CSDTEGTVVAPPTVIQVPSLGPPSEQDCMFGNGKGYRGKKATTVTGTPCQ
EWAAQEPHRHSTFIPGTNKWAGLEKNYCRNPDGDINGPWCYTMNPRKLFD
YCDIPLCASSSFDCGKPQVEPKKCPGSIVGGCVAHPHSWPWQVSLRTRFG
KHFCGGTLISPEWVLTAAHCLKKSSRPSSYKVILGAHQEVNLESHVQEIE
VSRLFLEPTQADIALLKLSRPAVITDKVMPACLPSPDYMVTARTECYITG
WGETQGTFGTGLLKEAQLLVIENEVCNHYKYICAEHLARGTDSCQGDSGG
PLVCFEKDKYILQGVTSWGLGCARPNKPGVYARVSRFVTWIEGMMRNN

Cas protein-gRNA complexes

In another aspect, provided herein is a complex comprising the single guide RNA as provided herein in complex with the Cas protein, e.g., the Cas12b protein, wherein the complex comprises increased stability as compared to a complex with an unmodified single guide RNA and a Cas12b protein, wherein the stability is measured by half-life of the complex ex vivo or in vitro.

In some embodiments, the complex comprises increased stability as compared to a complex with an unmodified single guide RNA and a Cas12b protein. In some embodiments, the complex comprises increased stability by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas12b protein. In some embodiments, the complex comprises increased stability by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas12b protein.

In some embodiments, wherein the stability of the complex is measured by half-life of the complex. In some embodiments, wherein the stability of the complex is measured by half-life of the complex ex vivo. In some embodiments, wherein the stability of the complex is measured by half-life of the complex in vitro.

In some embodiments, the complex comprises increased half-life by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas12b protein wherein half-life of the complex is measured ex vivo. In some embodiments, the single guide RNA exhibits increased half-life of the complex by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas12b protein, wherein half-life of the complex is measured ex vivo.

In some embodiments, the complex comprises increased half-life when measured in vitro by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas12b protein, wherein half-life of the complex is measured in vitro. In some embodiments, the single guide RNA exhibits increased half-life of the complex by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas12b protein, wherein half-life of the complex is measured in vitro.

In another aspect, provided herein is a cell comprising the complex as provided herein. In some embodiments, the cell may be an in vitro cell. In some embodiments, the cell may be an ex vivo cell. In some embodiments, the cell may be an in vivo cell. In some embodiments, the cell may be an isolated cell.

### Gene Modification Compositions

In another aspect, provided herein is a composition for gene modification comprising the single guide RNA as provided herein and a Cas12b protein or a nucleic acid sequence encoding the Cas12b protein. In some embodiments, the composition further comprises a vector that comprises the nucleic acid sequence encoding the Cas12b protein.

In some embodiments, the nucleic acid sequence may be a DNA, an RNA or mRNA, or a modified nucleic acid sequence. In some embodiments, the vector may be an expression vector. In some embodiments, the nucleic acid is operatively linked to a promoter of the vector. In some embodiments, the vector is a plasmid or a viral vector.

The term “vector,” as used herein, refers to a nucleic acid molecule capable of transporting another nucleic acid to which it has been linked. In some examples, a vector is an expression vector that is capable of directing the expression of nucleic acids to which they are operatively linked. The term “operably linked,” as used herein, means that the nucleotide sequence of interest is linked to regulatory sequence(s) in a manner that allows for expression of the nucleotide sequence. The term “regulatory sequence,” as used herein, includes, but is not limited to promoters, enhancers and other expression control elements. Such regulatory sequences are well known in the art and are described, for example, in Goeddel; Gene Expression Technology: Methods in Enzymology 185, Academic Press, San Diego, CA (1990). Examples of expression vectors include, but are not limited to, plasmid vectors, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors.

### Lipid Nanoparticles (LNPs)

In another aspect, provided herein is a lipid nanoparticle (LNP) comprising the composition as provided herein.

As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm.

In some embodiments, an LNP may be made from cationic, anionic, or neutral lipids. In some embodiments, an LNP comprises neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP comprises hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N [1-(2,3-dioleyloxy) propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N—(N′,N′-dimethylaminoethane)-carbamoyl]cholesterol), DOTAP-cholesterol, GAP-DMORIE-DPyPE, and GL67A-DOPE-DMPE (2-Bis(dimethylphosphino)ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce a LNP. In some embodiments, the polynucleotide may be combined with lipid(s) in a wide range of molar ratios to produce a LNP.

### Modifications of Target Genes

In another aspect, provided herein is a method for modifying a target polynucleotide sequence of a gene in a cell, wherein the single guide RNA directs the Cas12b protein to effect a modification in the targeted polynucleotide sequence complementary to the spacer of the modified single guide RNA.

In some embodiments, the targeted polynucleotide sequence is in a target gene that encodes a protein of interest. In some embodiments, the modification reduces or abolishes expression of the protein of interest encoded by the target gene in the cell.

In some embodiments, the modification reduces expression of the protein of interest encoded by the target gene in the cell. In some embodiments, the modification reduces expression of the protein of interest encoded by the target gene in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100%. In some embodiments, the modification reduces expression of the protein of interest encoded by the target gene in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold. In some embodiments, the modification abolishes expression of the protein of interest encoded by the target gene in the cell.

In some embodiments, the targeted polynucleotide sequence is in a PCSK9 gene. In some embodiments, the modification reduces or abolishes expression of functional PCSK9 protein encoded by the PCSK9 gene in the cell.

In some embodiments, the modification reduces expression of functional PCSK9 protein encoded by the PCSK9 gene in the cell. In some embodiments, the modification reduces expression of functional PCSK9 protein encoded by the PCSK9 gene in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100%. In some embodiments, the modification reduces expression of functional PCSK9 protein encoded by the PCSK9 gene in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold. In some embodiments, the modification abolishes expression of functional PCSK9 protein encoded by the PCSK9 gene in the cell.

In some embodiments, the targeted polynucleotide sequence is in an ANGPTL3 gene. In some embodiments, the modification results in reducing or abolishing expression of functional ANGPTL3 protein encoded by the ANGPTL3 gene in the cell.

In some embodiments, the modification reduces expression of functional ANGPTL3 protein encoded by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100%. In some embodiments, the modification reduces expression of functional ANGPTL3 protein encoded by the ANGPTL3 gene in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold. In some embodiments, the modification abolishes expression of functional ANGPTL3 protein encoded by the ANGPTL3 gene in the cell.

In some embodiments, the modification to the gene caused by the chemically modified single guide RNA directed Cas12b protein-guide RNA effector complex is a double strand break, a non-sense mutation, a frameshift mutation, a splice site alteration, or an inversion.

The term “modification” or “modification of a target polynucleotide sequence,” as used herein, refers to any change or alteration in the genome of a cell.

The term “double stranded break,” as used herein, refers to a change in which both strands in the double helix are severed.

The term “non-sense mutation,” as used herein, refers to a mutation in which a sense codon that corresponds to one of the amino acids specified by the genetic code is changed to a chain-terminating codon (e.g., stop codons UAA, UGA or UAG).

The term “frameshift mutation,” as used herein, refers to a genetic mutation caused by a deletion or insertion of a number of nucleotides in a DNA sequence that is not divisible by three. In some embodiments, the insertion or deletion may change the reading frame due to the triplet nature of gene expression by codons, resulting in a completely different translation from the original. In some embodiments, a frameshift mutation may alter the first stop codon encountered in the sequence.

The term “splice site alteration,” as used herein, refers to a genetic alteration in the DNA sequence that occurs at the boundary of an exon and an intron (splice site). In some embodiments, the mutation in the splice-donor and splice-acceptor sequences may disrupt RNA splicing resulting in the loss of exons or the inclusion of introns and an altered protein-coding sequence.

The term “inversion,” as used herein, refers to a chromosome rearrangement in which a segment of a chromosome is reversed end to end. In some embodiments, an inversion occurs when a single chromosome undergoes breakage and rearrangement within itself. In some embodiments, an inversion is a pericentric inversions that include the centromere and a break point in each arm. In some embodiments, an inversion is a paracentric inversion in which both breaks occur in one arm of the chromosome and the centromere is not included. In some embodiments, an inversion does not involve a loss of genetic information, but simply rearranges the linear gene sequence. In some embodiments, an inversion does not cause any abnormalities in carriers as long as the rearrangement is balanced with no extra or missing DNA. In some embodiments, in individuals which are heterozygous for an inversion, there is an increased production of abnormal chromatids.

In some embodiments, the modification of a target polynucleotide sequence may include, but are not limited to, insertion, deletion and correction.

The term “insertion,” as used herein, refers to an addition of one or more nucleotides in a DNA sequence. In some embodiments, an insertion can range from a small insertion of a few nucleotides to an insertion of large segments such as a cDNA or a gene.

The term “deletion,” as used herein, refers to a loss or removal of one or more nucleotides in a DNA sequence or a loss or removal of the function of a gene. In some embodiments, a deletion can include, for example, a loss of a few nucleotides, an exon, an intron, a gene segment, or the entire sequence of a gene. In some embodiments, deletion of a gene refers to the elimination or reduction of the function or expression of a gene or its gene product.

For example, in some embodiments, a deletion may include a gene knock-in, knock-out or knock-down. The term “knock-in, as used herein, refers to an addition of a DNA sequence or fragment thereof into a genome. In some embodiments, the DNA sequence or fragment thereof to be knocked-in may include an entire gene or genes or regulatory sequences associated with a gene or any fragment thereof. For example, in some embodiments, a knock-in strategy may involve substitution of an existing sequence with the provided sequence, e.g., substitution of a mutant allele with a wild-type copy or vice versa.

The term “knock-out,” as used herein, refers to the elimination of a gene or the expression of a gene. In some embodiments, a gene can be knocked out by a deletion or an addition of a nucleotide sequence that leads to a disruption of the reading frame, or by a replacement of a part of the gene with an irrelevant sequence. The term, “knock-down,” as used herein, refers to reduction in the expression of a gene or its gene product. In some embodiments, as a result of a gene knockdown, the protein activity or function may be attenuated or the protein levels may be reduced or eliminated.

The term “correction,” as used herein, refers to a change of one or more nucleotides of a genome in a cell by insertion, deletion or substitution. In some embodiments, a correction may result in a more favorable genotypic or phenotypic outcome in structure or function to the genomic site which was corrected. For example, in some embodiments, a correction may include the correction of a mutant or defective sequence to a wild-type sequence which restores structure or function to a gene or its gene product.

### Pharmaceutical Compositions

In another aspect, provided herein is a pharmaceutical composition for gene modification comprising the single guide RNA as provided herein and the Cas12b protein or a nucleic acid sequence encoding the Cas12b protein, for example an mRNA. “Pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

Pharmaceutical compositions described herein comprises the chemically modified guide RNA and an mRNA encoding the Cas12b protein in combination with one or more pharmaceutically or physiologically acceptable carriers, diluents or excipients. Such compositions can comprise a suspension of lipid nanoparticle constituted from the chemically modified guide RNA and the mRNA and lipid nanoparticle excipients, buffers such as neutral buffered saline, phosphate buffered saline and the like; stabilizers such as albumin; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The aforementioned pharmaceutical compositions comprising lipid nanoparticles, in some embodiments, can be suspended in a pharmaceutically acceptable buffer in the presence or in the absence of one or more, or a combination of lipid nanoparticle stabilizers in the suspension, or in the frozen and/or lyophilized form. The pharmaceutically acceptable buffers include, but not limited to, neutral buffered saline, phosphate buffered saline and the like, and neutral or near neutral TRIS buffer and the stabilizers include, but not limited to carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained. Thus, a pharmaceutically acceptable excipient, carrier, or diluent” refers to an excipient, carrier, or diluent that can be administered to a subject, together with an agent, and which does not destroy the requisite or desired pharmacological activity thereof and is nontoxic when administered in doses sufficient to deliver a therapeutic amount of the agent.

Pharmaceutical compositions can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In embodiments, the pharmaceutical composition facilitates administration of the compound to an organism.

The administration of compositions described herein can be carried out in any convenient manner, including by aerosol inhalation, injection, ingestion, transfusion, implantation or transplantation. Examples of routes of administration include parenteral, e.g., intravenous or intra-arterial, intradermal, subcutaneous, oral (e.g., inhalation), transdermal (topical), transmucosal, nasal, pulmonary, ocular, gastrointestinal, and rectal administration. Alternate routes of administration include intraperitoneal, intra-articular, intracardiac, intracisternal, intradermal, intralesional, intraocular, intrapleural, intrathecal, intrauterine, intraventricular, intracranial, intrathecal, and the like.

A pharmaceutical composition used in the therapeutic methods of the disclosure is formulated to be compatible with its intended route of administration.

### Disease and Treatment

In another aspect, provided herein is a method for treating or preventing a condition in a subject in need thereof, the method comprising administering to the subject the complex as provided herein, the composition as provided herein, or the lipid nanoparticle as provided herein, wherein the single guide RNA directs the Cas12b protein to effect a modification in a target polynucleotide sequence in a cell of the subject, thereby treating or preventing the condition.

In some embodiments, the target polynucleotide sequence is in a PCSK9 gene. In some embodiments, the modification reduces expression of functional PCSK9 protein encoded by the PCSK9 gene in the subject. In some embodiments, the condition is atherosclerotic vascular disease. In some embodiments, the target polynucleotide sequence is in an ANGPTL3 gene. In some embodiments, the modification reduces expression of functional ANGPTL3 protein encoded by the ANGPTL3 gene in the subject. In some embodiments, the condition is an atherosclerotic vascular disease, hypertriglyceridemia, or diabetes.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. Treating” may refer to administration of the pharmaceutical compositions described herein to a subject after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The disclosure is directed towards treating a patient's suffering from cancer. The term “prophylaxis” is used herein to refer to a measure or measures taken for the prevention or partial prevention of a disease or condition.

As used herein, the terms “prevent,” “preventing,” “prevention,” and the like, refer to reducing the probability of developing a disease or condition in a subject, who does not have, but is at risk of or susceptible to developing a disease or condition. The prevention may also be partial, such that the occurrence of pathology of a condition in a subject is less than that which would have occurred without the present disclosure. The term “ameliorate” as used herein can refer to decrease, suppress, attenuate, diminish, arrest, or stabilize the development or progression of a disease.

By “treating or preventing a condition” is meant ameliorating any of the conditions or signs or symptoms associated with the disorder before or after it has occurred. For example, as compared with an equivalent untreated control, alleviating a symptom of a disorder may involve reduction or degree of prevention at least 3%, 5%, 10%, 20%, 40%, 50%, 60%, 80%, 90%, 95%, 98%, 99%, 99.5%, 99.9%, or 100% as measured by any standard technique. In some embodiments, alleviating a symptom of a disorder may involve reduction or degree of prevention by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with an equivalent untreated control.

A patient who is being treated for a condition, a disease or a disorder is one who a medical practitioner has diagnosed as having such a condition. Diagnosis may be by any suitable means. Diagnosis and monitoring may involve, for example, detecting the presence of diseased, dying or dead cells in a biological sample (e.g., tissue biopsy, blood test, or urine test), detecting the presence of plaques, detecting the level of a surrogate marker in a biological sample, or detecting symptoms associated with a condition. A patient in whom the development of a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

A “subject” or “subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hypercholesterolemia. In some embodiments, the subject has atherosclerotic vascular disease. In some embodiments, the subject has hypertriglyceridemia. In some embodiments, the subject has diabetes. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

“Subjects” and “patients” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

“Administering” and its grammatical equivalents as used herein can refer to providing one or more pharmaceutical compositions described herein to a subject or a patient. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, infusion (inf.), oral routes (p.o.), topical (top.) administration, or rectal (p.r.) administration. One or more such routes can be employed. Parenteral administration can be, for example, by bolus injection or by gradual perfusion over time.

The therapeutic methods of the disclosure may be carried out on subjects displaying pathology resulting from a disease or a condition, subjects suspected of displaying pathology resulting from a disease or a condition, and subjects at risk of displaying pathology resulting from a disease or a condition. For example, subjects that have a genetic predisposition to a disease or a condition can be treated prophylactically. Subjects exhibiting symptoms associated with a condition, a disease or a disorder may be treated to decrease the symptoms or to slow down or prevent further progression of the symptoms. The physical changes associated with the increasing severity of a disease or a condition are shown herein to be progressive. Thus, in embodiments of the disclosure, subjects exhibiting mild signs of the pathology associated with a condition or a disease may be treated to improve the symptoms and/or prevent further progression of the symptoms.

In some embodiments, the subject exhibits an elevated blood LDL cholesterol level, a reduced blood HDL cholesterol level, and/or a reduced blood triglycerides level as compared to before the administration.

In some embodiments, after the administration, the subject exhibits a reduced blood low-density lipoprotein (LDL) cholesterol level by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared to before the administration. In some embodiments, after the administration, the subject exhibits a reduced blood low-density lipoprotein (LDL) cholesterol level by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to before the administration.

In some embodiments, after the administration, the subject exhibits a reduced blood triglycerides level by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared to before the administration. In some embodiments, after the administration, the subject exhibits a reduced blood triglycerides level by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to before the administration.

The term “low-density lipoprotein (LDL),” as used herein, refers to a microscopic blob made up of an outer rim of lipoprotein and a cholesterol center. In some embodiments, LDL has a highly hydrophobic core composed of a polyunsaturated fatty acid known as linoleate and hundreds to thousands esterified and unesterified cholesterol molecules. In some embodiments, the core of LDL also carries triglycerides and other fats and is surrounded by a shell of phospholipids and unesterified cholesterol.

The term “high-density lipoprotein (HDL),” as used herein, refers to the smallest lipoprotein particles. In embodiments, plasma enzyme lecithin-cholesterol acyltransferase (LCAT) converts the free cholesterol into cholesteryl, which is then sequestered into the core of the lipoprotein particle, eventually causing the newly synthesized HDL to assume a spherical shape. In embodiments, HDL particles increase in size as they circulate through the bloodstream and incorporate more cholesterol and phospholipid molecules from cells and other lipoproteins.

The term “cholesterol,” as used herein, refers to a lipid with a unique structure composed of four linked hydrocarbon rings forming the bulky steroid structure. The term “triglyceride,” as used herein, refers to a tri-ester composed of a glycerol bound to three fatty acid molecules. In some embodiments, the fatty acids are saturated or unsaturated fatty acids.

In some embodiments, the condition is an atherosclerotic vascular disease, hypertriglyceridemia, or diabetes.

The term “atherosclerosis” or “atherosclerotic vascular disease,” as used herein, refers to a disease in which the inside of an artery narrows due to the buildup of plaque. In some embodiments, it may result in coronary artery disease, stroke, peripheral artery disease, or kidney problems.

The term “hypertriglyceridemia,” as used herein, refers to high (hyper-) blood levels (-emia) of triglycerides, the most abundant fatty molecule in most organisms. In some embodiments, elevated levels of triglycerides are associated with atherosclerosis, even in the absence of hypercholesterolemia (high cholesterol levels), and predispose to cardiovascular disease. In some embodiments, very high triglyceride levels increase the risk of acute pancreatitis. In some embodiments, hypertriglyceridemia is associated with overeating, obesity, diabetes mellitus and insulin resistance, excess alcohol consumption, kidney failure, nephrotic syndrome, genetic predisposition (e.g., familial combined hyperlipidemia, i.e., Type II hyperlipidemia), lipoprotein lipase deficiency, lysosomal acid lipase deficiency, cholesteryl ester storage disease, certain medications (e.g., isotretinoin, hydrochlorothiazide diuretics, beta blockers, protease inhibitors), hypothyroidism (underactive thyroid), systemic lupus erythematosus and associated autoimmune responses, glycogen storage disease type 1, propofol, or HIV medications.

The term “diabetes,” as used herein, refers to a group of metabolic disorders characterized by a high blood sugar level over a prolonged period of time. In some embodiments, diabetes is type 1 diabetes that results from the pancreas's failure to produce enough insulin due to loss of beta cells. In some embodiments, diabetes is type 2 diabetes characterized by insulin resistance, a condition in which cells fail to respond to insulin properly. In some embodiments, diabetes is gestational diabetes that occurs when pregnant women without a previous history of diabetes develop high blood sugar levels.

As used herein, the term “effective amount” can be an amount sufficient to effect beneficial or desired results, such as beneficial or desired clinical results. An effective amount is also an amount that produces a prophylactic effect, e.g., an amount that delays, reduces, or eliminates the appearance of a pathological or undesired condition. An effective amount can be administered in one or more administrations. In terms of treatment, an “effective amount” of a composition of the disclosure is an amount that is sufficient to palliate, ameliorate, stabilize, reverse or slow the progression of a disease or a condition, e.g., an atherosclerotic vascular disease, hypertriglyceridemia, or diabetes. An “effective amount” may be of any of the compositions of the disclosure used alone or in conjunction with one or more agents used to treat a condition. An “effective amount” of a therapeutic agent within the meaning of the present disclosure will be determined by a patient's attending physician or veterinarian. Such amounts are readily ascertained by one of ordinary skill in the art and will a therapeutic effect when administered in accordance with the present disclosure. Factors which influence what a therapeutically effective amount will be include, the specific activity of the therapeutic agent being used, the type of disorder, time elapsed since the initiation of the disorder, and the age, physical condition, existence of other disease states, and nutritional status of the individual being treated. Additionally, other medication the patient may be receiving will affect the determination of the therapeutically effective amount of the therapeutic agent to administer.

The phrase “therapeutically effective amounts” used herein refers to the amount of agent needed to treat, ameliorate, or prevent a targeted disease or condition. An effective initial method to determine a “therapeutically effective amount” may be by carrying out cell culture assays (for example, using neuronal cells) or using animal models (for example, mice, rats, rabbits, dogs or pigs). A dose may be formulated in animal models to achieve a concentration range that includes the IC50 (i.e., the concentration of the composition which achieves a half-maximal inhibition of symptoms) as determined in cell culture. Such information can be used to more accurately determine useful doses in humans. In addition to determining the appropriate concentration range for a composition provided herein to be therapeutically effective, animal models may also yield other relevant information such as preferable routes of administration that will give maximum effectiveness. Adjusting the dose to achieve maximal efficacy in humans based on the methods described above and other methods is well within the capabilities of the ordinarily skilled artisan. A “patient” as used in herein refers to the subject who is receiving treatment by administration of the composition of interest.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In embodiments, the treatment is only on an as-needed basis, e.g., upon appearance of signs or symptoms of a condition or a disease, e.g., an atherosclerotic vascular disease, hypertriglyceridemia, or diabetes.

The compositions described herein, may be administered to a subject in need thereof, in a therapeutically effective amount, to treat conditions related to high circulating cholesterol levels. Conditions related to high circulating cholesterol level that may be treated using the compositions and methods described herein include, without limitation: hypercholesterolemia, elevated total cholesterol levels, elevated low-density lipoprotein (LDL) levels, elevated blood LDL-cholesterol levels, reduced blood high-density lipoprotein cholesterol level, liver steatosis, coronary heart disease, vascular disease, ischemia, stroke, peripheral vascular disease, thrombosis, type 2 diabetes, hypertriglyceridemia, high elevated blood pressure, atherosclerosis, obesity, Alzheimer's disease, neurodegeneration, and combinations thereof. The compositions and methods disclosed herein are effective in reducing the circulating cholesterol level in the subject, thus treating the conditions. The compositions and methods disclosed herein are effective in reducing blood LDL cholesterol level, and/or a reducing blood triglycerides level as compared to before the administration.

Toxicity and therapeutic efficacy of the compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

### Kits

One aspect of the disclosure relates to kits including the compositions comprising a single guide RNA as provided herein, the complex as provided herein, the composition as provided herein, or the lipid nanoparticle as provided herein for treating or preventing a condition. The kits can further include one or more additional therapeutic regimens or agents for treating or preventing a condition.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include the composition of the disclosure, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLE

These examples that follow are provided for illustrative purposes only and not to limit the scope of the claims provided herein.

### In Vitro Analysis of Cas12b

Plasmids encoding Cas12b orthologs derived from Bacillus hisashii (BhCas12b) and Bacillus sp. V3-13 (BvCas12b) (Addgene #236181, 122446) were transfected in HEK293 at a 1:1 ratio with guides cloned to target ANGPTL3 (Addgene #122447, 122448). The protospacers are detailed in Table 2. Guide RNAs were screened for on-target activity using one or more techniques: 1) sanger sequencing, to identify evidence of insertions or deletions; 2) CEL I enzymatic mutation detection assay (surveyor nuclease assay), according to manufacturer's instructions (IDT).

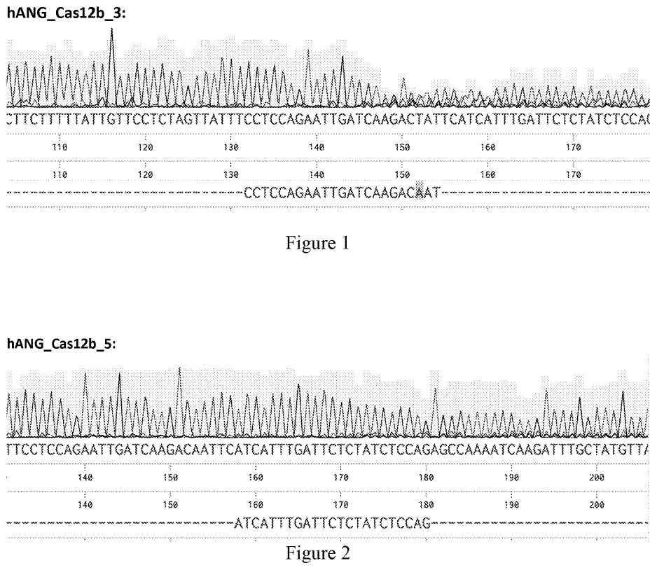

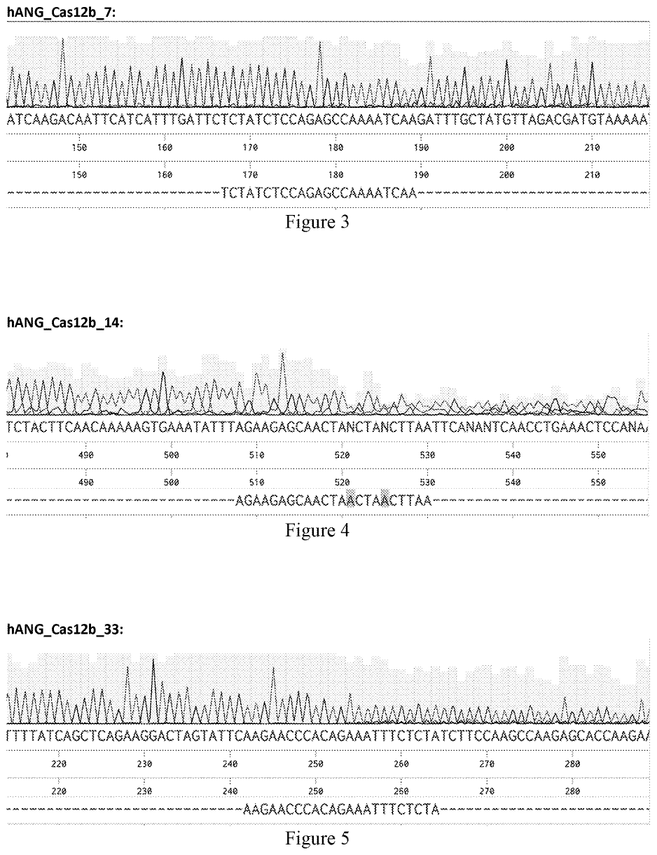

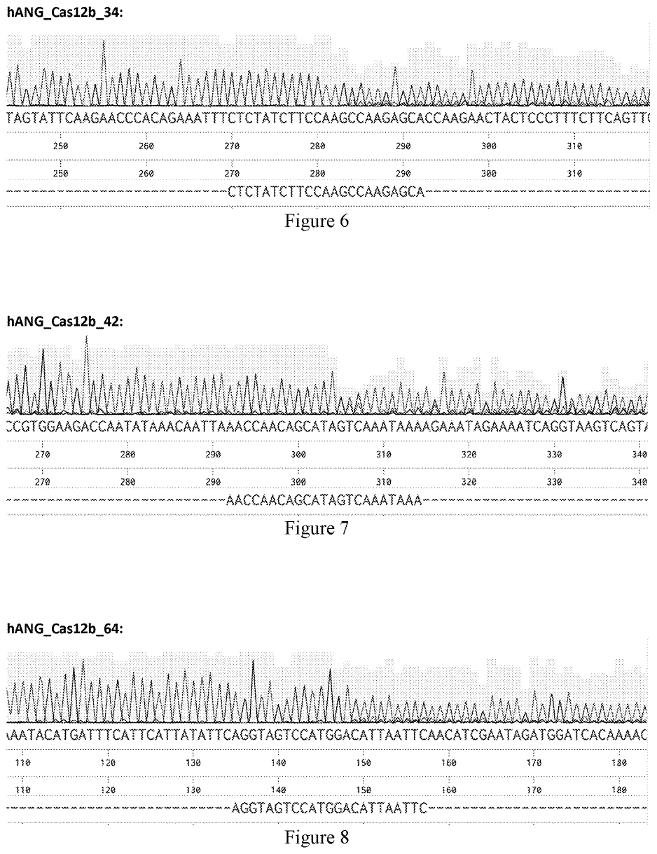

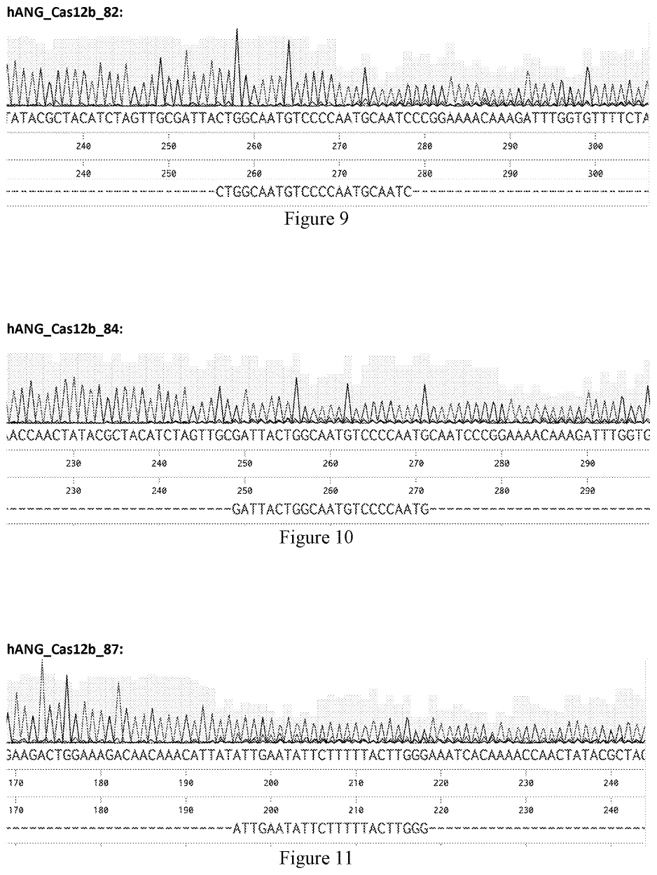

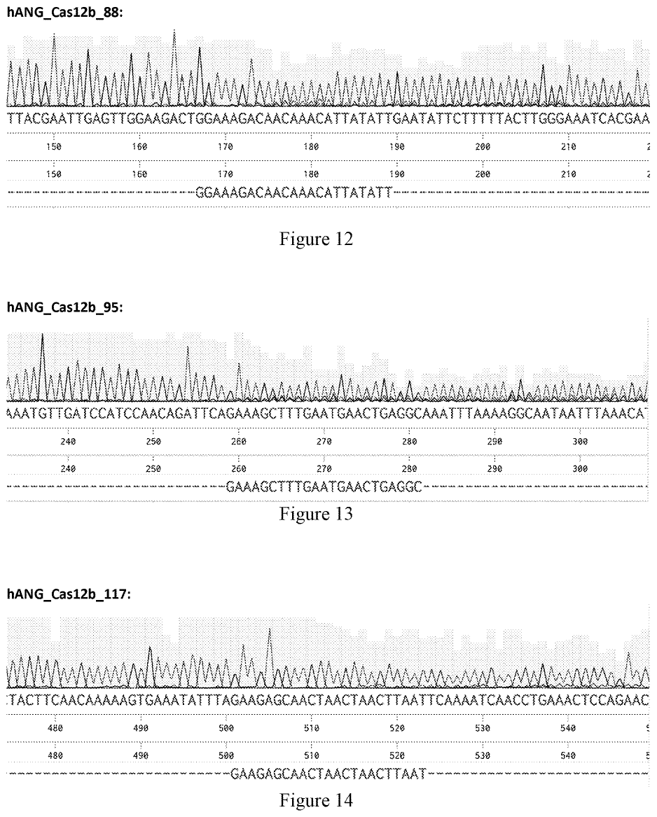

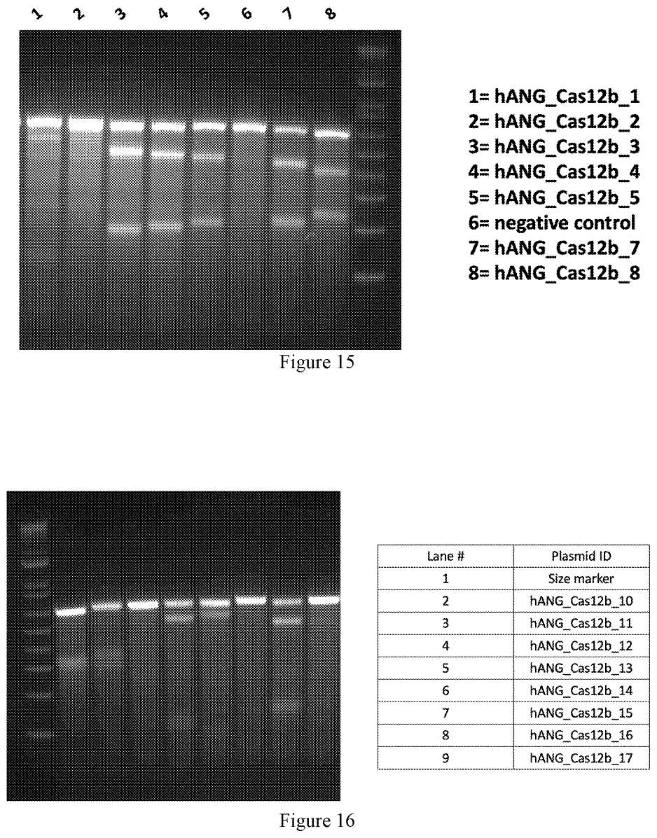

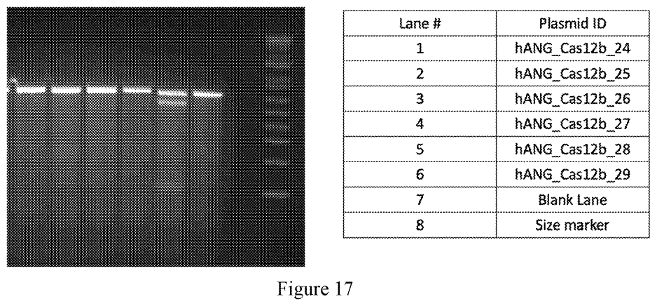

Several sites were identified to have significant on-target editing efficiency. By sanger sequencing, notable examples include hANG_Cas12b_3, hANG_Cas12b_5, hANG_Cas12b_7, hANG_Cas12b_14, hANG_Cas12b_33, hANG_Cas12b_34, hANG_Cas12b_42, hANG_Cas12b_64, hANG_Cas12b_82, hANG_Cas12b_84, hANG_Cas12b_87, hANG_Cas12b_88, hANG_Cas12b_95, and hANG_Cas12b_117 (FIGS. 1-14). By surveyor nuclease assay, notable examples include hANG_Cas12b_1, hANG_Cas12b_3, hANG_Cas12b_4, hANG_Cas12b_5, hANG_Cas12b_7, hANG_Cas12b_8, hANG_Cas12b_10, hANG_Cas12b_11, hANG_Cas12b_13, hANG_Cas12b_14, hANG_Cas12b_16, and hANG_Cas12b_28 (FIGS. 15-17).

TABLE 2
Plasmid guides used for in vitro analysis

| | | SEQ | | |
|---|---|---|---|---|
| | | ID | | Editor |
| Guide | Target | NO: | Protospacer (5′-3′) | Screened |
| hANG_Cas12b_1 | hANGPTL3 | 112 | AAAATCAAGATAAAAATGTTCAC | BvCas12b |
| hANG_Cas12b_2 | hcANGPTL3 | 113 | AGCTCCTTCTTTTTATTGTTCCT | BvCas12b |
| hANG_Cas12b_3 | hANGPTL3 | 114 | CCTCCAGAATTGATCAAGACAAT | BvCas12b |
| hANG_Cas12b_4 | hANGPTL3 | 115 | ATCAAGACAATTCATCATTTGAT | BvCas12b |
| hANG_Cas12b_5 | hANGPTL3 | 116 | ATCATTTGATTCTCTATCTCCAG | BvCas12b |
| hANG_Cas12b_6 | hANGPTL3 | 117 | GATTCTCTATCTCCAGAGCCAAA | BvCas12b |
| hANG_Cas12b_7 | hANGPTL3 | 118 | TCTATCTCCAGAGCCAAAATCAA | BvCas12b |
| hANG_Cas12b_8 | hcANGPTL3 | 119 | GCTATGTTAGACGATGTAAAAAT | BvCas12b |
| hANG_Cas12b_10 | hcANGPTL3 | 120 | ATGACATATTTCAAAAACTCAAC | BhCas12b |
| hANG_Cas12b_11 | hcANGPTL3 | 92 | CAAAAACTCAACATATTTGATCA | BhCas12b |
| hANG_Cas12b_12 | hcANGPTL3 | 122 | GATCAGTCTTTTTATGATCTATC | BhCas12b |
| hANG_Cas12b_13 | hcANGPTL3 | 93 | TACTTCAACAAAAAGTGAAATAT | BhCas12b |
| hANG_Cas12b_14 | hcANGPTL3 | 94 | AGAAGAGCAACTAACTAACTTAA | BhCas12b, |
| | | | | BvCas12b |
| hANG_Cas12b_15 | hANGPTL3 | 125 | AAAATCAACCTGAAACTCCAGAA | BhCas12b |
| hANG_Cas12b_16 | hcANGPTL3 | 95 | TGGAGGAAATAACTAGAGGAACA | BhCas12b |
| hANG_Cas12b_17 | hANGPTL3 | 127 | TCTTGATCAATTCTGGAGGAAAT | BhCas12b |
| hANG_Cas12b_22 | hANGPTL3 | 128 | ATTTGGCCCTTCGTCTTATGGAC | BhCas12b |
| hANG_Cas12b_23 | hANGPTL3 | 129 | CACTGGTTTGCAGCGATAGATCA | BhCas12b |
| hANG_Cas12b_24 | hcANGPTL3 | 130 | TTGACTTGTAGTTTATATGTAGT | BhCas12b |
| hANG_Cas12b_25 | hcANGPTL3 | 131 | TTTACCTCTTCATTTTTGACTTG | BhCas12b |
| hANG_Cas12b_26 | hcANGPTL3 | 132 | TTTCTTCTAGGAGGCTTTCAAGT | BhCas12b |
| hANG_Cas12b_27 | hcANGPTL3 | 133 | CACTTTTTGTTGAAGTAGAATTT | BhCas12b |
| hANG_Cas12b_28 | hcANGPTL3 | 134 | AGTTAGTTAGTTGCTCTTCTAAA | BhCas12b |
| hANG_Cas12b_29 | hcANGPTL3 | 135 | TGAATTAAGTTAGTTAGTTGCTC | BhCas12b |
| hANG_Cas12b_31 | hANGPTL3 | 136 | TTTTATCAGCTCAGAAGGACTAG | BhCas12b |
| hANG_Cas12b_33 | hcANGPTL3 | 96 | AAGAACCCACAGAAATTTCTCTA | BhCas12b |
| hANG_Cas12b_34 | hcANGPTL3 | 138 | CTCTATCTTCCAAGCCAAGAGCA | BhCas12b, |
| | | | | BvCas12b |
| hANG_Cas12b_35 | hANGPTL3 | 139 | CTGTGGGTTCTTGAATACTAGTC | BvCas12b |
| hANG_Cas12b_36 | hANGPTL3 | 140 | AACTGAAGAAAGGGAGTAGTTCT | BhCas12b, |
| | | | | BvCas12b |
| hANG_Cas12b_37 | hANGPTL3 | 141 | CATTCAACTGAAGAAAGGGAGTA | BhCas12b |
| hANG_Cas12b_38 | hANGPTL3 | 142 | CTTATTTCATTCAACTGAAGAAA | BhCas12b |
| hANG_Cas12b_41 | hANGPTL3 | 143 | TTATCTTGATTTTCAATTTCAAG | BhCas12b |
| hANG_Cas12b_42 | hcANGPTL3 | 90 | AACCAACAGCATAGTCAAATAAA | BhCas12b |
| hANG_Cas12b_43 | hcANGPTL3 | 145 | TCTATTTCTTTTATTTGACTATG | BhCas12b |
| hANG_Cas12b_45 | hANGPTL3 | 146 | GACTATGCTGTTGGTTTAATTGT | BhCas12b, |
| | | | | BvCas12b |
| hANG_Cas12b_46 | hANGPTL3 | 147 | TTTATATTGGTCTTCCACGGTCT | BhCas12b, |
| | | | | BvCas12b |
| hANG_Cas12b_47 | hANGPTL3 | 148 | GTCTTCCACGGTCTGGAGAAGGT | BhCas12b, |
| | | | | BvCas12b |
| hANG_Cas12b_48 | hANGPTL3 | 149 | TCTTGTTTTTCTACAAAAGTCTG | BhCas12b |
| hANG_Cas12b_49 | hcANGPTL3 | 150 | TATTGATTCTAGGCATTCCTGCT | BhCas12b |
| hANG_Cas12b_54 | hANGPTL3 | 151 | TAGGCATTCCTGCTGAATGTACC | BhCas12b |
| hANG_Cas12b_55 | hANGPTL3 | 152 | CTGCTGAATGTACCACCATTTAT | BhCas12b |
| hANG_Cas12b_56 | hANGPTL3 | 153 | ATAACAGAGGTGAACATACAAGT | BhCas12b |
| hANG_Cas12b_57 | hcANGPTL3 | 154 | AGCAGGAATGCCTAGAATCAATA | BhCas12b, |
| | | | | BvCas12b |
| hANG_Cas12b_58 | hcANGPTL3 | 155 | CATTCATTATATTCAGGTAGTCC | BhCas12b |
| hANG_Cas12b_59 | hcANGPTL3 | 156 | ATTATATTCAGGTAGTCCATGGA | BhCas12b |
| hANG_Cas12b_63 | hcANGPTL3 | 157 | TATTCAGGTAGTCCATGGACATT | BvCas12b |
| hANG_Cas12b_64 | hcANGPTL3 | 91 | AGGTAGTCCATGGACATTAATTC | BhCas12b, |
| | | | | BvCas12b |
| hANG_Cas12b_65 | hcANGPTL3 | 159 | ATTCAACATCGAATAGATGGATC | BhCas12b, |
| | | | | BvCas12b |
| hANG_Cas12b_69 | hcANGPTL3 | 160 | AACATCGAATAGATGGATCACAA | BhCas12b, |
| | | | | BvCas12b |
| hANG_Cas12b_70 | hANGPTL3 | 161 | AATGTAGTCCCCTTACCATCAAG | BhCas12b |
| hANG_Cas12b_71 | hcANGPTL3 | 162 | GTAGTTCTCCCACGTTTCATTGA | BvCas12b |
| hANG_Cas12b_72 | hcANGPTL3 | 163 | AAGTTTTGTGATCCATCTATTCG | BhCas12b, |
| | | | | BvCas12b |
| hANG_Cas12b_73 | hcANGPTL3 | 164 | GATGTTGAATTAATGTCCATGGA | BvCas12b |
| hANG_Cas12b_74 | hcANGPTL3 | 165 | ATGTCCATGGACTACCTGAATAT | BvCas12b |
| hANG_Cas12b_78 | hcANGPTL3 | 166 | AGTTGGAAGACTGGAAAGACAAC | BhCas12b |
| hANG_Cas12b_79 | hcANGPTL3 | 167 | TATTGAATATTCTTTTTACTTGG | BhCas12b |
| hANG_Cas12b_80 | hcANGPTL3 | 168 | AATATTCTTTTTACTTGGGAAAT | BhCas12b |
| hANG_Cas12b_81 | hcANGPTL3 | 169 | TTTTTACTTGGGAAATCACGAAA | BhCas12b |
| hANG_Cas12b_82 | hcANGPTL3 | 97 | CTGGCAATGTCCCCAATGCAATC | BhCas12b, |
| | | | | BvCas12b |
| hANG_Cas12b_84 | hcANGPTL3 | 98 | CATTGGGGACATTGCCAGTAATC | BhCas12b, |
| | | | | BvCas12b |
| hANG_Cas12b_85 | hANGPTL3 | 172 | GGGACATTGCCAGTAATCGCAAC | BhCas12b |
| hANG_Cas12b_86 | hANGPTL3 | 173 | CCAGTAATCGCAACTAGATGTAG | BvCas12b |
| hANG_Cas12b_87 | hcANGPTL3 | 99 | CCCAAGTAAAAAGAATATTCAAT | BhCas12b, |
| | | | | BvCas12b |
| hANG_Cas12b_88 | hcANGPTL3 | 100 | AATATAATGTTTGTTGTCTTTCC | BhCas12b, |
| | | | | BvCas12b |
| hANG_Cas12b_89 | hANGPTL3 | 176 | GTAAAACATAATTAGATTGCTTC | BhCas12b, |
| | | | | BvCas12b |
| hANG_Cas12b_89 | hANGPTL3 | 176 | GTAAAACATAATTAGATTGCTTC | BvCas12b |
| hANG_Cas12b_90 | hcANGPTL3 | 177 | GATTGCTTCACTATGGAGTATAT | BhCas12b, |
| | | | | BvCas12b |
| hANG_Cas12b_91 | hcANGPTL3 | 178 | CTTCACTATGGAGTATATCTTCT | BhCas12b |
| hANG_Cas12b_93 | hANGPTL3 | 179 | TCTTGGAAGTCTCAAAATGGAAG | BhCas12b |
| hANG_Cas12b_95 | hcANGPTL3 | 180 | GCCTCAGTTCATTCAAAGCTTTC | BhCas12b |
| hANG_Cas12b_99 | hANGPTL3 | 181 | TGAGACTTCCAAGATAATCCTCT | BhCas12b |
| hANG_Cas12b_101 | hcANGPTL3 | 182 | ACCATTTAGGTTGTTTTCTCCAC | BhCas12b |
| hANG_Cas12b_104 | hcANGPTL3 | 183 | ATTGTTCCTCTAGTTATTTCCTC | BvCas12b |
| hANG_Cas12b_105 | hcANGPTL3 | 184 | TTGTTCCTCTAGTTATTTCCTCC | BvCas12b |
| hANG_Cas12b_111 | hANGPTL3 | 185 | TCCATAAGACGAAGGGCCAAATT | BvCas12b |
| hANG_Cas12b_114 | hANGPTL3 | 186 | TATGATCTATCGCTGCAAACCAG | BhCas12b |
| hANG_Cas12b_115 | hANGPTL3 | 187 | ATGATCTATCGCTGCAAACCAGT | BvCas12b |
| hANG_Cas12b_117 | hcANGPTL3 | 101 | GAAGAGCAACTAACTAACTTAAT | BhCas12b |
| hANG_Cas12b_130 | hcANGPTL3 | 189 | GAGTTGAGTTCAAGTGACATATT | BhCas12b |

Synthesis of Guide RNAs (sgRNA or gRNA)

With high on-target editing efficiency in HEK293 cells, the hANG_Cas12b_42 protospacer, 5′-AACCAACAGCATAGTCAAATAAA-3′ (SEQ ID NO: 90), was used for further sequence optimization. A structure-based approach was used to: 1) identify bases where 2′-O-methyl could be added to offer stability to the gRNA, while not causing steric hindrance with either the protein or gRNA; 2) introduce changes to the gRNA sequence to improve stability and/or improve nuclease activity (FIG. 18). The designed guide sequences are described in Table 1.

Primary hepatocytes were transfected with Cas12b mRNA and gRNA as described in detailed methods. Table 3 shows the results of Cas12b nuclease mediated in vitro gene editing in human primary hepatocytes. Many gRNA sequences showed editing efficiency that is equivalent or similar to a gRNA considered having minimal modifications, GB0001. These include GB0002, GB0004, GB0006, GB0007, GB0008, and GB0009. It is contemplated that these chemically-modified gRNAs offer improved stability and/or half-life as compared to an unmodified gRNA having the same nucleotide sequence, with little to no loss of editing efficiency.

TABLE 3
Cas12b nuclease mediated in vitro gene
editing in human primary hepatocytes

| | | Editing % | | |
|---|---|---|---|---|
| gRNA | Cell Type | 2500 ng/mL | 500 ng/mL | 100 ng/mL |
| GB0001 | Human PH | ND | 47.74 | 15.30 |
| GB0002 | Human PH | 62.23 | 51.14 | 21.06 |
| GB0003 | Human PH | 55.66 | 35.58 | 11.15 |
| GB0004 | Human PH | 60.21 | 34.68 | 12.16 |
| GB0005 | Human PH | ND | 51.15 | 8.54 |
| GB0006 | Human PH | 55.54 | 20.38 | 5.34 |
| GB0007 | Human PH | 59.60 | 46.97 | 15.64 |
| GB0008 | Human PH | 59.05 | 38.78 | 18.87 |
| GB0009 | Human PH | 58.16 | 24.30 | 8.05 |
| GB0010 | Human PH | 37.30 | 19.84 | 6.79 |
| GB0011 | Human PH | 39.84 | 12.02 | 3.35 |
| GB0012 | Human PH | 2.00 | 10.97 | 5.25 |
| GB0013 | Human PH | 43.59 | 11.68 | 2.87 |
| GB0014 | Human PH | 38.54 | 19.01 | 5.98 |
| GB0015 | Human PH | 52.80 | 25.72 | 12.98 |
| GB0016 | Human PH | 40.09 | 24.97 | 3.58 |
| GB0017 | Human PH | 34.16 | 14.40 | 6.94 |
| GB0018 | Human PH | 37.00 | 12.10 | 2.39 |
| GB0019 | Human PH | 40.03 | 13.29 | 6.36 |
| GB0020 | Human PH | 38.45 | 0.09 | 0.11 |
| GB0021 | Human PH | 0.05 | 0.09 | 0.06 |

### Detailed Methods

gRNA synthesis: The guide RNA GB0001 to GB0021 set forth in Table 1 were synthesized under solid phase oligonucleotide synthesis and deprotection conditions using controlled pore glass support and commercially available phosphoramidite monomers and oligonucleotide synthesis reagents (see, e.g., Methods in Molecular Biology, 1993, 20, 81-114; ACS Chem. Biol. 2015, 10, 1181-1187, of which entire contents are incorporated herein by reference). The deprotected guide RANs were purified by HPLC and the integrity of each guide RNA was confirmed by mass spectrometric analysis. The observed mass of each guide RNA was conformed to calculated mass.

mRNA synthesis: mRNA for Cas12b was produced by methods well known in the art. One of such methods used herein was in vitro transcription (IVT) using T7 polymerase or additional RNA polymerase variants. Typically, IVT of mRNA uses a linearized DNA template that comprises a T7 polymerase promoter, mRNA coding sequence (CDS), 3′ and 5′ untranslated regions (UTRs), poly A tail, and additional replication and transcription regulatory elements. Prior to IVT, the DNA template was in the form of a plasmid, PCR product, or additional double-stranded DNA construct. A typical IVT reaction includes T7 polymerase, DNA template, RNase inhibitor, cap analog, inorganic pyrophosphatase, and naturally occurring ribonucleotides (rNTPs) such as GTP, ATP, CTP, UTP, or substitutions of natural rNTPs with modified rNTPs such as pseudouridine, N1-methylpseudouridine, 5-methyl cytidine, 5-methoxyuridine, N6-methyl adenosine, and N4-acetylcytidine. The cap analog was a dinucleotide or trinucleotide cap structure with the first initiating nucleotide containing standard 2′-hydroxyl group, 2′-O-methyl group, or additional 2′ chemical modification. Cap analog also was added after the IVT reaction using a vaccinia capping enzyme. After IVT, in some cases DNase was added to the transcription mixture to remove DNA template; alternatively, residual DNA was removed by ion exchange column chromatography. Purification and concentration of mRNA were performed with ion exchange chromatography, affinity chromatography, precipitation, ion-pairing reverse-phase chromatography, enzymatic reactions, size exclusion chromatography, and/or tangential flow filtration. Similar IVT and purification process were used to produce mRNA encoding Cas12b. The DNA template, reaction conditions, and purification parameters were optimized for the specific gene of interest, i.e., ANGPTL3. In some examples, capped and polyadenylated mRNA were obtained from commercially sources (TriLink, for e.g.).

Primary Hepatocyte Cell Culture Conditions: Primary human liver hepatocytes (PHH) were cultured per the manufacturer's protocol. In accordance therewith, the cells were thawed and resuspended in hepatocyte thawing medium followed by centrifugation at 100 g for 10 min at 4° C. The supernatant was discarded, and the pelleted cells resuspended in hepatocyte plating medium. Each vial contained approximately 5 million cells that were used for plating one 24-well plate. Plated cells were allowed to settle and adhere for 4-6 h in a tissue culture incubator at 37° C. under 5% CO₂ atmosphere. After incubation, cells were checked for monolayer formation. The incubating media was then replaced with fresh hepatocyte maintenance media (complete INVITROGRO medium obtained from BioIVT, the cell line provider). The cells thus became ready for transfection.

Cloning Guide Plasmids: Plasmids were generated by annealing a DNA oligonucleotide containing the desired protospacer sequence. After expansion of the plasmid in bacteria, plasmids were purified and sequence confirmed.

Transfection of HEK293 cells: Cells were transfected using Mirus Transit-2020 transfection reagent per manufacturer's instructions. A plasmid encoding Cas12b and a plasmid containing the gRNA sequence were transfected at a 1:1 ratio. Cells were allowed to proliferate for 48-72 hours, and gDNA was isolated using the Qiagen DNEasy Blood & Tissue kit per manufacturer's instructions.

Transfection of Primary Cells: MessengerMAX from Thermo Fisher was used for transfection. Solution A: desired amount of guide RNA is mixed with 1:1 wt ratio of mRNA in OptiMEM. Solution B: MessengerMAX in OptiMEM. After mixing solutions A and B, the mixture was incubated at room temperature for 20 min. 60 μL of the incubated solution was added dropwise to each cell wells. The cells were then allowed to remain at 37° C. for 3 days. Cells were harvested and prepared for genomic DNA extraction using a Thermo Kingfisher extraction instrument. Extracted genomic DNA was processed by PCR to analyze for gene editing. Amplified PCR product was then subjected to next generation sequencing (NGS) on an Illumina MiSeq.

Next Generation Sequencing: Next generation sequencing, or deep sequencing, was performed on the region of interest to determine the extent of gene editing. Samples were prepare using the Nextera XT DNA library preparation kit (Illumina) according to the manufacturer's protocol. Briefly, two rounds of PCR were performed first to amplify the region of interest and second to add DNA sequences required for deep sequencing and sample identification to the initial product. The final amplicon was sequenced on the Illumina MiSeq instrument according to the manufacturer's protocol.

Bioinformatic Analysis: Paired-end reads were analyzed with the CRISPResso2 pipeline (https://www.nature.com/articles/s41587-019-0032-3). Briefly, low-quality reads were filtered out, adapter sequences were trimmed from the reads, and the paired-end reads were merged and aligned to the amplicon sequence. The editing percentage was calculated as the number of reads supporting an insertion or a deletion, over the total number of aligned reads. For Cas12b, the editing percentage was calculated as the number of reads supporting an insertion or a deletion, over the total number of aligned reads.

All patents and publications mentioned in this specification are herein incorporated by reference to the same extent as if each independent patent and publication was specifically and individually indicated to be incorporated by reference.

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of chemically modified guide polynucleotides (e.g., guide RNAs, single guide RNAs, crRNAs, tracrRNAs, etc.) including compositions that comprise such guide polynucleotides, designs and modifications thereto; and specific examples and embodiments describing the synthesis, manufacture, use, and efficacy of the foregoing individually and in combination including as pharmaceutical compositions for treating disease and for in vivo and in vitro delivery of active agents to mammalian cells under described conditions.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:
1. A chemically modified single guide RNA for a Cas12b protein that comprises (i) a spacer sequence and (ii) a tracr sequence that serves as a binding scaffold for the Cas12b protein, wherein the spacer sequence is complementary to a polynucleotide sequence of a gene of interest and wherein the single guide RNA comprises one or more chemical modifications.
2. The chemically modified single guide RNA of clause 1, wherein one or more of the chemical modifications is in the spacer sequence.
3. The chemically modified single guide RNA of clause 1, wherein one or more of the chemical modifications is in the tracr sequence.
4. The chemically modified single guide RNA of clause 3, wherein one or more of the chemical modifications is in a stem loop structure of the tracr sequence.
5. The chemically modified single guide RNA of clause 1, wherein the chemical modifications are in the spacer and tracr sequences.
6. The chemically modified single guide RNA of any one of clauses 1-5, wherein the single guide RNA comprises one or more unmodified nucleotides.
7. The chemically modified single guide RNA of clause 6, wherein one or more of the unmodified nucleotides comprises a 2′-hydroxyl that is in a region of the tracr sequence that serves as a binding scaffold for the Cas12b protein and is in close proximity to the Cas12b protein when the Cas12b protein is bound to the tracr sequence.
8. The chemically modified single guide RNA of clause 6, wherein one or more of the unmodified nucleotides comprises a 2′-hydroxyl that binds with the Cas12b protein when the Cas12b protein is bound to the tracr sequence.
9. The chemically modified single guide RNA of clause 6, wherein one or more of the unmodified nucleotides comprises a 2′-hydroxyl that is in close proximity with a unmodified nucleotide in the single guide RNA.
10. The chemically modified single guide RNA of clause 6, wherein the one or more unmodified nucleotides is at one or more nucleotide positions selected from positions 4, 6-15, 17-20, 22-24, 27, 28, 31-43, 46-48, 51-61, 64-66, 70, 71, 73, 74, 77, 78, 80, 81, and 85-116 as numbered in SEQ ID NO: 1 or a corresponding position or combination of corresponding positions thereof.
11. The chemically modified single guide RNA of clause 6, wherein the one or more unmodified nucleotides is at one or more nucleotide positions selected from positions 4, 6-15, 17-20, 22-24, 27, 28, 31, 32, 34, 36, 38, 40-43, 46, 48, 52-61, 64, 66, 70, 71, 73, 74, 77, 78, 80, 81, 85, 88, 90, 91, 93, 94 and 96-116 as numbered in SEQ ID NO: 1 or a corresponding position or combination of corresponding positions thereof.
12. The chemically modified single guide RNA of clause 6, wherein the one or more unmodified nucleotides is at one or more nucleotide positions selected from positions 8-10, 12-15, 22-24, 32-38, 40, 41, 43, 44, 53-56, 63, 66-69 and 88-116 as numbered in SEQ ID NO: 1 or a corresponding position or combination of corresponding positions thereof.
13. The chemically modified single guide RNA of clause 6, wherein the one or more unmodified nucleotides is at one or more nucleotide positions selected from positions 8-10, 12-15, 22-24, 32-44, 53-56, 63, 66-69 and 88-116 as numbered in SEQ ID NO: 1 or a corresponding position or combination of corresponding positions thereof.
14. The chemically modified single guide RNA of any one of clauses 1-9, wherein the one or more chemical modifications is at one or more nucleotide positions selected from positions 1-3, 5, 16, 21, 25, 29, 30, 44, 45, 49, 50, 62, 63, 67-69, 72, 75, 76, 79, 82-84 and 117-120 as numbered in SEQ ID NO: 1 or a corresponding position or combination of corresponding positions thereof.
15. The chemically modified single guide RNA of any one of clauses 1-9, wherein the one or more chemical modifications is at one or more nucleotide positions selected from positions 1, 2, 3, 5, 16, 21, 25, 29, 30, 33, 35, 37, 39, 44, 45, 47, 49-51, 62, 63, 65, 67-69, 72, 75, 76, 79, 82-84, 86, 87, 89, 92, 95 and 117-120 as numbered in SEQ ID NO: 1 or a corresponding position or combination of corresponding positions thereof.
16. The chemically modified single guide RNA of any one of clauses 1-9, wherein the one or more chemical modifications is at one or more nucleotide positions selected from positions 1-7, 11, 16-21, 25-31, 45-52, 57-62, 64, 65, 70-87 and 117-120 as numbered in SEQ ID NO: 1 or a corresponding position or combination of corresponding positions thereof.
17. The chemically modified single guide RNA of any one of clauses 1-9, wherein the one or more chemical modification is at one or more nucleotide positions selected from positions 1-7, 11, 16-21, 25-31, 39, 42, 45-52, 57-62, 64, 65, 70-87, and 117-120 as numbered in SEQ ID NO: 1 or a corresponding position or combination of corresponding positions thereof.
18. The chemically modified single guide RNA of any one of clauses 1-17 wherein one or more of the chemical modifications comprises a 2′-OMe modification.
19. The chemically modified single guide RNA of any one of clauses 1-17 wherein one or more of the chemical modifications comprises a nebularine.
20. The chemically modified single guide RNA of any one of clauses 1-17, wherein one or more of the chemical modifications comprises a deoxynebularine.
21. The chemically modified single guide RNA of any one of clauses 1-17, wherein one or more of the chemical modifications comprises a 2′-O-methylnebularine.
22. The chemically modified single guide RNA of any one of clauses 1-17, wherein one or more of the chemical modifications comprises a phosphorothioate linkage.
23. The chemically modified single guide RNA of any one of clauses 1-17, wherein the single guide RNA comprises a phosphorothioate linkage at a 5′ end or at a 3′ end.
24. The chemically modified single guide RNA of clause 23, wherein the single guide RNA comprises two and no more than two contiguous phosphorothioate linkages at the 5′ end.
25. The chemically modified single guide RNA of clause 23, wherein the single guide RNA comprises two and no more than two contiguous phosphorothioate linkages at the 3′ end.
26. The chemically modified single guide RNA of clause 23, wherein the single guide RNA comprises two and no more than two contiguous phosphorothioate linkages at the 5′ end and 3′ end.
27. The chemically modified single guide RNA of clause 23, wherein the single guide RNA comprises three phosphorothioate linkages at the 3′ end.
28. The chemically modified single guide RNA of clause 23, wherein the single guide RNA comprises three phosphorothioate linkages at the 5′ end.
29. The chemically modified single guide RNA of clause 23, wherein the single guide RNA comprises three and no more than three contiguous phosphorothioate linkages at the 5′ end and 3′ end.
30. The chemically modified single guide RNA of clause 23, wherein the single guide RNA comprises three phosphorothioate linkages at the 5′ end and two phosphorothioate linkages at the 3′ end.
31. The chemically modified single guide RNA of clause 23, wherein the single guide RNA comprises two phosphorothioate linkages at the 5′ end and three phosphorothioate linkages at the 3′ end.
32. The chemically modified single guide RNA of clause 27, wherein the single guide RNA comprises the 5′-NsNsN-3′, 5′-NsNsNsS-3′, 5′-nsnsnsn-3′, or 5′-nsnsn-3′ at the 3′ end, wherein N independently is an unmodified nucleotide adenosine, cytidine, guanosine or uridine; and n is a modified nucleotide adenosine, cytidine, guanosine or uridine, wherein the modification of the nucleotide is a 2′-H, 2′-OMe or nucleobase modification; and each s is a phosphorothioate linkage.
33. The chemically modified single guide RNA of clause 27, wherein the single guide RNA comprises the sequence 5′-UsUsU-3′, 5′-UsUsUsU-3′, 5′-usususu-3′, or 5′-ususu-3′ at the 3′ end, wherein U is an unmodified uridine; and u is a modified uridine, wherein the modification of the uridine is a 2′-H, 2′-OMe or nucleobase modification; and each s is a phosphorothioate linkage.
34. The chemically modified single guide RNA of any one of clauses 1-33, wherein each one of the last four nucleotides at the 3′end of the single guide RNA comprises a 2′-OMe modification.
35. The chemically modified single guide RNA of any one of clauses 1-34, wherein the target polynucleotide sequence is in an ANGPTL3, PCSK9, APOC3, or Lp(a) gene.
36. The chemically modified single guide RNA of any one of clauses 1-34, wherein the target polynucleotide sequence is in an ANTPLT3 gene.
37. The chemically modified single guide RNA of any one of clauses 1-36, wherein the single guide RNA binds the Cas12b protein with increased binding affinity as compared to an unmodified single guide RNA.
38. The chemically modified single guide RNA of any one of clauses 1-37, wherein the tracr sequence comprises between 90 to 100 nucleotides.
39. The chemically modified single guide RNA of any one of clauses 1-37, wherein the tracr sequence comprises between 95 to 105 nucleotides.
40. The chemically modified single guide RNA of any one of clauses 1-37, wherein the tracr sequence comprises 97 nucleotides.
41. The chemically modified single guide RNA of any one of clauses 1-40, wherein the spacer sequence comprises between 20-30 nucleotides.
42. The chemically modified single guide RNA of any one of clauses 1-40, wherein the spacer sequence comprises between 20-25 nucleotides.
43. The chemically modified single guide RNA of any one of clauses 1-40, wherein the spacer sequence comprises 23 or 24 nucleotides.
44. A chemically modified single guide RNA for Cas12b protein that comprises (i) a spacer sequence having a length of 23 nucleotides and (ii) a tracr sequence having a length of 97 nucleotides, wherein the spacer sequence is complementary to a polynucleotide sequence of a gene of interest, wherein the tracr sequence serves as binding scaffold for the Cas12b protein, and wherein the single guide RNA comprises a chemical modification.
45. A chemically modified single guide RNA comprising a sequence selected from any one of SEQ ID NOs: 2-89, wherein the single guide RNA comprises one or more chemical modifications.
46. A chemically modified single guide RNA comprising a sequence of SEQ ID NO: 2, wherein the guide RNA comprises chemical modifications of the guide RNA of GB0002, GB0003, GB0007 or GB0008 of Table 1.
47. A chemically modified single guide RNA comprising a gRNA sequence of Table 1 wherein a, u, g, and c indicate 2′-OMe modified adenine, uridine, guanine, and cytidine, wherein s indicates a phosphorothioate linkage, wherein X indicates a nebularine, wherein x indicates a 2′-O-methylnebularine and wherein dX indicates a 2′-deoxynebularine.
48. A ribonucleoprotein complex comprising the chemically modified single guide RNA of any one of clauses 1-47 in complex with the Cas12b protein, wherein the complex comprises increased stability as compared to a complex with an unmodified single guide RNA and a Cas12b protein, wherein the stability is measured by half-life of the complex ex vivo or in vitro.
49. A cell comprising the ribonucleoprotein complex of clause 48.
50. A composition for gene modification comprising the chemically modified single guide RNA of any one of clauses 1-47 and a Cas12b protein or a nucleic acid sequence encoding the Cas12b protein.
51. The composition of clause 50 further comprising a vector that comprises the nucleic acid sequence encoding the Cas12b protein.
52. The composition of clause 50 or 51 further comprising a pharmaceutically acceptable carrier.
53. A lipid nanoparticle comprising the composition of any one of clauses 50-52.
54. A method for modifying a target polynucleotide sequence in a cell comprising introducing into the cell the ribonucleoprotein complex of clause 48, the composition of any one of clauses 50-52, or the lipid nanoparticle of clause 53, wherein the single guide RNA directs the Cas12b protein to effect a modification in the target polynucleotide sequence in the cell.
55. The method of clause 54, wherein the target polynucleotide sequence is in an ANGPTL3, PCSK9, APOC3, or Lp(a) gene.
56. The method of clause 54, wherein the target polynucleotide sequence is in an ANGPTL3 gene.
57. The method of any one of clauses 54-56, wherein the modification results in less off-target effect in the cell as compared to an unmodified single guide RNA.
58. The method of any one of clauses 54-57, wherein the single guide RNA exhibits increased stability in the cell compared to an unmodified single guide RNA, wherein the stability is measured by half-life of the single guide RNA in the cell.
59. The method of any one of clauses 54-58, wherein the modification is a double stranded break, a non-sense mutation, a frameshift mutation, a splice site alteration, or an inversion.
60. The method of any one of clauses 54-58, wherein the modification reduces or abolishes expression of functional PCSK9 protein encoded by the PCSK9 gene in the cell.
61. The method of any one of clauses 54-58, wherein the modification reduces or abolishes expression of functional ANGPTL3 protein encoded by the ANGPTL3 gene in the cell.
62. A method for treating or preventing a condition in a subject in need thereof, the method comprising administering to the subject the ribonucleoprotein complex of clause 48, the composition of any one of clauses 50-52, or the lipid nanoparticle of clause 53, wherein the single guide RNA directs the Cas12b protein to effect a modification in a target polynucleotide sequence in a cell of the subject, thereby treating or preventing the condition.
63. The method of clause 62, wherein the target polynucleotide sequence is in an ANGPTL3, PCSK9, APOC3, or Lp(a) gene.
64. The method of clause 62, wherein the target polynucleotide sequence is in an ANGPTL3 gene and wherein the chemically modified single guide RNA comprises a gRNA from Table 1.
65. The method of clause 62, wherein the modification reduces expression of functional PCSK9 protein encoded by the PCSK9 gene in the subject.
66. The method of clause 62, wherein the modification reduces expression of functional ANGPTL3 protein encoded by the ANGPTL3 gene in the subject.
67. The method of clause 63 or 65 wherein the condition is atherosclerotic vascular disease.
68. The method of clause 64 or 66 wherein the condition is an atherosclerotic vascular disease, hypertriglyceridemia, or diabetes.
69. A Cas12b gene editing system that comprises:
   (a) a single guide RNA comprising a spacer that is 20-30 nucleotides in length and complimentary to a DNA sequence of an ANGPTL3 gene; and
   (b) a Cas12b nuclease protein or a nucleic acid expressing a Cas12b nuclease protein, wherein the single guide RNA and the Cas12b protein form a ribonucleoprotein complex that is capable of mediating in vitro ANGPTL3 gene editing in human primary hepatocytes at or above the editing percentages set forth in Table 3.

It will also be appreciated from reviewing the present disclosure, that it is contemplated that the one or more aspects or features presented in one of or a group of related clauses may also be included in other clauses or in combination with the one or more aspects or features in other clauses.

What is claimed is:

1. A method for editing an ANGPTL3 gene in a cell, the method comprising introducing to the cell
(a) a Cas12b protein, or a nucleic acid encoding the Cas12b protein, and
(b) a chemically modified single guide RNA for the Cas12b protein, comprising:
(i) a spacer sequence complementary to a polynucleotide sequence located on a strand of the ANGPTL3 gene, and wherein the spacer sequence is designed to hybridize to the polynucleotide sequence located on the ANGPTL3 gene, and
(ii) a scaffold sequence that serves as a binding scaffold for the Cas12b protein,
wherein the spacer sequence comprises 15 to 30 nucleotides in length,
wherein the scaffold sequence has a nucleotide base sequence, wherein the nucleotide base sequence has at least 90% identity to positions 1 to 97 of SEQ ID NO: 1,
wherein the scaffold sequence comprises a plurality of different chemical modifications,
wherein the plurality of different chemical modifications are at two or more nucleotide positions selected from positions 1-7, 11, 16-21, 25-31, 39, 42, 45-52, 57-62, 64, 65, and 70-87 as numbered in SEQ ID NO: 1 or a corresponding position or combination of corresponding positions thereof, and
wherein the plurality of different chemical modifications comprise a 2′-O-methyladenosine, a 2′-O-methylcytidine, a 2′-O-methylguanosine, a 2′-O-methyluridine, a nebularine, a 2′-O-methylnebularine, or a phosphorothioate linkage.

2. The method of claim 1, wherein the plurality of different chemical modifications are at one or more nucleotide positions selected from positions 1-7, 11, 16-21, 25-31, 45-52, 57-62, 64, 65, and 70-87 as numbered in SEQ ID NO: 1 or a corresponding position or combination of corresponding positions thereof.

3. The method of claim 1, wherein the plurality of different chemical modifications are at two or more nucleotide positions selected from positions 1-3, 5, 16, 21, 25-26, 29, 30, 33, 35, 37, 39, 44, 45, 47, 49-51, 62, 63, 65, 67-69, 72, 75, 76, 79, 82-84, 86, 87, 89, 92, and 95 as numbered in SEQ ID NO: 1 or a corresponding position or combination of corresponding positions thereof.

4. The method of claim 1, wherein the plurality of different chemical modifications are at two or more nucleotide positions selected from positions 1-3, 5, 16, 21, 25-26, 29, 30, 44, 45, 49, 50, 62, 63, 67-69, 72, 75, 76, 79, and 82-84 as numbered in SEQ ID NO: 1 or a corresponding position or combination of corresponding positions thereof.

5. The method of claim 1, wherein the plurality of different chemical modifications are at two or more nucleotide positions selected from positions 16, 21, 25, 29, 30, 44, 45, 49, 50, 62, 63, 67-69, 72, 75, 76, 79, and 82-84 as numbered in SEQ ID NO: 1 or a corresponding position or combination of corresponding positions thereof.

6. The method of claim 1, wherein the scaffold sequence comprises the plurality of different chemical modifications
(i) at positions 1-7, 11, 16-21, 25-31, 39, 42, 45-52, 57-62, 64, 65, and 70-87,
(ii) at positions 1-7, 11, 16-21, 25-31, 45-52, 57-62, 64, 65, and 70-87,
(iii) at positions 1-3, 5, 16, 21, 25-26, 29, 30, 44, 45, 49, 50, 62, 63, 67-69, 72, 75, 76, 79, and 82-84, or
(iv) at positions 1-3, 5, 16, 21, 25-26, 29, 30, 44, 45, 49, 50, 62, 63, 67-69, 72, 75, 76, 79, and 82-84,
as numbered in SEQ ID NO: 1 or a corresponding position or combination of corresponding positions thereof.

7. The method of claim 1, wherein the chemically modified single guide RNA comprises one or more unmodified nucleotides.

8. The method of claim 7, wherein the one or more unmodified nucleotides are at one or more nucleotide positions selected from positions 4, 6-15, 17-20, 22-24, 27, 28, 31-43, 46-48, 51-61, 64-66, 70, 71, 73, 74, 77, 78, 80, 81, and 85-116 as numbered in SEQ ID NO: 1 or a corresponding position or combination of corresponding positions thereof.

9. The method of claim 7, wherein the one or more unmodified nucleotides comprise a 2′-hydroxyl group,
wherein the 2′-hydroxyl group is in a region of the scaffold sequence that serves as a binding scaffold for the Cas12b protein, and/or
wherein the 2′-hydroxyl group is in close proximity to the Cas12b protein when the Cas12b protein is bound to the scaffold sequence.

10. The method of claim 1, wherein the plurality of different chemical modifications comprise the phosphorothioate linkage, and wherein the chemically modified single guide RNA comprises two and no more than two contiguous phosphorothioate linkages at the 5′ end and/or at the 3′ end of the chemically modified single guide RNA.

11. The method of claim 1, wherein the chemically modified single guide RNA comprises the phosphorothioate linkage, and wherein the chemically modified single guide RNA comprises three and no more than three contiguous phosphorothioate linkages at the 5′ end and/or the 3′ end of the chemically modified single guide RNA.

12. The method of claim 1, wherein the chemically modified single guide RNA comprises a sequence of 5′-NsNsN-3′, 5′-NsNsNsN-3′, 5′-nsnsnsn-3′, or 5′-nsnsn-3′ at the 3′ end of the chemically modified single guide RNA, wherein N independently is an unmodified nucleotide adenosine, cytidine, guanosine or uridine; and n independently is a modified nucleotide adenosine, cytidine, guanosine or uridine, wherein the modification of the nucleotide is a 2′-H, 2′-OMe or nucleobase modification; and each s is a phosphorothioate linkage.

13. The method of claim 1, wherein the chemically modified single guide RNA comprises a sequence of 5′-UsUsU-3′, 5′-UsUsUsU-3′, 5′-usususu-3′, or 5′-ususu-3′ at the 3′ end of the chemically modified single guide RNA, wherein U is an unmodified uridine; and u is a modified uridine, wherein the modification of the uridine is a 2′-H, 2′-OMe or nucleobase modification; and each s is a phosphorothioate linkage.

14. The method of claim 1, wherein each one of the last four nucleotides at the 3′ end of the chemically modified single guide RNA comprises a 2′-OMe modification.

15. The method of claim 1, wherein the ANGPTL3 gene comprises a sequence of SEQ ID NO: 108.

16. The method of claim 1, wherein the ANGPTL3 gene comprises a protospacer sequence of any one of SEQ ID NOs: 90-101.

17. The method of claim 1, wherein the chemically modified single guide RNA comprises a sequence with at least 90% identity to a sequence selected from any one of SEQ ID NOs: 2-89.

18. The method of claim 1, wherein the chemically modified single guide RNA comprises a nucleotide sequence of gsususCuGUCUUUUGGUcAGGAcAACcgUCuaGCUAUAAGUGCUGcaGGGugUGA GAAACUCCuaUUGcugGAcGAugUCuCUuacGAGGCAUUAGCACAACCAACAGC AUAGUCAAAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

19. The method of claim 1, wherein the chemically modified single guide RNA is about 90 to about 135 nucleotides in length.

20. The method of claim 1, wherein the chemically modified single guide RNA comprises a nucleotide sequence of gsususCuGUCUUUUGGUcAGGAcAACcgUCuaGCuAuAaGuGCUGcaGgGuguGAGA AACUCCuaUuGcugGAcGAugUCuCUuacGagGcAUuAGcACAACCAACAGCAUAG UCAAAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

21. The method of claim 1, wherein the chemically modified single guide RNA comprises a nucleotide sequence of gsususcuguCUUuUGGUcaggacAACcgucuagCUAUAAGuGCuGCagggugugAGAAacuc cuAuuGCUGgacgaugucucuuacgagGCAUUAGCACAACCAACAGCAUAGUCAAAus asasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

22. The method of claim 1, wherein the chemically modified single guide RNA comprises a nucleotide sequence of gsususcuguCUUuUGGUcaggacAACcgucuagCUAUAAGUGCUGCagggugugAGAAacu ccuAuuGCUGgacgaugucucuuacgagGCAUUAGCACAACCAACAGCAUAGUCAAAu sasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

23. The method of claim 1, wherein the chemically modified single guide RNA comprises a nucleotide sequence of gsususCuGUCUUUUGGUcAGGAcAACcgUCuaGCUAUAAGUGCUGcaGGGugUGA GAAACUCCuaUUGcugGAcGAugUCuCUuacGAGGCAUUAGCACAACCAACAGC AuAGUcAAAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

24. The method of claim 1, wherein the chemically modified single guide RNA comprises a nucleotide sequence of gsususCuGUCUUUUGGUcAGGAcAACcgUCuaGCuAuAaGuGCUGcaGgGuguGAGA AACUCCuaUuGcugGAcGAugUCuCUuacGagGcAUuAGcACAACCAACAGCAuAG UcAAAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

25. The method of claim 1, wherein the chemically modified single guide RNA comprises a nucleotide sequence of gsususCUGUCUUUUGGUCAGGACAACcgucuagCUAUAAGuGCuGCagggugugAGA AacuccuAuuGCUGgacgaugucucuuacgagGCAUUAGCACAACCAACAGCAUAGUCA AAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

26. The method of claim 1, wherein the chemically modified single guide RNA comprises a nucleotide sequence of gsususcuguCUUuUGGUcaggacAACcgucuagCUAUAAGuGCuGCAGGGUGUGAGAA ACUCCUAuuGCUGgacgaugucucuuacgagGCAUUAGCACAACCAACAGCAUAGUC AAAusasasa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

27. The method of claim 1, wherein the chemically modified single guide RNA comprises a nucleotide sequence of gsususcuguCUUuUGGUcaggacAACcgucuagCUAUAAGuGCuGCagggugugAGAAacuc cuAuuGCUGgacgaugucucuuacgagGCAUUAGCACaACCAAcaGCAuaGUCAAAusasa sa, wherein uppercase A, U, G, and C represent unmodified adenosine, uridine, guanosine and cytidine respectively, lowercase a, u, g, and c represent 2′-O-methyl modified adenosine, uridine, guanosine and cytidine respectively, and the lowercase s represents a phosphorothioate linkage.

28. The method of claim 1, wherein the Cas12b protein is a Cas12b nuclease.

29. The method of claim 1, wherein the cell is a hepatocyte.

30. The method of claim 1, wherein the cell is a mammalian hepatocyte.

31. The method of claim 1, wherein the cell is a human hepatocyte.

32. The method of claim 1, wherein editing the ANGPTL3 gene results in insertion or deletion of nucleotide in the ANGPTL3 gene.

33. A method for lowering blood low-density lipoprotein cholesterol level (LDL-C) in a subject in need thereof, the method comprising administering to the subject
a) a Cas12b protein, or a nucleic acid encoding the Cas12b protein, and
b) a chemically modified single guide RNA for a Cas12b protein, comprising:
(i) a spacer sequence complementary to a polynucleotide sequence located on a strand of a human ANGPTL3 gene, and wherein the spacer sequence is designed to hybridize to the polynucleotide sequence located on the human ANGPTL3 gene, and
(ii) a scaffold sequence that serves as a binding scaffold for the Cas12b protein,
wherein the spacer sequence comprises 15 to 30 nucleotides in length,
wherein the scaffold sequence has a nucleotide base sequence, wherein the nucleotide base sequence has at least 90% identity to positions 1 to 97 of SEQ ID NO: 1,
wherein the scaffold sequence comprises a plurality of different chemical modifications,
wherein the plurality of different chemical modifications are at two or more nucleotide positions selected from positions 1-7, 11, 16-21, 25-31, 39, 42, 45-52, 57-62, 64, 65, and 70-87 as numbered in SEQ ID NO: 1 or a corresponding position or combination of corresponding positions thereof, and
wherein the plurality of different chemical modifications comprise a 2′-O-methyladenosine, a 2′-O-methylcytidine, a 2′-O-methylguanosine, a 2′-O-methyluridine, a nebularine, a 2′-O-methylnebularine, or a phosphorothioate linkage.

34. The method of claim 33, wherein the plurality of different chemical modifications are at one or more nucleotide positions selected from positions 1-7, 11, 16-21, 25-31, 45-52, 57-62, 64, 65, and 70-87 as numbered in SEQ ID NO: 1 or a corresponding position or combination of corresponding positions thereof.

35. The method of claim 33, wherein the plurality of different chemical modifications are at two or more nucleotide positions selected from positions 1-3, 5, 16, 21, 25-26, 29, 30, 33, 35, 37, 39, 44, 45, 47, 49-51, 62, 63, 65, 67-69, 72, 75, 76, 79, 82-84, 86, 87, 89, 92, and 95 as numbered in SEQ ID NO: 1 or a corresponding position or combination of corresponding positions thereof.

36. The method of claim 33, wherein the plurality of different chemical modifications are at two or more nucleotide positions selected from positions 1-3, 5, 16, 21, 25-26, 29, 30, 44, 45, 49, 50, 62, 63, 67-69, 72, 75, 76, 79, and 82-84 as numbered in SEQ ID NO: 1 or a corresponding position or combination of corresponding positions thereof.

37. The method of claim 33, wherein the plurality of different chemical modifications are at two or more nucleotide positions selected from positions 16, 21, 25, 29, 30, 44, 45, 49, 50, 62, 63, 67-69, 72, 75, 76, 79, and 82-84 as numbered in SEQ ID NO: 1 or a corresponding position or combination of corresponding positions thereof.

38. The method of claim 33, wherein the scaffold sequence comprises the plurality of different chemical modifications
(i) at positions 1-7, 11, 16-21, 25-31, 39, 42, 45-52, 57-62, 64, 65, and 70-87,
(ii) at positions 1-7, 11, 16-21, 25-31, 45-52, 57-62, 64, 65, and 70-87,
(iii) at positions 1-3, 5, 16, 21, 25-26, 29, 30, 44, 45, 49, 50, 62, 63, 67-69, 72, 75, 76, 79, and 82-84, or
(iv) at positions 1-3, 5, 16, 21, 25-26, 29, 30, 44, 45, 49, 50, 62, 63, 67-69, 72, 75, 76, 79, and 82-84,
as numbered in SEQ ID NO: 1 or a corresponding position or combination of corresponding positions thereof.

39. The method of claim 33, wherein the chemically modified single guide RNA comprises one or more unmodified nucleotides.

40. The method of claim 39, wherein the one or more unmodified nucleotides are at one or more nucleotide positions selected from positions 4, 6-15, 17-20, 22-24, 27, 28, 31-43, 46-48, 51-61, 64-66, 70, 71, 73, 74, 77, 78, 80, 81, and 85-116 as numbered in SEQ ID NO: 1 or a corresponding position or combination of corresponding positions thereof.

41. The method of claim 33, wherein the ANGPTL3 gene comprises a sequence of SEQ ID NO: 108.

42. The method of claim 33, wherein the ANGPTL3 gene comprises a protospacer sequence of any one of SEQ ID NOs: 90-101.

43. The method of claim 33, wherein the chemically modified single guide RNA comprises a sequence with at least 90% identity to a sequence selected from any one of SEQ ID NOs: 2-89.

44. The method of claim 33, wherein the Cas12b protein is a Cas12b nuclease.
